(19) United States
(12) Patent Application Publication
(10) Pub. No.: US 2026/0114180 A1
(43) Pub. Date: Apr. 23, 2026

(54) COMPOSITION, ORGANIC ELECTROLUMINESCENT DEVICE, AND DISPLAY APPARATUS

(71) Applicant: Fuyang Sineva New Material Technology Co., LTD, Anhui (CN)

(72) Inventors: Zhiqiang LI, Anhui (CN); Zhanqi WANG, Beijing (CN); Jinbo LU, Anhui (CN); Changgang HUANG, Beijing (CN)

(21) Appl. No.: 19/115,137

(22) PCT Filed: Sep. 25, 2023
(86) PCT No.: PCT/CN2023/121156

§ 371 Date: Mar. 25, 2025

(30) Foreign Application Priority Data
Sep. 30, 2022 (CN) 202211211813.6

(51) Int. Cl. H10K 85/60; C07B 59/00; C09K 11/06; H10K 50/11; H10K 50/18

(52) U.S. Cl. CPC H10K 85/6572; C07B 59/004; C09K 11/06; H10K 85/622; H10K 85/623; H10K 85/626; H10K 85/633; H10K 85/636; H10K 85/654; H10K 85/6574; H10K 85/6576; C07B 2200/05; C09K 2211/1466; H10K 50/11; H10K 50/181

(57) ABSTRACT

The present application provides a composition, an organic electroluminescent device, and a display apparatus. The composition comprises two compounds shown in formula I; or the composition comprises a compound shown in formula I and two compounds shown in formula II. The compounds shown in formula I are obtained by fusing a group shown in formula I-A and any two adjacent carbon atoms on ring A of a group shown in formula I-B. The compounds shown in formula II are obtained by fusing a group shown in formula I-C and any two adjacent carbon atoms on ring E of a group shown in formula I-D. According to the present application, by designing specific components of the composition, and using the composition as a light-emitting layer material of the organic electroluminescent device, the prepared organic electroluminescent device has excellent comprehensive performance.

The present application claims priority to Chinese Patent Application No. CN 202211211813.6 filed on Sep. 30, 2022, and the disclosure of which is incorporated herein by reference in its entirety.

### TECHNICAL FIELD

The present application belongs to the technical field of organic electroluminescent materials, and specifically relates to a composition, an organic electroluminescent device and a display apparatus.

### BACKGROUND

Organic electroluminescence (EL) is a luminescent phenomenon in which organic materials directly convert electrical energy to light energy under the influence of the electric field. Organic electroluminescent device is a spontaneous light-emitting device utilizing with this principle, which has characteristics of self-illumination, bright and vivid colors, thin thickness, light weight, fast response speed, wide viewing angles, low driving voltage, resilience to harsh natural conditions, and the ability to be made into flexible panels. These attributes have gradually established them as the most advantageous technology in the new generation of flat-panel displays.

The structure of an organic electroluminescent device (OLED) includes an anode, a cathode, and an organic layer sandwiched between them. In order to enhance the efficiency and stability of organic electroluminescent elements, the organic material layer includes multiple layers with different materials, such as a hole injection layer (HIL), a hole transport layer (HTL), an electron blocking layer, an emissive layer, an electron transport layer (ETL), and an electron injection layer (EIL), etc. The hole injection layer plays the role of a layer that facilitates the injection of holes from the anode of the OLED into the hole transport layer. The hole injection layer is generally directly adjacent to the anode, and one or more hole transport layers are on the cathode side and adjacent to the hole injection layer directly. A hole transport layer is a layer that transports holes, generally a layer located between the cathode and the organic layer closest to the anode. The electron blocking layer blocks electrons from the cathode direction and has a shallower LUMO compared to the hole transport layer, that is, the absolute value of the LUMO of the electron blocking layer is less than the absolute value of the LUMO of the hole transport layer.

In order to obtain the organic electroluminescent device with excellent performance, the research and development of organic materials have attracted a lot of attention. Therefore, there is an urgent need to develop more kinds of materials with higher performance in this field to meet the higher requirements for OLEDs.

### SUMMARY

The following is a summary of the subject described in detail herein. This summary is not intended to limit the protection scope of the claims.

In view of the shortcomings of the prior art, an object of the present application is to provide a composition, an organic electroluminescent device, and a display apparatus. In the present application, a composition with excellent performance is obtained by designing the specific components of the composition, and an organic electroluminescent device with excellent performance can be fabricated by using this composition as the material of the emissive layer of the organic electroluminescent device.

To achieve the object, the present application adopts the following technical solutions.

In a first aspect, the present application provides a composition, and the composition includes two compounds represented by Formula I;
or the composition includes one compound represented by Formula I and two compounds represented by Formula II;
the compound represented by Formula I is obtained by fusing a group represented by Formula I-A with any two adjacent carbon atoms on ring A in the group represented by Formula I-B;

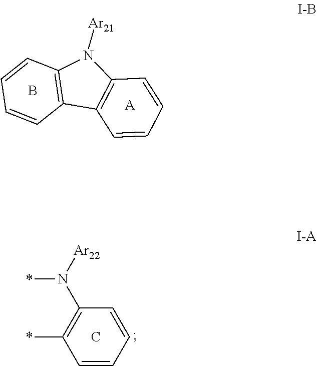

wherein “*” represents a fusion site;
Ar₂₁ and Ar₂₂ are each independently selected from C6 to C30 aryl or C6 to C20 heteroaryl;
the hydrogen atom in the compound represented by Formula I can be substituted with at least one of —F, —CN, C6 to C20 aryl, C1 to C6 alkyl, or C1 to C6 alkoxy;
the compound represented by Formula I meets at least one of the following conditions:
(1) the compound represented by Formula I contains no deuterium atom;
(2) hydrogen atoms on at least one of the ring A, ring B, and ring C in the compound represented by Formula I are all substituted with deuterium atoms;
(3) at least one hydrogen atom in Ar₂₁ of the compound represented by Formula I is substituted with a deuterium atom;
(4) at least one hydrogen atom in Ar₂₂ of the compound represented by Formula I is substituted with a deuterium atom;
(5) a hydrogen atom in the compound represented by Formula I is substituted with C6 to C20 aryl, and at least one hydrogen atom in the C6 to C20 aryl is substituted with a deuterium atom; and
(6) a hydrogen atom in the compound represented by Formula I is substituted with C1 to C6 alkyl and/or C1 to C6 alkoxy, and all hydrogen atoms in C1 to C6 alkyl and/or C1 to C6 alkoxy are substituted with deuterium atoms;
the compound represented by Formula II is obtained by fusing a group represented by Formula I-C with any two adjacent carbon atoms on ring E in the group represented by Formula I-D;

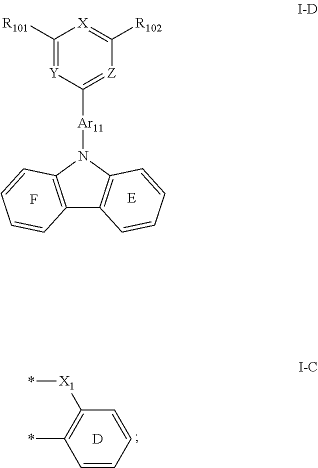

wherein “*” represents a fusion site;
Ar₁₁ is selected from any one of a single bond, phenylene, naphthylene, or biphenylene;
R₁₀₁ and R₁₀₂ are each independently selected from H, C6 to C30 aryl, or C6 to C20 heteroaryl;
X, Y, and Z are each independently selected from N or CR₃₀₄, wherein R₃₀₄ is selected from any one of H, phenyl, biphenyl, naphthyl, 9,9-dimethylfluorenyl, dibenzofuranyl, or dibenzothienyl, and at least one of X, Y and Z is N;
X₁ is selected from O, S,

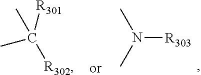

wherein R₃₀₁ and R₃₀₂ are each independently selected from C1 to C5 alkyl or phenyl, and R₃ is selected from phenyl or biphenyl, and the dashed line represents a linkage site;
the hydrogen atom in the compound represented by Formula II may be substituted with at least one of —F, —CN, C6 to C20 aryl, C1 to C6 alkyl, or C1 to C6 alkoxy;
the compound represented by Formula II meets at least one of the following conditions:
(a) the compound represented by Formula II contains no deuterium atom;
(b) hydrogen atoms on at least one of the ring F, ring D, and ring E in the compound represented by Formula II are all substituted with deuterium atoms;
(c) at least one hydrogen atom in R₁₀₁ of the compound represented by Formula II is substituted with a deuterium atom;
(d) at least one hydrogen atom in R₁₀₂ of the compound represented by Formula II is substituted with a deuterium atom;
(e) at least one hydrogen atom in Ar₁₁ of the compound represented by Formula II is substituted with a deuterium atom;
(f) hydrogen atoms in R₃₀₁ and R₃₀₂ in the compound represented by Formula II are all substituted with deuterium atoms;
(g) at least one hydrogen atom in R₃₀₃ group of the compound represented by Formula II is substituted with a deuterium atom;
(h) R₃₀₄ in the compound represented by Formula II is a deuterium atom;
(i) in the compound represented by Formula II, when R₃₀₄ is selected from any one of phenyl, biphenyl, naphthyl, 9,9-dimethylfluorenyl, dibenzofuranyl, or dibenzothienyl, at least one hydrogen atom in the phenyl, biphenyl, naphthyl, 9,9-dimethylfluorenyl, dibenzofuranyl, or dibenzothienyl is substituted with a deuterium atom;
(j) a hydrogen atom in the compound represented by Formula II is substituted with C6 to C20 aryl, and at least one hydrogen atom in the C6 to C20 aryl is substituted with a deuterium atom; and
(k) a hydrogen atom in the compound represented by Formula II is substituted with C1 to C6 alkyl and/or C1 to C6 alkoxy, and all hydrogen atoms in C1 to C6 alkyl and/or C1 to C6 alkoxy are substituted with deuterium atoms.

In the present application, a composition with specific component is obtained by designing the specific components of the composition and further using the combination of at least two specific compounds, and an organic electroluminescent device with excellent performance can be fabricated by using this composition as the material of the emissive layer of the organic electroluminescent device.

In the present application, the C6 to C30 is selected from C6, C10, C12, C18, C24, or C30, etc.

The C6 to C20 is selected from C6, C10, C12, C18, or C20, etc.

The C1 to C6 is selected from C1, C2, C3, C4, C5, or C6.

The C1 to C5 is selected from C1, C2, C3, C4, or C5.

It should be noted that the two compounds represented by Formula I in the present application means that the two compounds represented by Formula I both are consistent with the general formula of Formula I, but the specific structural formulas of the two compounds represented by Formula I are different; similarly, the two compounds represented by Formula II means that the two compounds represented by Formula II both are consistent with the general formula of Formula II, but the specific structural formulas of the two compounds represented by Formula II are different.

The following are optional technical solutions of the present application, but not as a limitation of the technical solutions provided in the present application. The objects and beneficial effects of the present application can be better achieved and realized by the following optional technical solutions.

As an optional technical solution of the present application, the composition includes two compounds represented by Formula I, and the composition further includes at least one compound represented by Formula II.

In one embodiment, the composition includes two compounds represented by Formula I and one compound represented by Formula II.

In one embodiment, the composition includes two compounds represented by Formula I and two compounds represented by Formula II.

In the present application, the composition includes two compounds represented by Formula I (denoted as Compound 1-1 and Compound 1-2), and a volume ratio of Compound 1-1 to Compound 1-2 is (2:8)-(8:2), which may be, for example, 2:8, 3:7, 4:6, 5:5, 6:4, 7:3, or 8:2, etc.; or
the composition includes two compounds represented by Formula I (denoted as Compound 1-1 and Compound 1-2) and one compound represented by Formula II (denoted as Compound 2-1), and a volume ratio of Compound 1-1 to Compound 1-2 to Compound 2-1 is 1:(1-2):(1-6), which may be, for example, 1:1:1, 1:1:2, 1:1:3, 1:1:4, 1:2:1, 1:2:2, 1:2:3, 1:2:4, 1:2:5, or 1:2:6, etc.; or
the composition includes one compound represented by Formula I (denoted as Compound 1-1) and two compounds represented by Formula II (denoted as Compound 2-1 and Compound 2-2), and a volume ratio of Compound 1-1 to Compound 2-1 to Compound 2-2 is (1-6):(1-2):1, which may be, for example, 1:1:1, 2:1:1, 3:1:1, 4:1:1, 1:2:1, 2:2:1, 3:2:1, 4:2:1, 5:2:1, or 6:2:1, etc.; or
the composition includes two compounds represented by Formula I (denoted as Compound 1-1 and Compound 1-2) and two compounds represented by Formula II (denoted as Compound 2-1 and Compound 2-2), and a volume ratio of Compound 1-1 to Compound 1-2 is (2:8)-(8:2), which may be, for example, 2:8, 3:7, 4:6, 5:5, 6:4, 7:3, or 8:2, etc.; a volume ratio of Compound 2-1 to Compound 2-2 is (2:8)-(8:2), which may be, for example, 2:8, 3:7, 4:6, 5:5, 6:4, 7:3, or 8:2, etc., and a volume sum of Compound 1-1 and Compound 1-2 and a volume sum of Compound 2-1 and Compound 2-2 has a ratio of (2:8)-(8:2), which may be, for example, 2:8, 3:7, 4:6, 5:5, 6:4, 7:3, or 8:2, etc.

In one embodiment, in the composition, at least one compound represented by Formula I meets at least one of conditions (2) to (6).

In one embodiment, in the composition, at least one compound represented by Formula I meets at least one of conditions (2) to (6), and the compound represented by Formula II meets condition (a).

In one embodiment, the composition includes a compound containing deuterium atoms represented by Formula I and a compound without deuterium atoms represented by Formula II.

The compound containing a deuterium atom represented by Formula I is selected from any one of Compound I-1-D, Compound I-2-D, or Compound I-3-D.

As an optional technical solution of the present application, Ar₂₁ and Ar₂₂ are each independently selected from any one or a combination of at least two of phenyl, naphthyl, fluorenyl, anthryl, phenanthryl, biphenyl, dibenzofuranyl, dibenzothienyl, terphenyl, or quaterphenyl.

In one embodiment, the Ar₂₁ is selected from phenyl, biphenyl, or terphenyl.

In one embodiment, Ar₂₂ is selected from biphenyl, terphenyl, or quaterphenyl.

In one embodiment, Ar₂₁ is phenyl, and Ar₂₂ is selected from biphenyl, terphenyl, or quaterphenyl.

In one embodiment, Ar₂₁ is biphenyl, and the Ar₂₂ is selected from biphenyl or terphenyl.

In one embodiment, R₁₀₁ and the R₁₀₂ are each independently selected from H, phenyl, naphthyl, triphenylenyl, fluoranthenyl, fluorenyl, anthryl, phenanthryl, biphenyl, naphthyl, dibenzofuranyl, or dibenzothienyl,

In one embodiment, R₁₀₁ and the R₁₀₂ are each independently selected from phenyl, biphenyl, or triphenylenyl.

In one embodiment, R₃₀₃ is selected from phenyl.

In one embodiment, Ar₁₁ is selected from any one of a single bond, phenylene, or naphthylene.

As an optional technical solution of the present application, the compound represented by Formula I has a structure as represented by Formula I-1, Formula I-2, or Formula I-3:

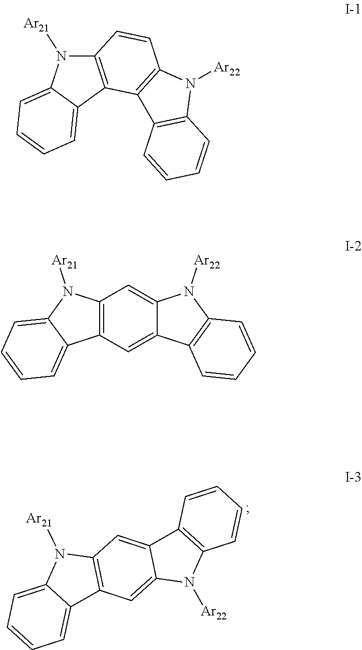

wherein Ar₂₁ and Ar₂₂ have the same protection scope as described above.

In one embodiment, the compound represented by Formula I meets condition (3) and/or condition (4).

As an optional technical solution of the present application, the compound represented by Formula I is selected from any one of Compound I-1-D, Compound I-2-D, or Compound I-3-D:

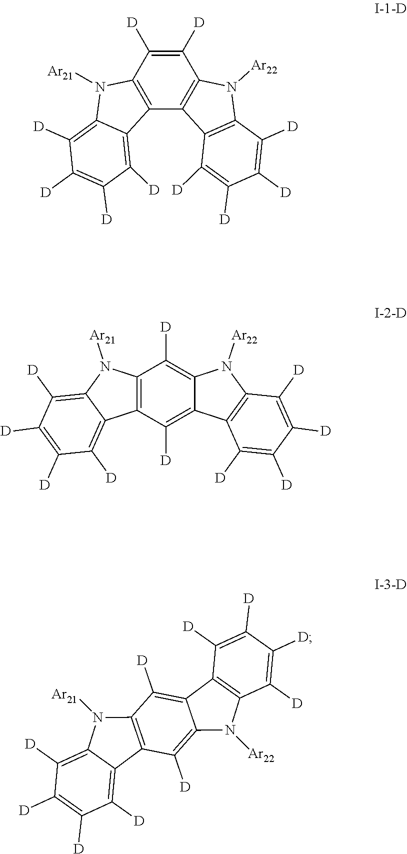

wherein Ar₂₁ and Ar₂₂ have the same protection scope as described above, and the hydrogen atoms in Ar₂₁ and Ar₂₂ are not substituted with deuterium atoms.

As an optional technical solution of the present application, the compound represented by Formula I is selected from any one of the following compounds:

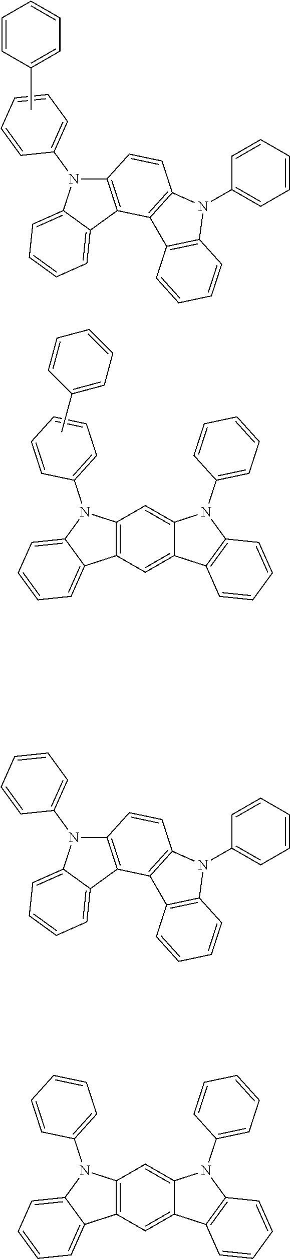

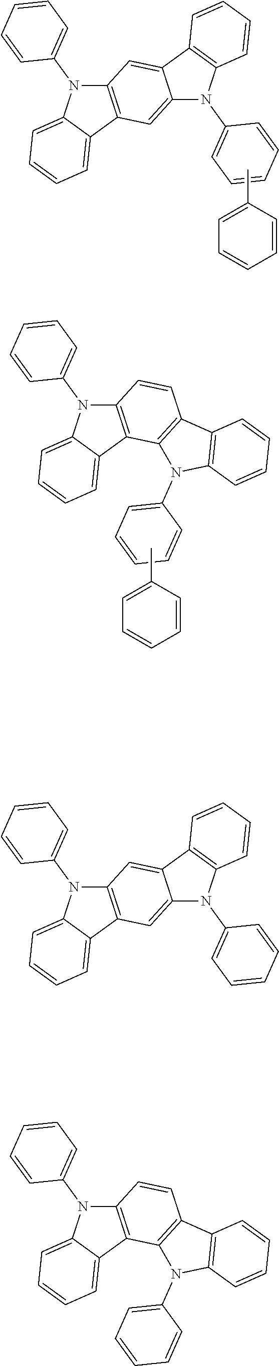

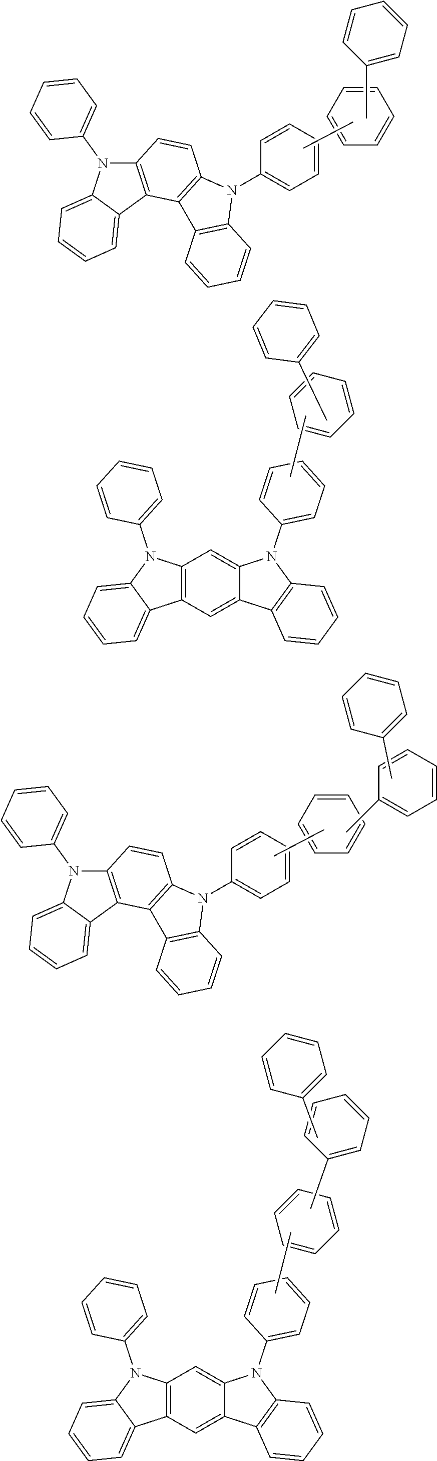

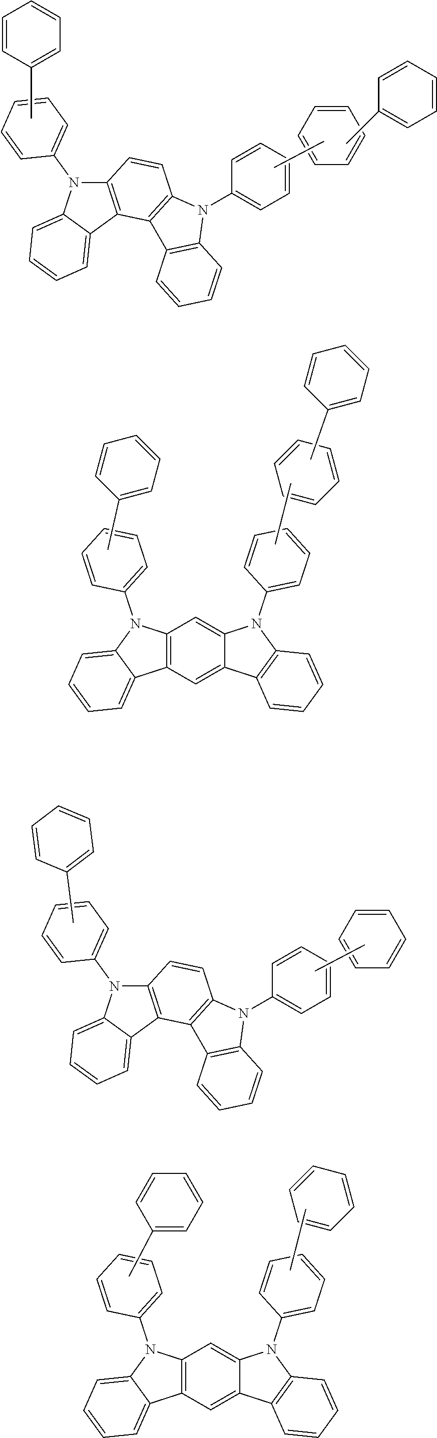

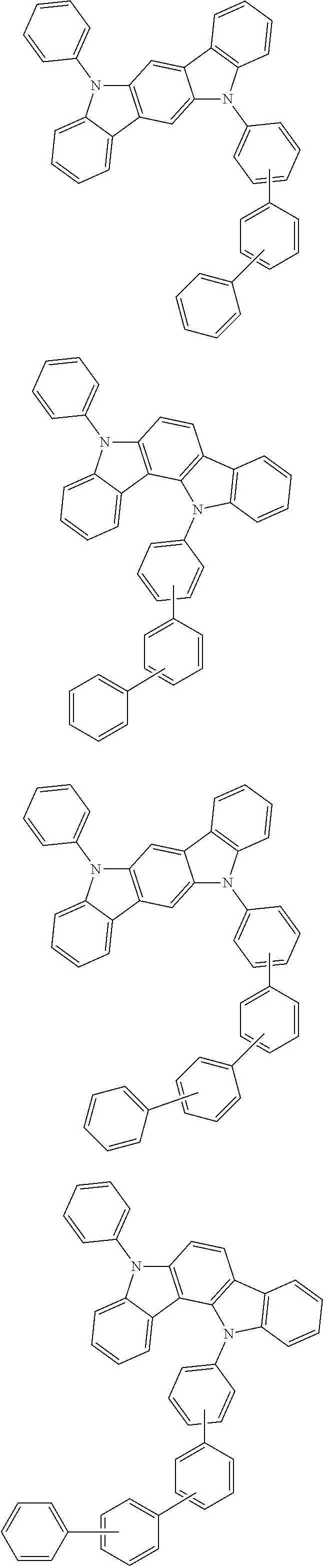

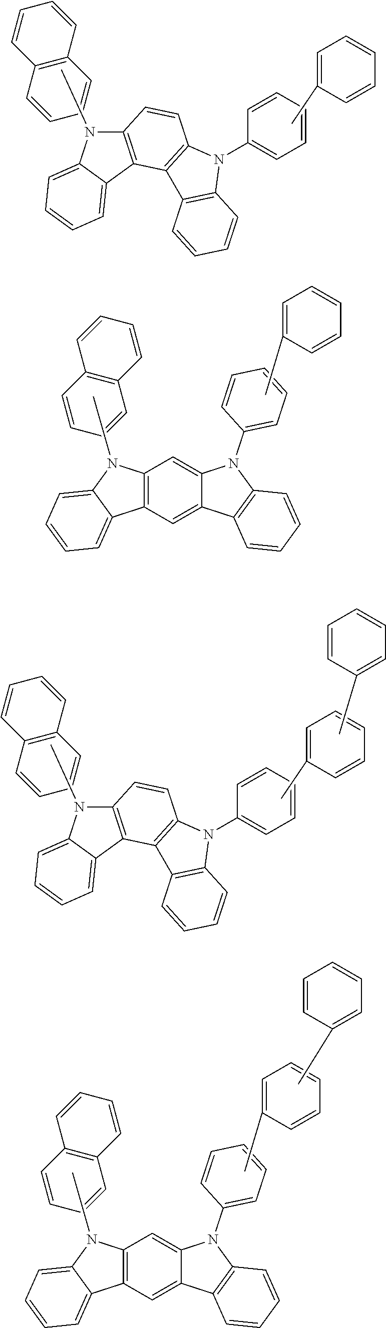

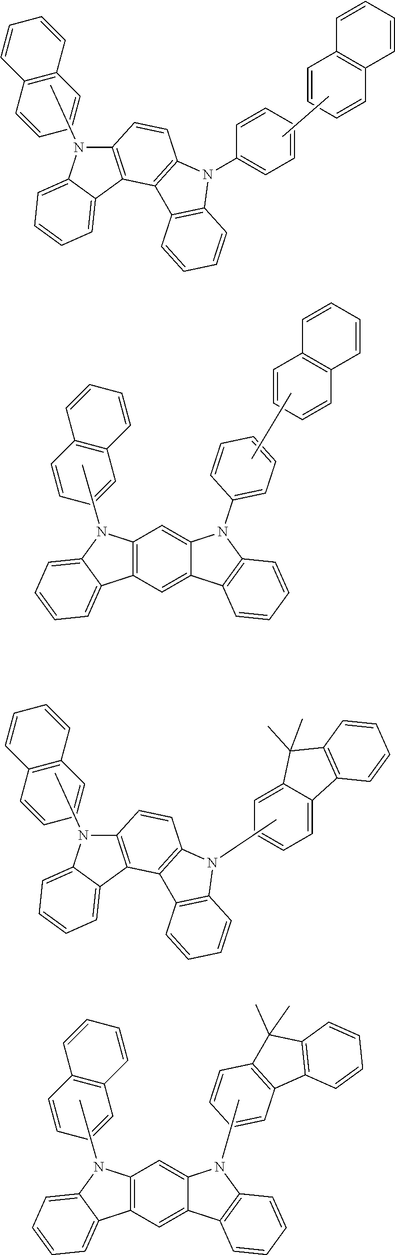

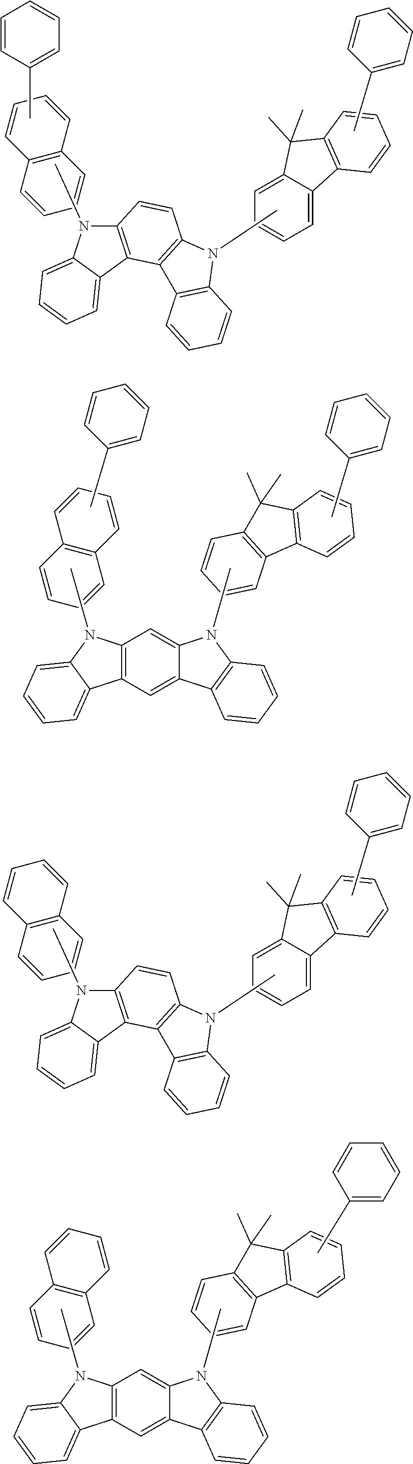

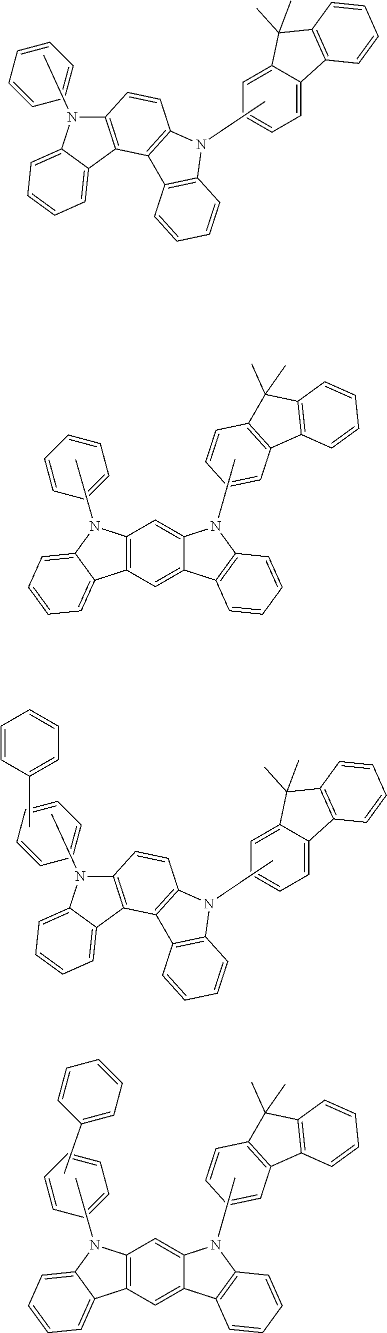

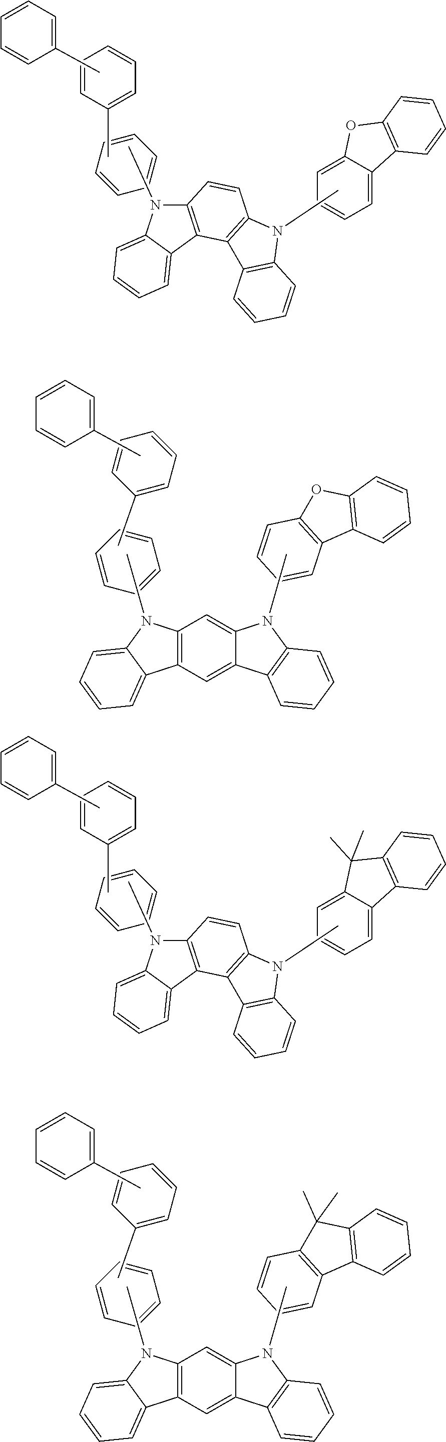

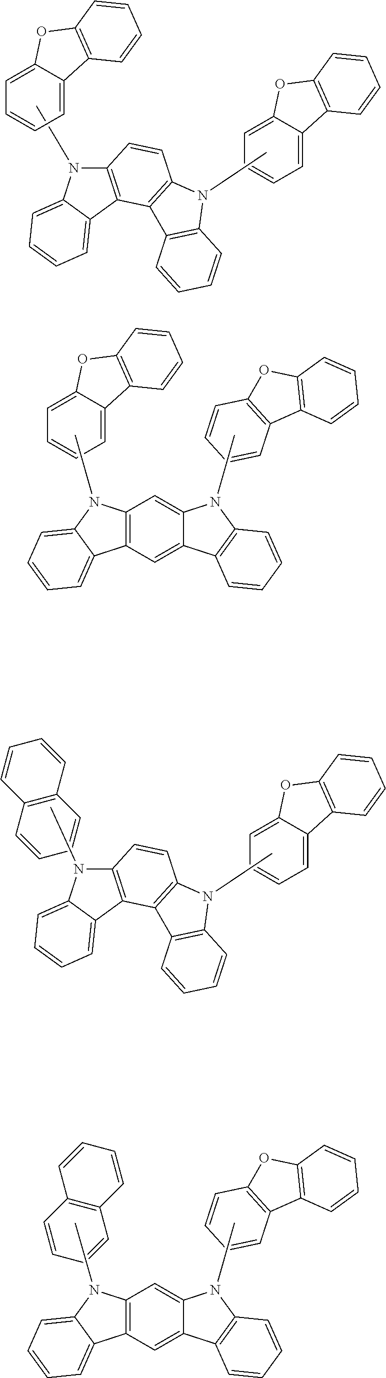

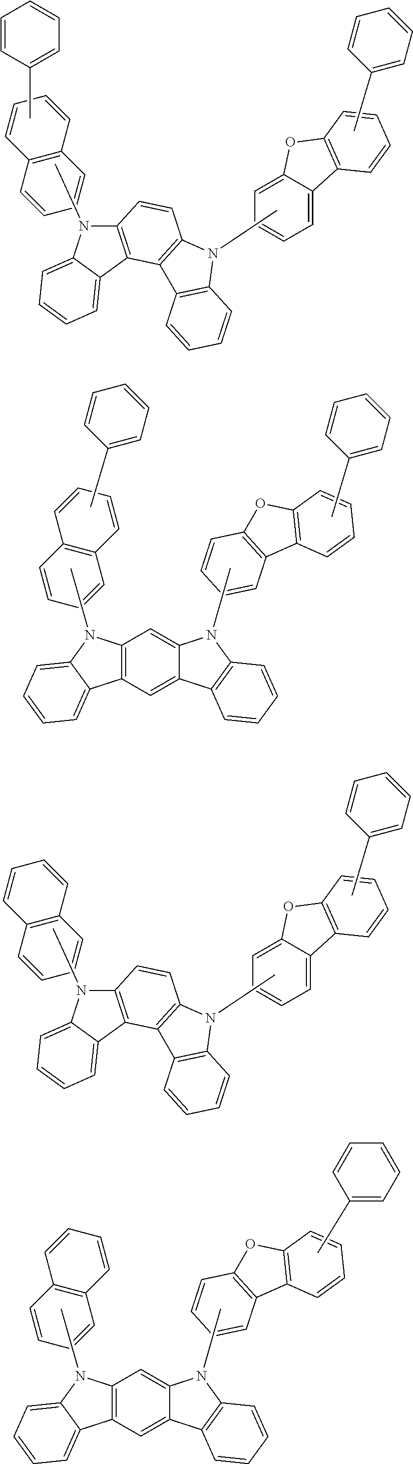

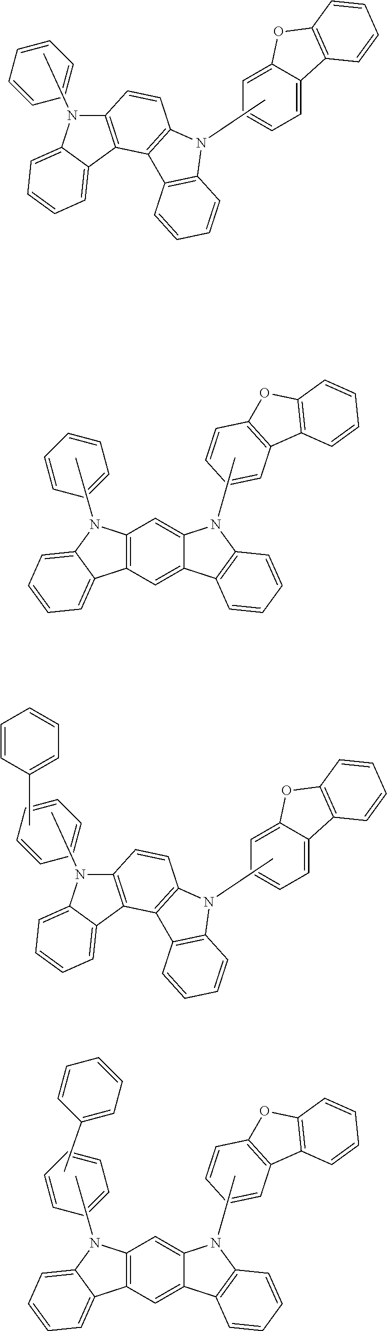

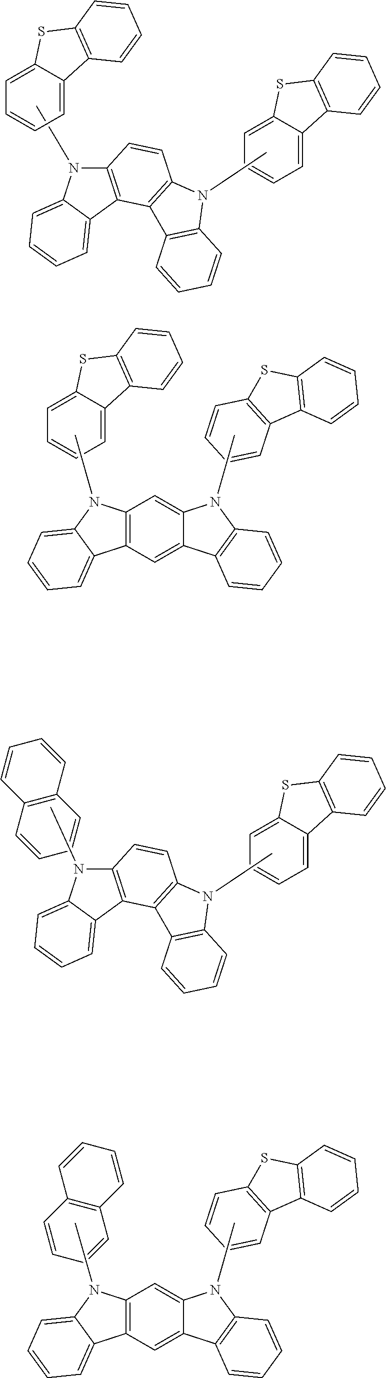

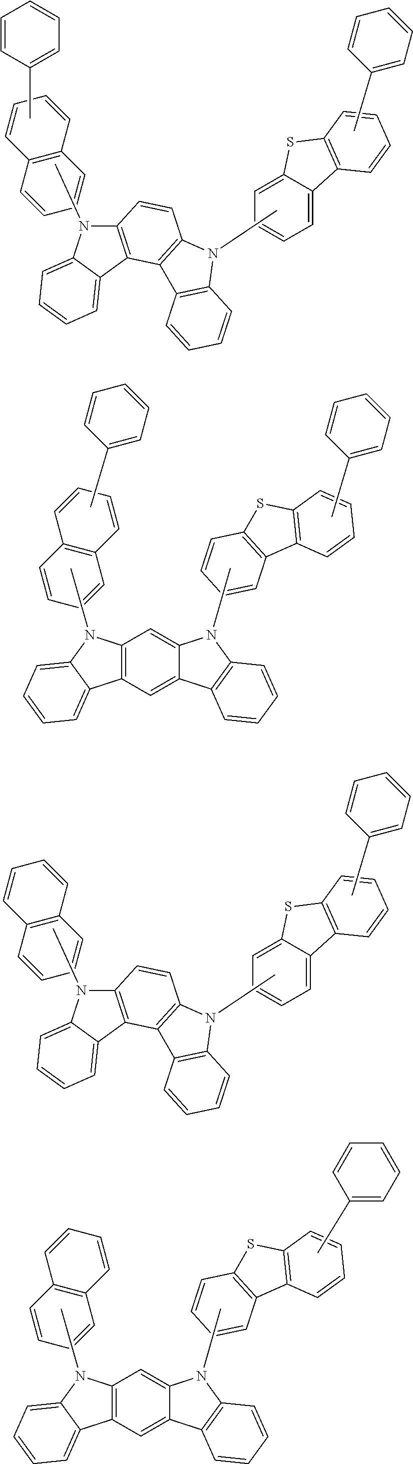

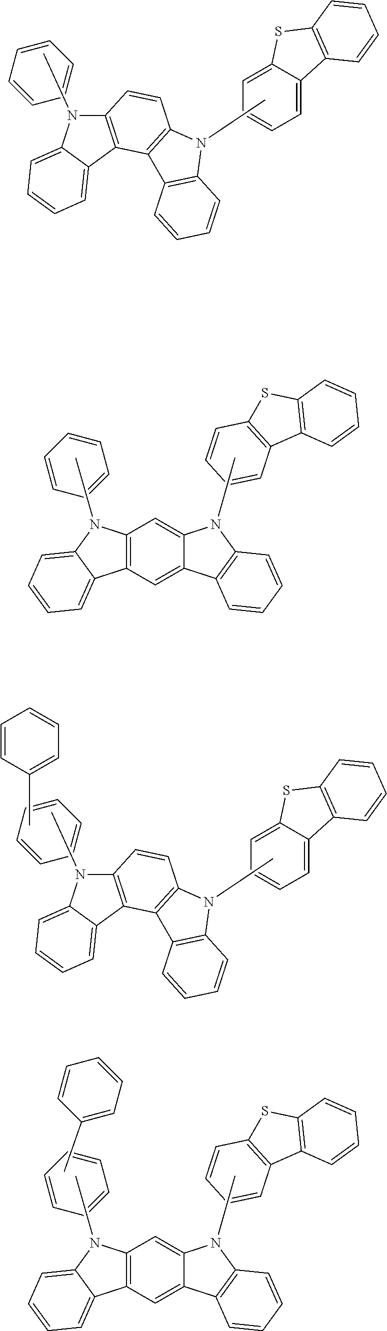

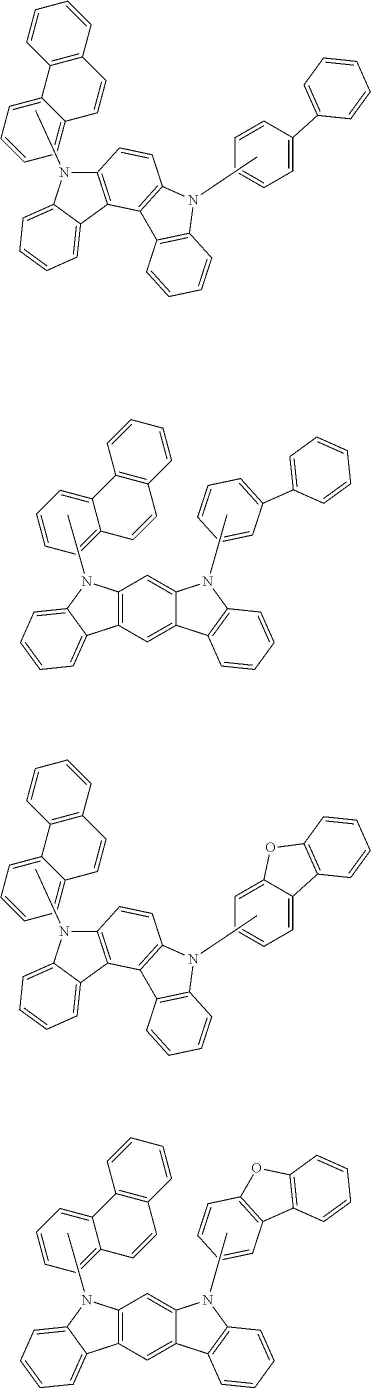

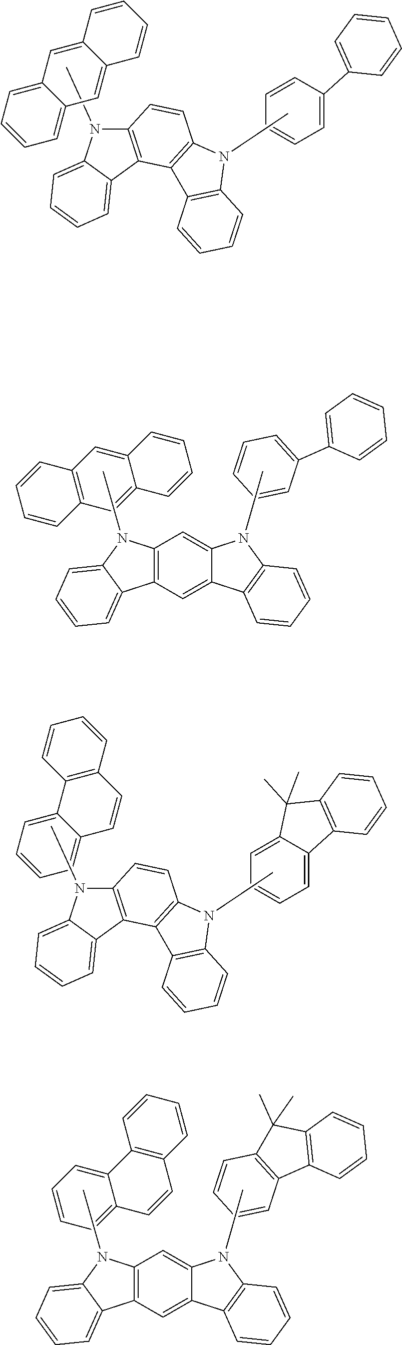

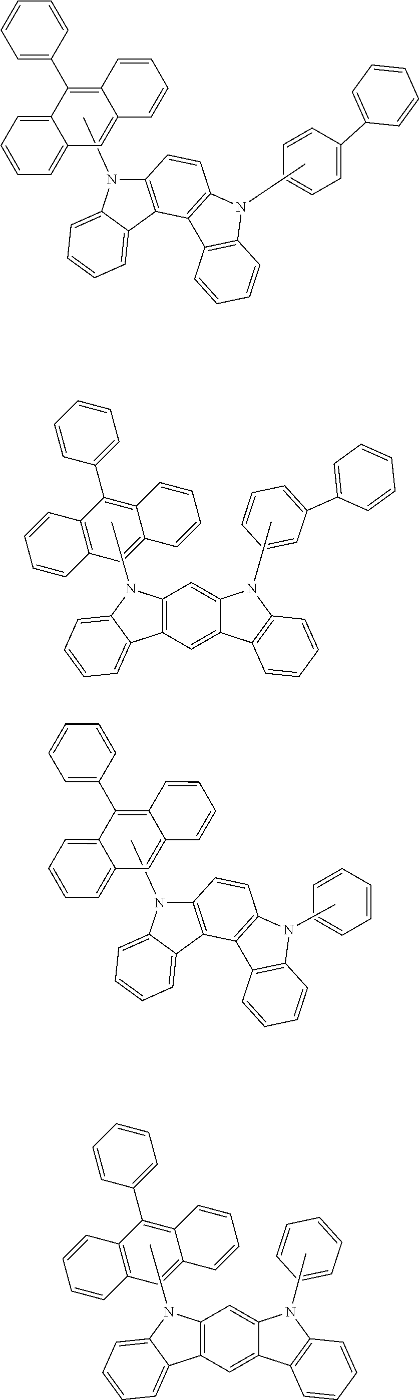

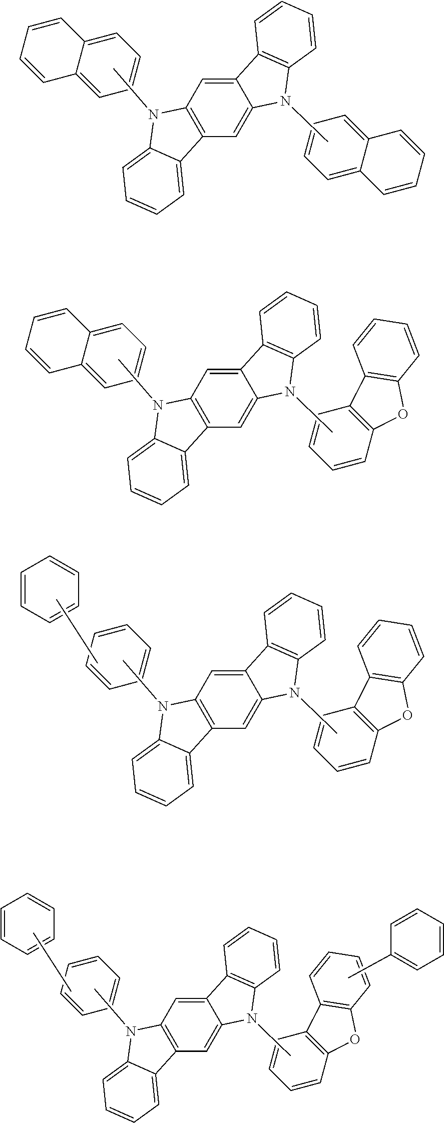

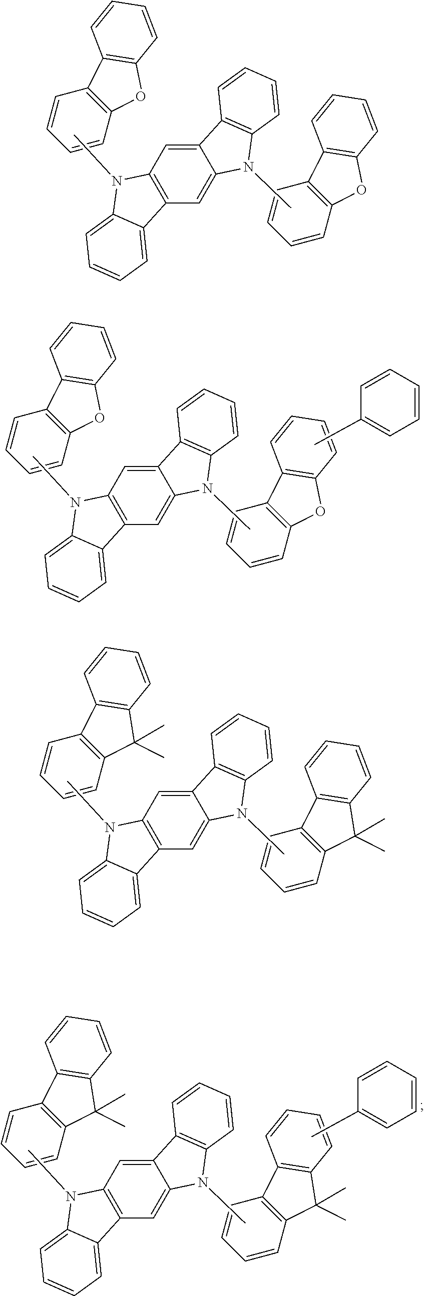

wherein hydrogen atoms in the above compounds can be substituted with deuterium atoms.

As an optional technical solution of the present application, the compound represented by Formula I is selected from any one of Compound H-1 to Compound H-44:

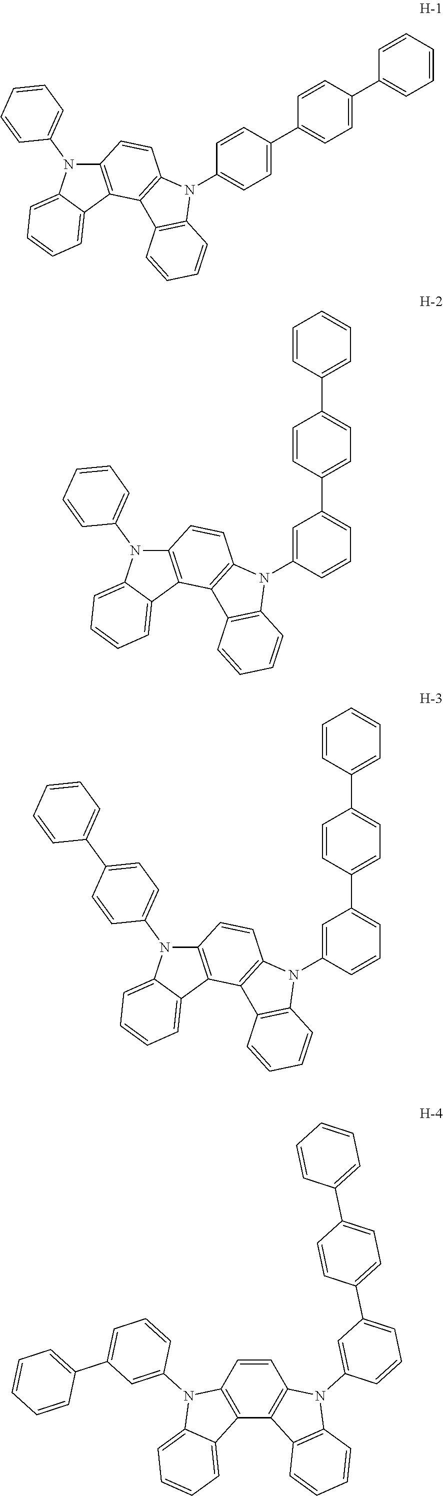 [SMILES: c1ccc(-c2ccc(-c3ccc(-n4c5ccccc5c5c6c7ccccc7n(-c7ccccc7)c6ccc54)cc3)cc2)cc1 ; c1ccc(-c2ccc(-c3cccc(-n4c5ccccc5c5c6c7ccccc7n(-c7ccccc7)c6ccc54)c3)cc2)cc1 ; c1ccc(-c2ccc(-c3cccc(-n4c5ccccc5c5c6c7ccccc7n(-c7ccc(-c8ccccc8)cc7)c6ccc54)c3)cc2)cc1 ; c1ccc(-c2ccc(-c3cccc(-n4c5ccccc5c5c6c7ccccc7n(-c7cccc(-c8ccccc8)c7)c6ccc54)c3)cc2)cc1]

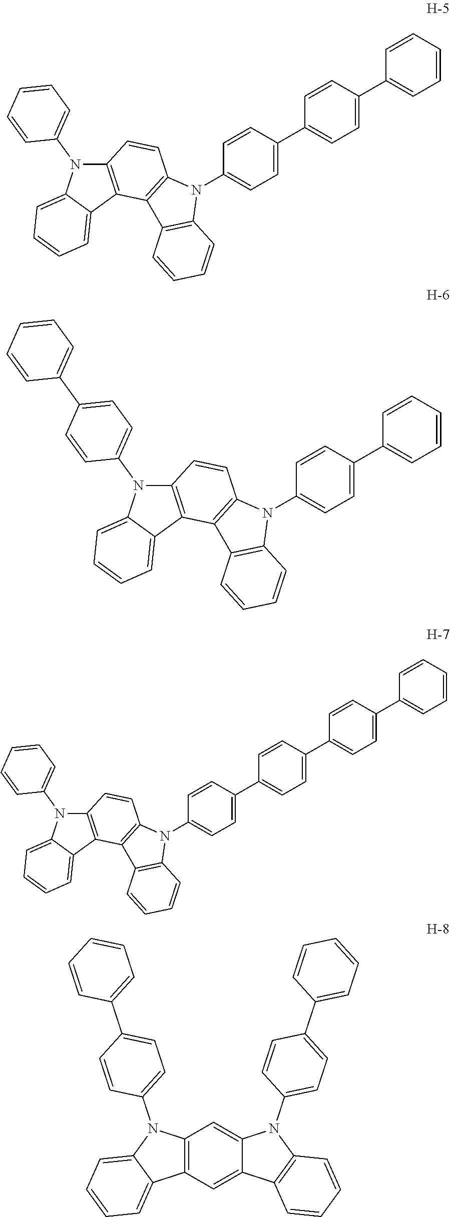

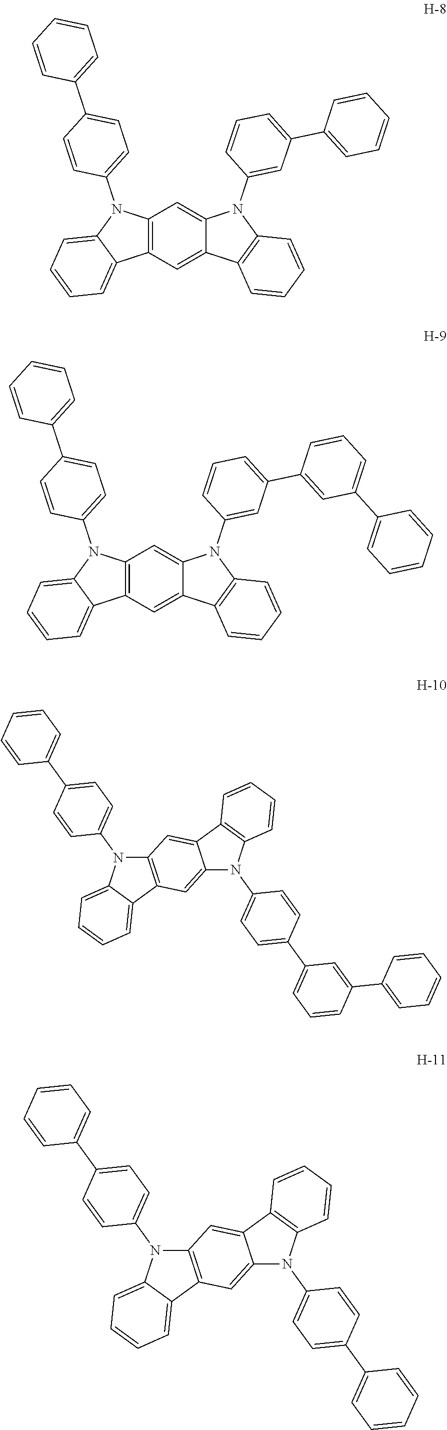 [SMILES: c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6cccc(-c7cccc(-c8ccccc8)c7)c6)c5cc43)cc2)cc1 ; c1ccc(-c2ccc(-n3c4ccccc4c4cc5c(cc43)c3ccccc3n5-c3ccc(-c4cccc(-c5ccccc5)c4)cc3)cc2)cc1 ; c1ccc(-c2ccc(-n3c4ccccc4c4cc5c(cc43)c3ccccc3n5-c3ccc(-c4ccccc4)cc3)cc2)cc1]

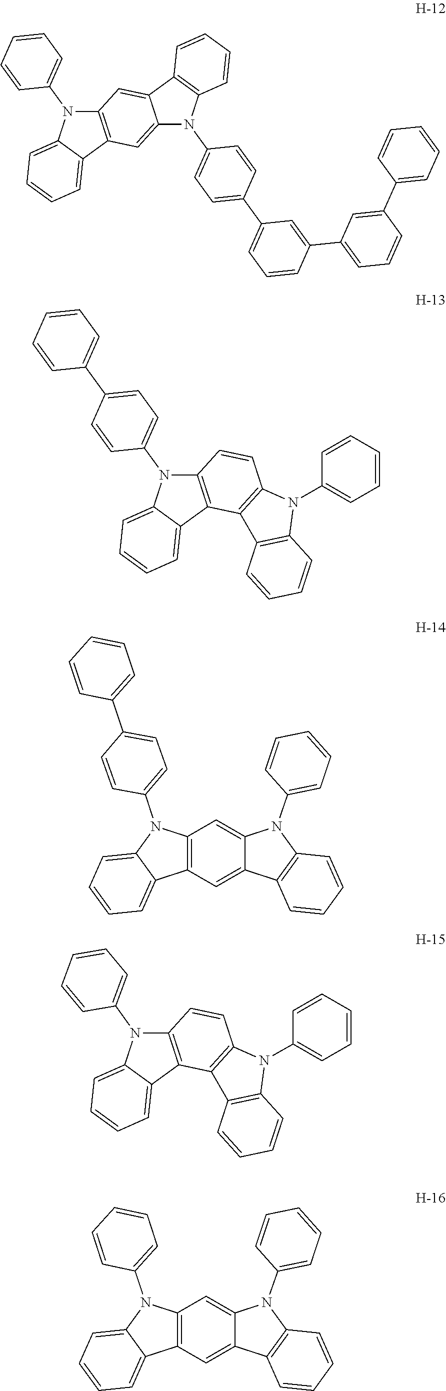

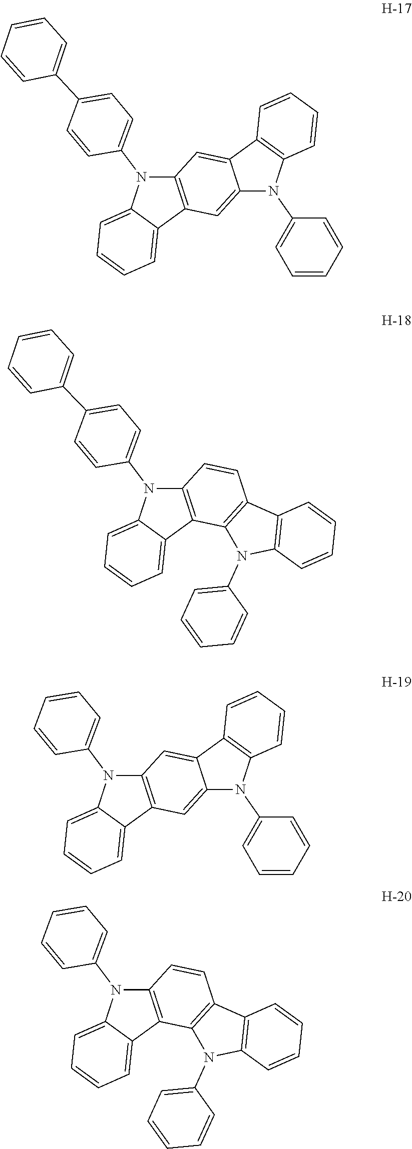

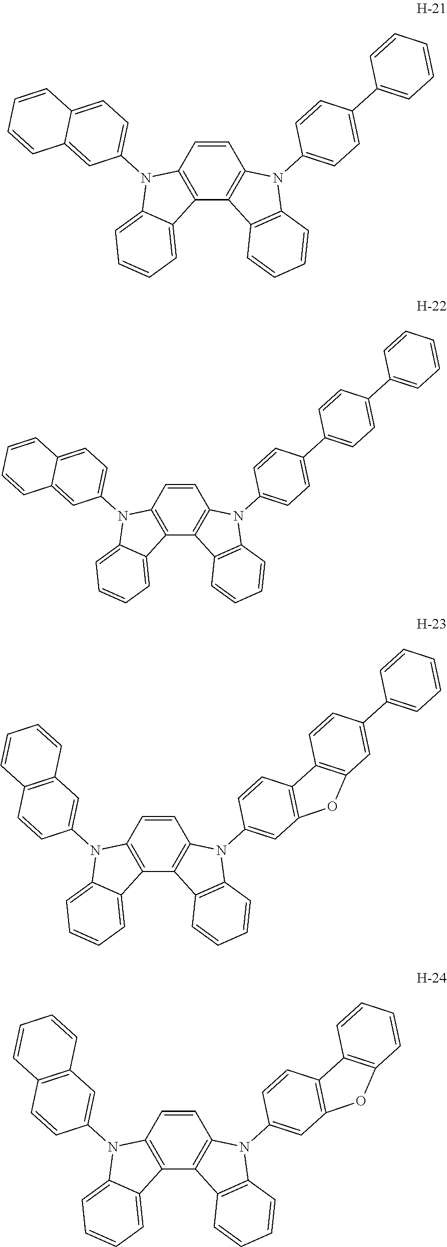 [SMILES: c1ccc(-c2ccc(-n3c4ccccc4c4c5c6ccccc6n(-c6ccc7ccccc7c6)c5ccc43)cc2)cc1 ; c1ccc(-c2ccc(-c3ccc(-n4c5ccccc5c5c6c7ccccc7n(-c7ccc8ccccc8c7)c6ccc54)cc3)cc2)cc1 ; c1ccc(-c2ccc3c(c2)oc2cc(-n4c5ccccc5c5c6c7ccccc7n(-c7ccc8ccccc8c7)c6ccc54)ccc23)cc1 ; c1ccc2cc(-n3c4ccccc4c4c5c6ccccc6n(-c6ccc7c(c6)oc6ccccc67)c5ccc43)ccc2c1]

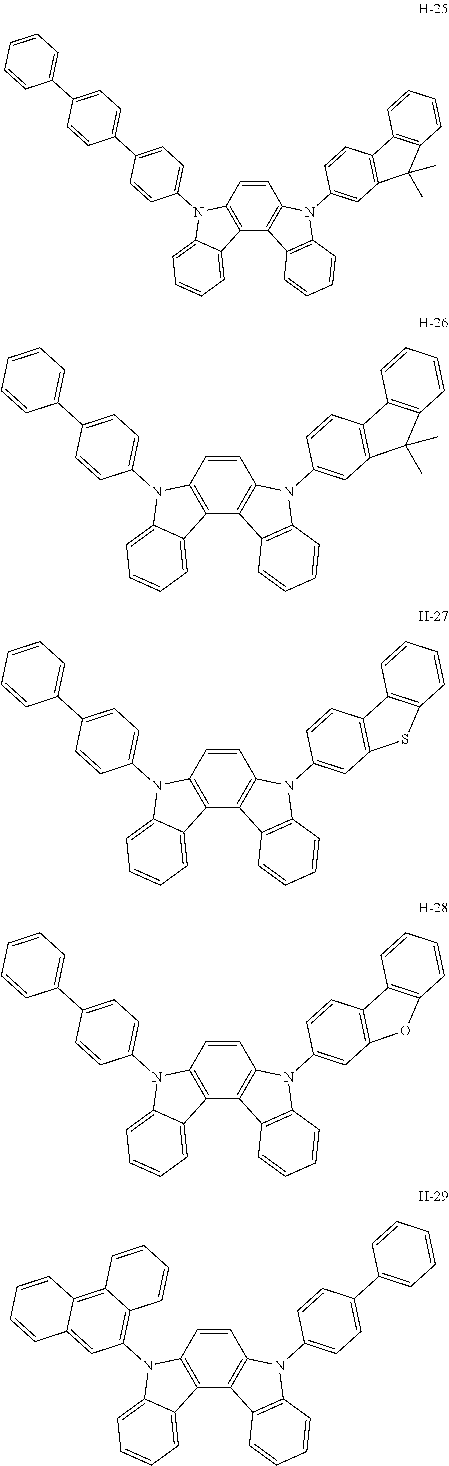 [SMILES: CC1(C)c2ccccc2-c2ccc(-n3c4ccccc4c4c5c6ccccc6n(-c6ccc(-c7ccc(-c8ccccc8)cc7)cc6)c5ccc43)cc21 ; CC1(C)c2ccccc2-c2ccc(-n3c4ccccc4c4c5c6ccccc6n(-c6ccc(-c7ccccc7)cc6)c5ccc43)cc21 ; c1ccc(-c2ccc(-n3c4ccccc4c4c5c6ccccc6n(-c6ccc7c(c6)sc6ccccc67)c5ccc43)cc2)cc1 ; c1ccc(-c2ccc(-n3c4ccccc4c4c5c6ccccc6n(-c6ccc7c(c6)oc6ccccc67)c5ccc43)cc2)cc1 ; c1ccc(-c2ccc(-n3c4ccccc4c4c5c6ccccc6n(-c6cc7ccccc7c7ccccc67)c5ccc43)cc2)cc1]

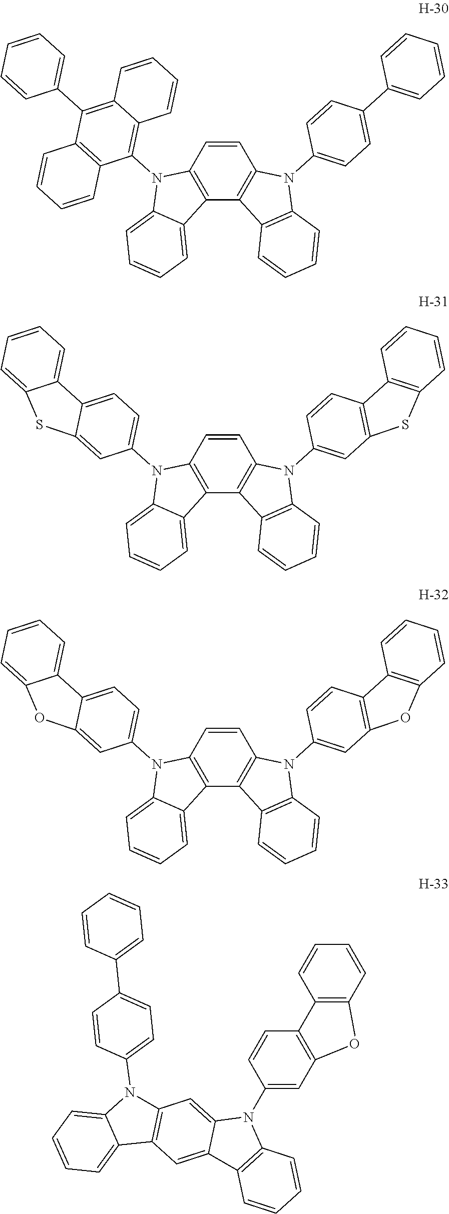 [SMILES: c1ccc(-c2ccc(-n3c4ccccc4c4c5c6ccccc6n(-c6c7ccccc7c(-c7ccccc7)c7ccccc67)c5ccc43)cc2)cc1 ; c1ccc2c(c1)sc1cc(-n3c4ccccc4c4c5c6ccccc6n(-c6ccc7c(c6)sc6ccccc67)c5ccc43)ccc12 ; c1ccc2c(c1)oc1cc(-n3c4ccccc4c4c5c6ccccc6n(-c6ccc7c(c6)oc6ccccc67)c5ccc43)ccc12 ; c1ccc(-c2ccc(-n3c4ccccc4c4cc5c6ccccc6n(-c6ccc7c(c6)oc6ccccc67)c5cc43)cc2)cc1]

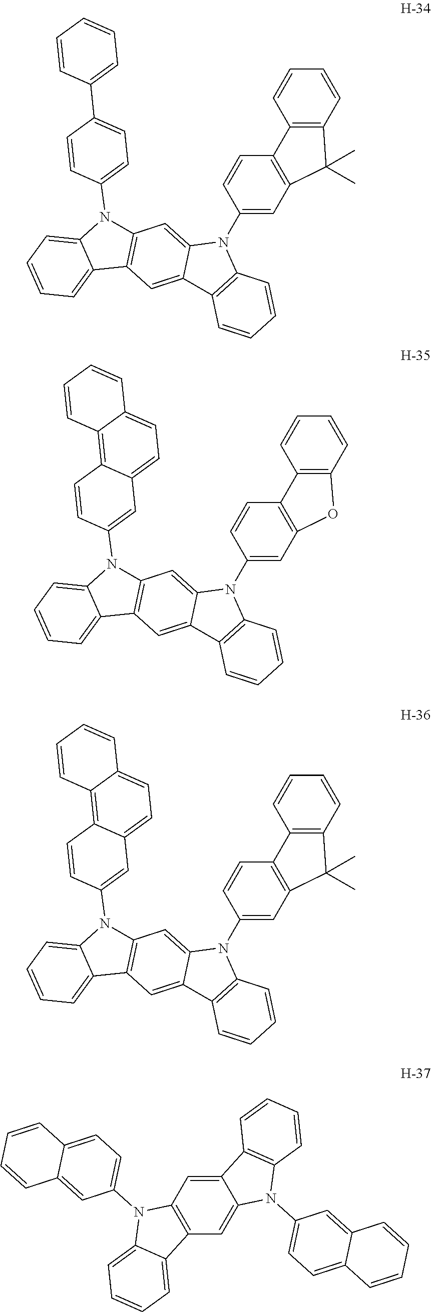 [SMILES: CC1(C)c2ccccc2-c2ccc(-n3c4ccccc4c4cc5c6ccccc6n(-c6ccc(-c7ccccc7)cc6)c5cc43)cc21 ; c1ccc2c(c1)ccc1cc(-n3c4ccccc4c4cc5c6ccccc6n(-c6ccc7c(c6)oc6ccccc67)c5cc43)ccc12 ; CC1(C)c2ccccc2-c2ccc(-n3c4ccccc4c4cc5c6ccccc6n(-c6ccc7c(ccc8ccccc87)c6)c5cc43)cc21 ; c1ccc2cc(-n3c4ccccc4c4cc5c(cc43)c3ccccc3n5-c3ccc4ccccc4c3)ccc2c1]

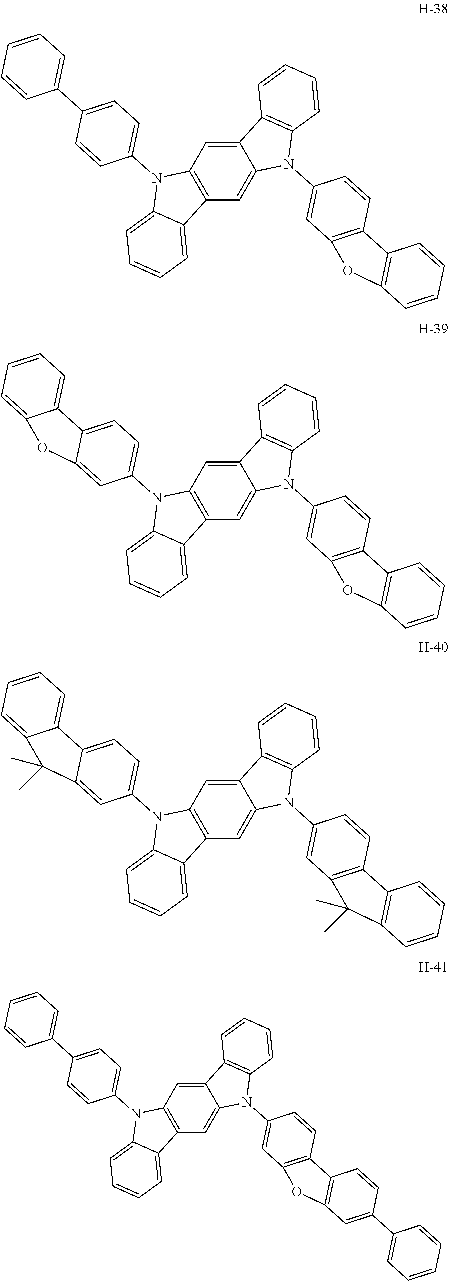 [SMILES: c1ccc(-c2ccc(-n3c4ccccc4c4cc5c(cc43)c3ccccc3n5-c3ccc4c(c3)oc3ccccc34)cc2)cc1 ; c1ccc2c(c1)oc1cc(-n3c4ccccc4c4cc5c(cc43)c3ccccc3n5-c3ccc4c(c3)oc3ccccc34)ccc12 ; CC1(C)c2ccccc2-c2ccc(-n3c4ccccc4c4cc5c(cc43)c3ccccc3n5-c3ccc4c(c3)C(C)(C)c3ccccc3-4)cc21 ; c1ccc(-c2ccc(-n3c4ccccc4c4cc5c(cc43)c3ccccc3n5-c3ccc4c(c3)oc3cc(-c5ccccc5)ccc34)cc2)cc1]

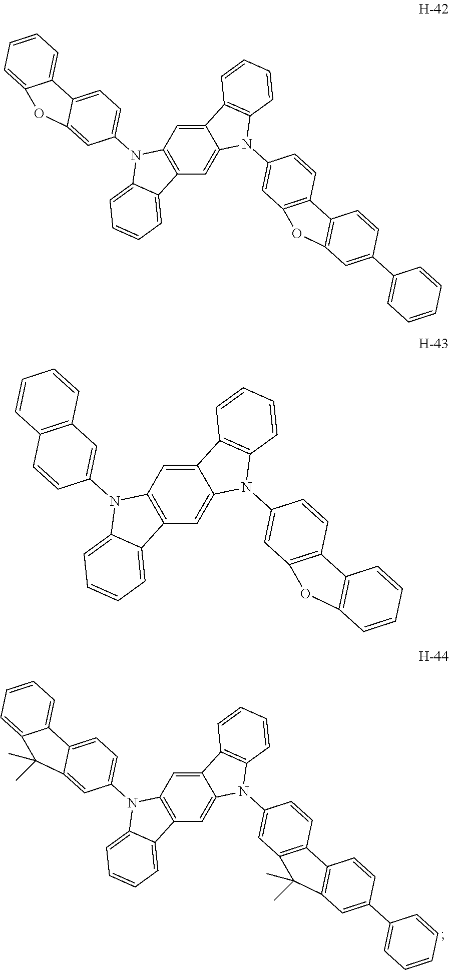 [SMILES: c1ccc(-c2ccc3c(c2)oc2cc(-n4c5ccccc5c5cc6c(cc54)c4ccccc4n6-c4ccc5c(c4)oc4ccccc45)ccc23)cc1 ; c1ccc2cc(-n3c4ccccc4c4cc5c(cc43)c3ccccc3n5-c3ccc4c(c3)oc3ccccc34)ccc2c1 ; CC1(C)c2ccccc2-c2ccc(-n3c4ccccc4c4cc5c(cc43)c3ccccc3n5-c3ccc4c(c3)C(C)(C)c3cc(-c5ccccc5)ccc3-4)cc21]

wherein hydrogen atoms in the Compound H-1 to Compound H-44 can be substituted with deuterium atoms.

As an optional technical solution of the present application, the compound represented by Formula II is selected from any one of the following compounds:

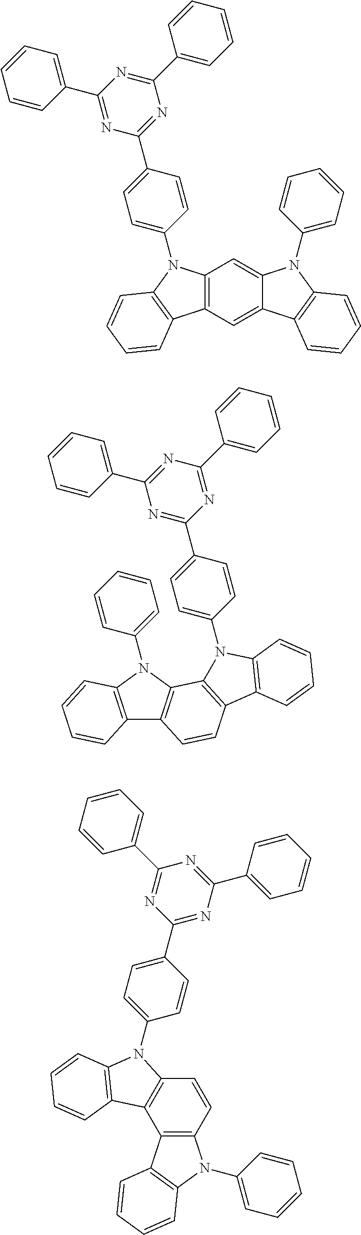 [SMILES: c1ccc(-c2nc(-c3ccccc3)nc(-c3ccc(-n4c5ccccc5c5cc6c7ccccc7n(-c7ccccc7)c6cc54)cc3)n2)cc1 ; c1ccc(-c2nc(-c3ccccc3)nc(-c3ccc(-n4c5ccccc5c5ccc6c7ccccc7n(-c7ccccc7)c6c54)cc3)n2)cc1 ; c1ccc(-c2nc(-c3ccccc3)nc(-c3ccc(-n4c5ccccc5c5c6c7ccccc7n(-c7ccccc7)c6ccc54)cc3)n2)cc1]

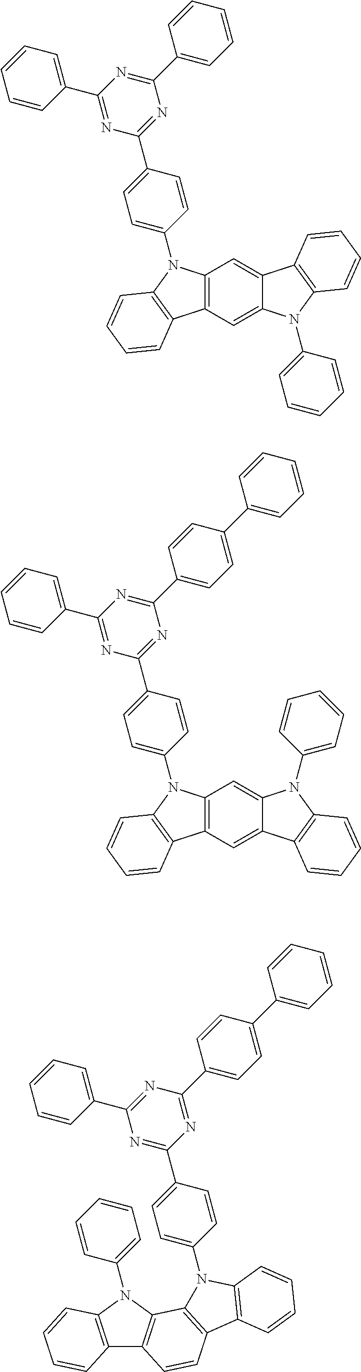 [SMILES: c1ccc(-c2nc(-c3ccccc3)nc(-c3ccc(-n4c5ccccc5c5cc6c(cc54)c4ccccc4n6-c4ccccc4)cc3)n2)cc1 ; c1ccc(-c2ccc(-c3nc(-c4ccccc4)nc(-c4ccc(-n5c6ccccc6c6cc7c8ccccc8n(-c8ccccc8)c7cc65)cc4)n3)cc2)cc1 ; c1ccc(-c2ccc(-c3nc(-c4ccccc4)nc(-c4ccc(-n5c6ccccc6c6ccc7c8ccccc8n(-c8ccccc8)c7c65)cc4)n3)cc2)cc1]

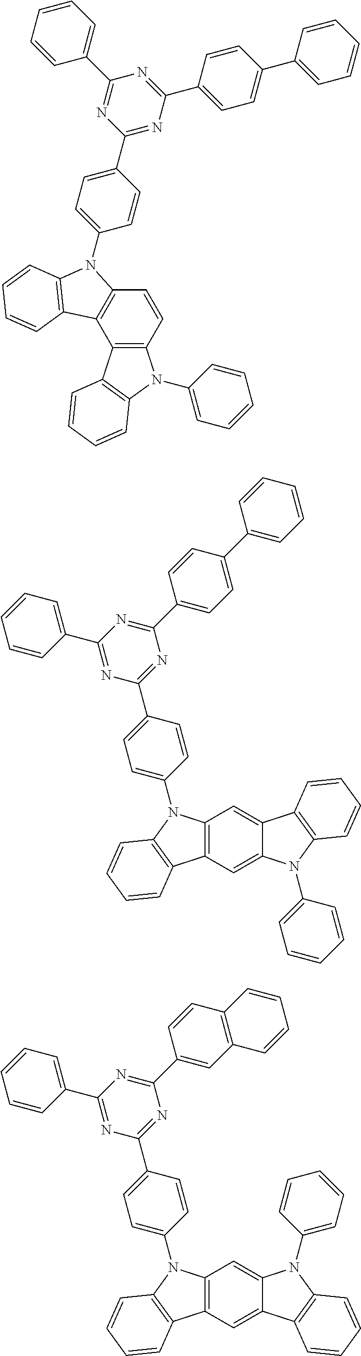

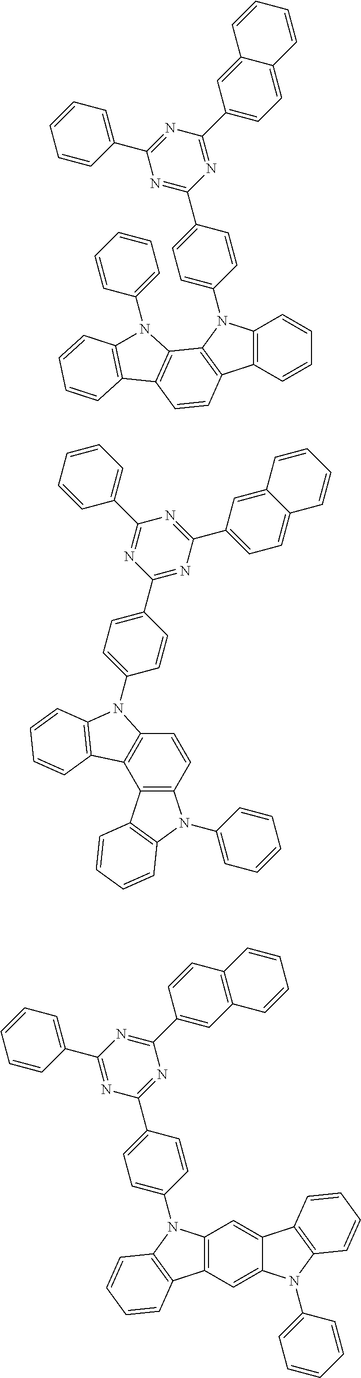

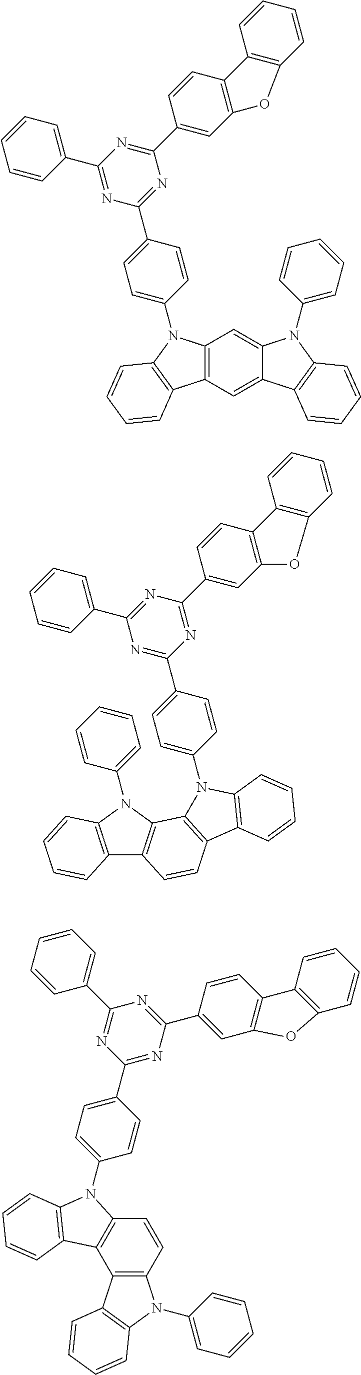

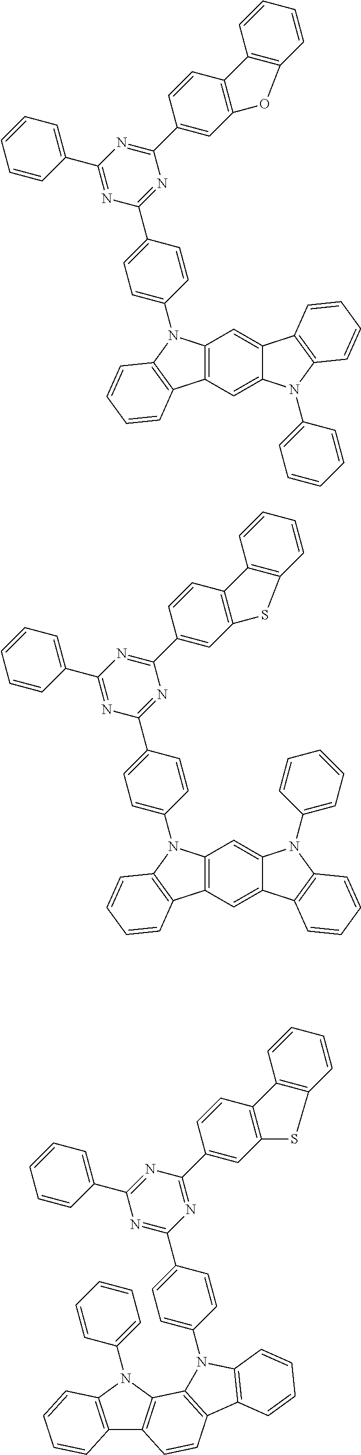

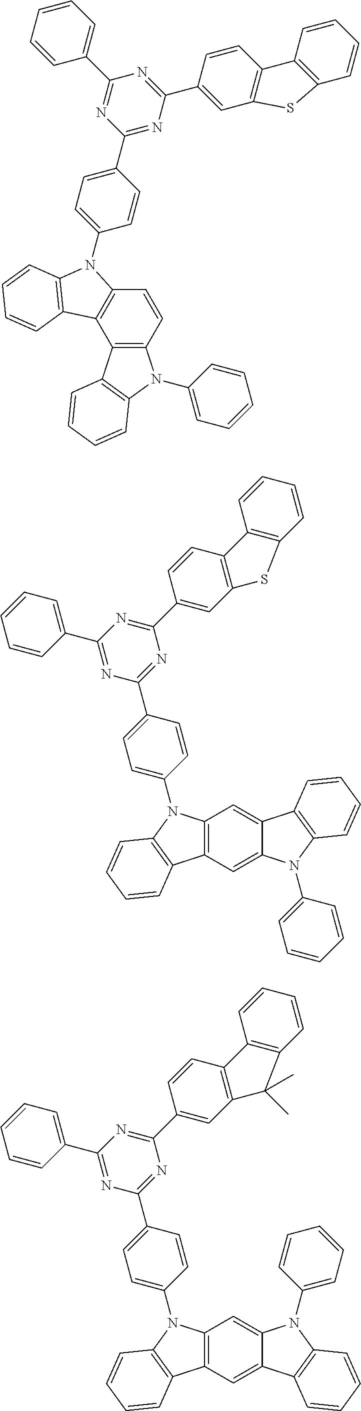

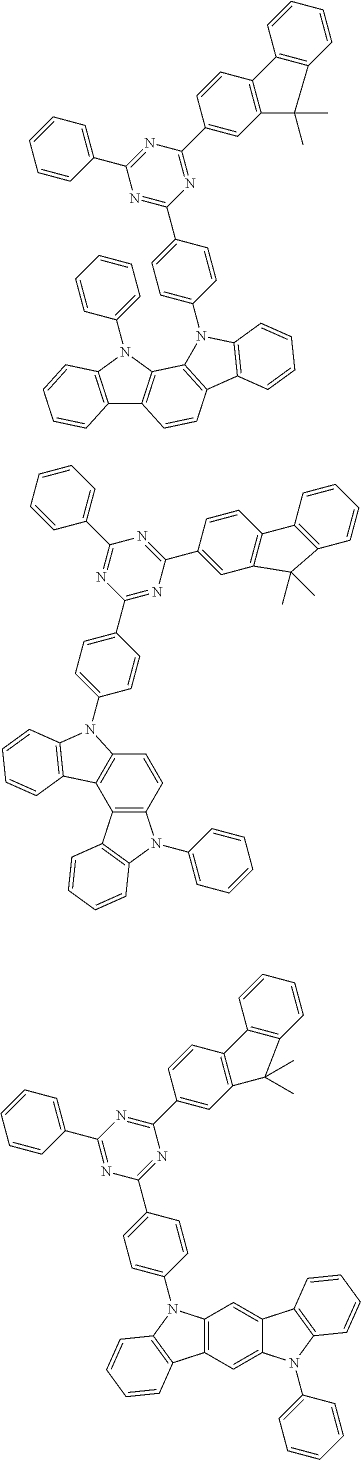

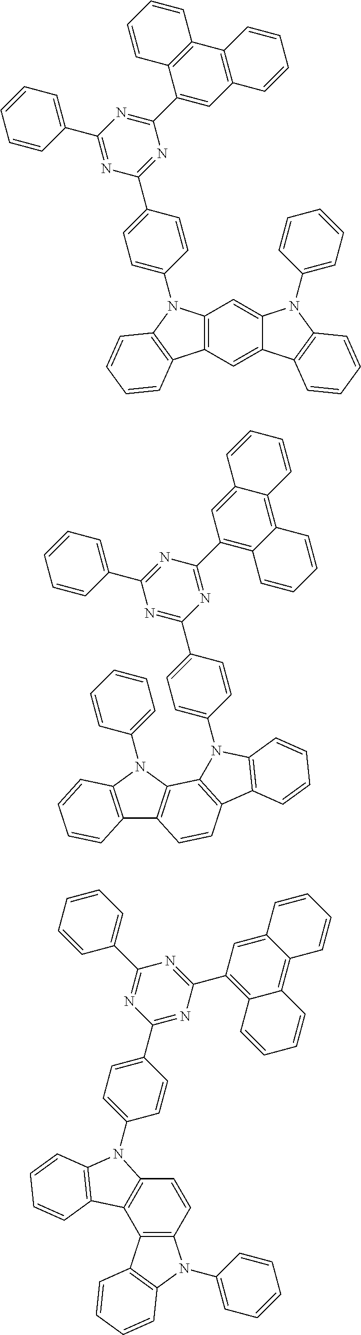 [SMILES: c1ccc(-c2nc(-c3ccc(-n4c5ccccc5c5cc6c7ccccc7n(-c7ccccc7)c6cc54)cc3)nc(-c3cc4ccccc4c4ccccc34)n2)cc1 ; c1ccc(-c2nc(-c3ccc(-n4c5ccccc5c5ccc6c7ccccc7n(-c7ccccc7)c6c54)cc3)nc(-c3cc4ccccc4c4ccccc34)n2)cc1 ; c1ccc(-c2nc(-c3ccc(-n4c5ccccc5c5c6c7ccccc7n(-c7ccccc7)c6ccc54)cc3)nc(-c3cc4ccccc4c4ccccc34)n2)cc1]

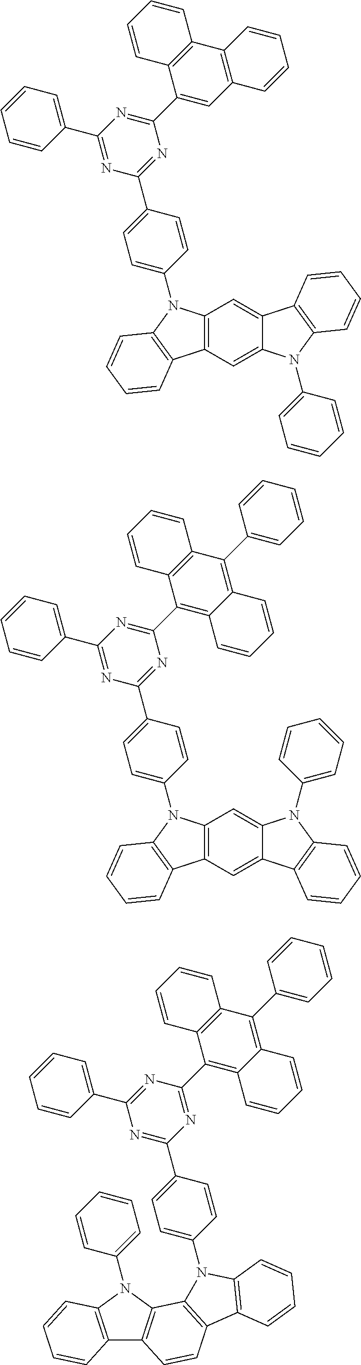

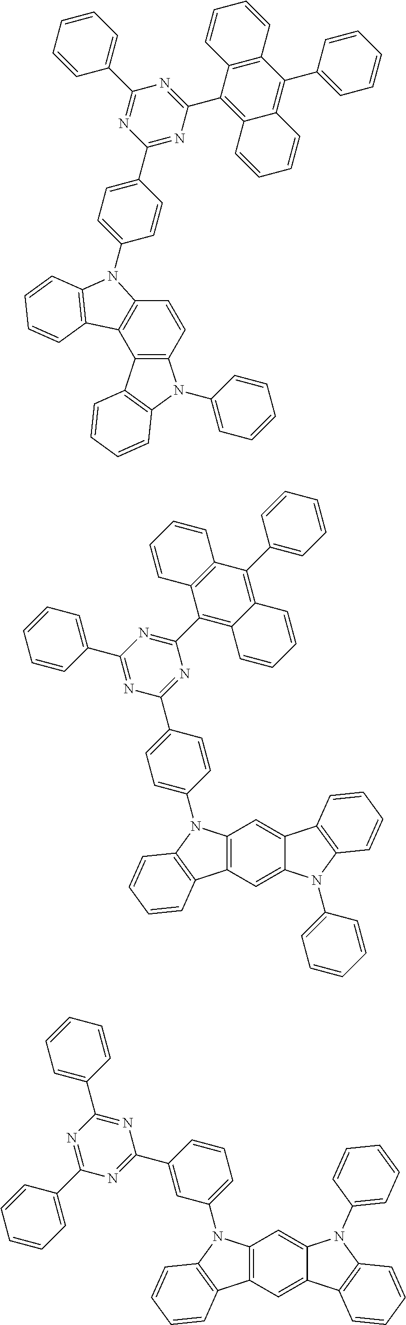 [SMILES: c1ccc(-c2nc(-c3ccc(-n4c5ccccc5c5c6c7ccccc7n(-c7ccccc7)c6ccc54)cc3)nc(-c3c4ccccc4c(-c4ccccc4)c4ccccc34)n2)cc1 ; c1ccc(-c2nc(-c3ccc(-n4c5ccccc5c5cc6c(cc54)c4ccccc4n6-c4ccccc4)cc3)nc(-c3c4ccccc4c(-c4ccccc4)c4ccccc34)n2)cc1 ; c1ccc(-c2nc(-c3ccccc3)nc(-c3cccc(-n4c5ccccc5c5cc6c7ccccc7n(-c7ccccc7)c6cc54)c3)n2)cc1]

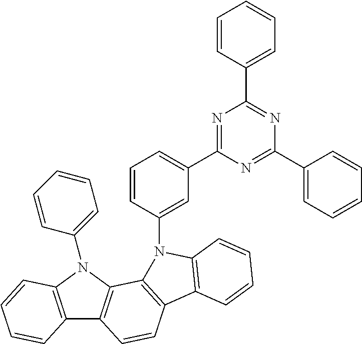 [SMILES: c1ccc(-c2nc(-c3ccccc3)nc(-c3cccc(-n4c5ccccc5c5ccc6c7ccccc7n(-c7ccccc7)c6c54)c3)n2)cc1]

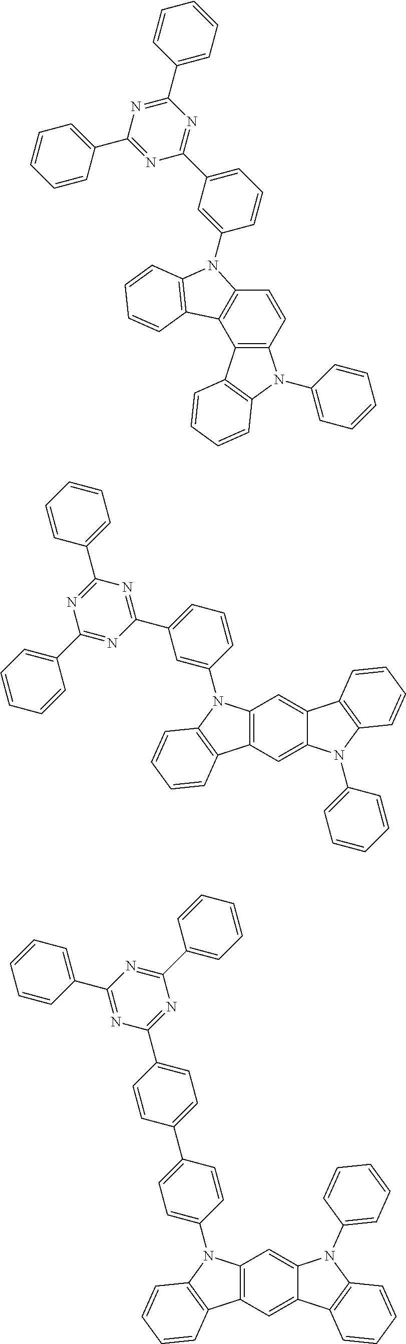 [SMILES: c1ccc(-c2nc(-c3ccccc3)nc(-c3cccc(-n4c5ccccc5c5c6c7ccccc7n(-c7ccccc7)c6ccc54)c3)n2)cc1 ; c1ccc(-c2nc(-c3ccccc3)nc(-c3cccc(-n4c5ccccc5c5cc6c(cc54)c4ccccc4n6-c4ccccc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1]

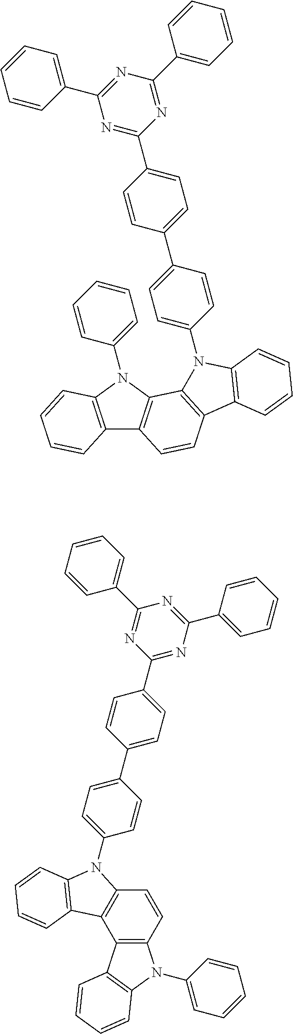 [SMILES: c1ccc(-c2nc(-c3ccccc3)nc(-c3ccc(-c4ccc(-n5c6ccccc6c6ccc7c8ccccc8n(-c8ccccc8)c7c65)cc4)cc3)n2)cc1 ; c1ccc(-c2nc(-c3ccccc3)nc(-c3ccc(-c4ccc(-n5c6ccccc6c6c7c8ccccc8n(-c8ccccc8)c7ccc65)cc4)cc3)n2)cc1]

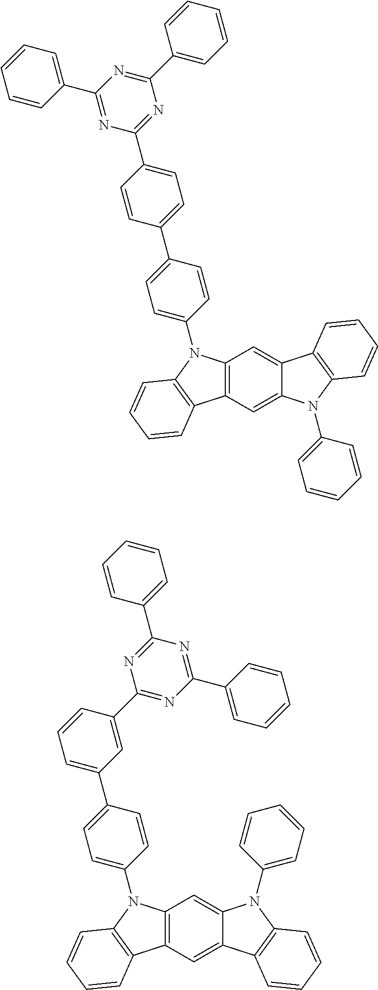 [SMILES: c1ccc(-c2nc(-c3ccccc3)nc(-c3ccc(-c4ccc(-n5c6ccccc6c6cc7c(cc65)c5ccccc5n7-c5ccccc5)cc4)cc3)n2)cc1 ; c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1]

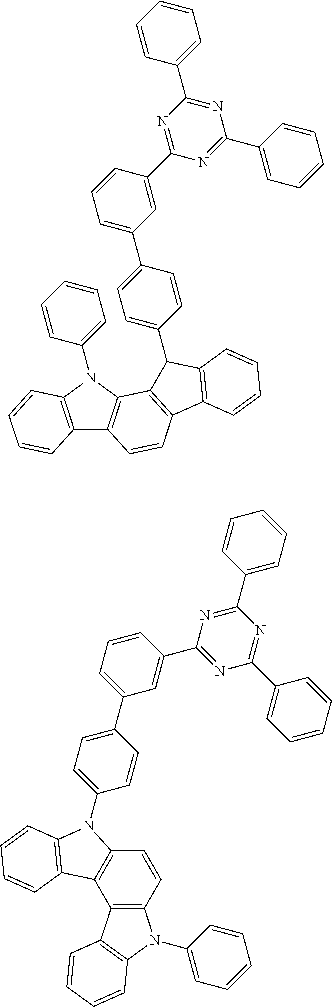 [SMILES: c1ccc(-c2nc(-c3ccccc3)nc(-c3cccc(-c4ccc(C5c6ccccc6-c6ccc7c8ccccc8n(-c8ccccc8)c7c65)cc4)c3)n2)cc1 ; c1ccc(-c2nc(-c3ccccc3)nc(-c3cccc(-c4ccc(-n5c6ccccc6c6c7c8ccccc8n(-c8ccccc8)c7ccc65)cc4)c3)n2)cc1]

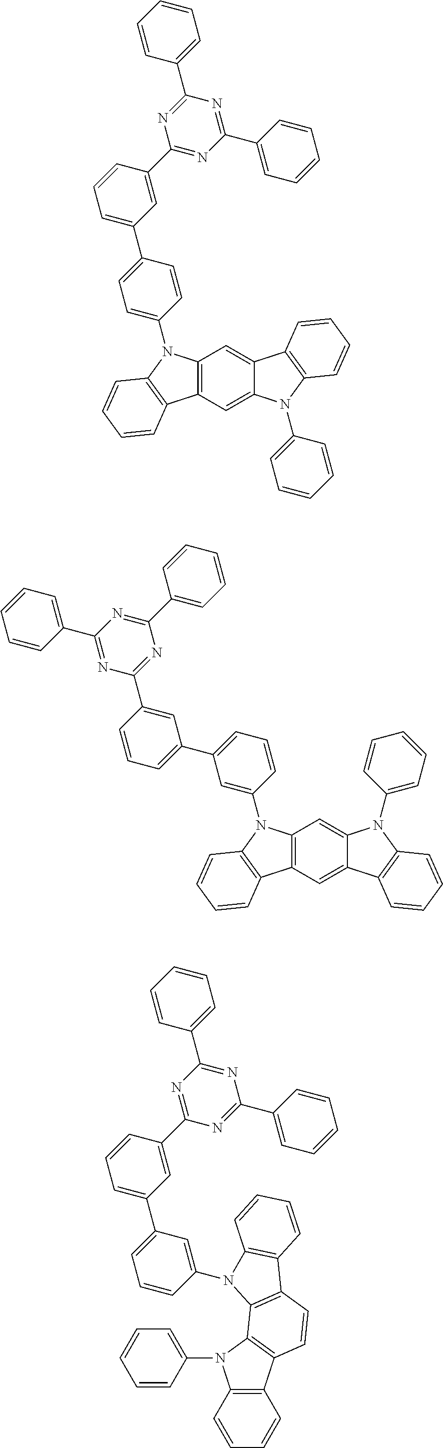 [SMILES: c1ccc(-c2nc(-c3ccccc3)nc(-c3cccc(-c4ccc(-n5c6ccccc6c6cc7c(cc65)c5ccccc5n7-c5ccccc5)cc4)c3)n2)cc1 ; c1ccc(-c2nc(-c3ccccc3)nc(-c3cccc(-c4cccc(-n5c6ccccc6c6cc7c8ccccc8n(-c8ccccc8)c7cc65)c4)c3)n2)cc1 ; c1ccc(-c2nc(-c3ccccc3)nc(-c3cccc(-c4cccc(-n5c6ccccc6c6ccc7c8ccccc8n(-c8ccccc8)c7c65)c4)c3)n2)cc1]

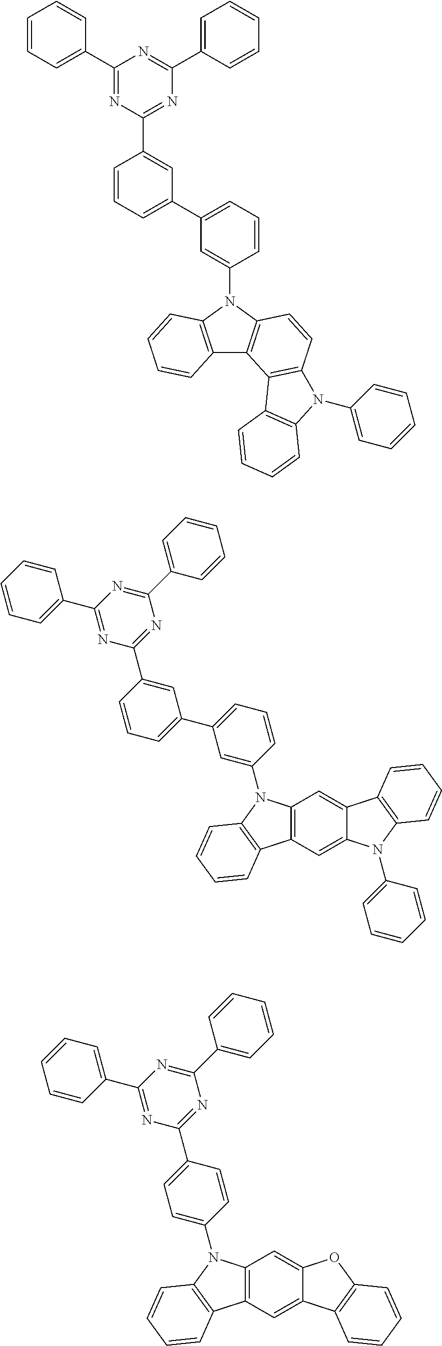 [SMILES: c1ccc(-c2nc(-c3ccccc3)nc(-c3cccc(-c4cccc(-n5c6ccccc6c6c7c8ccccc8n(-c8ccccc8)c7ccc65)c4)c3)n2)cc1 ; c1ccc(-c2nc(-c3ccccc3)nc(-c3cccc(-c4cccc(-n5c6ccccc6c6cc7c(cc65)c5ccccc5n7-c5ccccc5)c4)c3)n2)cc1 ; c1ccc(-c2nc(-c3ccccc3)nc(-c3ccc(-n4c5ccccc5c5cc6c(cc54)oc4ccccc46)cc3)n2)cc1]

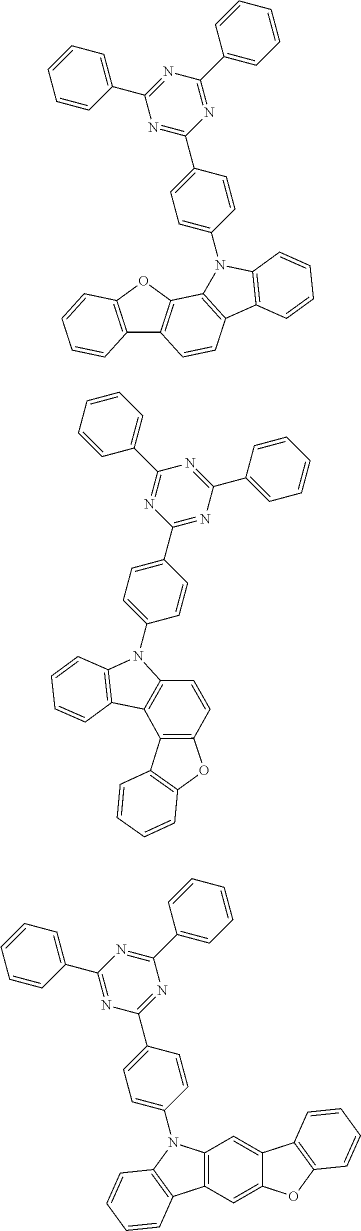

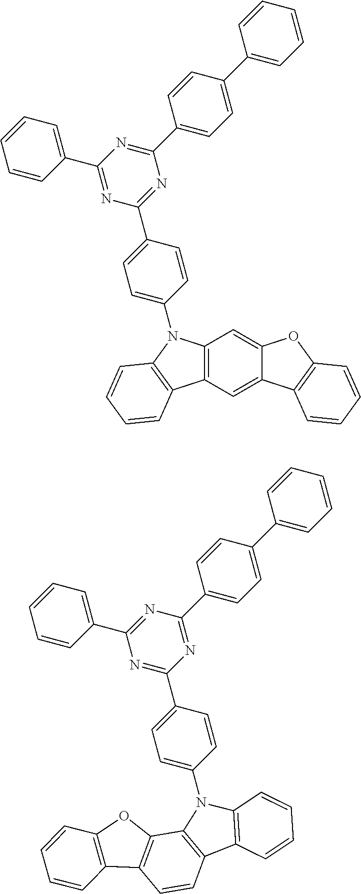 [SMILES: c1ccc(-c2ccc(-c3nc(-c4ccccc4)nc(-c4ccc(-n5c6ccccc6c6cc7c(cc65)oc5ccccc57)cc4)n3)cc2)cc1 ; c1ccc(-c2ccc(-c3nc(-c4ccccc4)nc(-c4ccc(-n5c6ccccc6c6ccc7c8ccccc8oc7c65)cc4)n3)cc2)cc1]

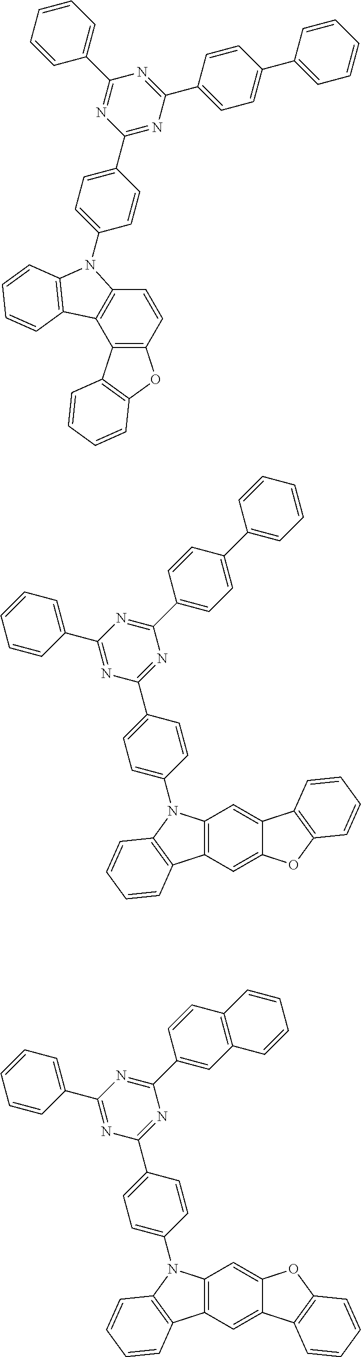 [SMILES: c1ccc(-c2ccc(-c3nc(-c4ccccc4)nc(-c4ccc(-n5c6ccccc6c6c7c(ccc65)oc5ccccc57)cc4)n3)cc2)cc1 ; c1ccc(-c2ccc(-c3nc(-c4ccccc4)nc(-c4ccc(-n5c6ccccc6c6cc7oc8ccccc8c7cc65)cc4)n3)cc2)cc1 ; c1ccc(-c2nc(-c3ccc(-n4c5ccccc5c5cc6c(cc54)oc4ccccc46)cc3)nc(-c3ccc4ccccc4c3)n2)cc1]

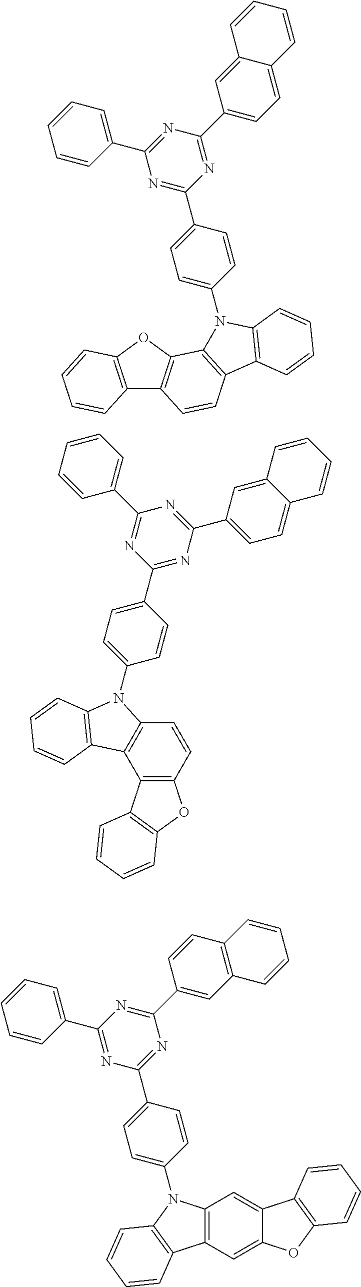

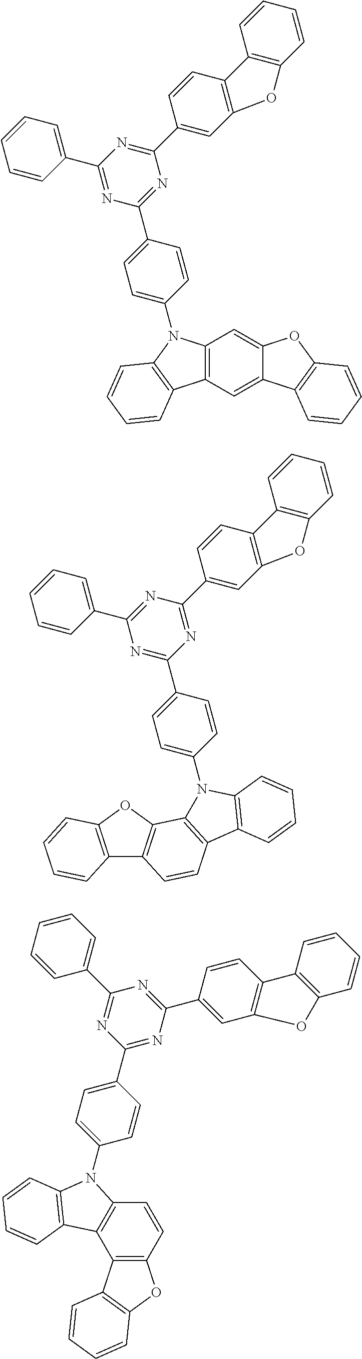

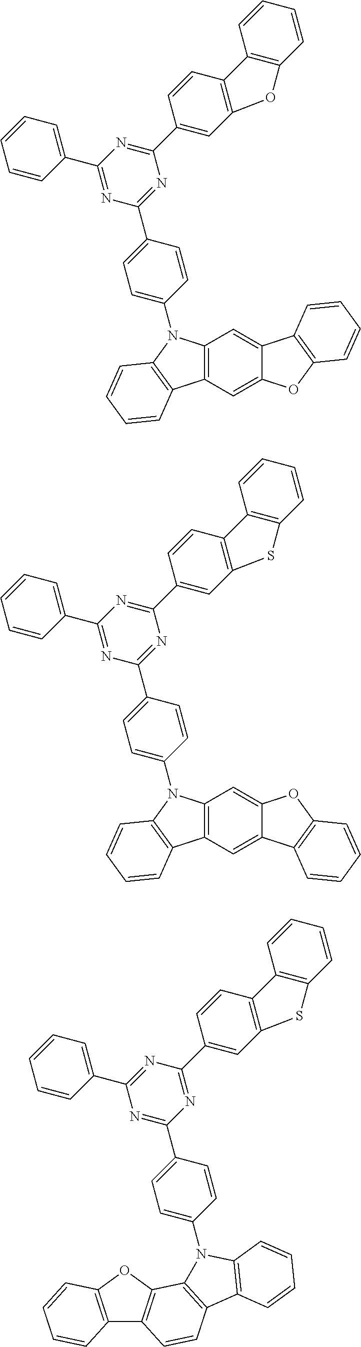

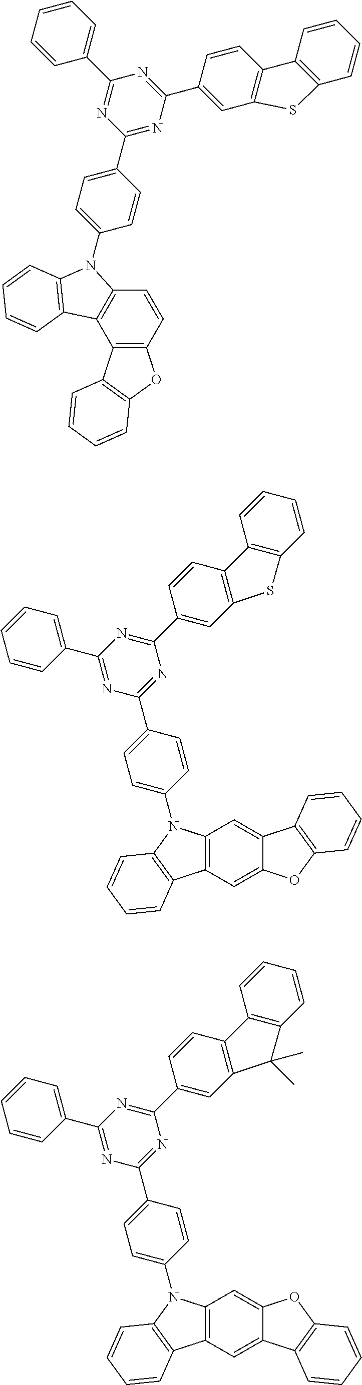 [SMILES: c1ccc(-c2nc(-c3ccc(-n4c5ccccc5c5c6c(ccc54)oc4ccccc46)cc3)nc(-c3ccc4c(c3)sc3ccccc34)n2)cc1 ; c1ccc(-c2nc(-c3ccc(-n4c5ccccc5c5cc6oc7ccccc7c6cc54)cc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)oc5ccccc57)cc4)n3)cc21]

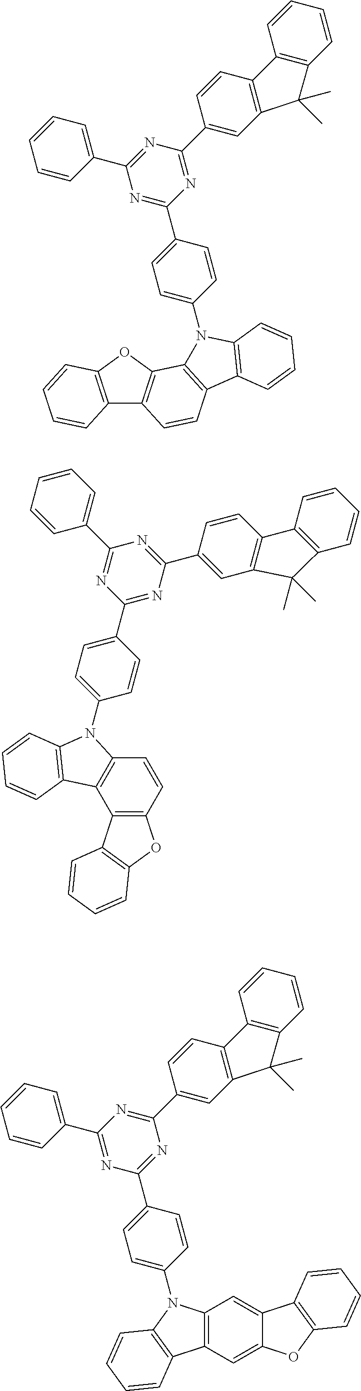

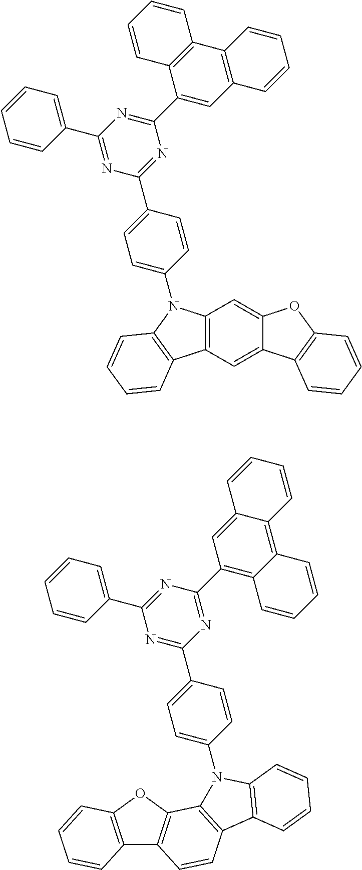 [SMILES: c1ccc(-c2nc(-c3ccc(-n4c5ccccc5c5cc6c(cc54)oc4ccccc46)cc3)nc(-c3cc4ccccc4c4ccccc34)n2)cc1 ; c1ccc(-c2nc(-c3ccc(-n4c5ccccc5c5ccc6c7ccccc7oc6c54)cc3)nc(-c3cc4ccccc4c4ccccc34)n2)cc1]

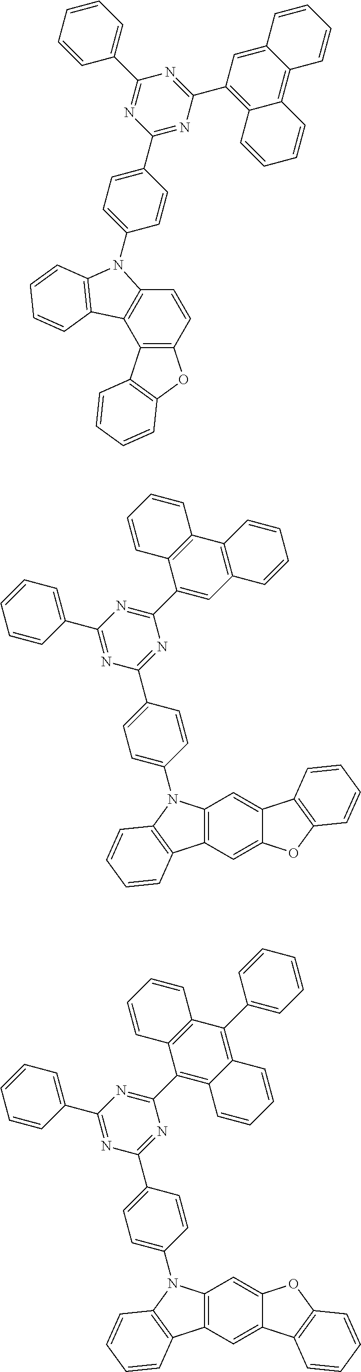 [SMILES: c1ccc(-c2nc(-c3ccc(-n4c5ccccc5c5c6c(ccc54)oc4ccccc46)cc3)nc(-c3cc4ccccc4c4ccccc34)n2)cc1 ; c1ccc(-c2nc(-c3ccc(-n4c5ccccc5c5cc6oc7ccccc7c6cc54)cc3)nc(-c3cc4ccccc4c4ccccc34)n2)cc1 ; c1ccc(-c2nc(-c3ccc(-n4c5ccccc5c5cc6c(cc54)oc4ccccc46)cc3)nc(-c3c4ccccc4c(-c4ccccc4)c4ccccc34)n2)cc1]

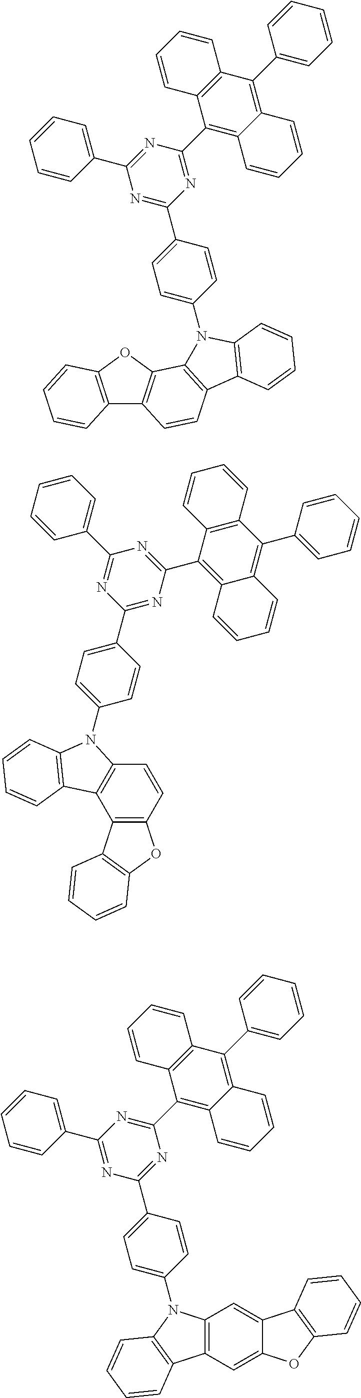 [SMILES: c1ccc(-c2nc(-c3ccc(-n4c5ccccc5c5ccc6c7ccccc7oc6c54)cc3)nc(-c3c4ccccc4c(-c4ccccc4)c4ccccc34)n2)cc1 ; c1ccc(-c2nc(-c3ccc(-n4c5ccccc5c5c6c(ccc54)oc4ccccc46)cc3)nc(-c3c4ccccc4c(-c4ccccc4)c4ccccc34)n2)cc1 ; c1ccc(-c2nc(-c3ccc(-n4c5ccccc5c5cc6oc7ccccc7c6cc54)cc3)nc(-c3c4ccccc4c(-c4ccccc4)c4ccccc34)n2)cc1]

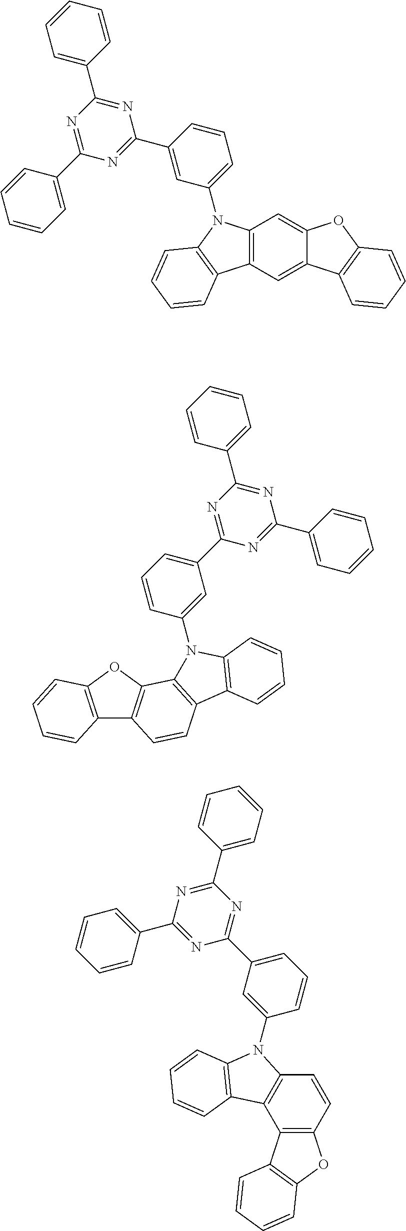 [SMILES: c1ccc(-c2nc(-c3ccccc3)nc(-c3cccc(-n4c5ccccc5c5cc6c(cc54)oc4ccccc46)c3)n2)cc1 ; c1ccc(-c2nc(-c3ccccc3)nc(-c3cccc(-n4c5ccccc5c5ccc6c7ccccc7oc6c54)c3)n2)cc1 ; c1ccc(-c2nc(-c3ccccc3)nc(-c3cccc(-n4c5ccccc5c5c6c(ccc54)oc4ccccc46)c3)n2)cc1]

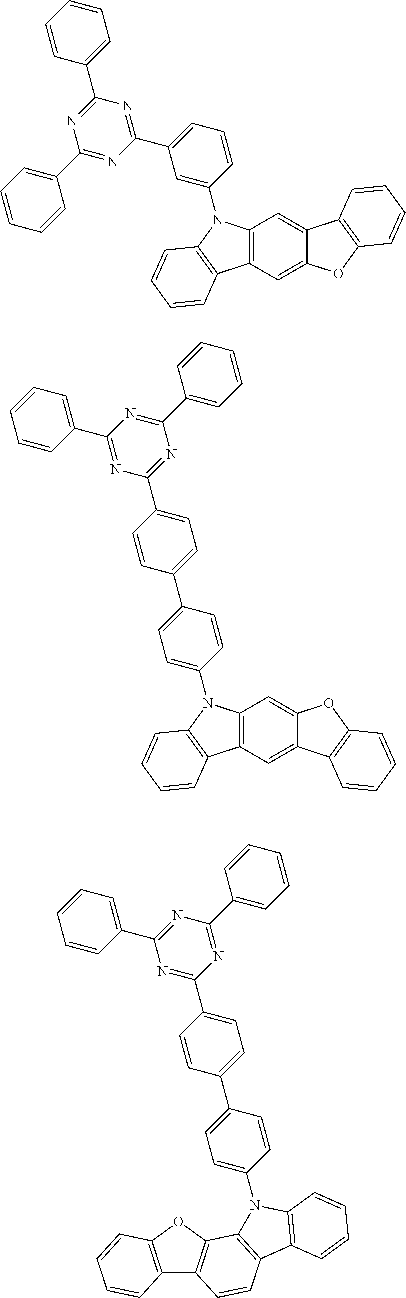

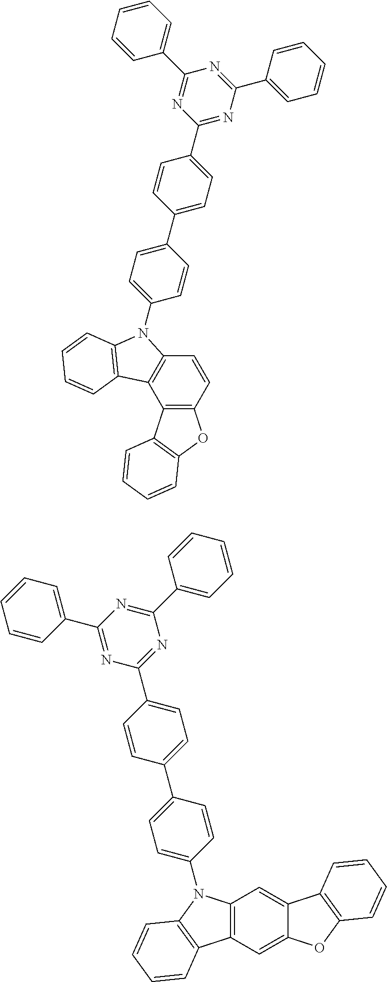 [SMILES: c1ccc(-c2nc(-c3ccccc3)nc(-c3ccc(-c4ccc(-n5c6ccccc6c6c7c(ccc65)oc5ccccc57)cc4)cc3)n2)cc1 ; c1ccc(-c2nc(-c3ccccc3)nc(-c3ccc(-c4ccc(-n5c6ccccc6c6cc7oc8ccccc8c7cc65)cc4)cc3)n2)cc1]

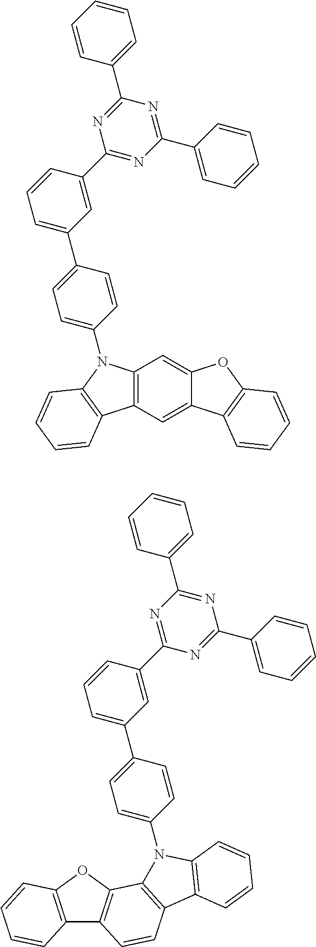 [SMILES: c1ccc(-c2nc(-c3ccccc3)nc(-c3cccc(-c4ccc(-n5c6ccccc6c6cc7c(cc65)oc5ccccc57)cc4)c3)n2)cc1 ; c1ccc(-c2nc(-c3ccccc3)nc(-c3cccc(-c4ccc(-n5c6ccccc6c6ccc7c8ccccc8oc7c65)cc4)c3)n2)cc1]

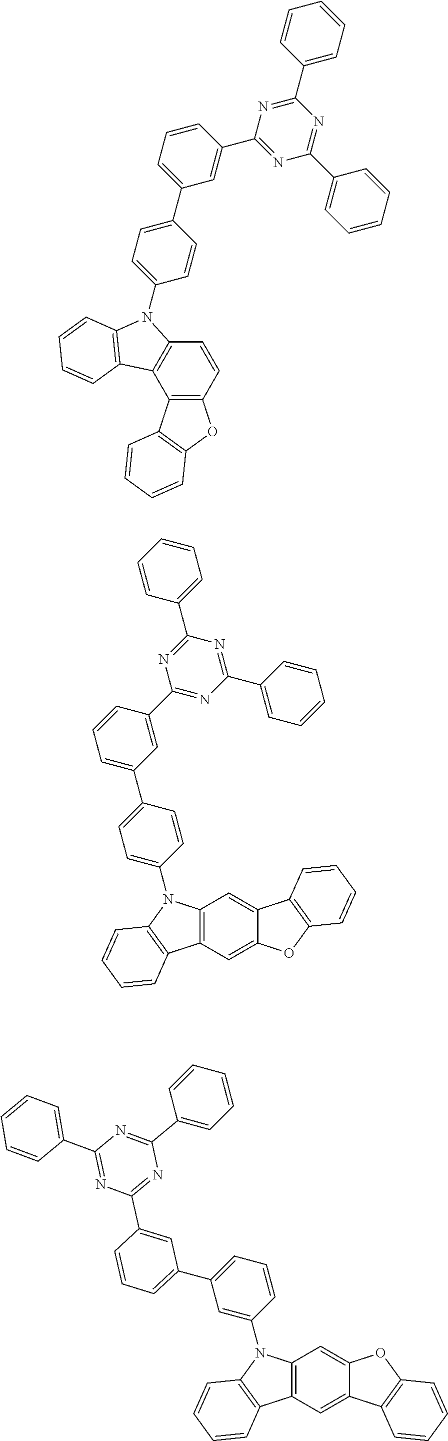

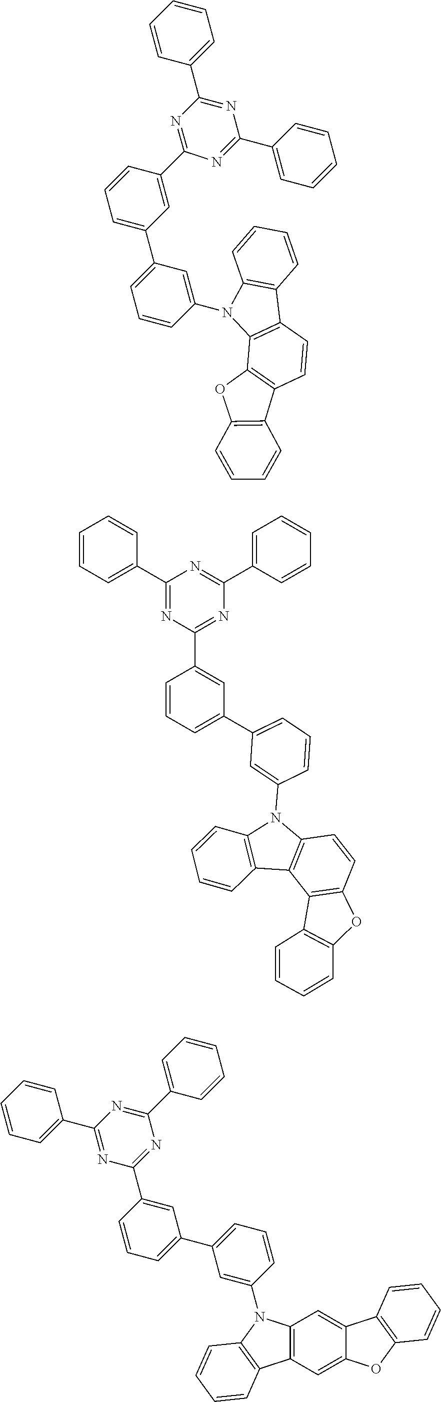

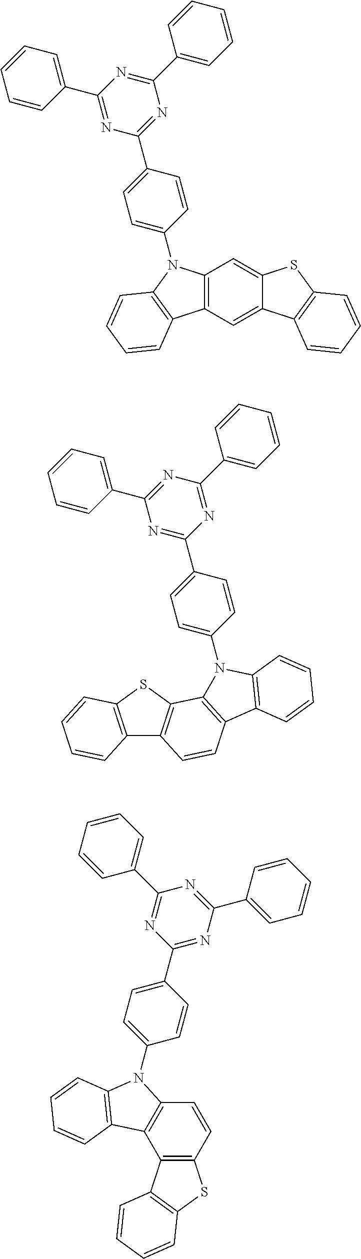 [SMILES: c1ccc(-c2nc(-c3ccccc3)nc(-c3ccc(-n4c5ccccc5c5cc6c(cc54)sc4ccccc46)cc3)n2)cc1 ; c1ccc(-c2nc(-c3ccccc3)nc(-c3ccc(-n4c5ccccc5c5ccc6c7ccccc7sc6c54)cc3)n2)cc1 ; c1ccc(-c2nc(-c3ccccc3)nc(-c3ccc(-n4c5ccccc5c5c6c(ccc54)sc4ccccc46)cc3)n2)cc1]

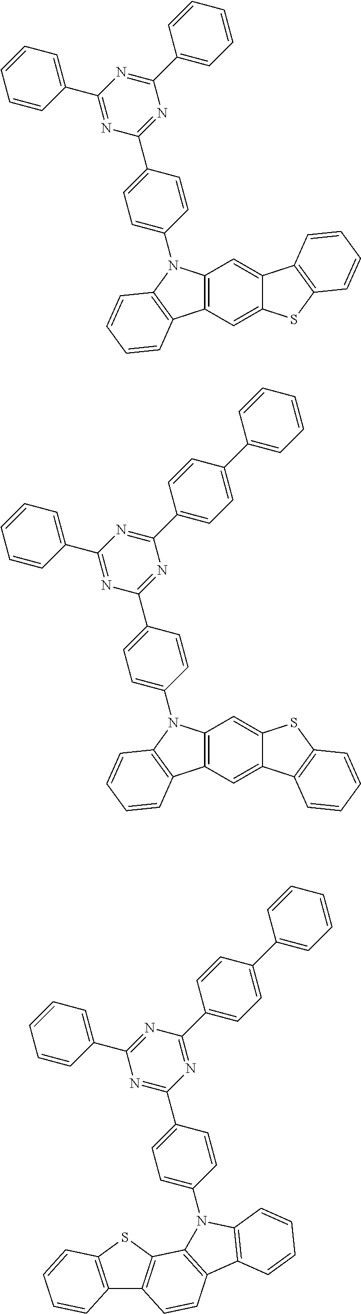 [SMILES: c1ccc(-c2nc(-c3ccccc3)nc(-c3ccc(-n4c5ccccc5c5cc6sc7ccccc7c6cc54)cc3)n2)cc1 ; c1ccc(-c2ccc(-c3nc(-c4ccccc4)nc(-c4ccc(-n5c6ccccc6c6cc7c(cc65)sc5ccccc57)cc4)n3)cc2)cc1 ; c1ccc(-c2ccc(-c3nc(-c4ccccc4)nc(-c4ccc(-n5c6ccccc6c6ccc7c8ccccc8sc7c65)cc4)n3)cc2)cc1]

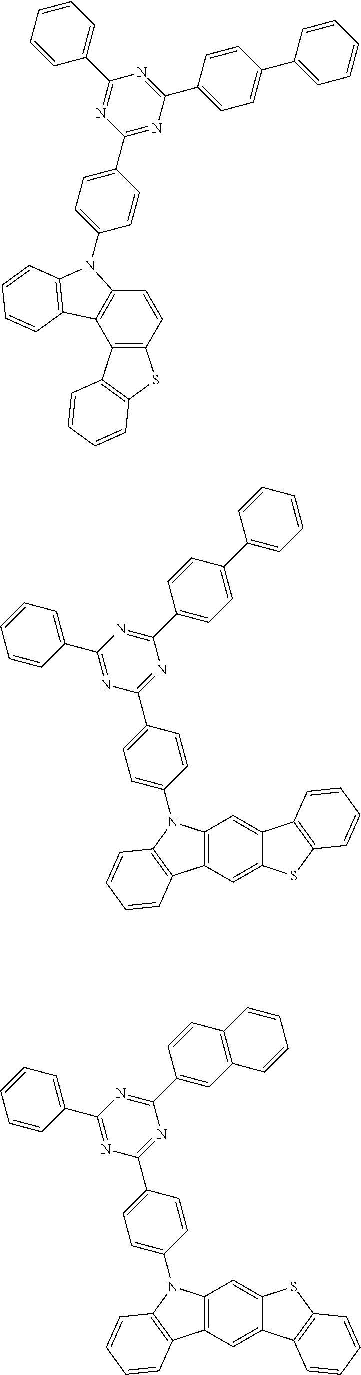 [SMILES: c1ccc(-c2ccc(-c3nc(-c4ccccc4)nc(-c4ccc(-n5c6ccccc6c6c7c(ccc65)sc5ccccc57)cc4)n3)cc2)cc1 ; c1ccc(-c2ccc(-c3nc(-c4ccccc4)nc(-c4ccc(-n5c6ccccc6c6cc7sc8ccccc8c7cc65)cc4)n3)cc2)cc1 ; c1ccc(-c2nc(-c3ccc(-n4c5ccccc5c5cc6c(cc54)sc4ccccc46)cc3)nc(-c3ccc4ccccc4c3)n2)cc1]

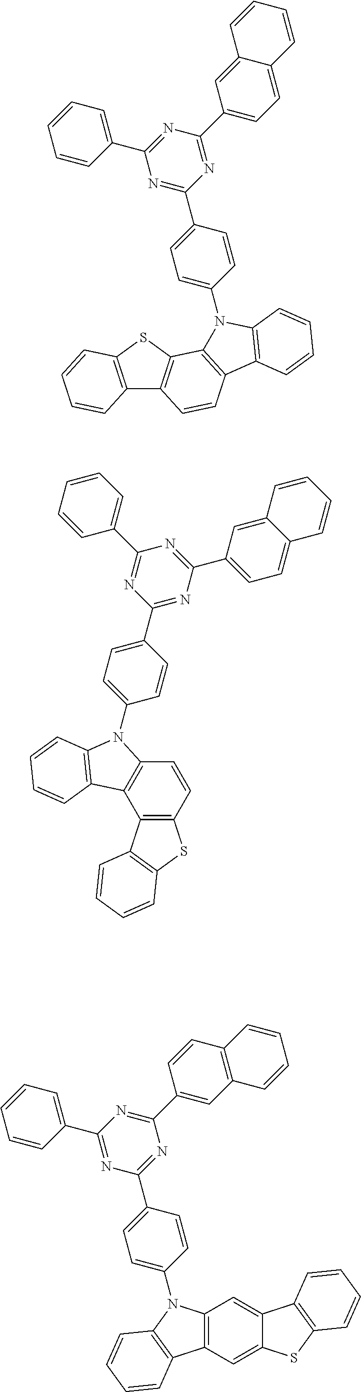 [SMILES: c1ccc(-c2nc(-c3ccc(-n4c5ccccc5c5ccc6c7ccccc7sc6c54)cc3)nc(-c3ccc4ccccc4c3)n2)cc1 ; c1ccc(-c2nc(-c3ccc(-n4c5ccccc5c5c6c(ccc54)sc4ccccc46)cc3)nc(-c3ccc4ccccc4c3)n2)cc1 ; c1ccc(-c2nc(-c3ccc(-n4c5ccccc5c5cc6sc7ccccc7c6cc54)cc3)nc(-c3ccc4ccccc4c3)n2)cc1]

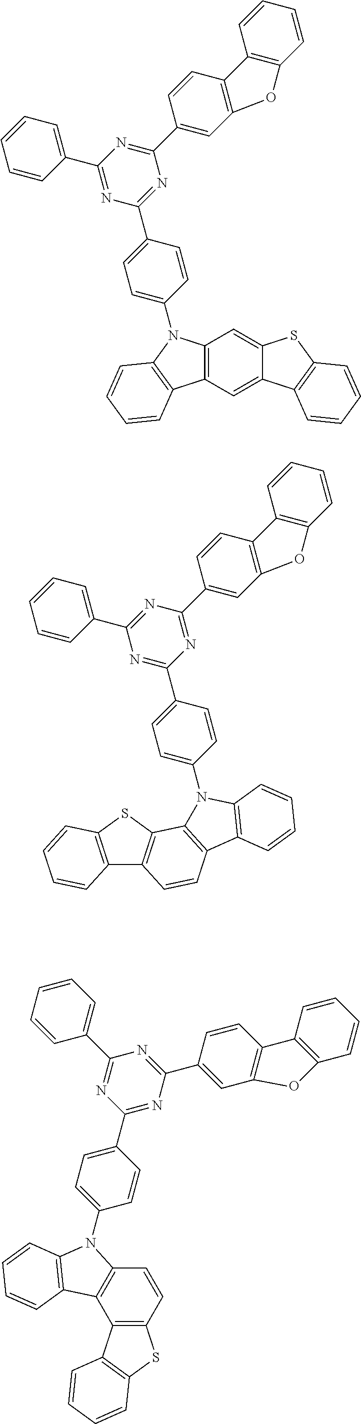

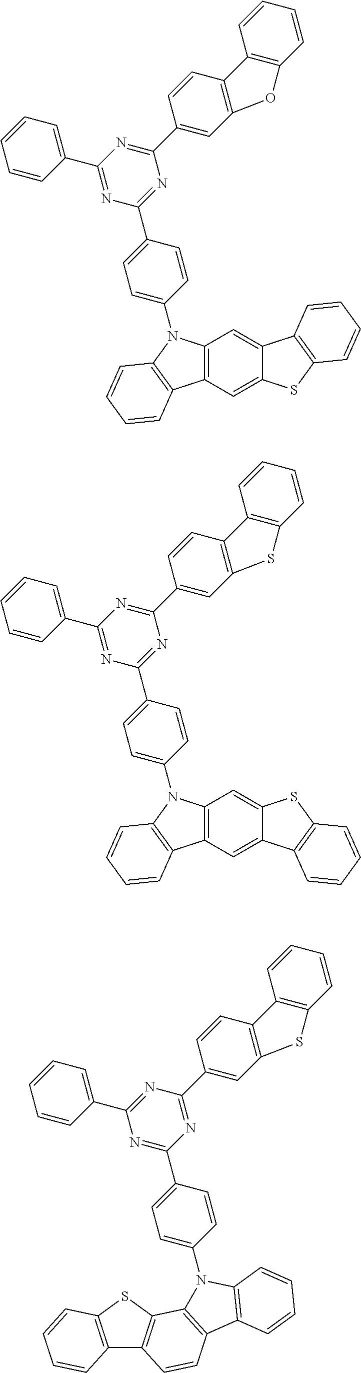 [SMILES: c1ccc(-c2nc(-c3ccc(-n4c5ccccc5c5cc6sc7ccccc7c6cc54)cc3)nc(-c3ccc4c(c3)oc3ccccc34)n2)cc1 ; c1ccc(-c2nc(-c3ccc(-n4c5ccccc5c5cc6c(cc54)sc4ccccc46)cc3)nc(-c3ccc4c(c3)sc3ccccc34)n2)cc1 ; c1ccc(-c2nc(-c3ccc(-n4c5ccccc5c5ccc6c7ccccc7sc6c54)cc3)nc(-c3ccc4c(c3)sc3ccccc34)n2)cc1]

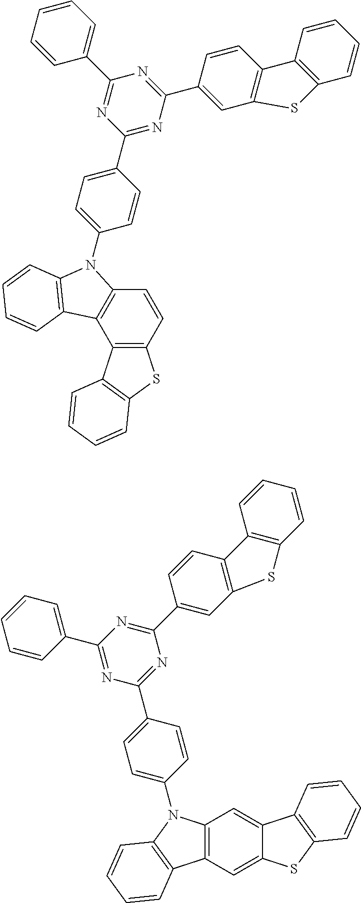 [SMILES: c1ccc(-c2nc(-c3ccc(-n4c5ccccc5c5c6c(ccc54)sc4ccccc46)cc3)nc(-c3ccc4c(c3)sc3ccccc34)n2)cc1 ; c1ccc(-c2nc(-c3ccc(-n4c5ccccc5c5cc6sc7ccccc7c6cc54)cc3)nc(-c3ccc4c(c3)sc3ccccc34)n2)cc1]

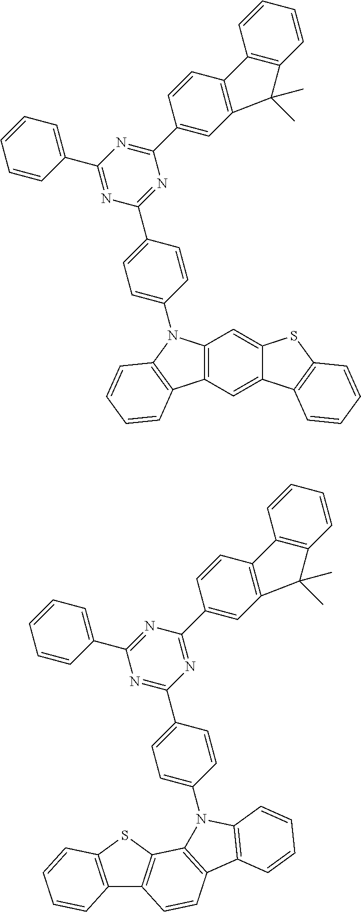 [SMILES: CC1(C)c2ccccc2-c2ccc(-c3nc(-c4ccccc4)nc(-c4ccc(-n5c6ccccc6c6cc7c(cc65)sc5ccccc57)cc4)n3)cc21 ; CC1(C)c2ccccc2-c2ccc(-c3nc(-c4ccccc4)nc(-c4ccc(-n5c6ccccc6c6ccc7c8ccccc8sc7c65)cc4)n3)cc21]

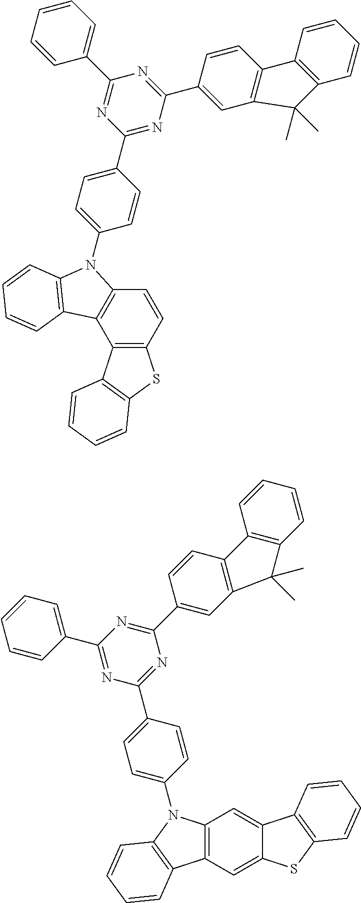 [SMILES: CC1(C)c2ccccc2-c2ccc(-c3nc(-c4ccccc4)nc(-c4ccc(-n5c6ccccc6c6c7c(ccc65)sc5ccccc57)cc4)n3)cc21 ; CC1(C)c2ccccc2-c2ccc(-c3nc(-c4ccccc4)nc(-c4ccc(-n5c6ccccc6c6cc7sc8ccccc8c7cc65)cc4)n3)cc21]

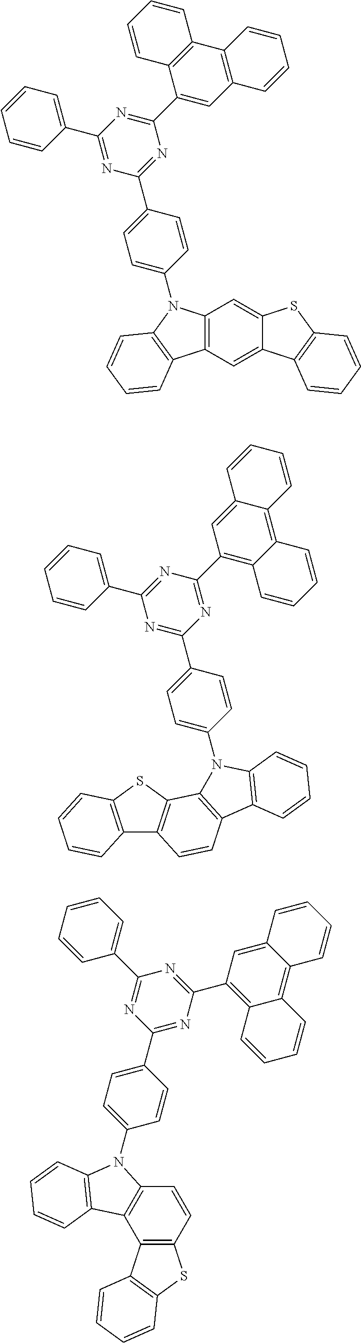

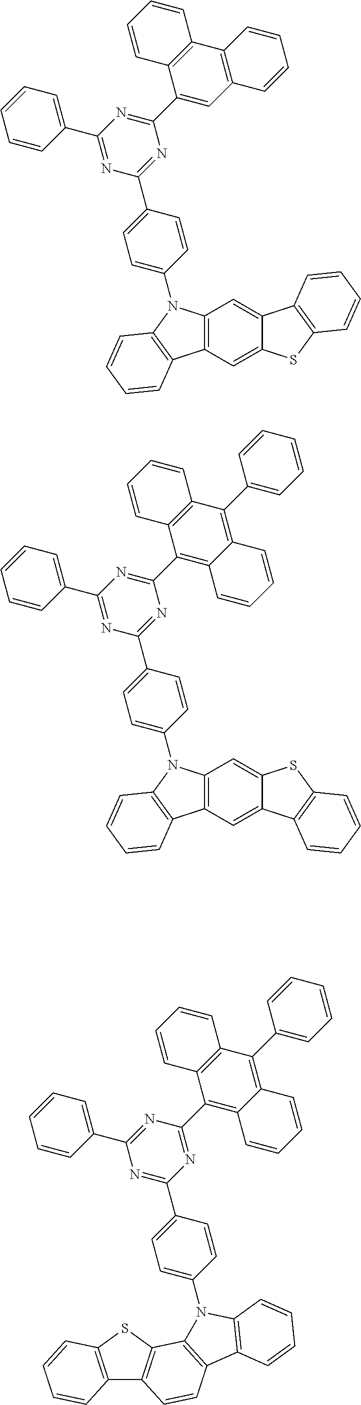 [SMILES: c1ccc(-c2nc(-c3ccc(-n4c5ccccc5c5cc6sc7ccccc7c6cc54)cc3)nc(-c3cc4ccccc4c4ccccc34)n2)cc1 ; c1ccc(-c2nc(-c3ccc(-n4c5ccccc5c5cc6c(cc54)sc4ccccc46)cc3)nc(-c3c4ccccc4c(-c4ccccc4)c4ccccc34)n2)cc1 ; c1ccc(-c2nc(-c3ccc(-n4c5ccccc5c5ccc6c7ccccc7sc6c54)cc3)nc(-c3c4ccccc4c(-c4ccccc4)c4ccccc34)n2)cc1]

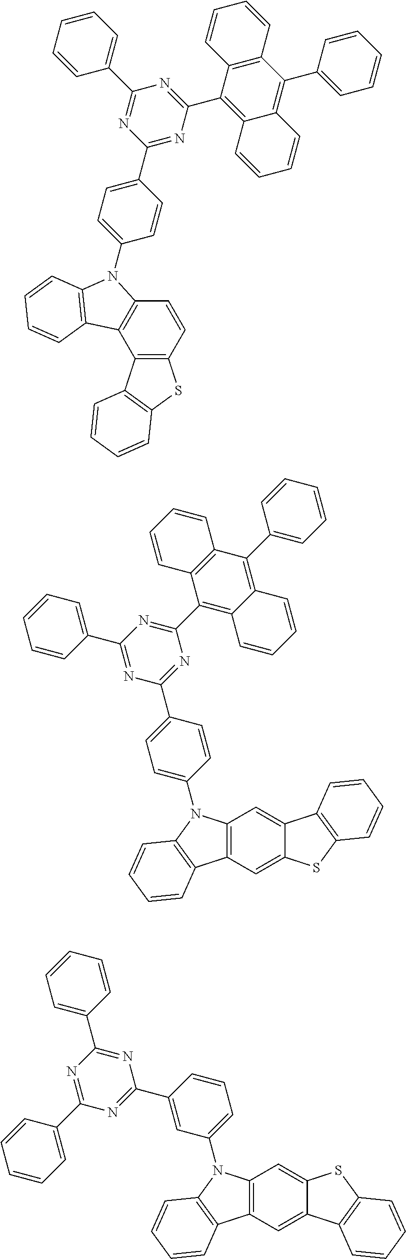 [SMILES: c1ccc(-c2nc(-c3ccc(-n4c5ccccc5c5c6c(ccc54)sc4ccccc46)cc3)nc(-c3c4ccccc4c(-c4ccccc4)c4ccccc34)n2)cc1 ; c1ccc(-c2nc(-c3ccc(-n4c5ccccc5c5cc6sc7ccccc7c6cc54)cc3)nc(-c3c4ccccc4c(-c4ccccc4)c4ccccc34)n2)cc1 ; c1ccc(-c2nc(-c3ccccc3)nc(-c3cccc(-n4c5ccccc5c5cc6c(cc54)sc4ccccc46)c3)n2)cc1]

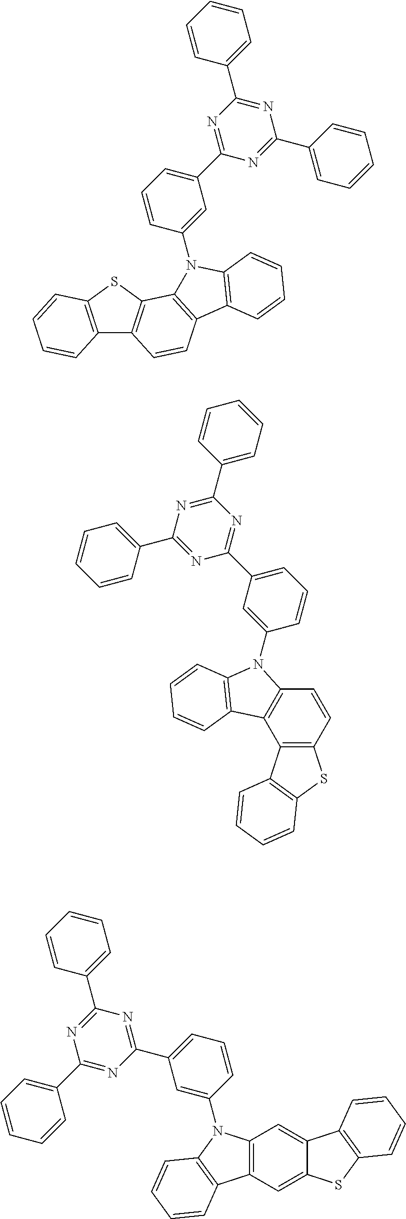 [SMILES: c1ccc(-c2nc(-c3ccccc3)nc(-c3cccc(-n4c5ccccc5c5ccc6c7ccccc7sc6c54)c3)n2)cc1 ; c1ccc(-c2nc(-c3ccccc3)nc(-c3cccc(-n4c5ccccc5c5c6c(ccc54)sc4ccccc46)c3)n2)cc1 ; c1ccc(-c2nc(-c3ccccc3)nc(-c3cccc(-n4c5ccccc5c5cc6sc7ccccc7c6cc54)c3)n2)cc1]

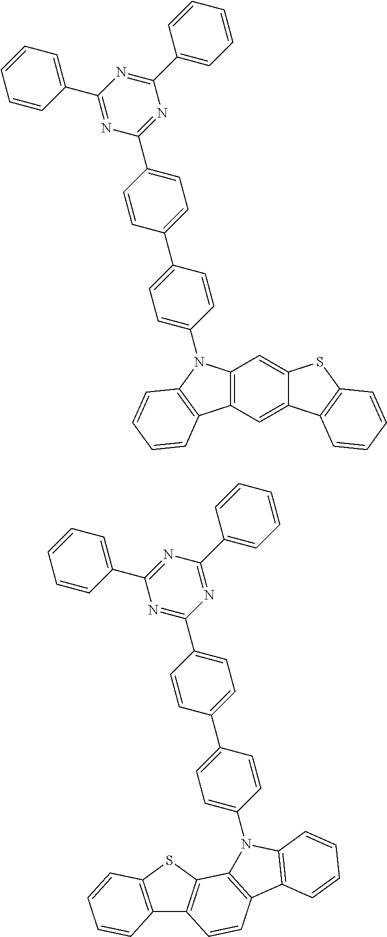 [SMILES: c1ccc(-c2nc(-c3ccccc3)nc(-c3ccc(-c4ccc(-n5c6ccccc6c6cc7c(cc65)sc5ccccc57)cc4)cc3)n2)cc1 ; c1ccc(-c2nc(-c3ccccc3)nc(-c3ccc(-c4ccc(-n5c6ccccc6c6ccc7c8ccccc8sc7c65)cc4)cc3)n2)cc1]

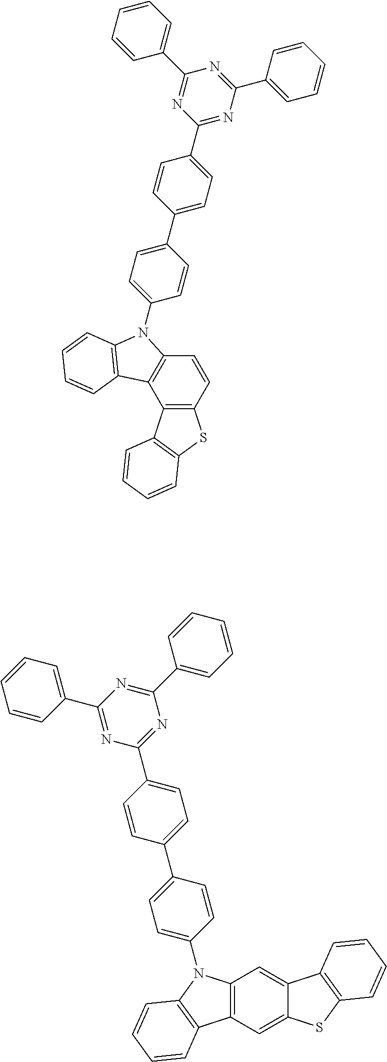 [SMILES: c1ccc(-c2nc(-c3ccccc3)nc(-c3ccc(-c4ccc(-n5c6ccccc6c6c7c(ccc65)sc5ccccc57)cc4)cc3)n2)cc1 ; c1ccc(-c2nc(-c3ccccc3)nc(-c3ccc(-c4ccc(-n5c6ccccc6c6cc7sc8ccccc8c7cc65)cc4)cc3)n2)cc1]

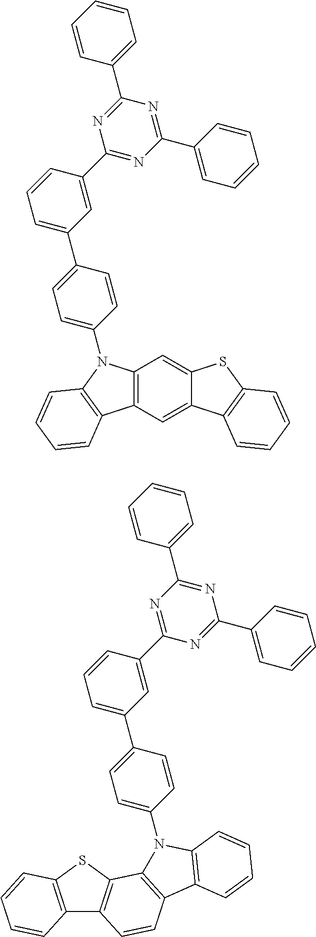 [SMILES: c1ccc(-c2nc(-c3ccccc3)nc(-c3cccc(-c4ccc(-n5c6ccccc6c6cc7c(cc65)sc5ccccc57)cc4)c3)n2)cc1 ; c1ccc(-c2nc(-c3ccccc3)nc(-c3cccc(-c4ccc(-n5c6ccccc6c6ccc7c8ccccc8sc7c65)cc4)c3)n2)cc1]

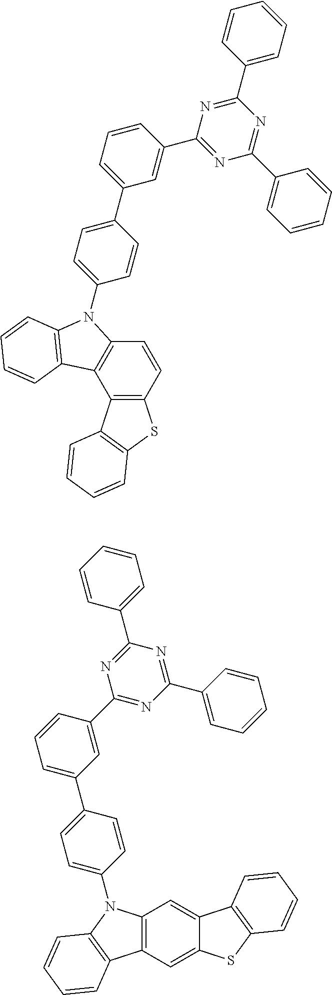 [SMILES: c1ccc(-c2nc(-c3ccccc3)nc(-c3cccc(-c4ccc(-n5c6ccccc6c6c7c(ccc65)sc5ccccc57)cc4)c3)n2)cc1 ; c1ccc(-c2nc(-c3ccccc3)nc(-c3cccc(-c4ccc(-n5c6ccccc6c6cc7sc8ccccc8c7cc65)cc4)c3)n2)cc1]

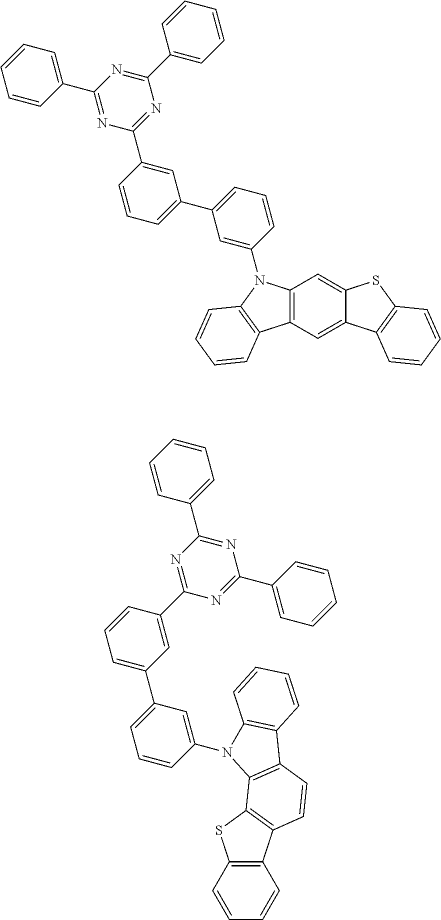 [SMILES: c1ccc(-c2nc(-c3ccccc3)nc(-c3cccc(-c4cccc(-n5c6ccccc6c6cc7c(cc65)sc5ccccc57)c4)c3)n2)cc1 ; c1ccc(-c2nc(-c3ccccc3)nc(-c3cccc(-c4cccc(-n5c6ccccc6c6ccc7c8ccccc8sc7c65)c4)c3)n2)cc1]

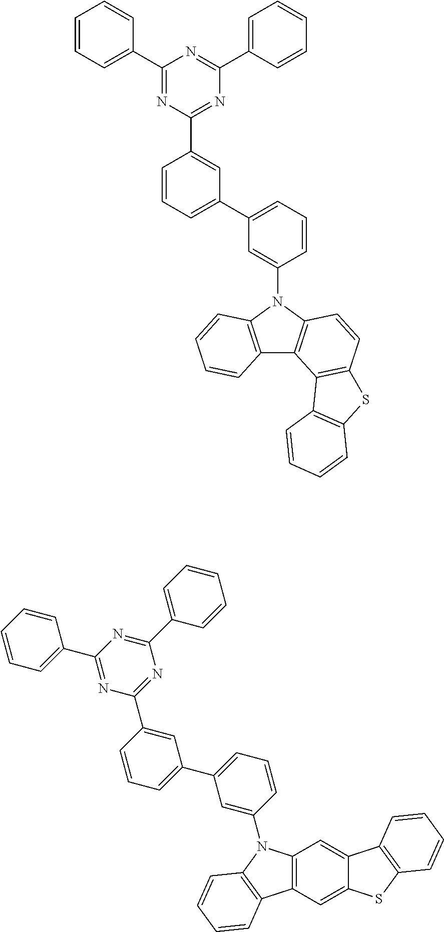 [SMILES: c1ccc(-c2nc(-c3ccccc3)nc(-c3cccc(-c4cccc(-n5c6ccccc6c6c7c(ccc65)sc5ccccc57)c4)c3)n2)cc1 ; c1ccc(-c2nc(-c3ccccc3)nc(-c3cccc(-c4cccc(-n5c6ccccc6c6cc7sc8ccccc8c7cc65)c4)c3)n2)cc1]

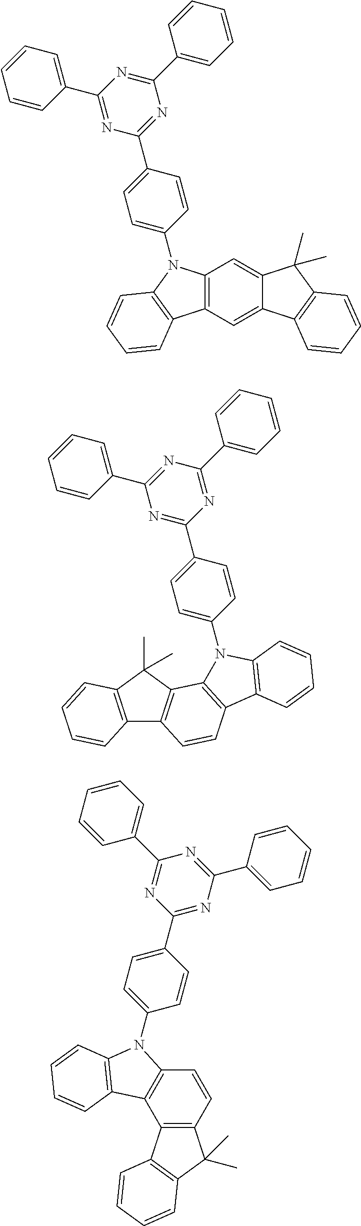

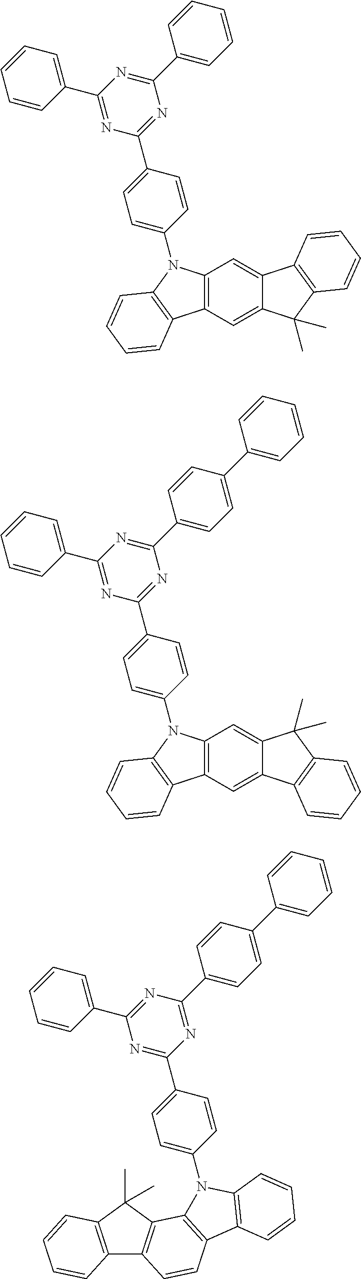

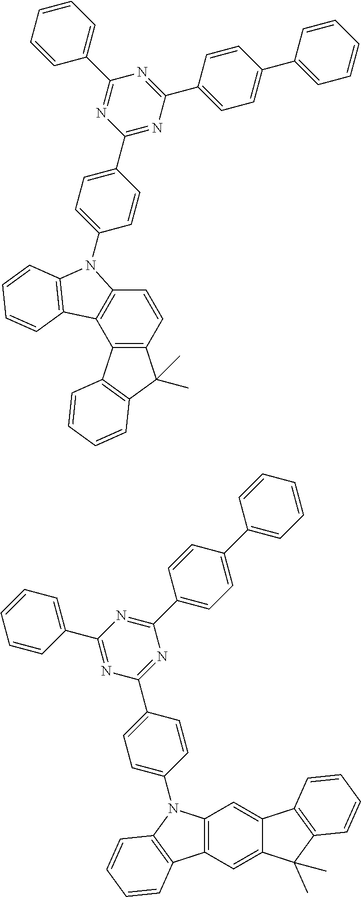 [SMILES: CC1(C)c2ccccc2-c2c1ccc1c2c2ccccc2n1-c1ccc(-c2nc(-c3ccccc3)nc(-c3ccc(-c4ccccc4)cc3)n2)cc1 ; CC1(C)c2ccccc2-c2cc3c(cc21)c1ccccc1n3-c1ccc(-c2nc(-c3ccccc3)nc(-c3ccc(-c4ccccc4)cc3)n2)cc1]

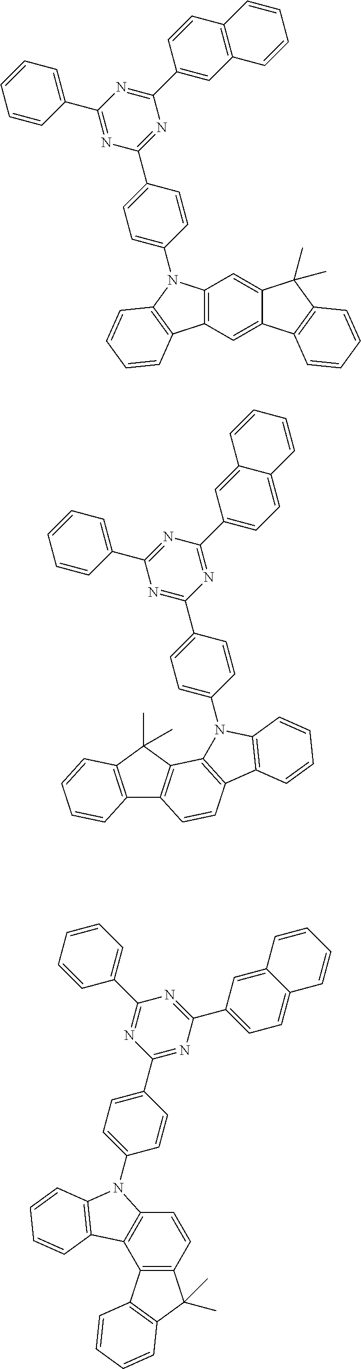 [SMILES: CC1(C)c2ccccc2-c2cc3c4ccccc4n(-c4ccc(-c5nc(-c6ccccc6)nc(-c6ccc7ccccc7c6)n5)cc4)c3cc21 ; CC1(C)c2ccccc2-c2ccc3c4ccccc4n(-c4ccc(-c5nc(-c6ccccc6)nc(-c6ccc7ccccc7c6)n5)cc4)c3c21 ; CC1(C)c2ccccc2-c2c1ccc1c2c2ccccc2n1-c1ccc(-c2nc(-c3ccccc3)nc(-c3ccc4ccccc4c3)n2)cc1]

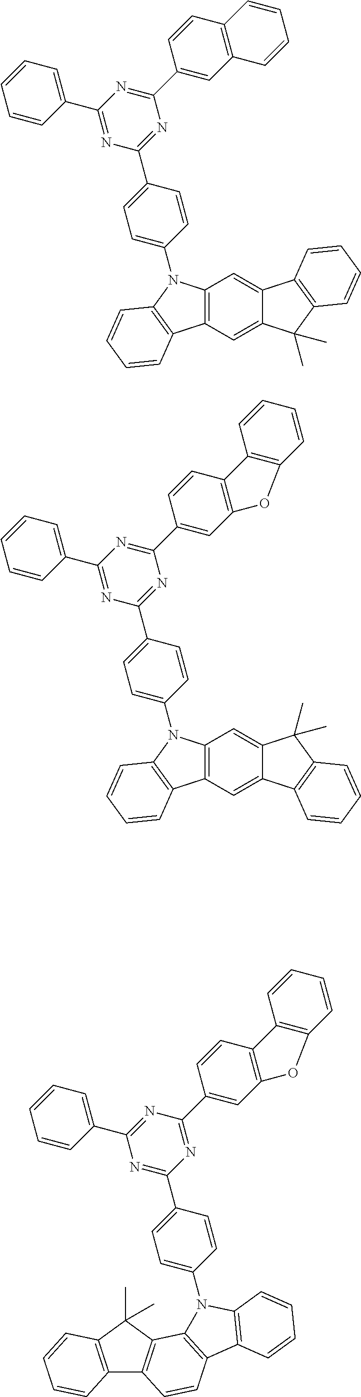

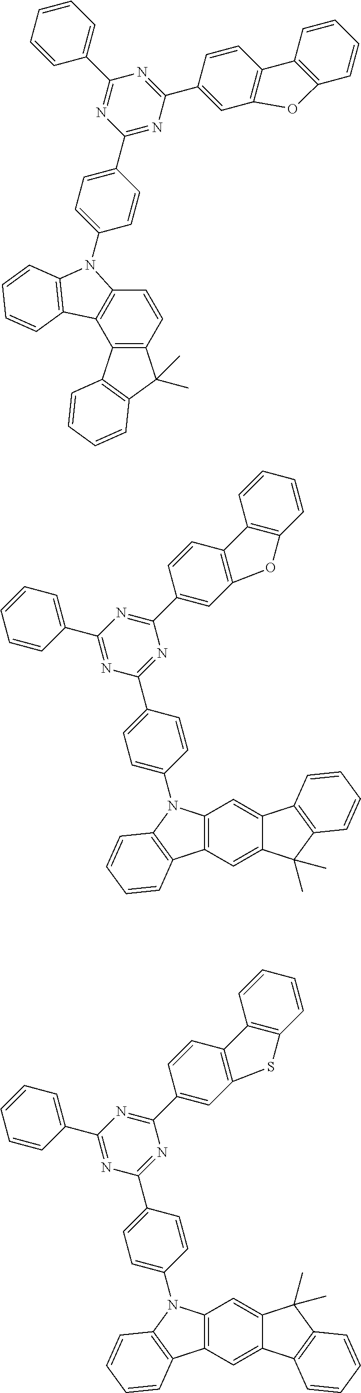 [SMILES: CC1(C)c2ccccc2-c2c1ccc1c2c2ccccc2n1-c1ccc(-c2nc(-c3ccccc3)nc(-c3ccc4c(c3)oc3ccccc34)n2)cc1 ; CC1(C)c2ccccc2-c2cc3c(cc21)c1ccccc1n3-c1ccc(-c2nc(-c3ccccc3)nc(-c3ccc4c(c3)oc3ccccc34)n2)cc1 ; CC1(C)c2ccccc2-c2cc3c4ccccc4n(-c4ccc(-c5nc(-c6ccccc6)nc(-c6ccc7c(c6)sc6ccccc67)n5)cc4)c3cc21]

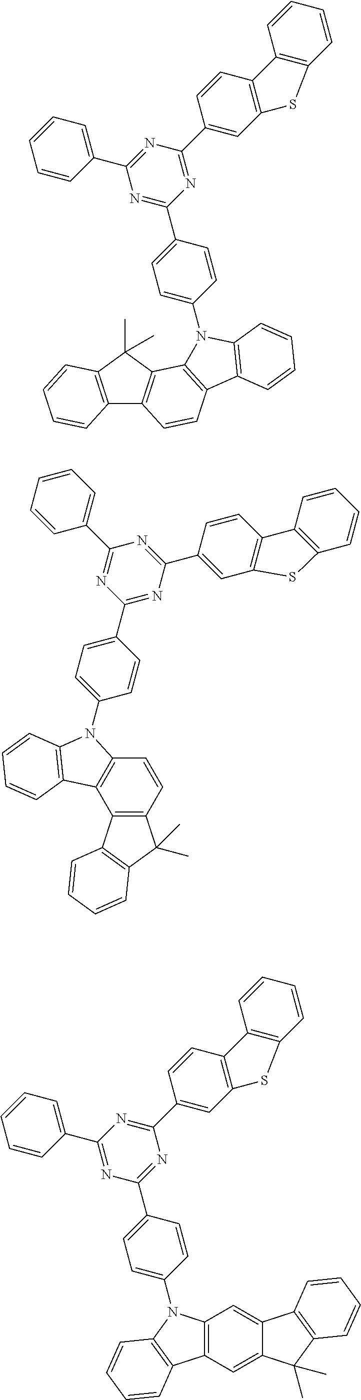

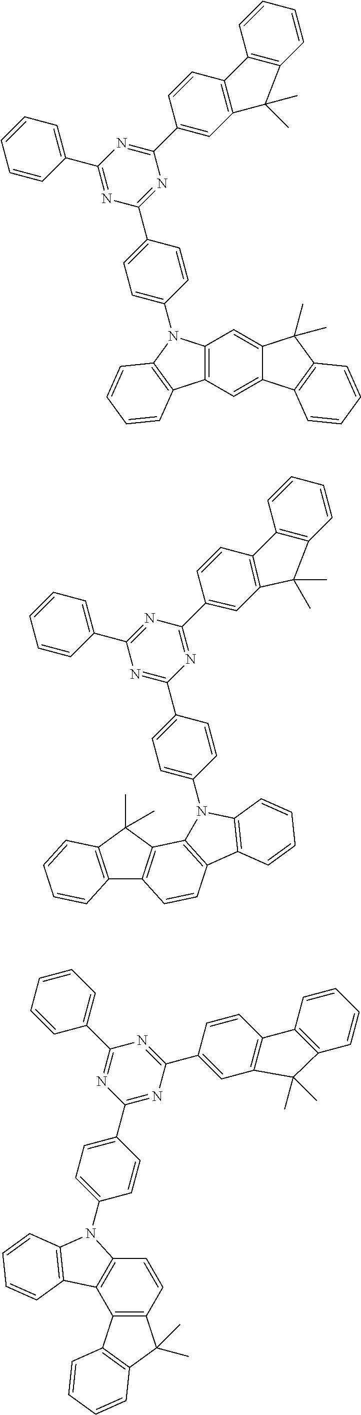 [SMILES: CC1(C)c2ccccc2-c2ccc(-c3nc(-c4ccccc4)nc(-c4ccc(-n5c6ccccc6c6cc7c(cc65)C(C)(C)c5ccccc5-7)cc4)n3)cc21 ; CC1(C)c2ccccc2-c2ccc(-c3nc(-c4ccccc4)nc(-c4ccc(-n5c6ccccc6c6ccc7c(c65)C(C)(C)c5ccccc5-7)cc4)n3)cc21 ; CC1(C)c2ccccc2-c2ccc(-c3nc(-c4ccccc4)nc(-c4ccc(-n5c6ccccc6c6c7c(ccc65)C(C)(C)c5ccccc5-7)cc4)n3)cc21]

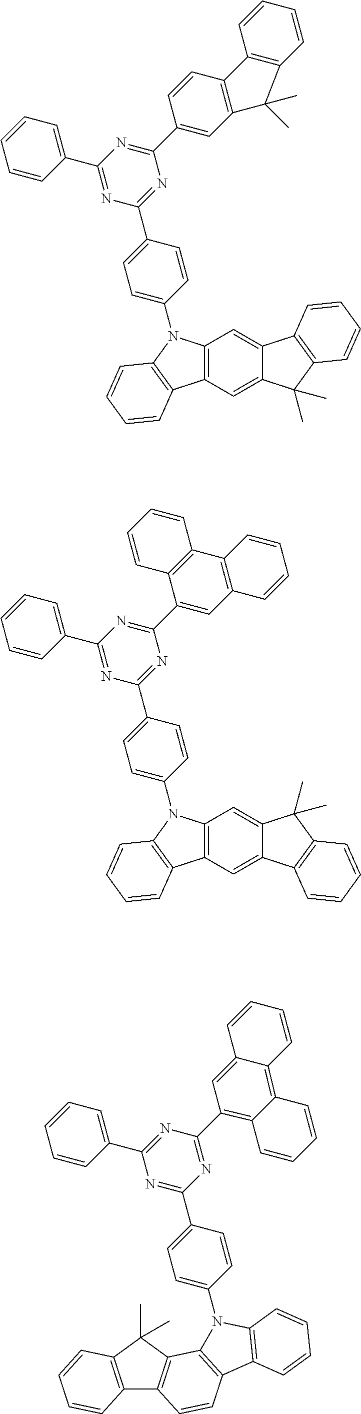 [SMILES: CC1(C)c2ccccc2-c2ccc(-c3nc(-c4ccccc4)nc(-c4ccc(-n5c6ccccc6c6cc7c(cc65)-c5ccccc5C7(C)C)cc4)n3)cc21 ; CC1(C)c2ccccc2-c2cc3c4ccccc4n(-c4ccc(-c5nc(-c6ccccc6)nc(-c6cc7ccccc7c7ccccc67)n5)cc4)c3cc21 ; CC1(C)c2ccccc2-c2ccc3c4ccccc4n(-c4ccc(-c5nc(-c6ccccc6)nc(-c6cc7ccccc7c7ccccc67)n5)cc4)c3c21]

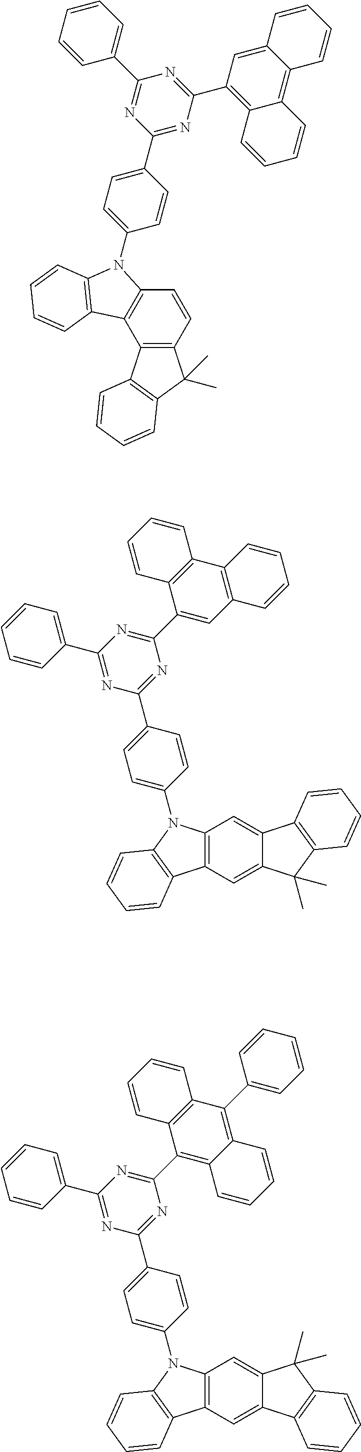 [SMILES: CC1(C)c2ccccc2-c2c1ccc1c2c2ccccc2n1-c1ccc(-c2nc(-c3ccccc3)nc(-c3cc4ccccc4c4ccccc34)n2)cc1 ; CC1(C)c2ccccc2-c2cc3c(cc21)c1ccccc1n3-c1ccc(-c2nc(-c3ccccc3)nc(-c3cc4ccccc4c4ccccc34)n2)cc1 ; CC1(C)c2ccccc2-c2cc3c4ccccc4n(-c4ccc(-c5nc(-c6ccccc6)nc(-c6c7ccccc7c(-c7ccccc7)c7ccccc67)n5)cc4)c3cc21]

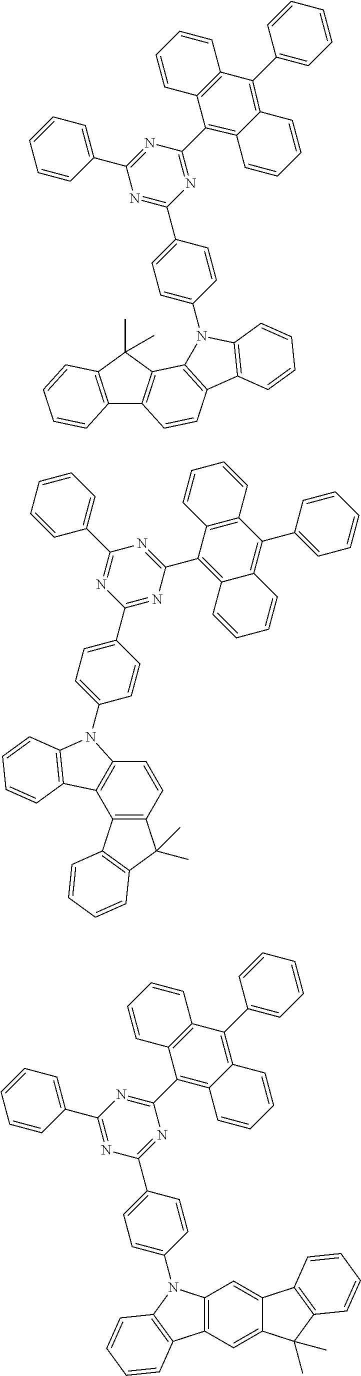 [SMILES: CC1(C)c2ccccc2-c2ccc3c4ccccc4n(-c4ccc(-c5nc(-c6ccccc6)nc(-c6c7ccccc7c(-c7ccccc7)c7ccccc67)n5)cc4)c3c21 ; CC1(C)c2ccccc2-c2c1ccc1c2c2ccccc2n1-c1ccc(-c2nc(-c3ccccc3)nc(-c3c4ccccc4c(-c4ccccc4)c4ccccc34)n2)cc1 ; CC1(C)c2ccccc2-c2cc3c(cc21)c1ccccc1n3-c1ccc(-c2nc(-c3ccccc3)nc(-c3c4ccccc4c(-c4ccccc4)c4ccccc34)n2)cc1]

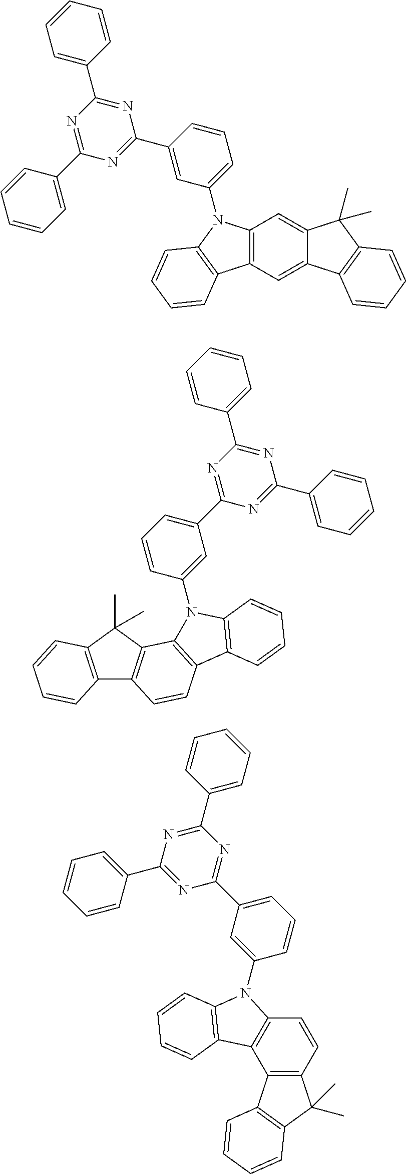

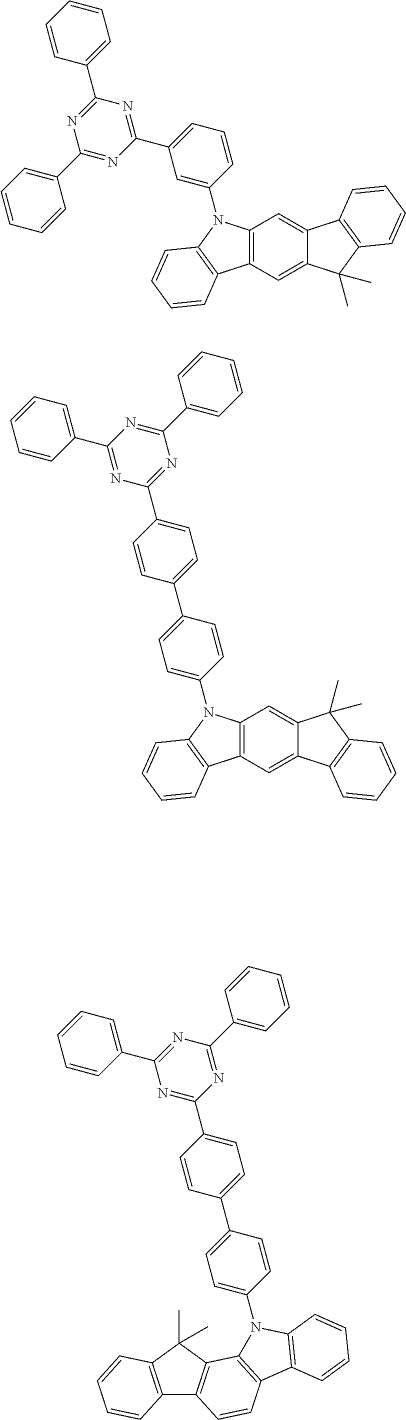 [SMILES: CC1(C)c2ccccc2-c2cc3c(cc21)c1ccccc1n3-c1cccc(-c2nc(-c3ccccc3)nc(-c3ccccc3)n2)c1 ; CC1(C)c2ccccc2-c2cc3c4ccccc4n(-c4ccc(-c5ccc(-c6nc(-c7ccccc7)nc(-c7ccccc7)n6)cc5)cc4)c3cc21 ; CC1(C)c2ccccc2-c2ccc3c4ccccc4n(-c4ccc(-c5ccc(-c6nc(-c7ccccc7)nc(-c7ccccc7)n6)cc5)cc4)c3c21]

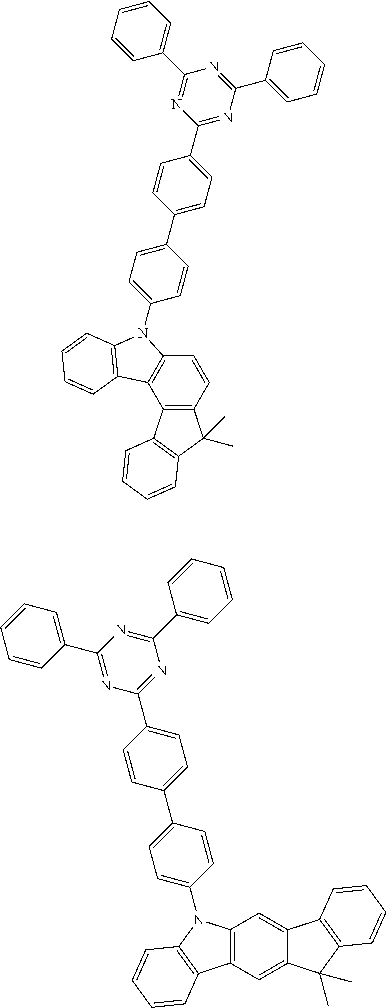 [SMILES: CC1(C)c2ccccc2-c2c1ccc1c2c2ccccc2n1-c1ccc(-c2ccc(-c3nc(-c4ccccc4)nc(-c4ccccc4)n3)cc2)cc1 ; CC1(C)c2ccccc2-c2cc3c(cc21)c1ccccc1n3-c1ccc(-c2ccc(-c3nc(-c4ccccc4)nc(-c4ccccc4)n3)cc2)cc1]

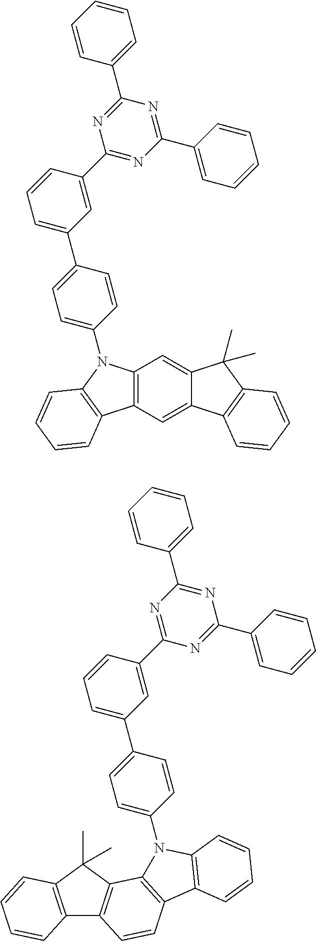 [SMILES: CC1(C)c2ccccc2-c2cc3c4ccccc4n(-c4ccc(-c5cccc(-c6nc(-c7ccccc7)nc(-c7ccccc7)n6)c5)cc4)c3cc21 ; CC1(C)c2ccccc2-c2ccc3c4ccccc4n(-c4ccc(-c5cccc(-c6nc(-c7ccccc7)nc(-c7ccccc7)n6)c5)cc4)c3c21]

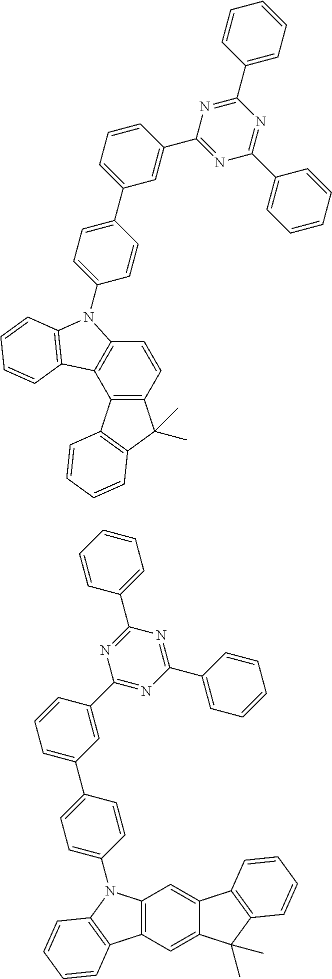

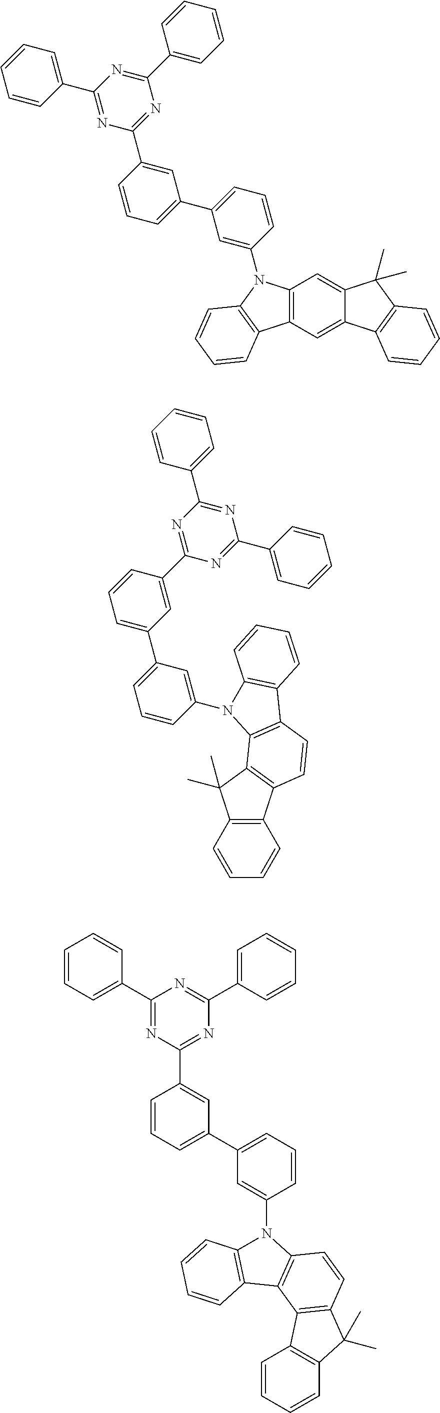 [SMILES: CC1(C)c2ccccc2-c2cc3c4ccccc4n(-c4cccc(-c5cccc(-c6nc(-c7ccccc7)nc(-c7ccccc7)n6)c5)c4)c3cc21 ; CC1(C)c2ccccc2-c2ccc3c4ccccc4n(-c4cccc(-c5cccc(-c6nc(-c7ccccc7)nc(-c7ccccc7)n6)c5)c4)c3c21 ; CC1(C)c2ccccc2-c2c1ccc1c2c2ccccc2n1-c1cccc(-c2cccc(-c3nc(-c4ccccc4)nc(-c4ccccc4)n3)c2)c1]

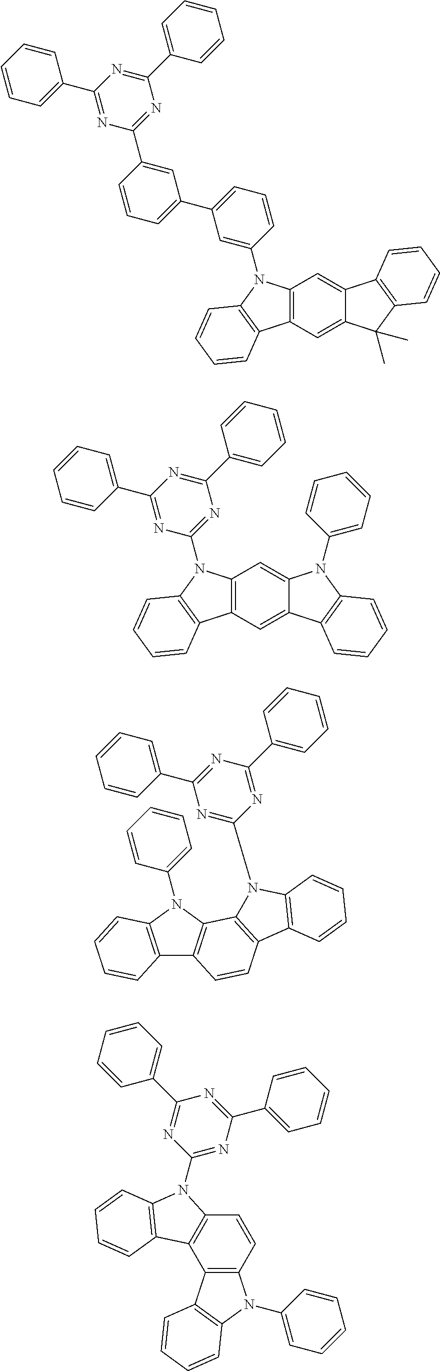

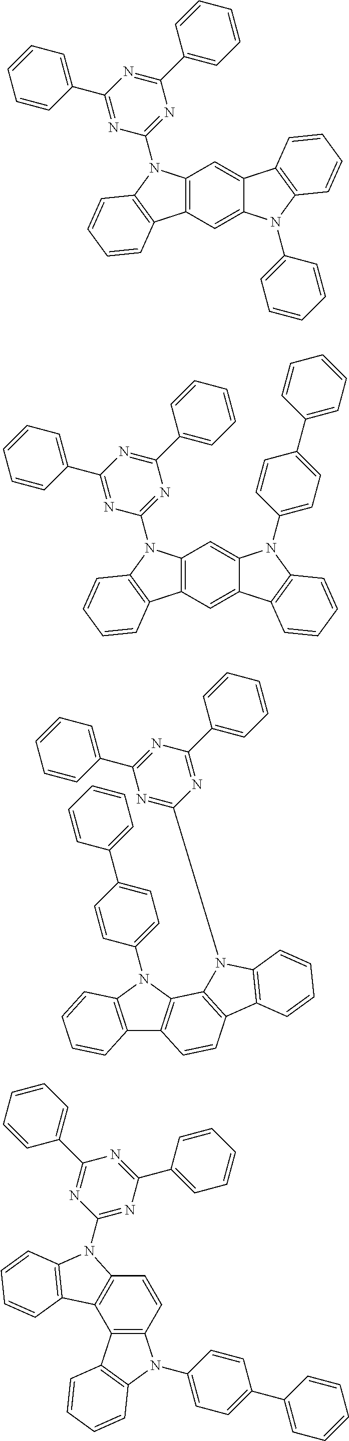

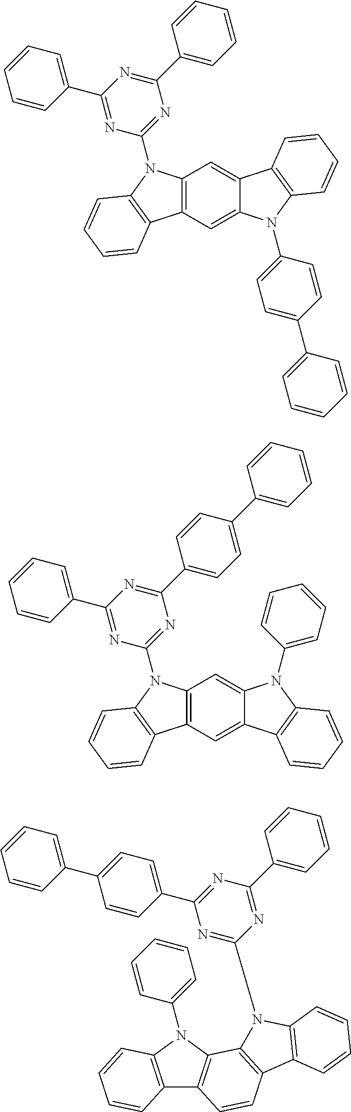

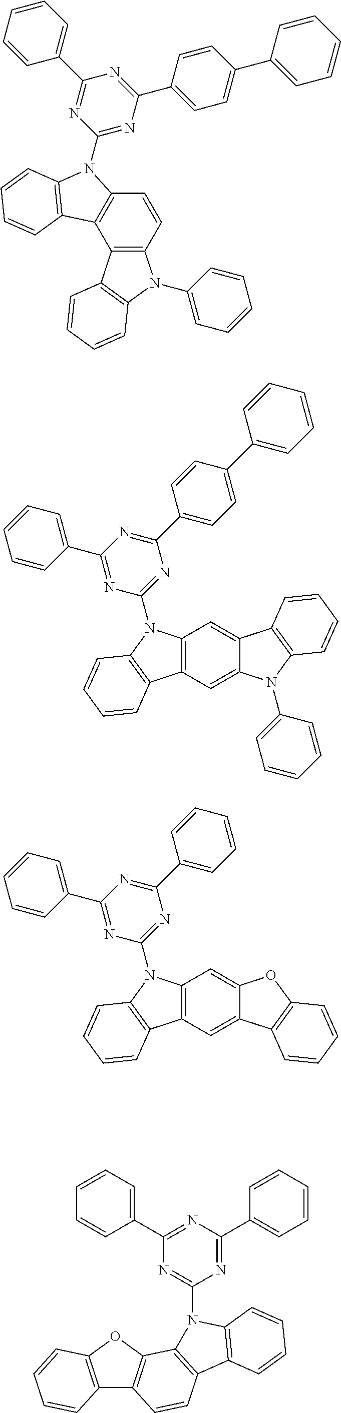 [SMILES: c1ccc(-c2ccc(-c3nc(-c4ccccc4)nc(-n4c5ccccc5c5c6c7ccccc7n(-c7ccccc7)c6ccc54)n3)cc2)cc1 ; c1ccc(-c2ccc(-c3nc(-c4ccccc4)nc(-n4c5ccccc5c5cc6c(cc54)c4ccccc4n6-c4ccccc4)n3)cc2)cc1 ; c1ccc(-c2nc(-c3ccccc3)nc(-n3c4ccccc4c4cc5c(cc43)oc3ccccc35)n2)cc1 ; c1ccc(-c2nc(-c3ccccc3)nc(-n3c4ccccc4c4ccc5c6ccccc6oc5c43)n2)cc1]

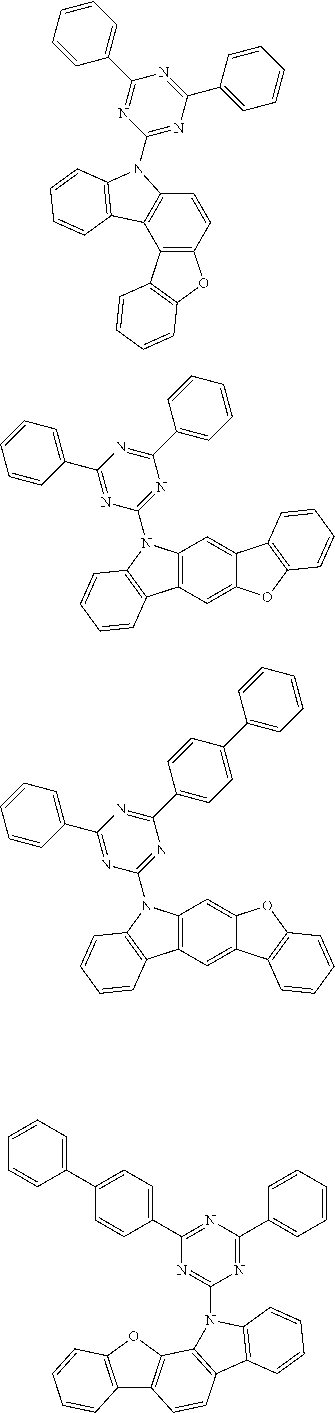

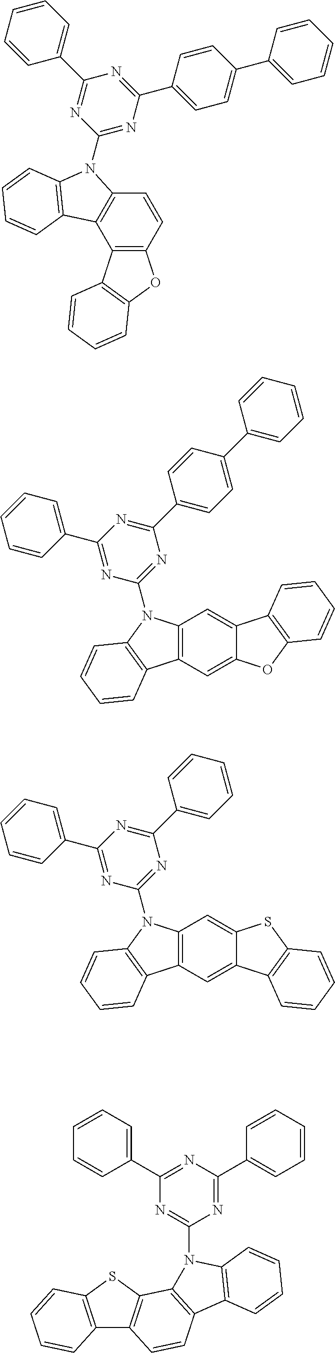 [SMILES: c1ccc(-c2ccc(-c3nc(-c4ccccc4)nc(-n4c5ccccc5c5c6c(ccc54)oc4ccccc46)n3)cc2)cc1 ; c1ccc(-c2ccc(-c3nc(-c4ccccc4)nc(-n4c5ccccc5c5cc6oc7ccccc7c6cc54)n3)cc2)cc1 ; c1ccc(-c2nc(-c3ccccc3)nc(-n3c4ccccc4c4cc5c(cc43)sc3ccccc35)n2)cc1 ; c1ccc(-c2nc(-c3ccccc3)nc(-n3c4ccccc4c4ccc5c6ccccc6sc5c43)n2)cc1]

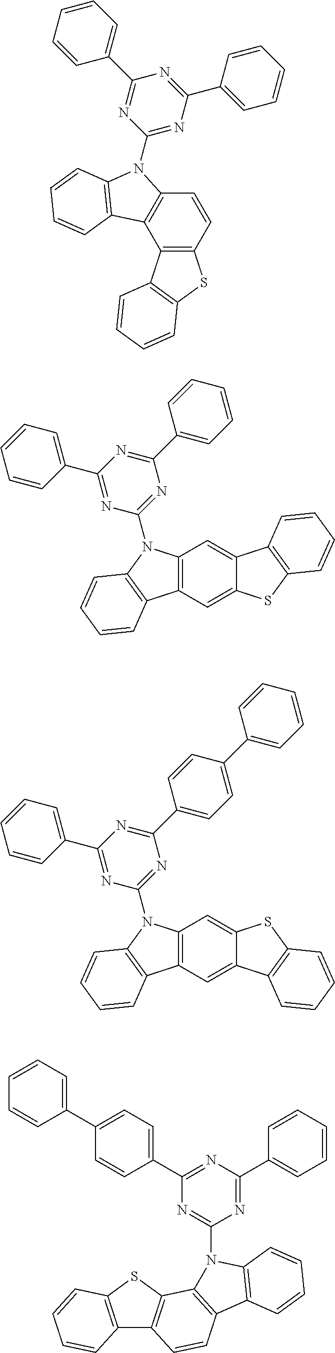 [SMILES: c1ccc(-c2nc(-c3ccccc3)nc(-n3c4ccccc4c4c5c(ccc43)sc3ccccc35)n2)cc1 ; c1ccc(-c2nc(-c3ccccc3)nc(-n3c4ccccc4c4cc5sc6ccccc6c5cc43)n2)cc1 ; c1ccc(-c2ccc(-c3nc(-c4ccccc4)nc(-n4c5ccccc5c5cc6c(cc54)sc4ccccc46)n3)cc2)cc1 ; c1ccc(-c2ccc(-c3nc(-c4ccccc4)nc(-n4c5ccccc5c5ccc6c7ccccc7sc6c54)n3)cc2)cc1]

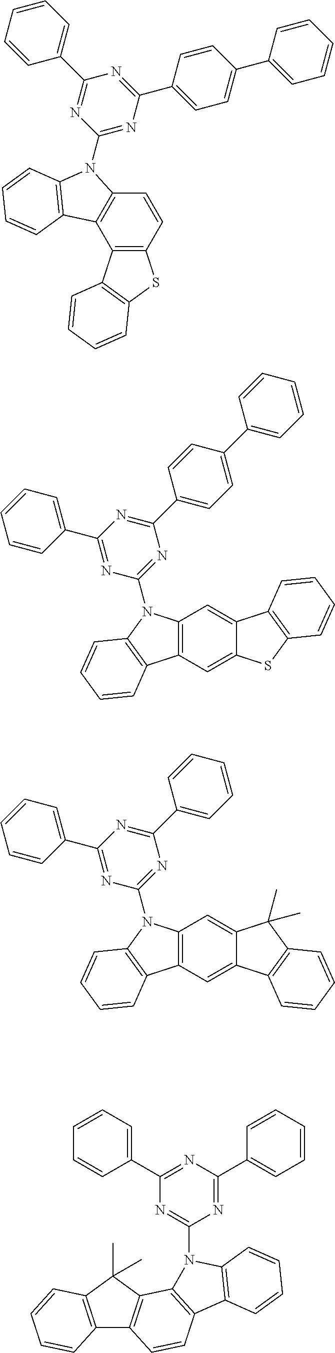 [SMILES: c1ccc(-c2ccc(-c3nc(-c4ccccc4)nc(-n4c5ccccc5c5c6c(ccc54)sc4ccccc46)n3)cc2)cc1 ; c1ccc(-c2ccc(-c3nc(-c4ccccc4)nc(-n4c5ccccc5c5cc6sc7ccccc7c6cc54)n3)cc2)cc1 ; CC1(C)c2ccccc2-c2cc3c4ccccc4n(-c4nc(-c5ccccc5)nc(-c5ccccc5)n4)c3cc21 ; CC1(C)c2ccccc2-c2ccc3c4ccccc4n(-c4nc(-c5ccccc5)nc(-c5ccccc5)n4)c3c21]

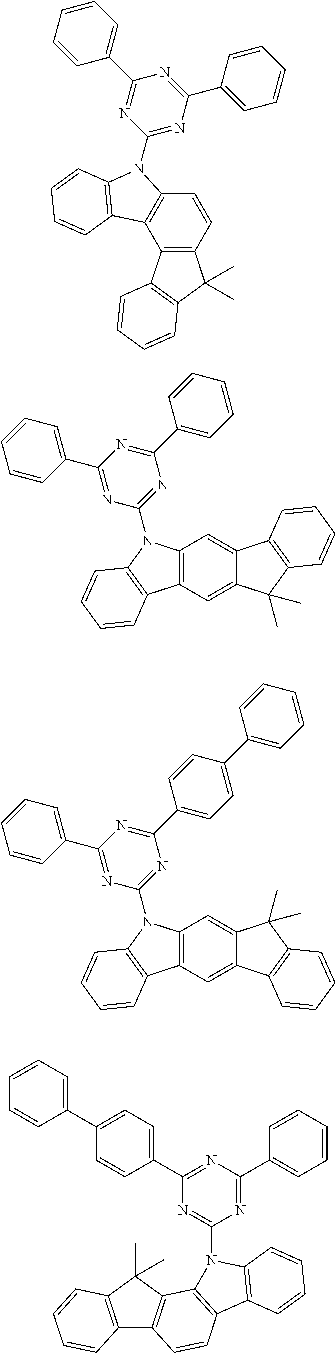

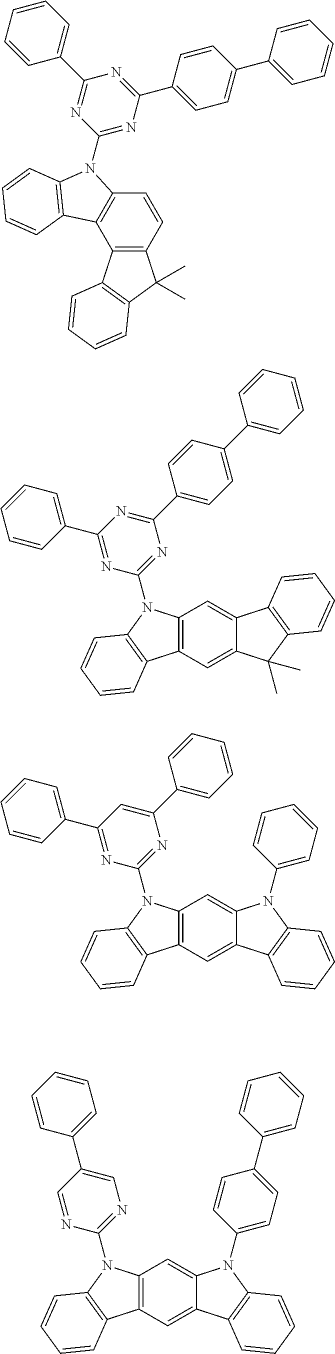 [SMILES: CC1(C)c2ccccc2-c2c1ccc1c2c2ccccc2n1-c1nc(-c2ccccc2)nc(-c2ccc(-c3ccccc3)cc2)n1 ; CC1(C)c2ccccc2-c2cc3c(cc21)c1ccccc1n3-c1nc(-c2ccccc2)nc(-c2ccc(-c3ccccc3)cc2)n1 ; c1ccc(-c2cc(-c3ccccc3)nc(-n3c4ccccc4c4cc5c6ccccc6n(-c6ccccc6)c5cc43)n2)cc1 ; c1ccc(-c2ccc(-n3c4ccccc4c4cc5c6ccccc6n(-c6ncc(-c7ccccc7)cn6)c5cc43)cc2)cc1]

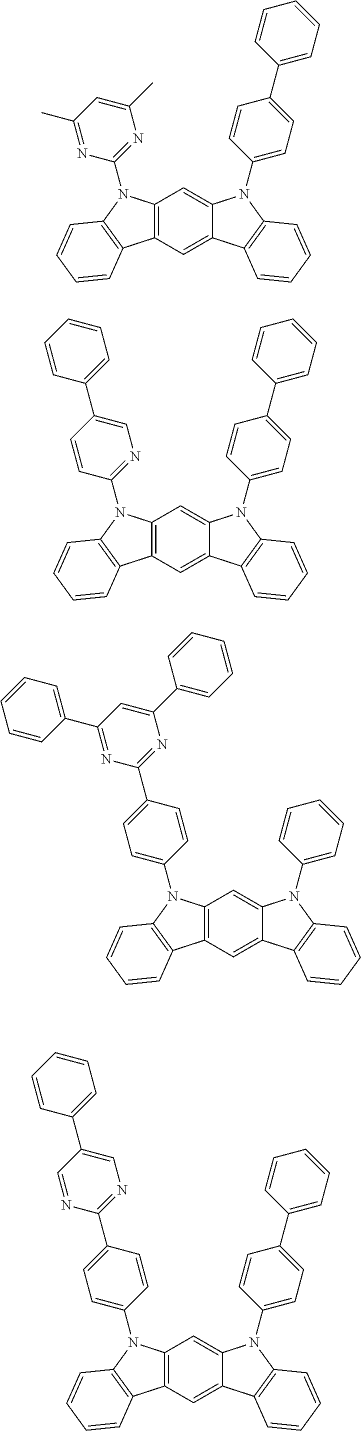

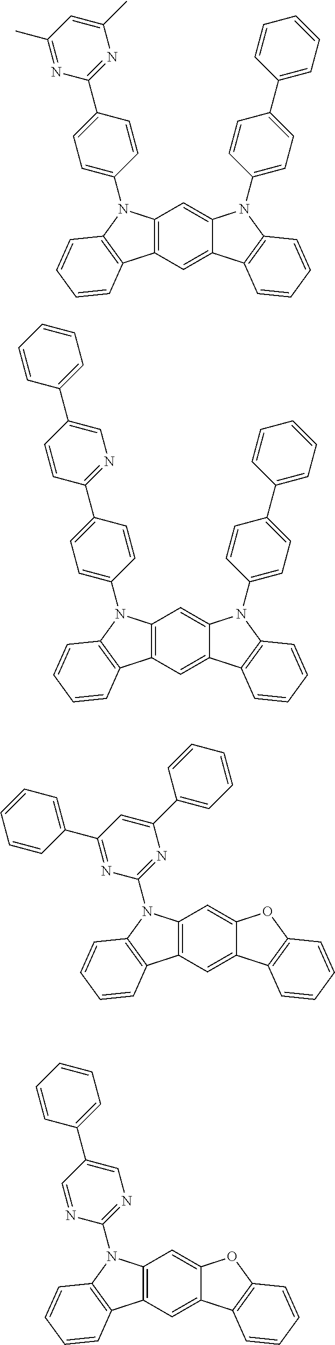

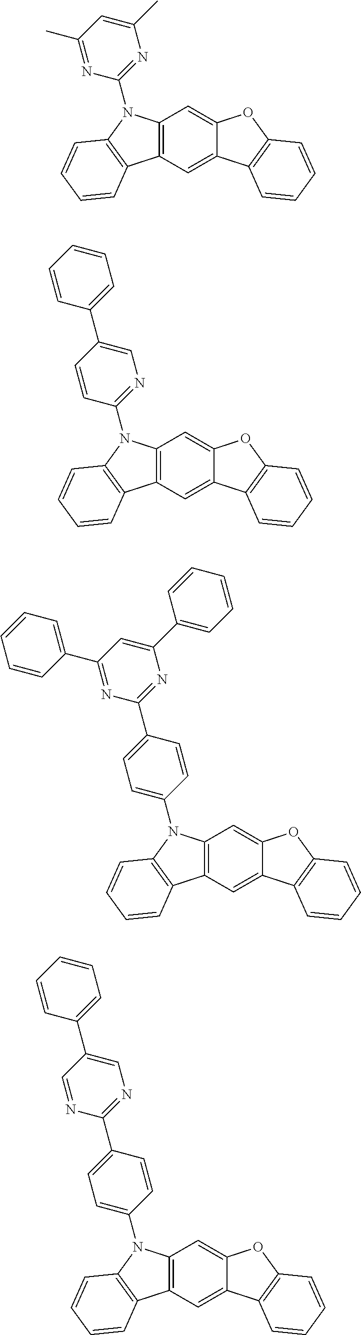

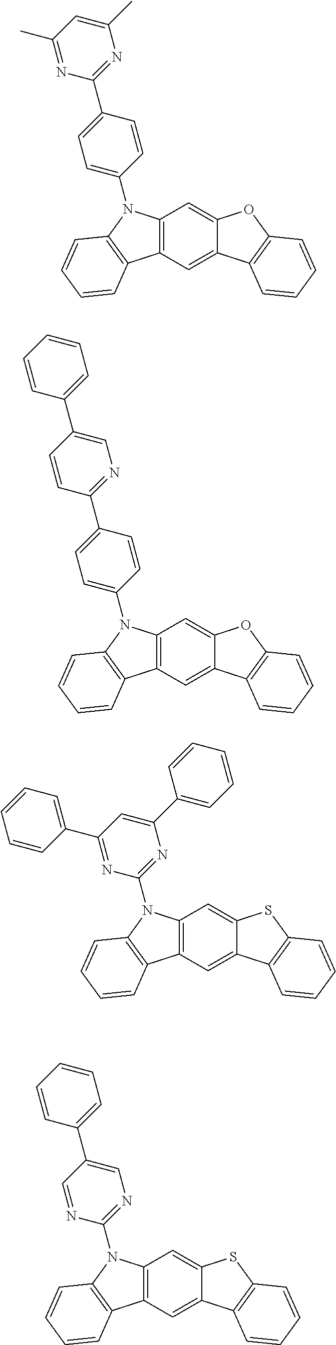

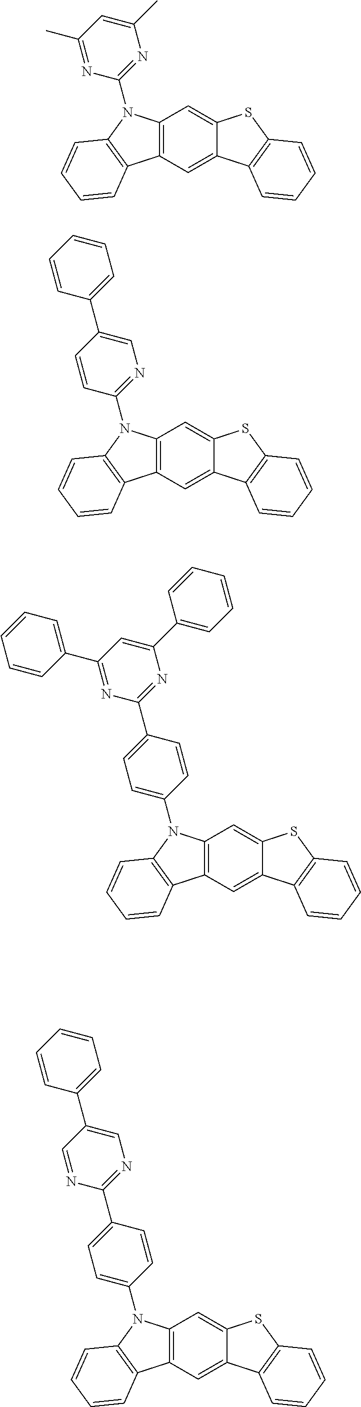

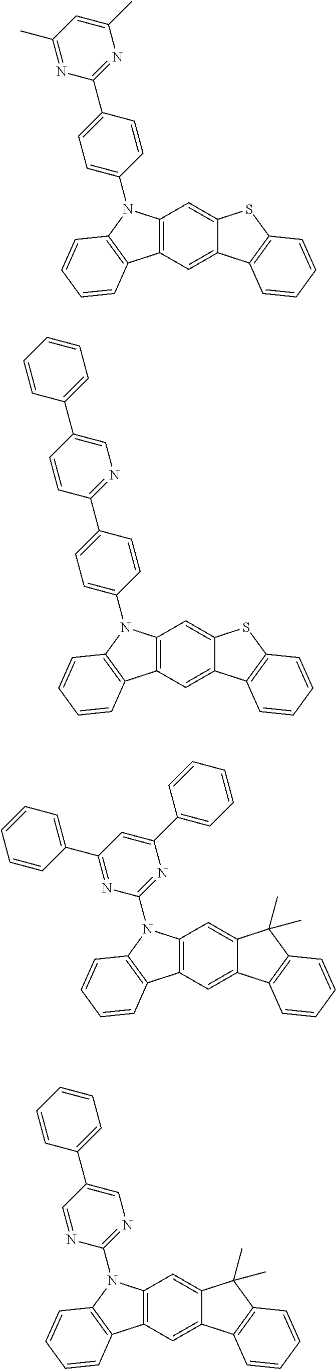 [SMILES: Cc1cc(C)nc(-c2ccc(-n3c4ccccc4c4cc5c(cc43)sc3ccccc35)cc2)n1 ; c1ccc(-c2ccc(-c3ccc(-n4c5ccccc5c5cc6c(cc54)sc4ccccc46)cc3)nc2)cc1 ; CC1(C)c2ccccc2-c2cc3c4ccccc4n(-c4nc(-c5ccccc5)cc(-c5ccccc5)n4)c3cc21 ; CC1(C)c2ccccc2-c2cc3c4ccccc4n(-c4ncc(-c5ccccc5)cn4)c3cc21]

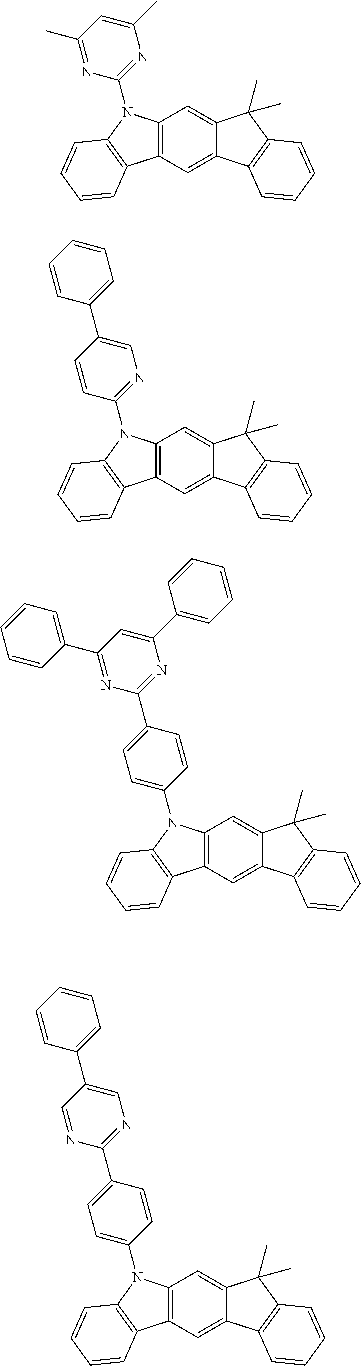

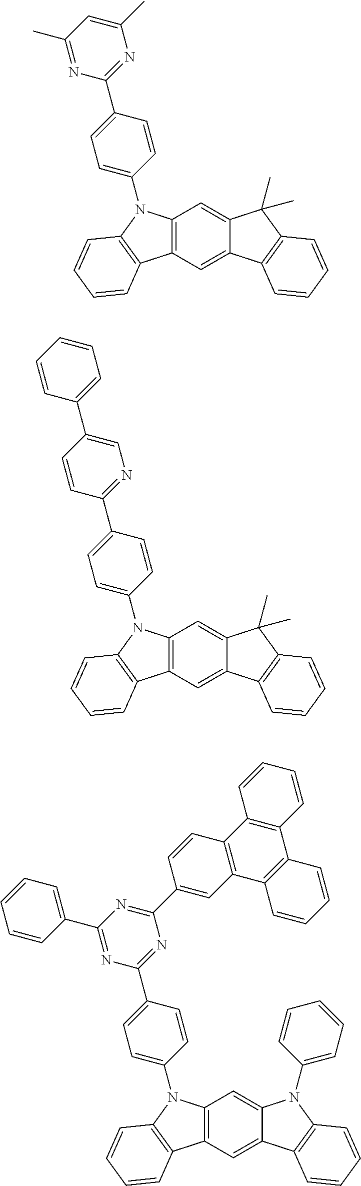 [SMILES: Cc1cc(C)nc(-c2ccc(-n3c4ccccc4c4cc5c(cc43)C(C)(C)c3ccccc3-5)cc2)n1 ; CC1(C)c2ccccc2-c2cc3c4ccccc4n(-c4ccc(-c5ccc(-c6ccccc6)cn5)cc4)c3cc21 ; c1ccc(-c2nc(-c3ccc(-n4c5ccccc5c5cc6c7ccccc7n(-c7ccccc7)c6cc54)cc3)nc(-c3ccc4c5ccccc5c5ccccc5c4c3)n2)cc1]

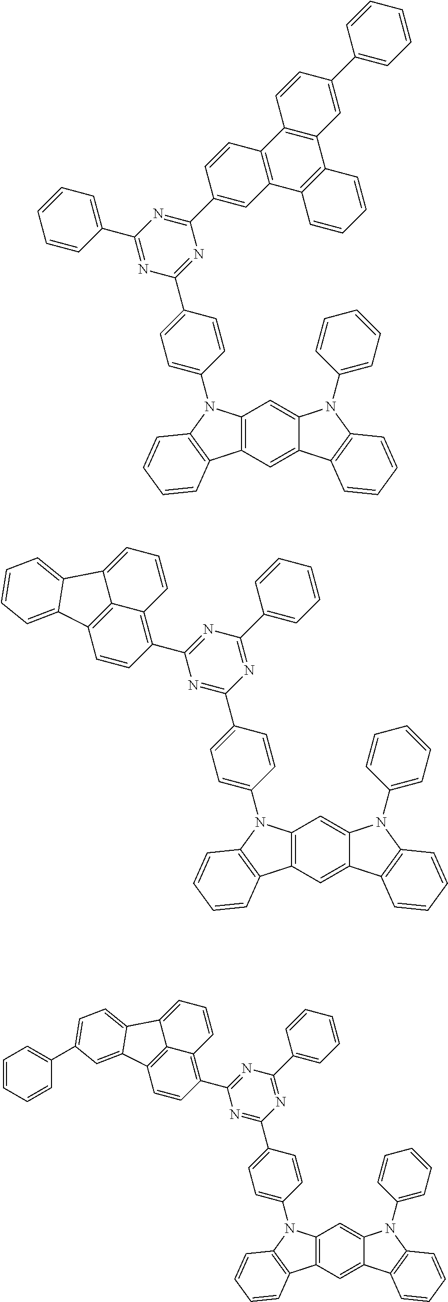 [SMILES: c1ccc(-c2ccc3c4ccc(-c5nc(-c6ccccc6)nc(-c6ccc(-n7c8ccccc8c8cc9c%10ccccc%10n(-c%10ccccc%10)c9cc87)cc6)n5)cc4c4ccccc4c3c2)cc1 ; c1ccc(-c2nc(-c3ccc(-n4c5ccccc5c5cc6c7ccccc7n(-c7ccccc7)c6cc54)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c9ccccc9n(-c9ccccc9)c8cc76)cc5)n4)c4cccc-3c24)cc1]

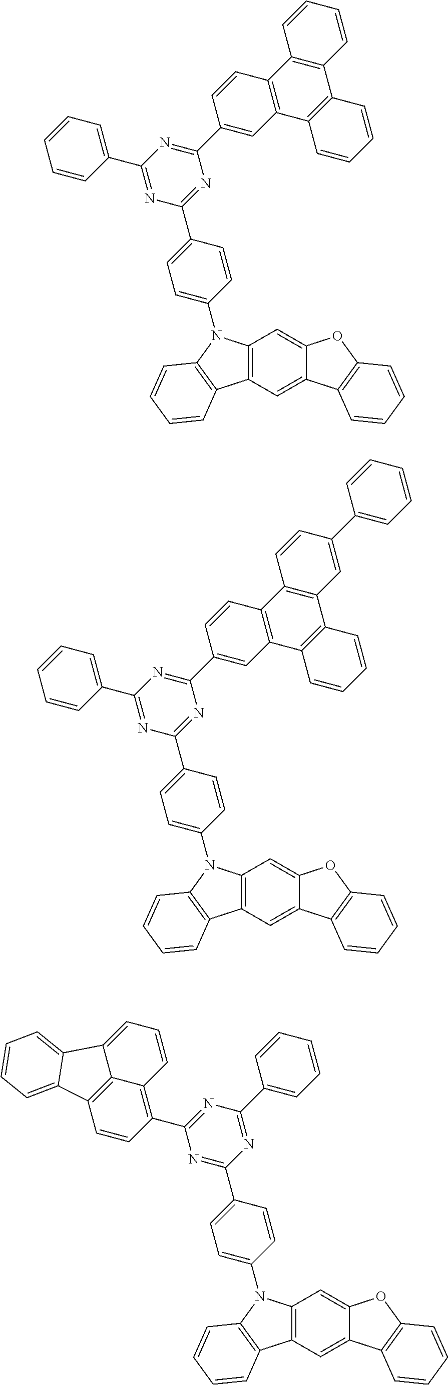

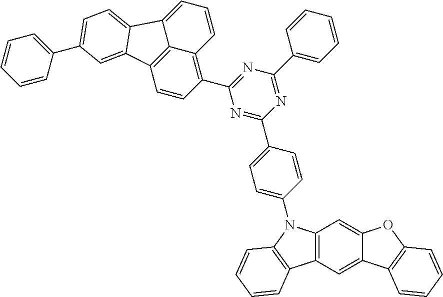 [SMILES: c1ccc(-c2ccc3c(c2)-c2ccc(-c4nc(-c5ccccc5)nc(-c5ccc(-n6c7ccccc7c7cc8c(cc76)oc6ccccc68)cc5)n4)c4cccc-3c24)cc1]

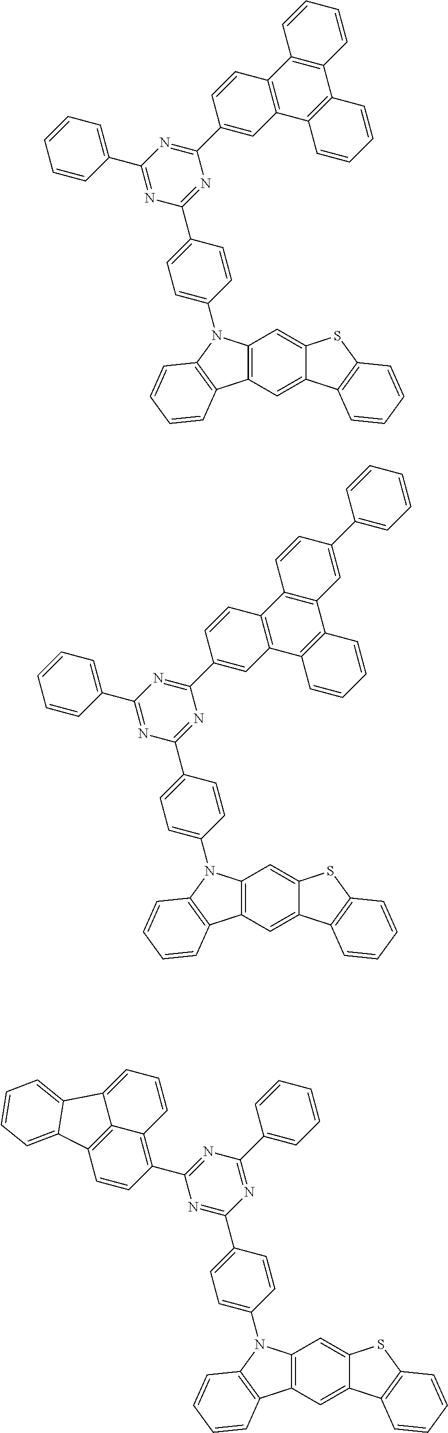

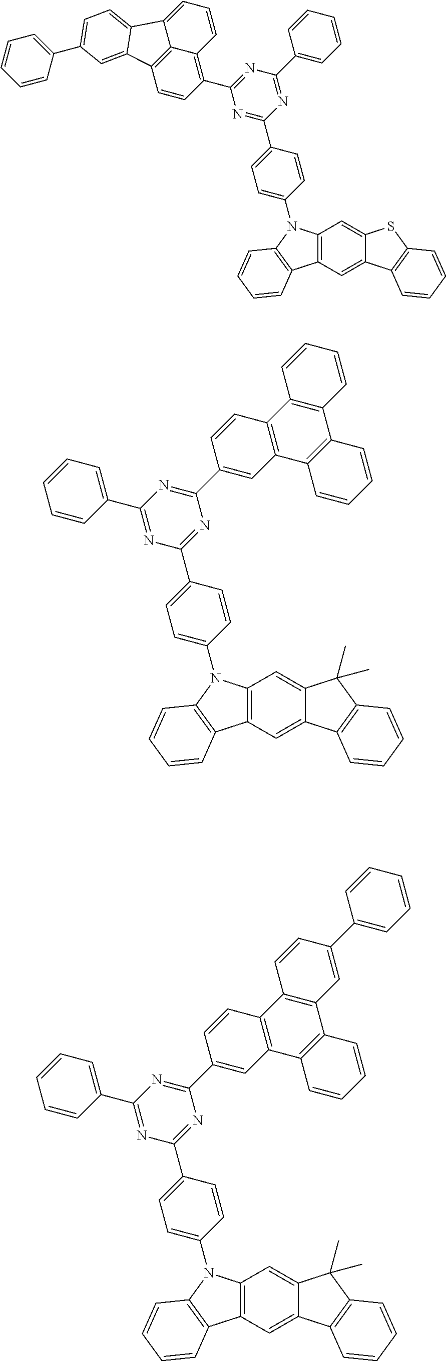 [SMILES: c1ccc(-c2ccc3c(c2)-c2ccc(-c4nc(-c5ccccc5)nc(-c5ccc(-n6c7ccccc7c7cc8c(cc76)sc6ccccc68)cc5)n4)c4cccc-3c24)cc1 ; CC1(C)c2ccccc2-c2cc3c4ccccc4n(-c4ccc(-c5nc(-c6ccccc6)nc(-c6ccc7c8ccccc8c8ccccc8c7c6)n5)cc4)c3cc21 ; CC1(C)c2ccccc2-c2cc3c4ccccc4n(-c4ccc(-c5nc(-c6ccccc6)nc(-c6ccc7c8ccc(-c9ccccc9)cc8c8ccccc8c7c6)n5)cc4)c3cc21]

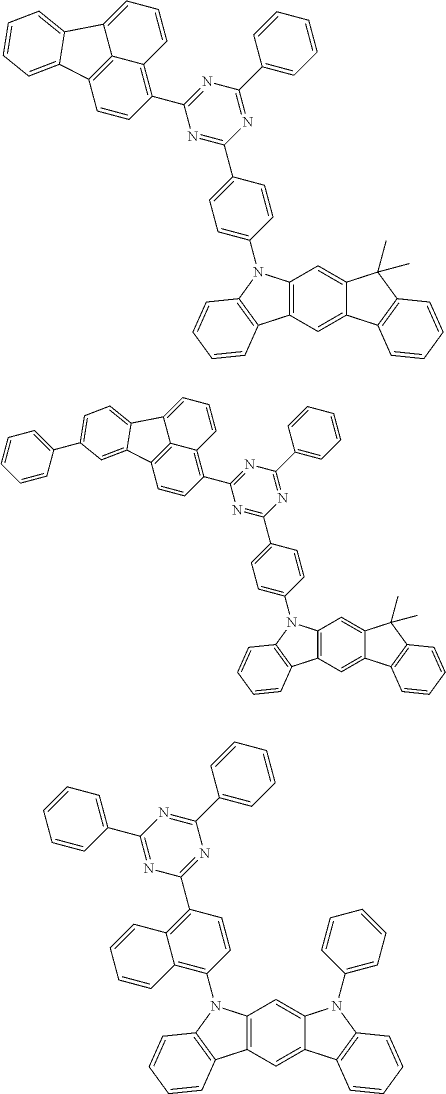 [SMILES: CC1(C)c2ccccc2-c2cc3c4ccccc4n(-c4ccc(-c5nc(-c6ccccc6)nc(-c6ccc7c8c(cccc68)-c6ccccc6-7)n5)cc4)c3cc21 ; CC1(C)c2ccccc2-c2cc3c4ccccc4n(-c4ccc(-c5nc(-c6ccccc6)nc(-c6ccc7c8c(cccc68)-c6ccc(-c8ccccc8)cc6-7)n5)cc4)c3cc21 ; c1ccc(-c2nc(-c3ccccc3)nc(-c3ccc(-n4c5ccccc5c5cc6c7ccccc7n(-c7ccccc7)c6cc54)c4ccccc34)n2)cc1]

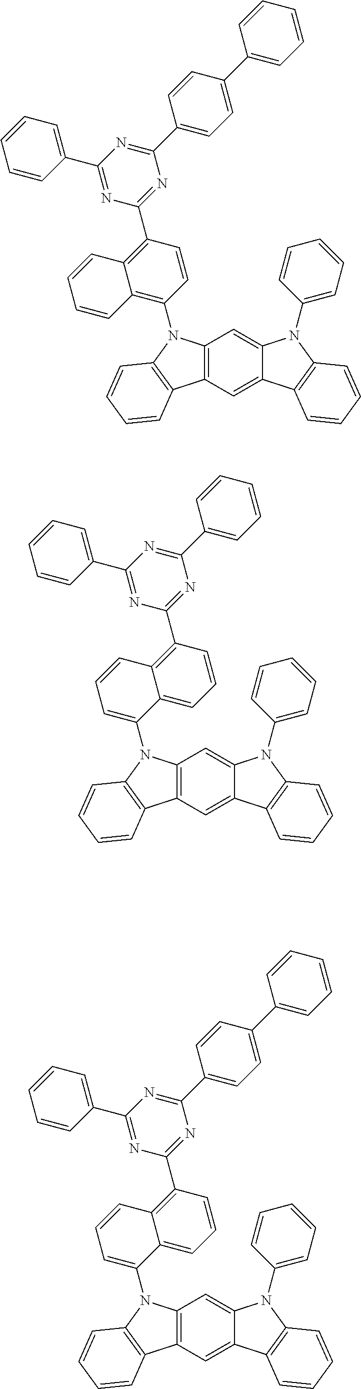 [SMILES: c1ccc(-c2ccc(-c3nc(-c4ccccc4)nc(-c4ccc(-n5c6ccccc6c6cc7c8ccccc8n(-c8ccccc8)c7cc65)c5ccccc45)n3)cc2)cc1 ; c1ccc(-c2nc(-c3ccccc3)nc(-c3cccc4c(-n5c6ccccc6c6cc7c8ccccc8n(-c8ccccc8)c7cc65)cccc34)n2)cc1 ; c1ccc(-c2ccc(-c3nc(-c4ccccc4)nc(-c4cccc5c(-n6c7ccccc7c7cc8c9ccccc9n(-c9ccccc9)c8cc76)cccc45)n3)cc2)cc1]

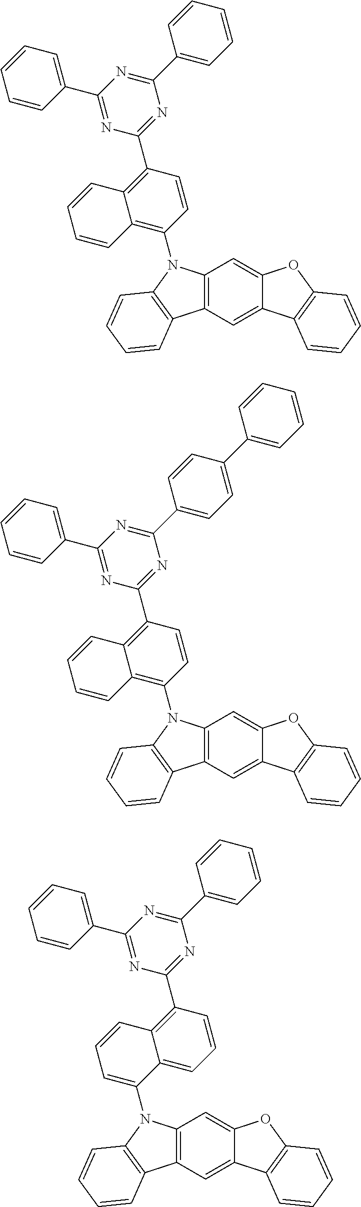

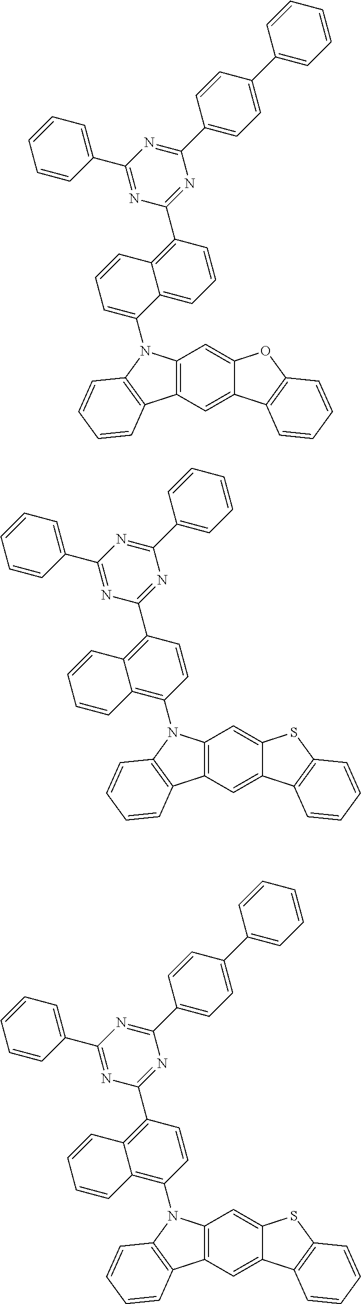 [SMILES: c1ccc(-c2ccc(-c3nc(-c4ccccc4)nc(-c4cccc5c(-n6c7ccccc7c7cc8c(cc76)oc6ccccc68)cccc45)n3)cc2)cc1 ; c1ccc(-c2nc(-c3ccccc3)nc(-c3ccc(-n4c5ccccc5c5cc6c(cc54)sc4ccccc46)c4ccccc34)n2)cc1 ; c1ccc(-c2ccc(-c3nc(-c4ccccc4)nc(-c4ccc(-n5c6ccccc6c6cc7c(cc65)sc5ccccc57)c5ccccc45)n3)cc2)cc1]

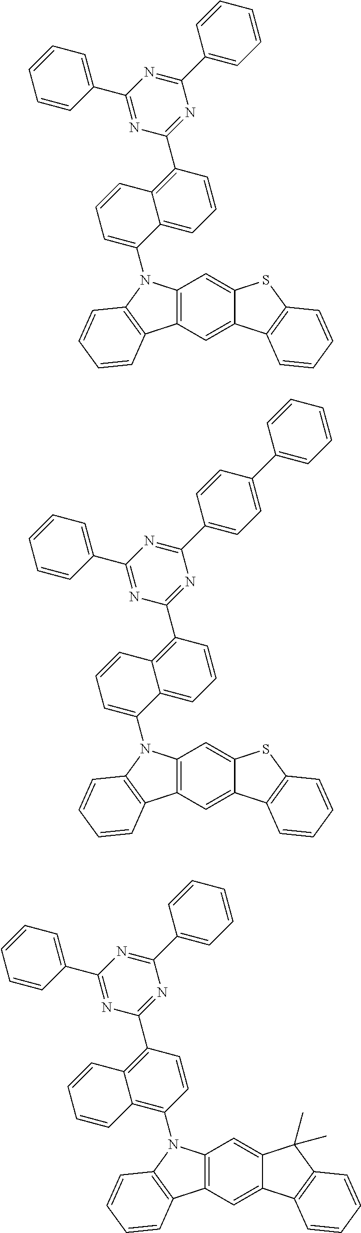

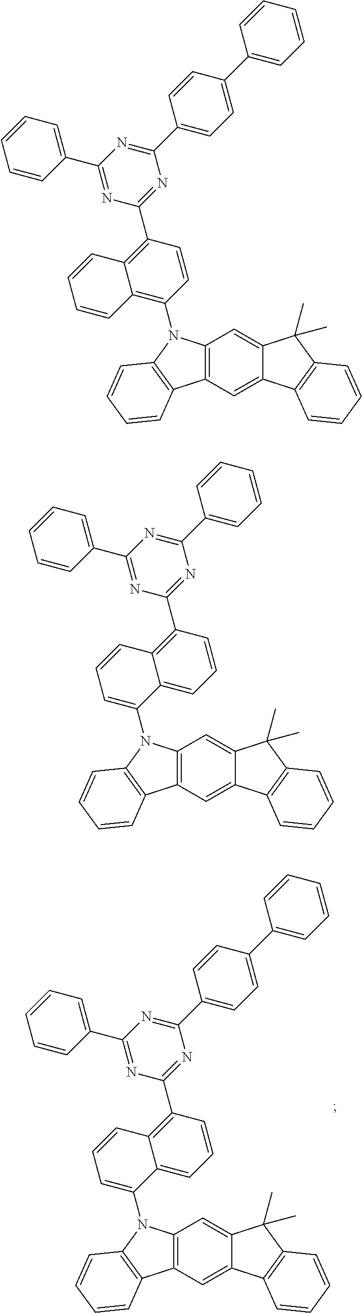 [SMILES: CC1(C)c2ccccc2-c2cc3c4ccccc4n(-c4ccc(-c5nc(-c6ccccc6)nc(-c6ccc(-c7ccccc7)cc6)n5)c5ccccc45)c3cc21 ; CC1(C)c2ccccc2-c2cc3c4ccccc4n(-c4cccc5c(-c6nc(-c7ccccc7)nc(-c7ccccc7)n6)cccc45)c3cc21 ; CC1(C)c2ccccc2-c2cc3c4ccccc4n(-c4cccc5c(-c6nc(-c7ccccc7)nc(-c7ccc(-c8ccccc8)cc7)n6)cccc45)c3cc21]

wherein a hydrogen atom in the above compounds can be substituted with a deuterium atom.

In a second aspect, the present application provides a compound, and the compound includes the following compounds:

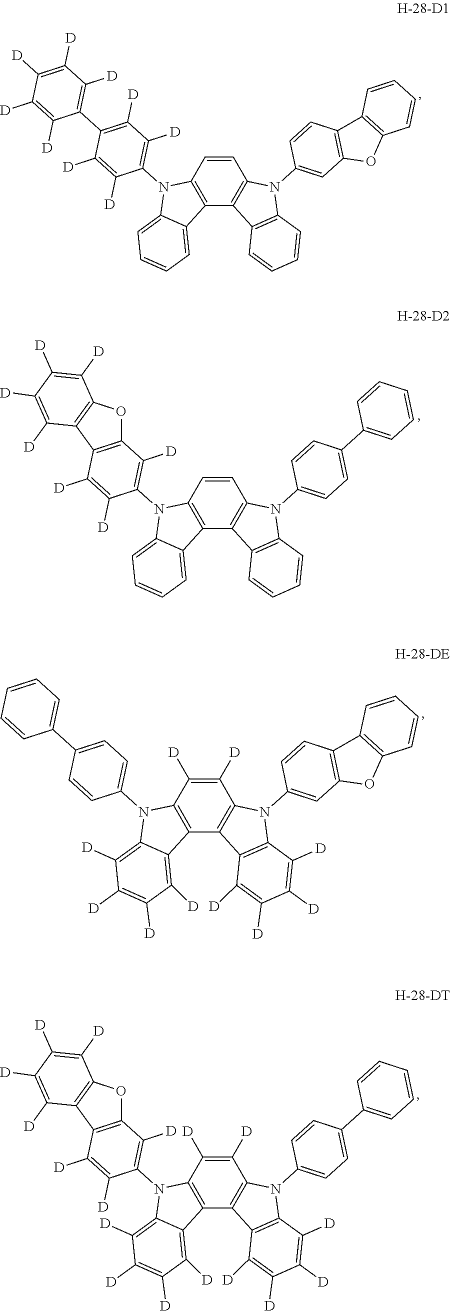

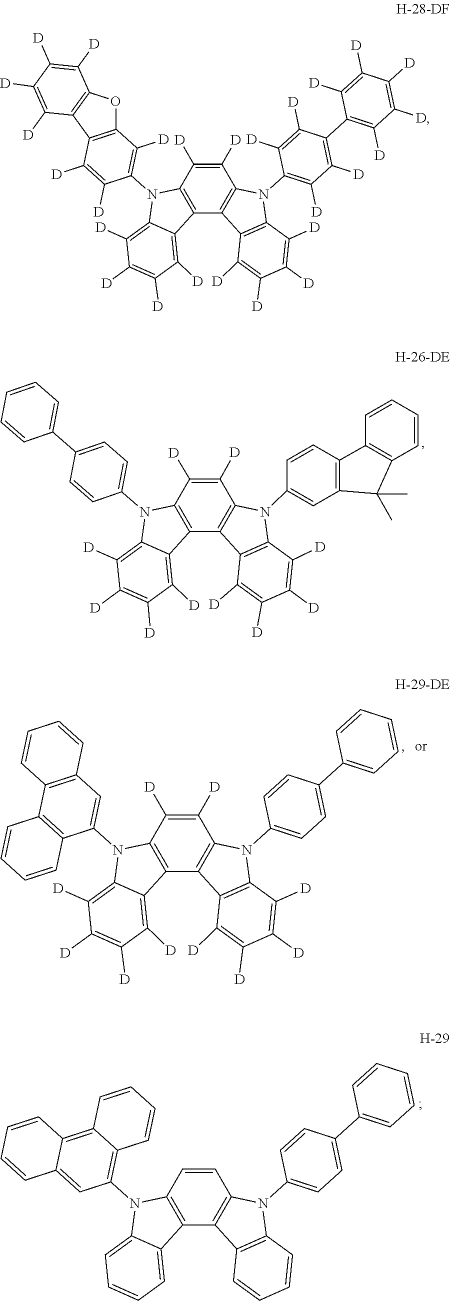

wherein the compound is used for preparing the composition as described in the first aspect.

In a third aspect, the present application provides an intermediate, and the intermediate includes the following compounds:

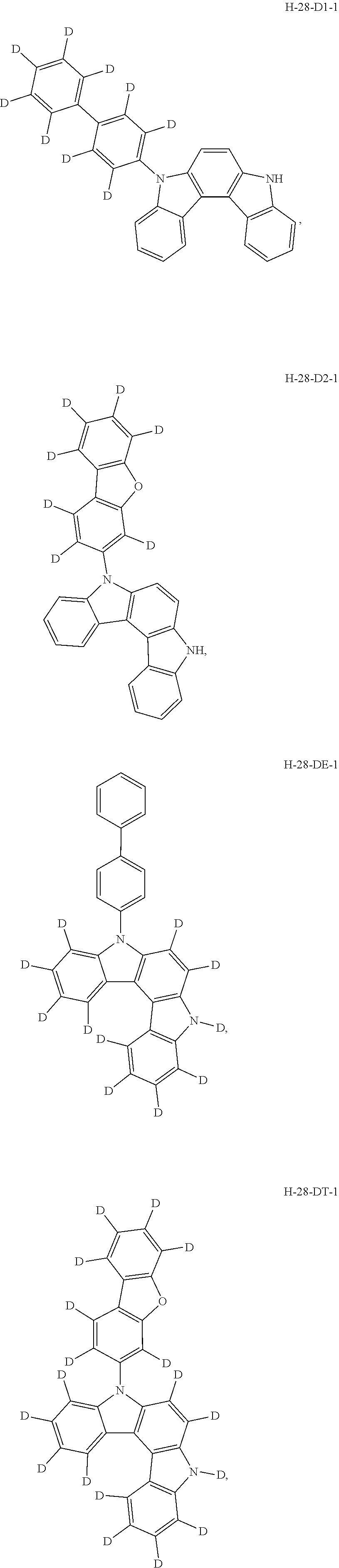

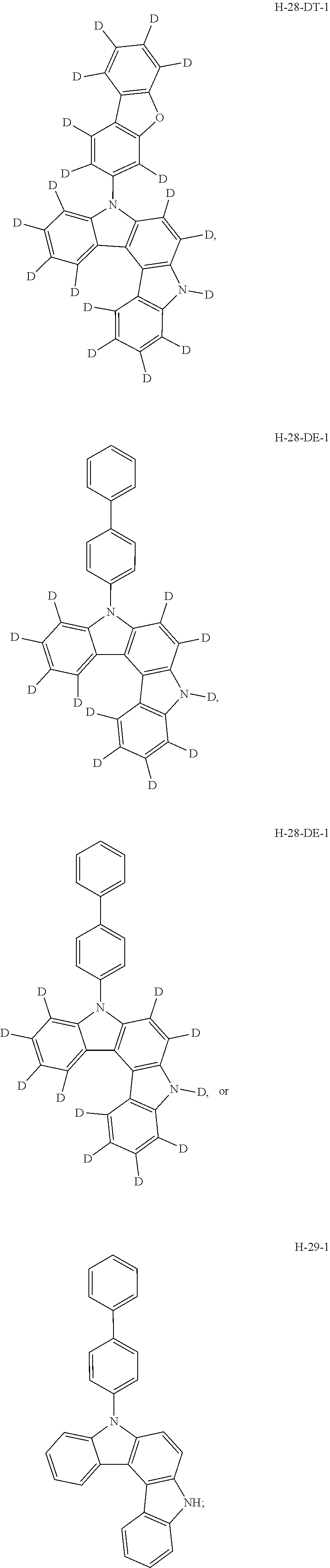

wherein the intermediate is used for preparing the compound represented by Formula I in a composition as described in the first aspect.

It should be noted that in the present application, the preparation methods of the compound represented by Formula I and the compound represented by Formula II are not limited in any particular way, and can be prepared by methods commonly used in the field, exemplarily referring to the preparation methods in CN112996793A, CN112805277A, and CN102212066A.

In a fourth aspect, the present application provides an organic electroluminescent device, and the organic electroluminescent device includes an anode, a cathode, and an organic thin film layer arranged between the anode and the cathode;
wherein a material of the organic thin film layer includes the composition as described in the first aspect.

In one embodiment, the organic thin film layer includes an emissive layer, and a material of the emissive layer includes the composition as described in the first aspect.

In one embodiment, the organic thin film layer includes a hole layer.

In one embodiment, the hole layer includes an electron blocking layer; and a material of the electron blocking layer includes a spirofluorene compound;
wherein the spirofluorene compound has a specific structure as represented by Formula III below:
wherein X is selected from O or S;

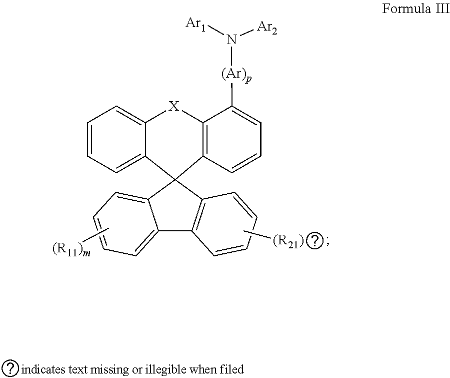

R₁₁ and R₂₁ are each independently selected from hydrogen, deuterium, fluorine, CN, substituted or unsubstituted C1 to C20 (which may be, for example, C1, C2, C3, C4, C5, C7, C8, C9, C10, C13, C15, C18, or C20, etc.) linear or branched alkyl, substituted or unsubstituted C1 to C20 (which may be, for example, C1, C2, C3, C4, C5, C7, C8, C9, C10, C13, C15, C18, or C20, etc.) alkoxy, or substituted or unsubstituted C6 to C40 (which may be, for example, C6, C8, C10, C12, C15, C18, C24, C30, C36, or C40, etc.) aryl;
Ar is selected from substituted or unsubstituted C6 to C40 (which may be, for example, C6, C8, C10, C12, C15, C18, C24, C30, C36, or C40, etc.) arylidene;
Ar₁ and Ar₂ are each independently selected from substituted or unsubstituted C6 to C40 (which may be, for example, C6, C8, C10, C12, C15, C18, C24, C30, C36, or C40, etc.) aryl, substituted or unsubstituted C12 to C40 (C12, C14, C16, C18, C20, C23, C25, C27, C30, C32, C35, C37, C39 or C40, etc.) oxa-aryl, or substituted or unsubstituted C12 to C40 (C12, C14, C16, C18, C20, C23, C25, C27, C30, C32, C35, C37, C39 or C40, etc.) thioaryl, and at least one of Ar₁ or Ar₂ is selected from any one of phenyl, naphthyl, triphenylenyl, or fluoranthenyl; p is selected from 0 or 1;
m and n are each independently selected from an integer from 0 to 4, which may be, for example, 0, 1, 2, 3, or 4.

It should be noted that the oxa-aryl refers to a structure having an oxygen-containing five-membered heterocyclic ring where the two aryl rings connected by a single bond are bridged by an O atom, for example, two benzene rings are connected by a single bond to form a biphenyl, and the carbon atoms on the two benzene rings forming the biphenyl are connected to the 0 atom at the same time to form a dibenzofuran.

The thioaryl refers to a structure having an sulfur-containing five-membered heterocyclic ring where the two aryl rings connected by a single bond are bridged by an S atom, for example, two benzene rings are connected by a single bond to form a biphenyl, and the carbon atoms on the two benzene rings forming the biphenyl are connected to the S atom at the same time to form a dibenzothienyl.

In one embodiment, the spirofluorene compound is selected from compounds represented by Formula III-1 or compounds represented by Formula III-2:

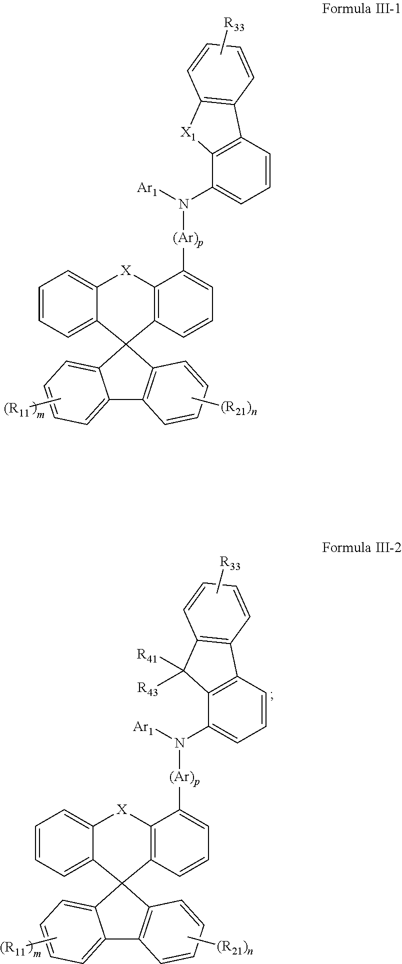

wherein X and X₁ are each independently selected from 0 or S;
R₁₁, R₂₁, and Ar have the same protection scope as described above;
Ar₁ is selected from any one of phenyl, naphthyl, triphenylenyl, or fluoranthenyl;
R₃₁ is selected from C1 to C20 (which may be, for example, C1, C2, C3, C4, C5, C7, C8, C9, C10, C13, C15, C18, or C20, etc.) linear or branched alkyl, C1 to C20 (which may be, for example, C1, C2, C3, C4, C5, C7, C8, C9, C10, C13, C15, C18, or C20, etc.) alkoxy, or C6 to C40 (which may be, for example, C6, C8, C10, C12, C15, C18, C24, C30, C36 or C40, etc.) aryl;
R₄₁ and R₄₂ are each independently selected from C1 to C20 (which may be, for example, C1, C2, C3, C4, C5, C7, C8, C9, C10, C13, C15, C18, or C20, etc.) linear or branched alkyl, C6 to C40 (which may be, for example, C6, C8, C10, C12, C15, C18, C24, C30, C36, or C40, etc.) aryl, and R₄₁ and R₄₂ are independent with each other or are connected by a single bond to form a ring.

In one embodiment, the spirofluorene compound is selected from any one of the following Compounds 1-140 and Compounds 1S-140S:

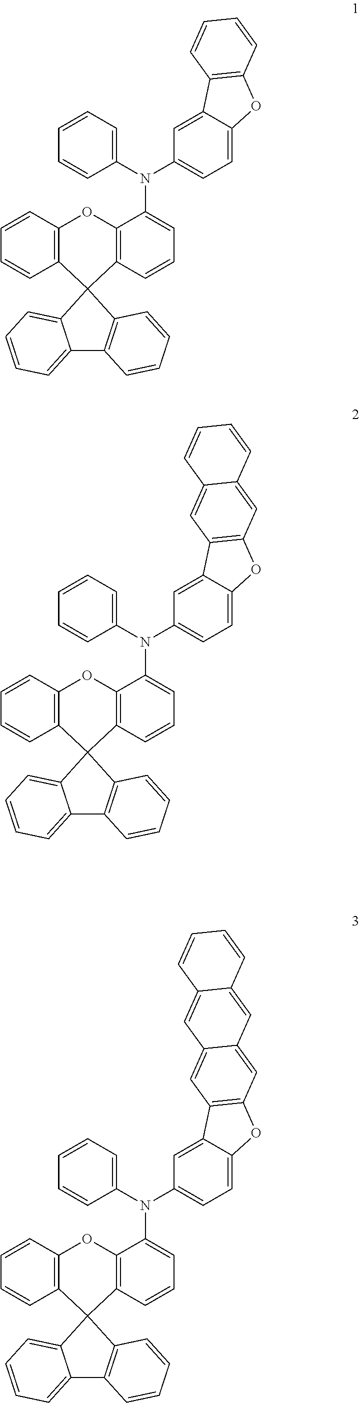 [SMILES: c1ccc(N(c2ccc3oc4ccccc4c3c2)c2cccc3c2Oc2ccccc2C32c3ccccc3-c3ccccc32)cc1 ; c1ccc(N(c2ccc3oc4cc5ccccc5cc4c3c2)c2cccc3c2Oc2ccccc2C32c3ccccc3-c3ccccc32)cc1 ; c1ccc(N(c2ccc3oc4cc5cc6ccccc6cc5cc4c3c2)c2cccc3c2Oc2ccccc2C32c3ccccc3-c3ccccc32)cc1]

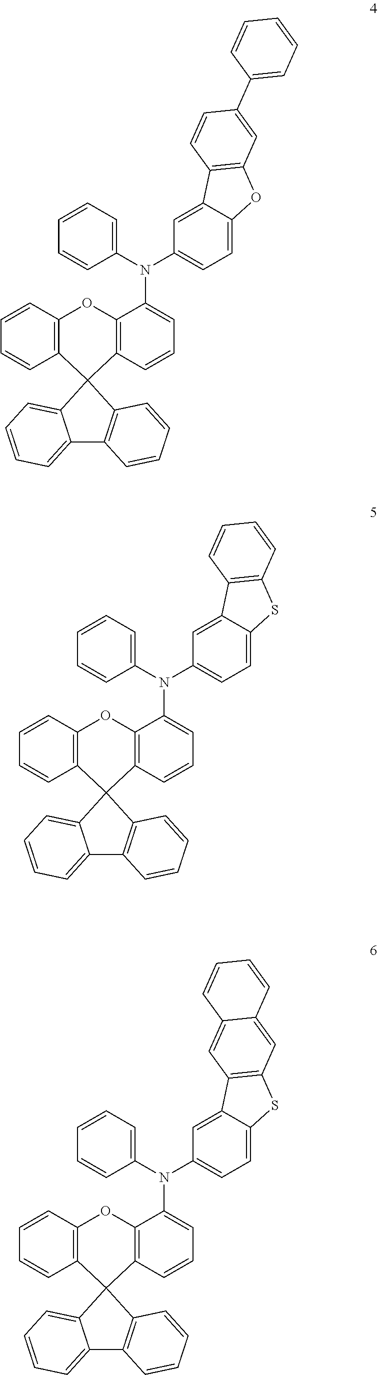 [SMILES: c1ccc(-c2ccc3c(c2)oc2ccc(N(c4ccccc4)c4cccc5c4Oc4ccccc4C54c5ccccc5-c5ccccc54)cc23)cc1 ; c1ccc(N(c2ccc3sc4ccccc4c3c2)c2cccc3c2Oc2ccccc2C32c3ccccc3-c3ccccc32)cc1 ; c1ccc(N(c2ccc3sc4cc5ccccc5cc4c3c2)c2cccc3c2Oc2ccccc2C32c3ccccc3-c3ccccc32)cc1]

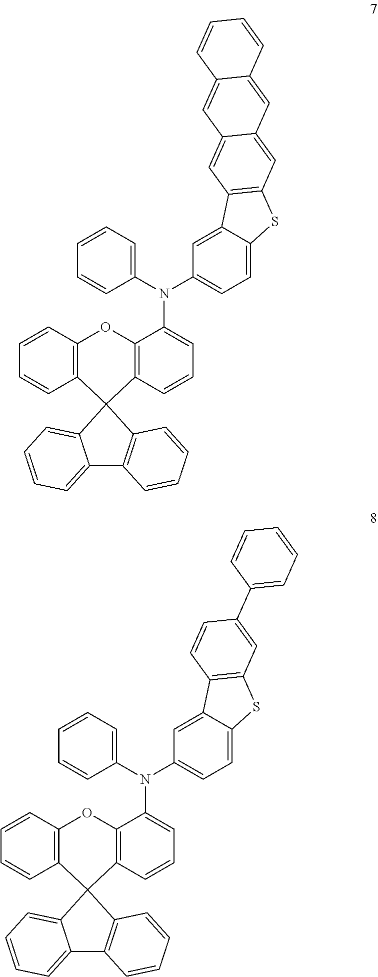 [SMILES: c1ccc(N(c2ccc3sc4cc5cc6ccccc6cc5cc4c3c2)c2cccc3c2Oc2ccccc2C32c3ccccc3-c3ccccc32)cc1 ; c1ccc(-c2ccc3c(c2)sc2ccc(N(c4ccccc4)c4cccc5c4Oc4ccccc4C54c5ccccc5-c5ccccc54)cc23)cc1]

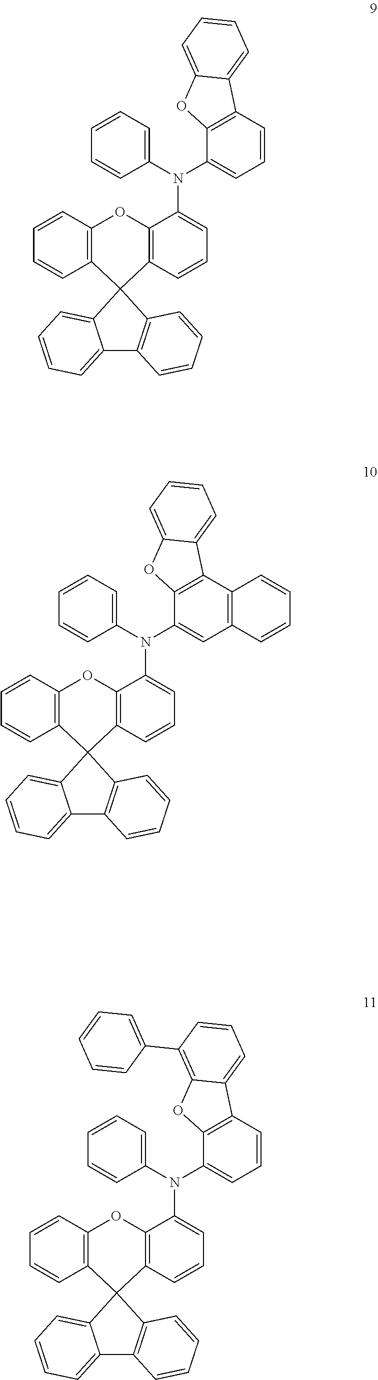 [SMILES: c1ccc(N(c2cccc3c2Oc2ccccc2C32c3ccccc3-c3ccccc32)c2cccc3c2oc2ccccc23)cc1 ; c1ccc(N(c2cccc3c2Oc2ccccc2C32c3ccccc3-c3ccccc32)c2cc3ccccc3c3c2oc2ccccc23)cc1 ; c1ccc(-c2cccc3c2oc2c(N(c4ccccc4)c4cccc5c4Oc4ccccc4C54c5ccccc5-c5ccccc54)cccc23)cc1]

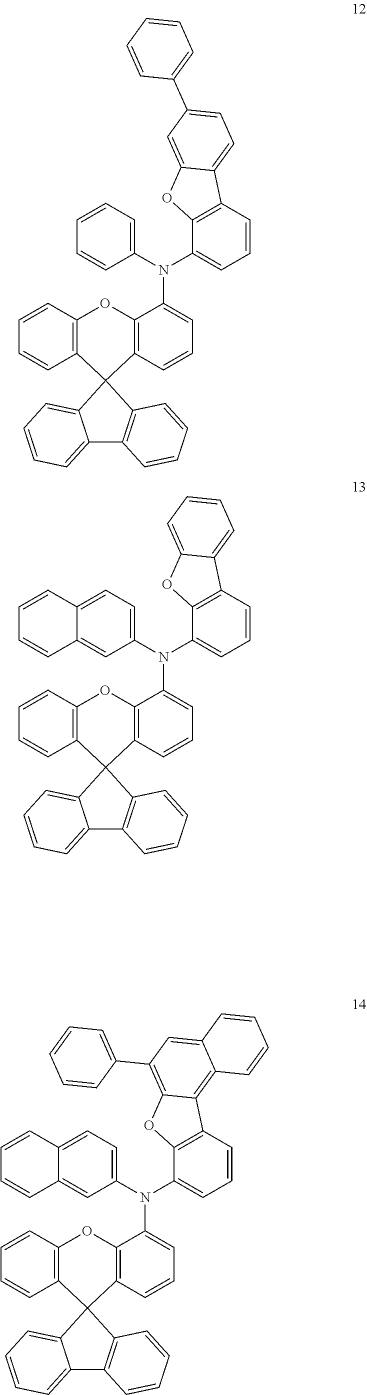

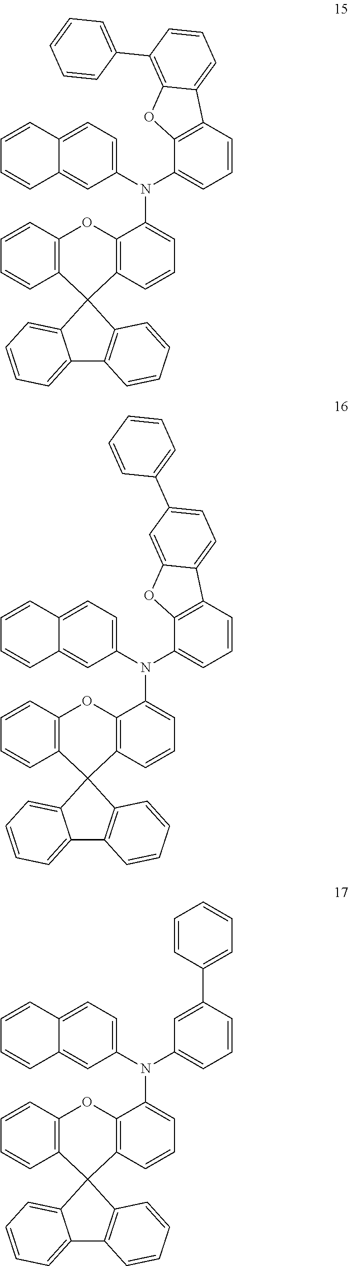

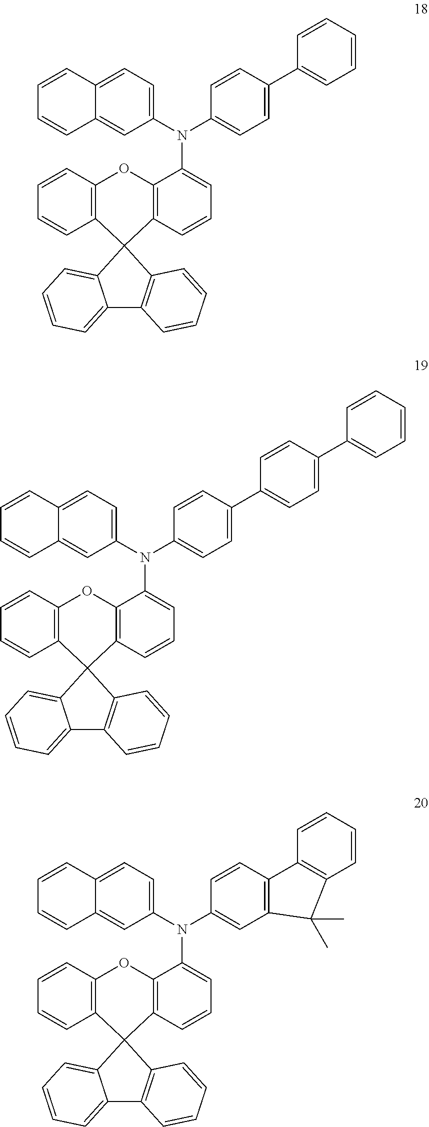 [SMILES: c1ccc(-c2ccc(N(c3ccc4ccccc4c3)c3cccc4c3Oc3ccccc3C43c4ccccc4-c4ccccc43)cc2)cc1 ; c1ccc(-c2ccc(-c3ccc(N(c4ccc5ccccc5c4)c4cccc5c4Oc4ccccc4C54c5ccccc5-c5ccccc54)cc3)cc2)cc1 ; CC1(C)c2ccccc2-c2ccc(N(c3ccc4ccccc4c3)c3cccc4c3Oc3ccccc3C43c4ccccc4-c4ccccc43)cc21]

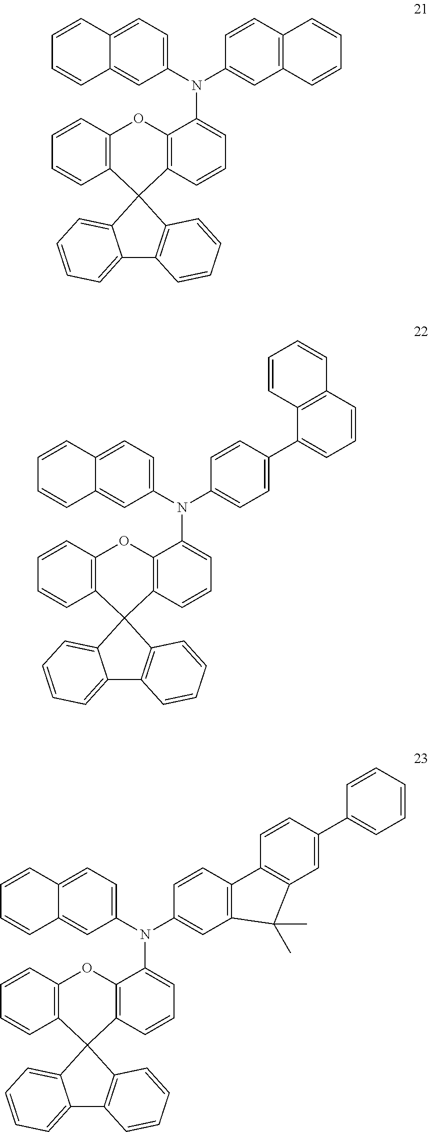 [SMILES: c1ccc2c(c1)Oc1c(N(c3ccc4ccccc4c3)c3ccc4ccccc4c3)cccc1C21c2ccccc2-c2ccccc21 ; c1ccc2c(c1)Oc1c(N(c3ccc(-c4cccc5ccccc45)cc3)c3ccc4ccccc4c3)cccc1C21c2ccccc2-c2ccccc21 ; CC1(C)c2cc(-c3ccccc3)ccc2-c2ccc(N(c3ccc4ccccc4c3)c3cccc4c3Oc3ccccc3C43c4ccccc4-c4ccccc43)cc21]

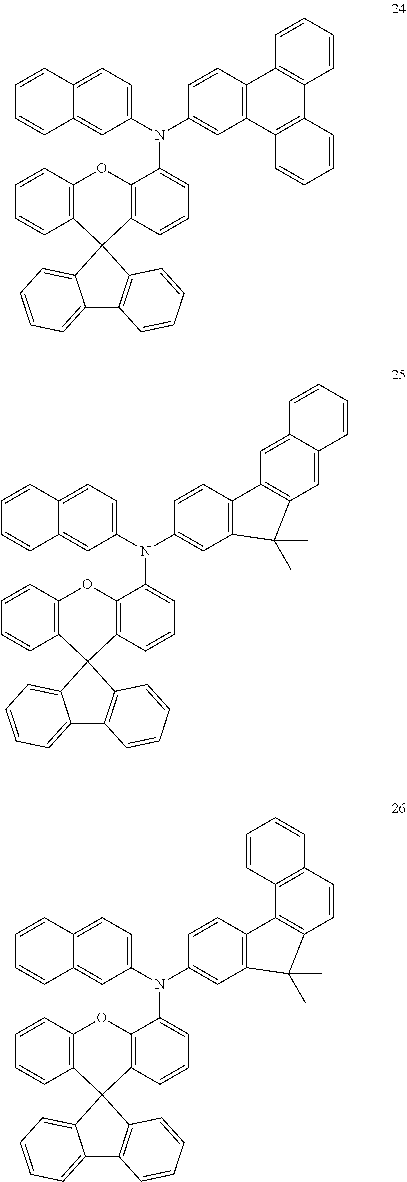 [SMILES: c1ccc2c(c1)Oc1c(N(c3ccc4ccccc4c3)c3ccc4c5ccccc5c5ccccc5c4c3)cccc1C21c2ccccc2-c2ccccc21 ; CC1(C)c2cc(N(c3ccc4ccccc4c3)c3cccc4c3Oc3ccccc3C43c4ccccc4-c4ccccc43)ccc2-c2cc3ccccc3cc21 ; CC1(C)c2cc(N(c3ccc4ccccc4c3)c3cccc4c3Oc3ccccc3C43c4ccccc4-c4ccccc43)ccc2-c2c1ccc1ccccc21]

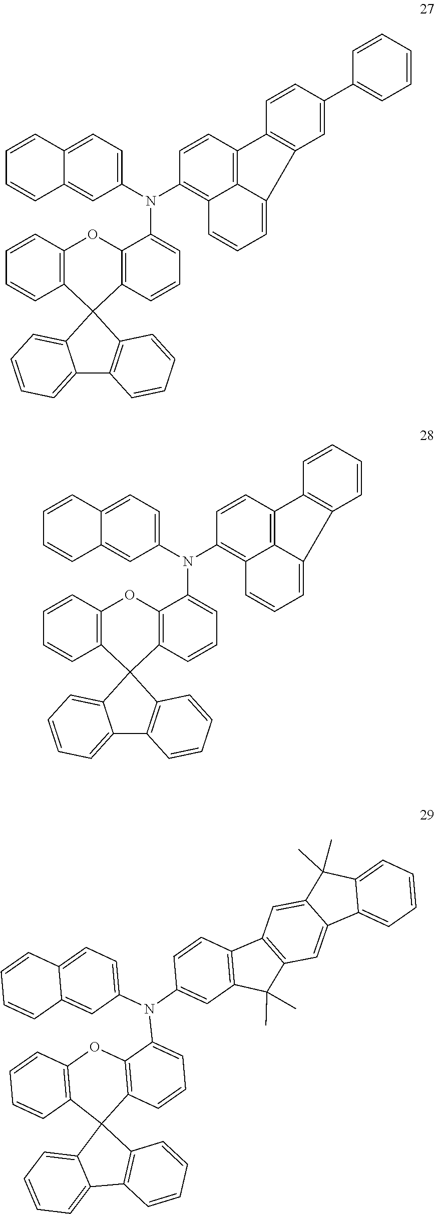 [SMILES: c1ccc(-c2ccc3c(c2)-c2cccc4c(N(c5ccc6ccccc6c5)c5cccc6c5Oc5ccccc5C65c6ccccc6-c6ccccc65)ccc-3c24)cc1 ; c1ccc2c(c1)Oc1c(N(c3ccc4ccccc4c3)c3ccc4c5c(cccc35)-c3ccccc3-4)cccc1C21c2ccccc2-c2ccccc21 ; CC1(C)c2ccccc2-c2cc3c(cc21)-c1ccc(N(c2ccc4ccccc4c2)c2cccc4c2Oc2ccccc2C42c4ccccc4-c4ccccc42)cc1C3(C)C]

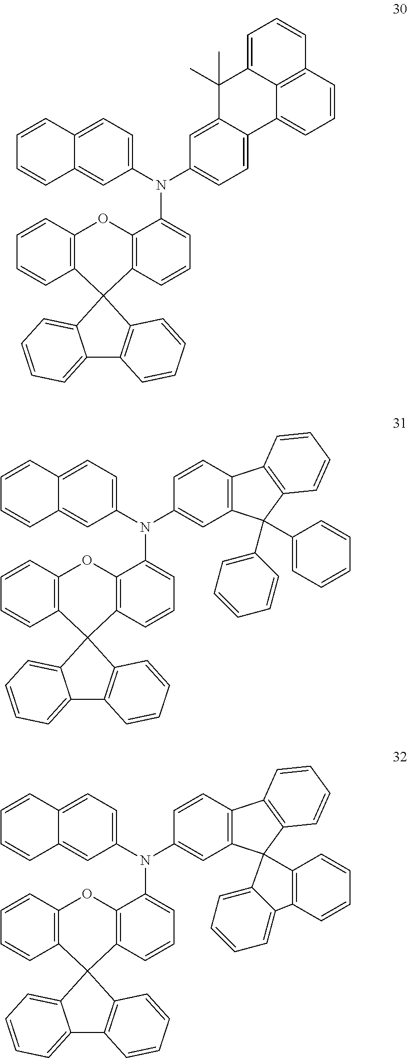 [SMILES: CC1(C)c2cc(N(c3ccc4ccccc4c3)c3cccc4c3Oc3ccccc3C43c4ccccc4-c4ccccc43)ccc2-c2cccc3cccc1c23 ; c1ccc(C2(c3ccccc3)c3ccccc3-c3ccc(N(c4ccc5ccccc5c4)c4cccc5c4Oc4ccccc4C54c5ccccc5-c5ccccc54)cc32)cc1 ; c1ccc2c(c1)Oc1c(N(c3ccc4c(c3)C3(c5ccccc5-c5ccccc53)c3ccccc3-4)c3ccc4ccccc4c3)cccc1C21c2ccccc2-c2ccccc21]

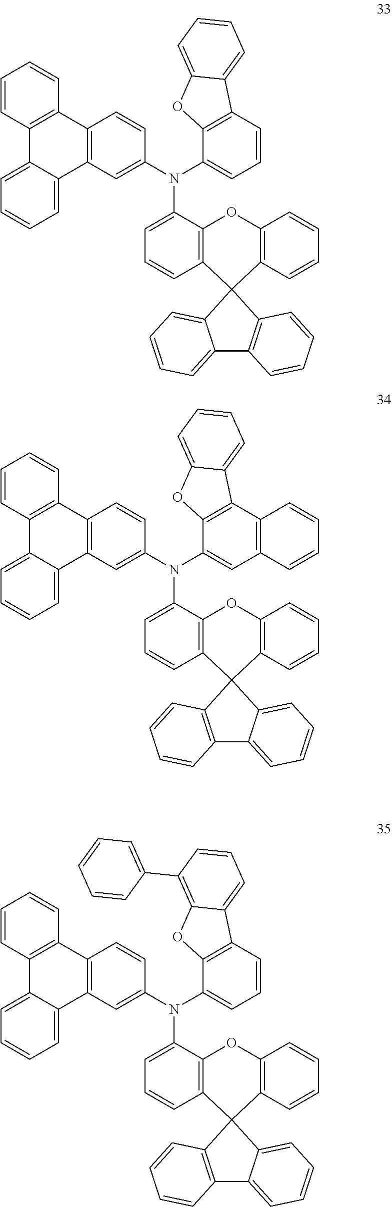

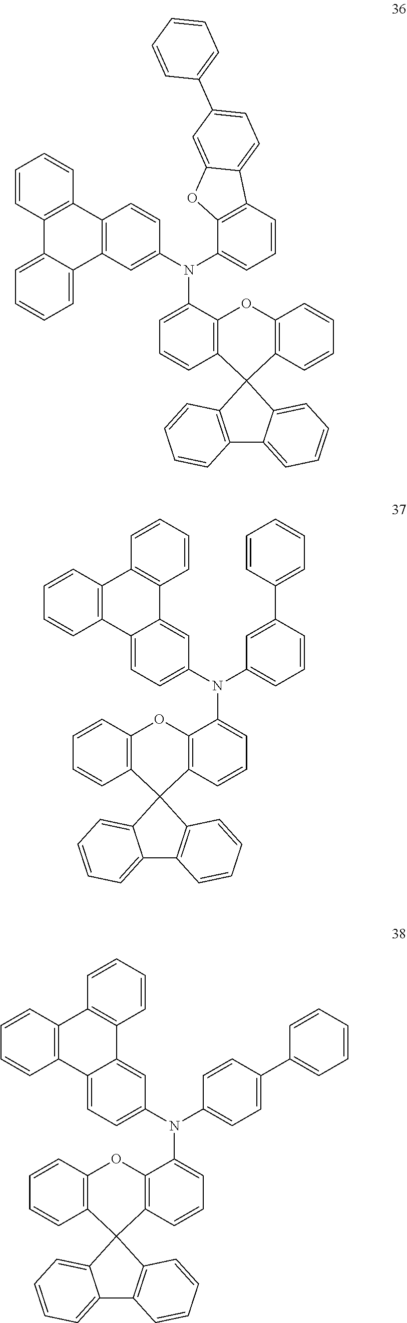 [SMILES: c1ccc(-c2ccc3c(c2)oc2c(N(c4ccc5c6ccccc6c6ccccc6c5c4)c4cccc5c4Oc4ccccc4C54c5ccccc5-c5ccccc54)cccc23)cc1 ; c1ccc(-c2cccc(N(c3ccc4c5ccccc5c5ccccc5c4c3)c3cccc4c3Oc3ccccc3C43c4ccccc4-c4ccccc43)c2)cc1 ; c1ccc(-c2ccc(N(c3ccc4c5ccccc5c5ccccc5c4c3)c3cccc4c3Oc3ccccc3C43c4ccccc4-c4ccccc43)cc2)cc1]

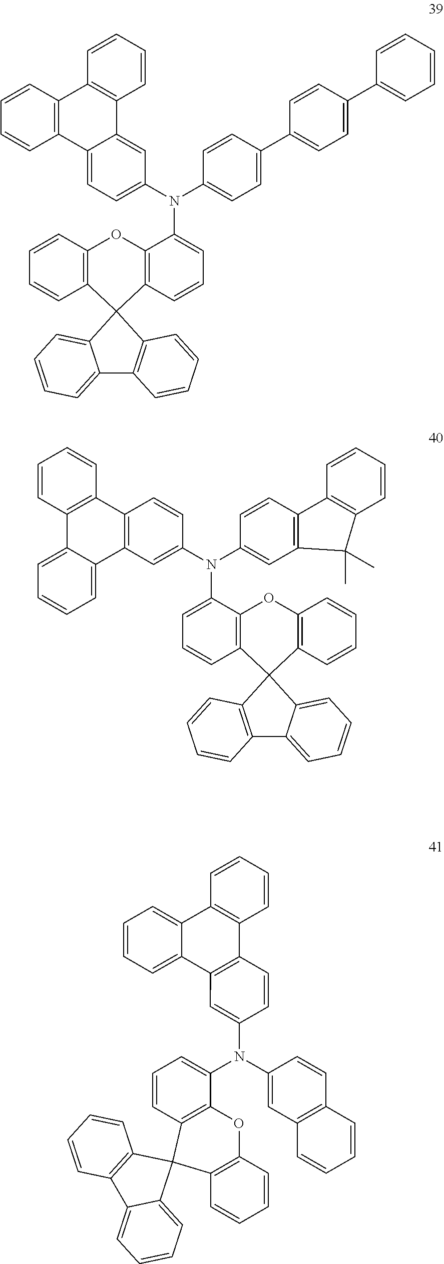 [SMILES: c1ccc(-c2ccc(-c3ccc(N(c4ccc5c6ccccc6c6ccccc6c5c4)c4cccc5c4Oc4ccccc4C54c5ccccc5-c5ccccc54)cc3)cc2)cc1 ; CC1(C)c2ccccc2-c2ccc(N(c3ccc4c5ccccc5c5ccccc5c4c3)c3cccc4c3Oc3ccccc3C43c4ccccc4-c4ccccc43)cc21 ; c1ccc2c(c1)Oc1c(N(c3ccc4ccccc4c3)c3ccc4c5ccccc5c5ccccc5c4c3)cccc1C21c2ccccc2-c2ccccc21]

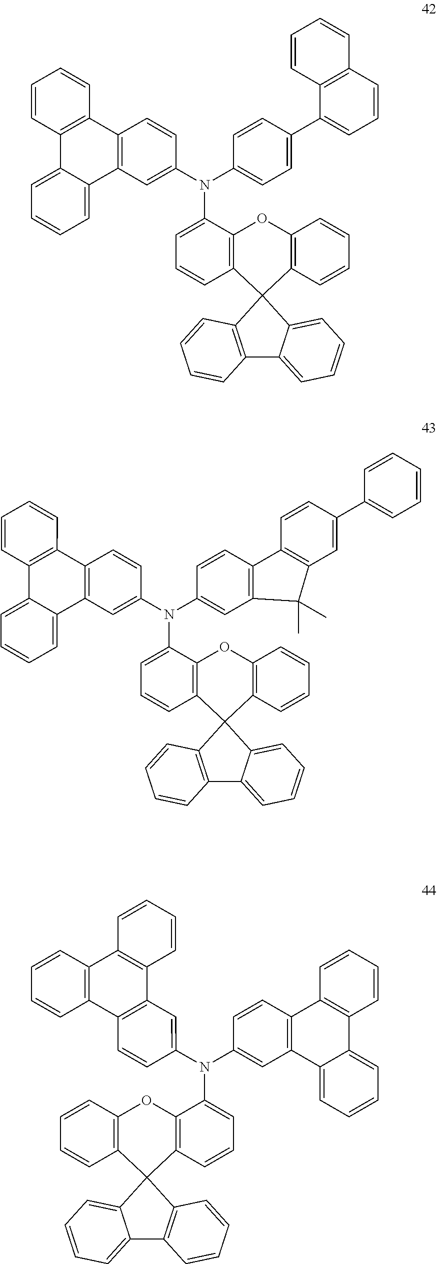 [SMILES: c1ccc2c(c1)Oc1c(N(c3ccc(-c4cccc5ccccc45)cc3)c3ccc4c5ccccc5c5ccccc5c4c3)cccc1C21c2ccccc2-c2ccccc21 ; CC1(C)c2cc(-c3ccccc3)ccc2-c2ccc(N(c3ccc4c5ccccc5c5ccccc5c4c3)c3cccc4c3Oc3ccccc3C43c4ccccc4-c4ccccc43)cc21 ; c1ccc2c(c1)Oc1c(N(c3ccc4c5ccccc5c5ccccc5c4c3)c3ccc4c5ccccc5c5ccccc5c4c3)cccc1C21c2ccccc2-c2ccccc21]

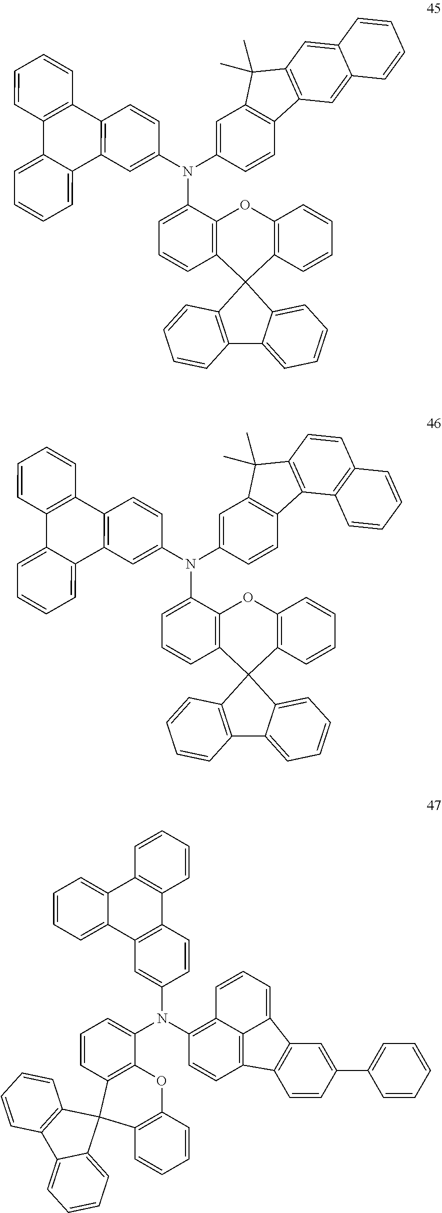 [SMILES: CC1(C)c2cc(N(c3ccc4c5ccccc5c5ccccc5c4c3)c3cccc4c3Oc3ccccc3C43c4ccccc4-c4ccccc43)ccc2-c2cc3ccccc3cc21 ; CC1(C)c2cc(N(c3ccc4c5ccccc5c5ccccc5c4c3)c3cccc4c3Oc3ccccc3C43c4ccccc4-c4ccccc43)ccc2-c2c1ccc1ccccc21 ; c1ccc(-c2ccc3c(c2)-c2cccc4c(N(c5ccc6c7ccccc7c7ccccc7c6c5)c5cccc6c5Oc5ccccc5C65c6ccccc6-c6ccccc65)ccc-3c24)cc1]

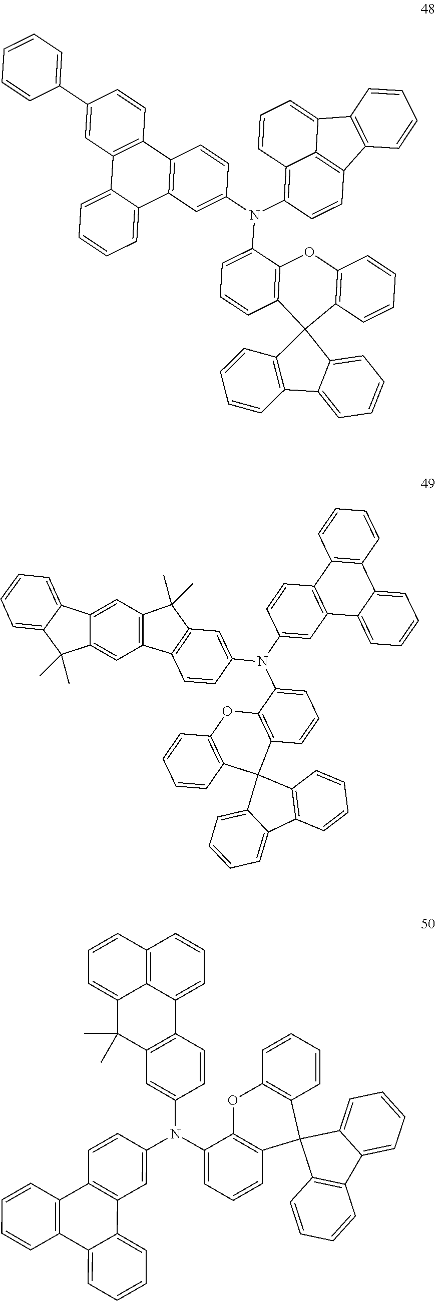 [SMILES: c1ccc(-c2ccc3c4ccc(N(c5cccc6c5Oc5ccccc5C65c6ccccc6-c6ccccc65)c5ccc6c7c(cccc57)-c5ccccc5-6)cc4c4ccccc4c3c2)cc1 ; CC1(C)c2ccccc2-c2cc3c(cc21)-c1ccc(N(c2ccc4c5ccccc5c5ccccc5c4c2)c2cccc4c2Oc2ccccc2C42c4ccccc4-c4ccccc42)cc1C3(C)C ; CC1(C)c2cc(N(c3ccc4c5ccccc5c5ccccc5c4c3)c3cccc4c3Oc3ccccc3C43c4ccccc4-c4ccccc43)ccc2-c2cccc3cccc1c23]

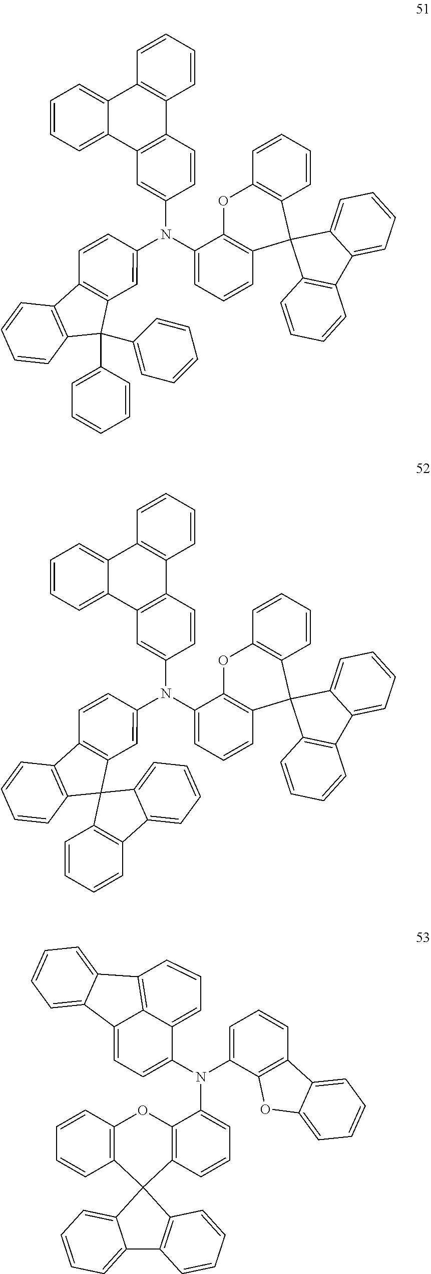 [SMILES: c1ccc(C2(c3ccccc3)c3ccccc3-c3ccc(N(c4ccc5c6ccccc6c6ccccc6c5c4)c4cccc5c4Oc4ccccc4C54c5ccccc5-c5ccccc54)cc32)cc1 ; c1ccc2c(c1)Oc1c(N(c3ccc4c(c3)C3(c5ccccc5-c5ccccc53)c3ccccc3-4)c3ccc4c5ccccc5c5ccccc5c4c3)cccc1C21c2ccccc2-c2ccccc21 ; c1ccc2c(c1)Oc1c(N(c3ccc4c5c(cccc35)-c3ccccc3-4)c3cccc4c3oc3ccccc34)cccc1C21c2ccccc2-c2ccccc21]

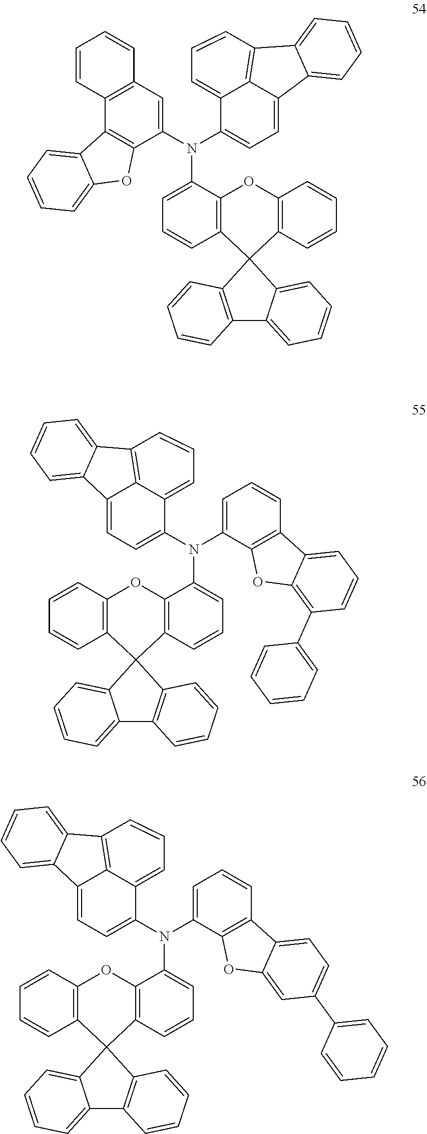 [SMILES: c1ccc2c(c1)Oc1c(N(c3ccc4c5c(cccc35)-c3ccccc3-4)c3cc4ccccc4c4c3oc3ccccc34)cccc1C21c2ccccc2-c2ccccc21 ; c1ccc(-c2cccc3c2oc2c(N(c4cccc5c4Oc4ccccc4C54c5ccccc5-c5ccccc54)c4ccc5c6c(cccc46)-c4ccccc4-5)cccc23)cc1 ; c1ccc(-c2ccc3c(c2)oc2c(N(c4cccc5c4Oc4ccccc4C54c5ccccc5-c5ccccc54)c4ccc5c6c(cccc46)-c4ccccc4-5)cccc23)cc1]

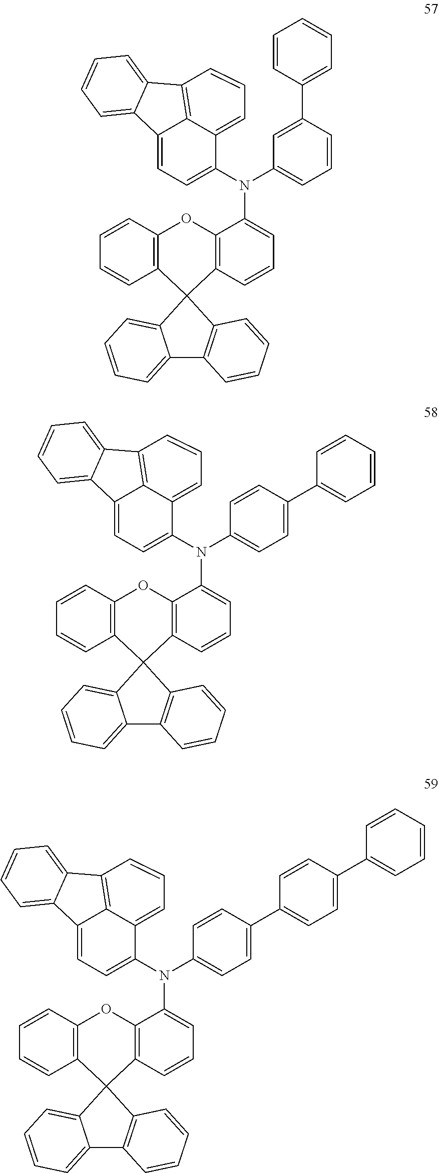 [SMILES: c1ccc(-c2cccc(N(c3cccc4c3Oc3ccccc3C43c4ccccc4-c4ccccc43)c3ccc4c5c(cccc35)-c3ccccc3-4)c2)cc1 ; c1ccc(-c2ccc(N(c3cccc4c3Oc3ccccc3C43c4ccccc4-c4ccccc43)c3ccc4c5c(cccc35)-c3ccccc3-4)cc2)cc1 ; c1ccc(-c2ccc(-c3ccc(N(c4cccc5c4Oc4ccccc4C54c5ccccc5-c5ccccc54)c4ccc5c6c(cccc46)-c4ccccc4-5)cc3)cc2)cc1]

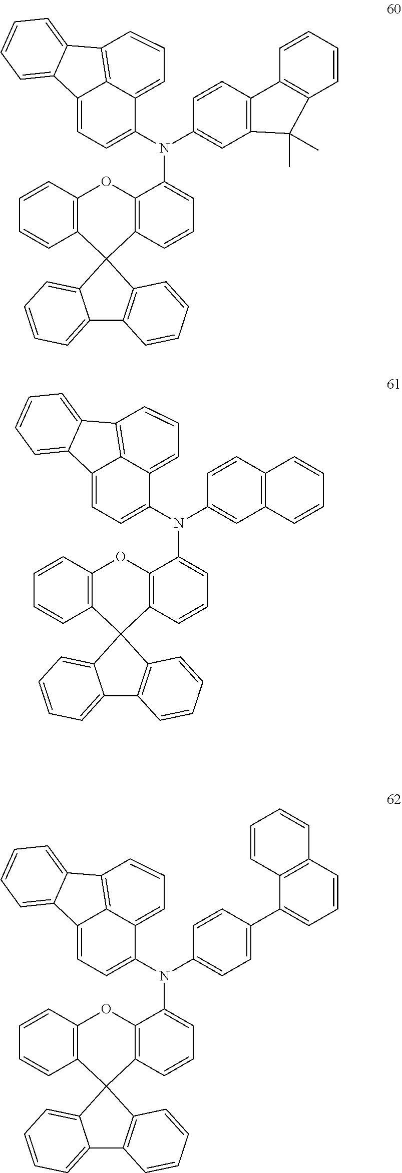 [SMILES: CC1(C)c2ccccc2-c2ccc(N(c3cccc4c3Oc3ccccc3C43c4ccccc4-c4ccccc43)c3ccc4c5c(cccc35)-c3ccccc3-4)cc21 ; c1ccc2c(c1)Oc1c(N(c3ccc4ccccc4c3)c3ccc4c5c(cccc35)-c3ccccc3-4)cccc1C21c2ccccc2-c2ccccc21 ; c1ccc2c(c1)Oc1c(N(c3ccc(-c4cccc5ccccc45)cc3)c3ccc4c5c(cccc35)-c3ccccc3-4)cccc1C21c2ccccc2-c2ccccc21]

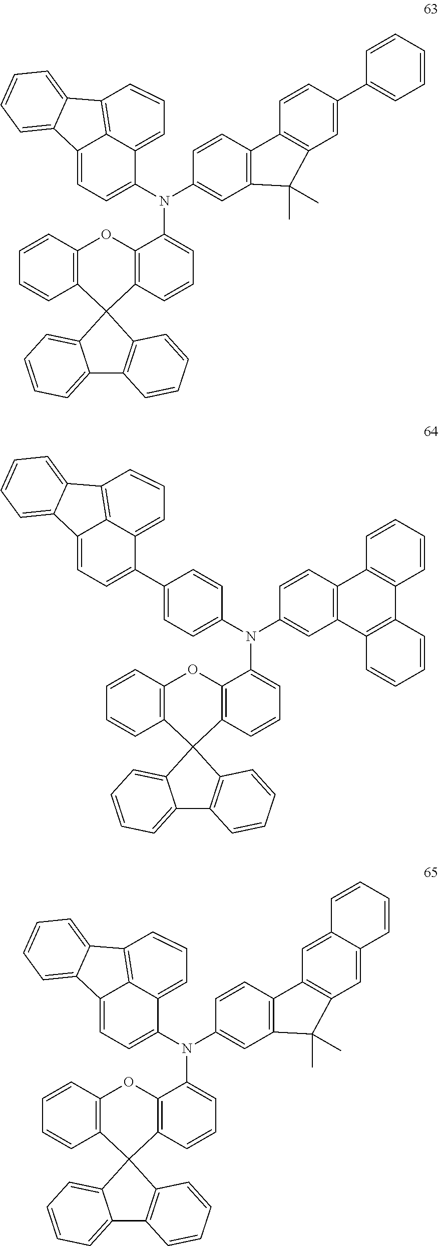 [SMILES: CC1(C)c2cc(-c3ccccc3)ccc2-c2ccc(N(c3cccc4c3Oc3ccccc3C43c4ccccc4-c4ccccc43)c3ccc4c5c(cccc35)-c3ccccc3-4)cc21 ; c1ccc2c(c1)Oc1c(N(c3ccc(-c4ccc5c6c(cccc46)-c4ccccc4-5)cc3)c3ccc4c5ccccc5c5ccccc5c4c3)cccc1C21c2ccccc2-c2ccccc21 ; CC1(C)c2cc(N(c3cccc4c3Oc3ccccc3C43c4ccccc4-c4ccccc43)c3ccc4c5c(cccc35)-c3ccccc3-4)ccc2-c2cc3ccccc3cc21]

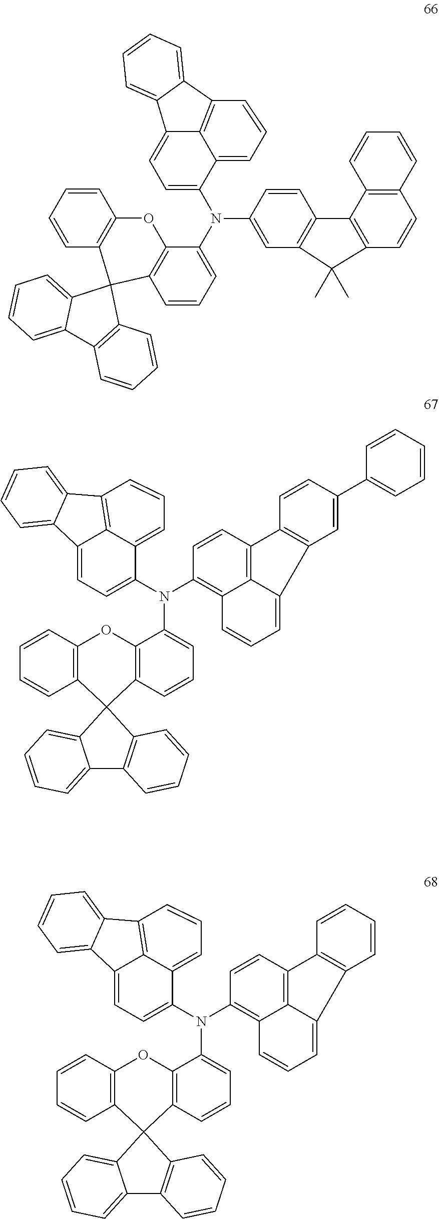 [SMILES: CC1(C)c2cc(N(c3cccc4c3Oc3ccccc3C43c4ccccc4-c4ccccc43)c3ccc4c5c(cccc35)-c3ccccc3-4)ccc2-c2c1ccc1ccccc21 ; c1ccc(-c2ccc3c(c2)-c2cccc4c(N(c5cccc6c5Oc5ccccc5C65c6ccccc6-c6ccccc65)c5ccc6c7c(cccc57)-c5ccccc5-6)ccc-3c24)cc1 ; c1ccc2c(c1)Oc1c(N(c3ccc4c5c(cccc35)-c3ccccc3-4)c3ccc4c5c(cccc35)-c3ccccc3-4)cccc1C21c2ccccc2-c2ccccc21]

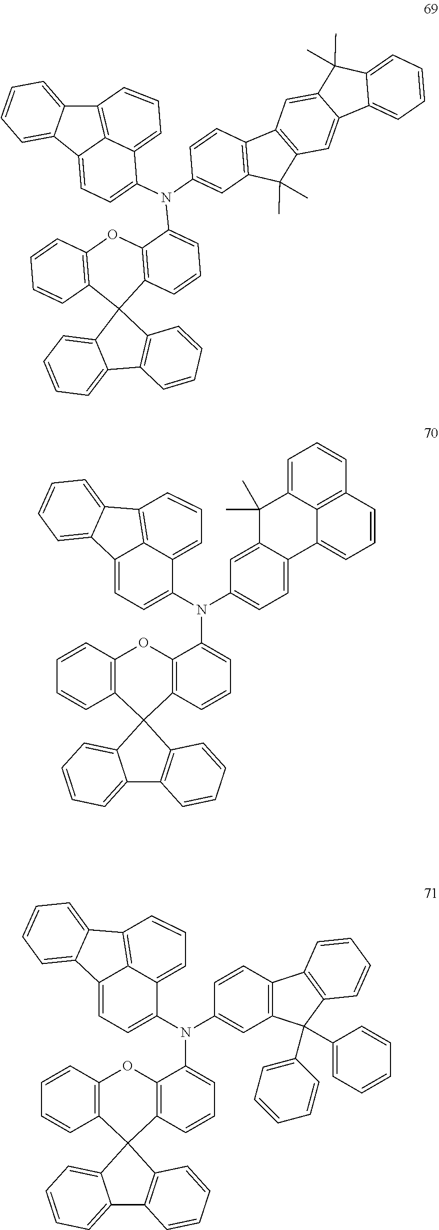 [SMILES: CC1(C)c2ccccc2-c2cc3c(cc21)-c1ccc(N(c2cccc4c2Oc2ccccc2C42c4ccccc4-c4ccccc42)c2ccc4c5c(cccc25)-c2ccccc2-4)cc1C3(C)C ; CC1(C)c2cc(N(c3cccc4c3Oc3ccccc3C43c4ccccc4-c4ccccc43)c3ccc4c5c(cccc35)-c3ccccc3-4)ccc2-c2cccc3cccc1c23 ; c1ccc(C2(c3ccccc3)c3ccccc3-c3ccc(N(c4cccc5c4Oc4ccccc4C54c5ccccc5-c5ccccc54)c4ccc5c6c(cccc46)-c4ccccc4-5)cc32)cc1]

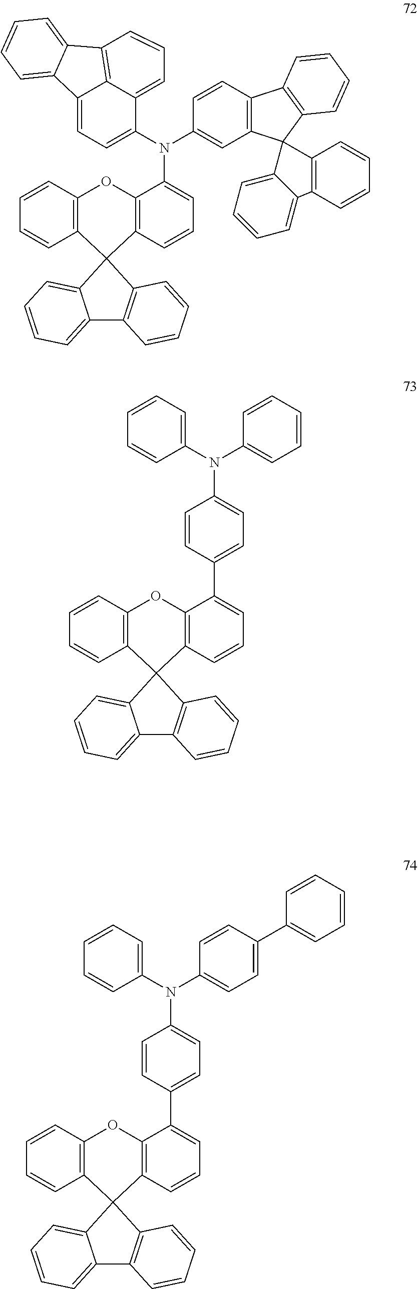 [SMILES: c1ccc2c(c1)Oc1c(N(c3ccc4c(c3)C3(c5ccccc5-c5ccccc53)c3ccccc3-4)c3ccc4c5c(cccc35)-c3ccccc3-4)cccc1C21c2ccccc2-c2ccccc21 ; c1ccc(N(c2ccccc2)c2ccc(-c3cccc4c3Oc3ccccc3C43c4ccccc4-c4ccccc43)cc2)cc1 ; c1ccc(-c2ccc(N(c3ccccc3)c3ccc(-c4cccc5c4Oc4ccccc4C54c5ccccc5-c5ccccc54)cc3)cc2)cc1]

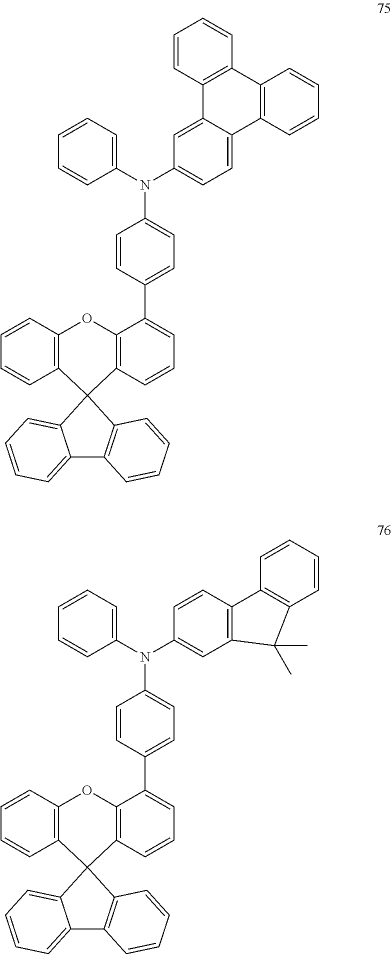 [SMILES: c1ccc(N(c2ccc(-c3cccc4c3Oc3ccccc3C43c4ccccc4-c4ccccc43)cc2)c2ccc3c4ccccc4c4ccccc4c3c2)cc1 ; CC1(C)c2ccccc2-c2ccc(N(c3ccccc3)c3ccc(-c4cccc5c4Oc4ccccc4C54c5ccccc5-c5ccccc54)cc3)cc21]

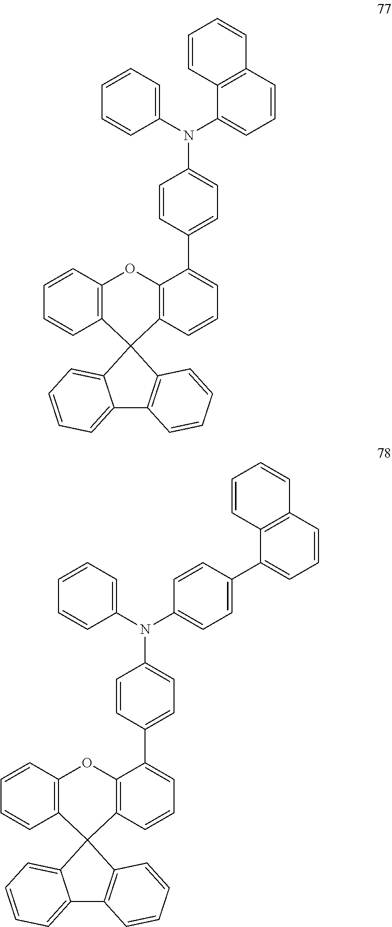 [SMILES: c1ccc(N(c2ccc(-c3cccc4c3Oc3ccccc3C43c4ccccc4-c4ccccc43)cc2)c2cccc3ccccc23)cc1 ; c1ccc(N(c2ccc(-c3cccc4c3Oc3ccccc3C43c4ccccc4-c4ccccc43)cc2)c2ccc(-c3cccc4ccccc34)cc2)cc1]

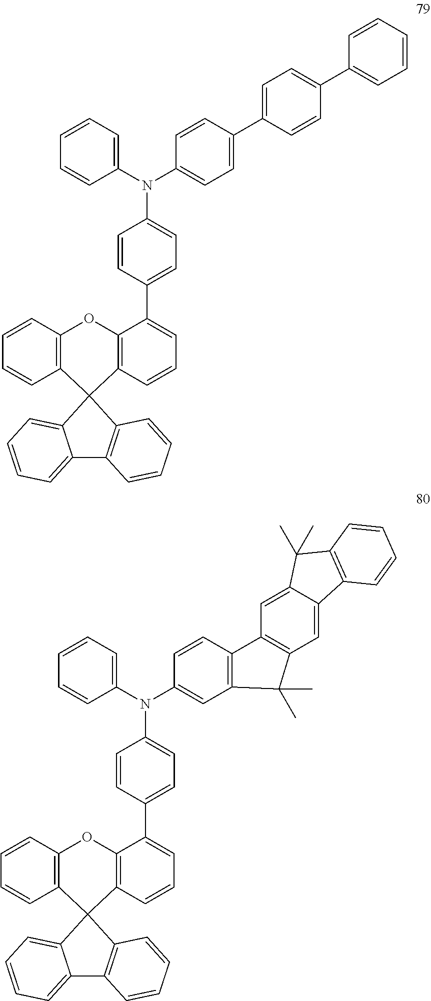 [SMILES: c1ccc(-c2ccc(-c3ccc(N(c4ccccc4)c4ccc(-c5cccc6c5Oc5ccccc5C65c6ccccc6-c6ccccc65)cc4)cc3)cc2)cc1 ; CC1(C)c2ccccc2-c2cc3c(cc21)-c1ccc(N(c2ccccc2)c2ccc(-c4cccc5c4Oc4ccccc4C54c5ccccc5-c5ccccc54)cc2)cc1C3(C)C]

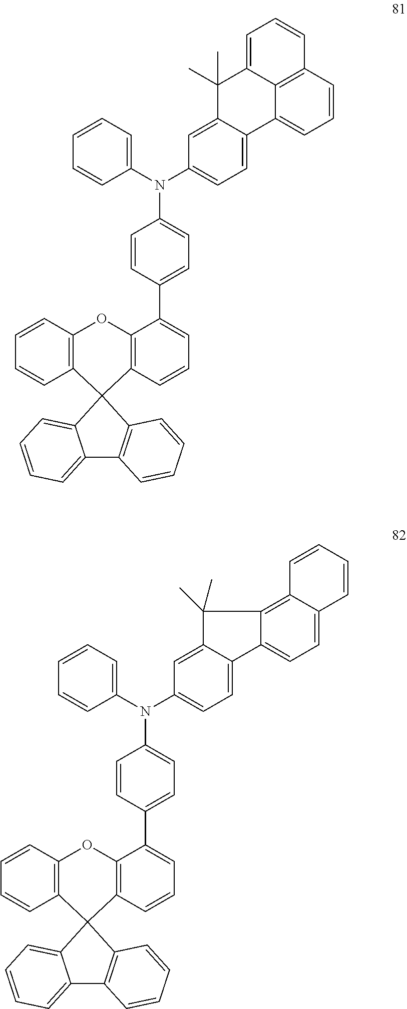 [SMILES: CC1(C)c2cc(N(c3ccccc3)c3ccc(-c4cccc5c4Oc4ccccc4C54c5ccccc5-c5ccccc54)cc3)ccc2-c2cccc3cccc1c23 ; CC1(C)c2cc(N(c3ccccc3)c3ccc(-c4cccc5c4Oc4ccccc4C54c5ccccc5-c5ccccc54)cc3)ccc2-c2ccc3ccccc3c21]

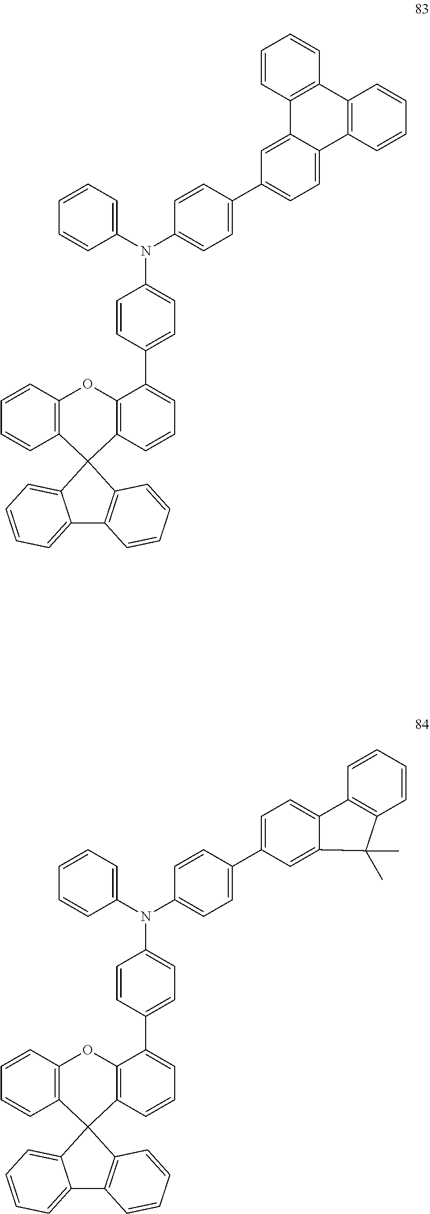 [SMILES: c1ccc(N(c2ccc(-c3ccc4c5ccccc5c5ccccc5c4c3)cc2)c2ccc(-c3cccc4c3Oc3ccccc3C43c4ccccc4-c4ccccc43)cc2)cc1 ; CC1(C)c2ccccc2-c2ccc(-c3ccc(N(c4ccccc4)c4ccc(-c5cccc6c5Oc5ccccc5C65c6ccccc6-c6ccccc65)cc4)cc3)cc21]

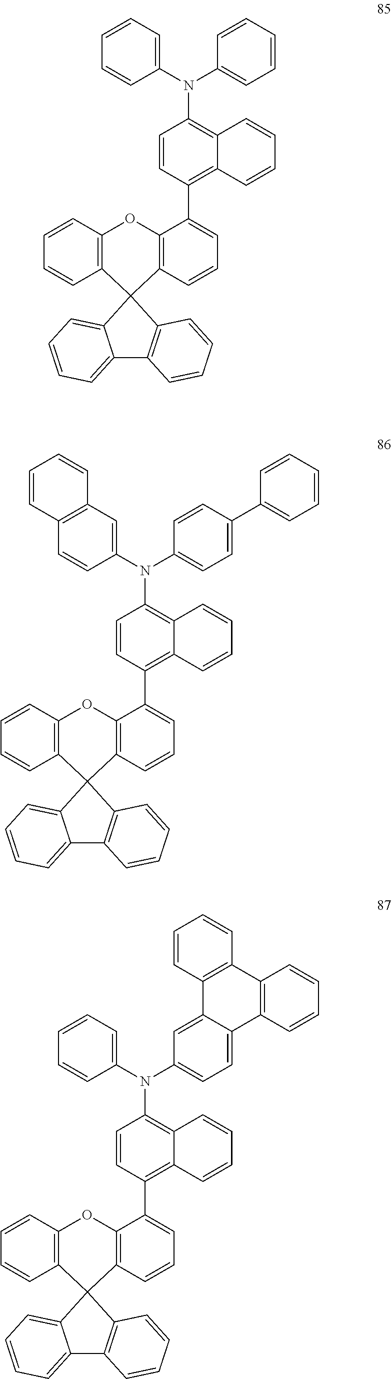 [SMILES: c1ccc(N(c2ccccc2)c2ccc(-c3cccc4c3Oc3ccccc3C43c4ccccc4-c4ccccc43)c3ccccc23)cc1 ; c1ccc(-c2ccc(N(c3ccc4ccccc4c3)c3ccc(-c4cccc5c4Oc4ccccc4C54c5ccccc5-c5ccccc54)c4ccccc34)cc2)cc1 ; c1ccc(N(c2ccc3c4ccccc4c4ccccc4c3c2)c2ccc(-c3cccc4c3Oc3ccccc3C43c4ccccc4-c4ccccc43)c3ccccc23)cc1]

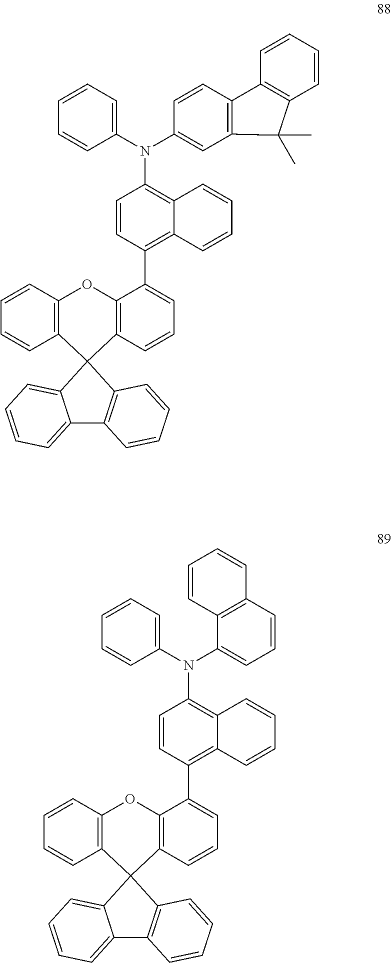 [SMILES: CC1(C)c2ccccc2-c2ccc(N(c3ccccc3)c3ccc(-c4cccc5c4Oc4ccccc4C54c5ccccc5-c5ccccc54)c4ccccc34)cc21 ; c1ccc(N(c2cccc3ccccc23)c2ccc(-c3cccc4c3Oc3ccccc3C43c4ccccc4-c4ccccc43)c3ccccc23)cc1]

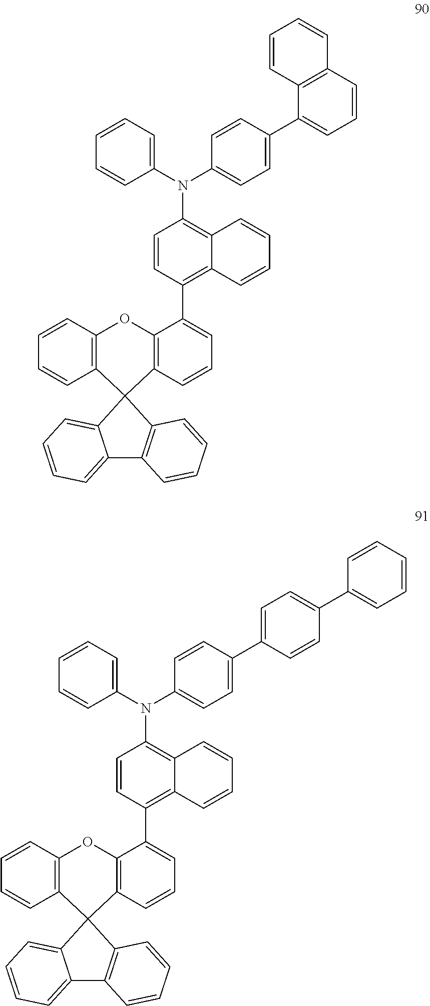 [SMILES: c1ccc(N(c2ccc(-c3cccc4ccccc34)cc2)c2ccc(-c3cccc4c3Oc3ccccc3C43c4ccccc4-c4ccccc43)c3ccccc23)cc1 ; c1ccc(-c2ccc(-c3ccc(N(c4ccccc4)c4ccc(-c5cccc6c5Oc5ccccc5C65c6ccccc6-c6ccccc65)c5ccccc45)cc3)cc2)cc1]

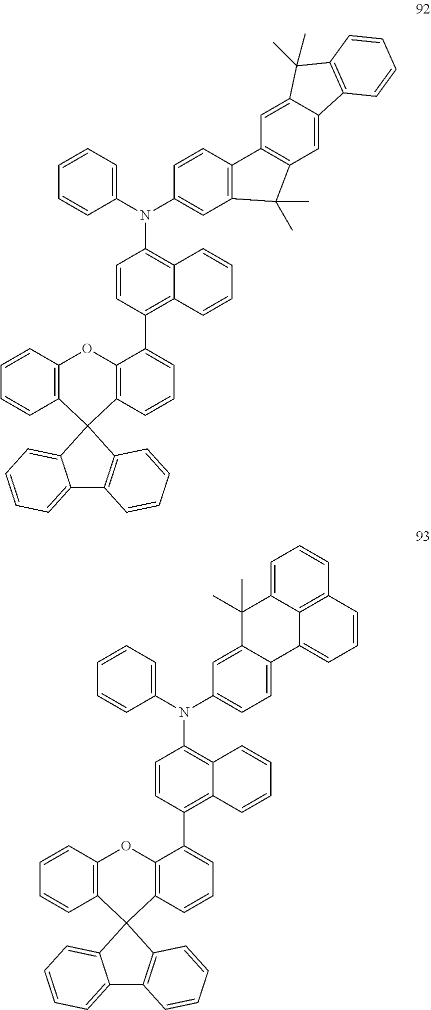 [SMILES: CC1(C)c2ccccc2-c2cc3c(cc21)-c1ccc(N(c2ccccc2)c2ccc(-c4cccc5c4Oc4ccccc4C54c5ccccc5-c5ccccc54)c4ccccc24)cc1C3(C)C ; CC1(C)c2cc(N(c3ccccc3)c3ccc(-c4cccc5c4Oc4ccccc4C54c5ccccc5-c5ccccc54)c4ccccc34)ccc2-c2cccc3cccc1c23]

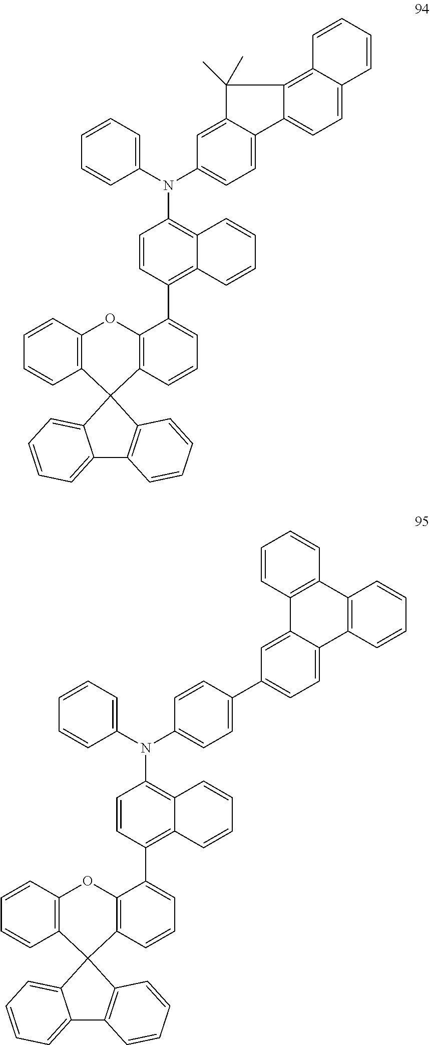 [SMILES: CC1(C)c2cc(N(c3ccccc3)c3ccc(-c4cccc5c4Oc4ccccc4C54c5ccccc5-c5ccccc54)c4ccccc34)ccc2-c2ccc3ccccc3c21 ; c1ccc(N(c2ccc(-c3ccc4c5ccccc5c5ccccc5c4c3)cc2)c2ccc(-c3cccc4c3Oc3ccccc3C43c4ccccc4-c4ccccc43)c3ccccc23)cc1]

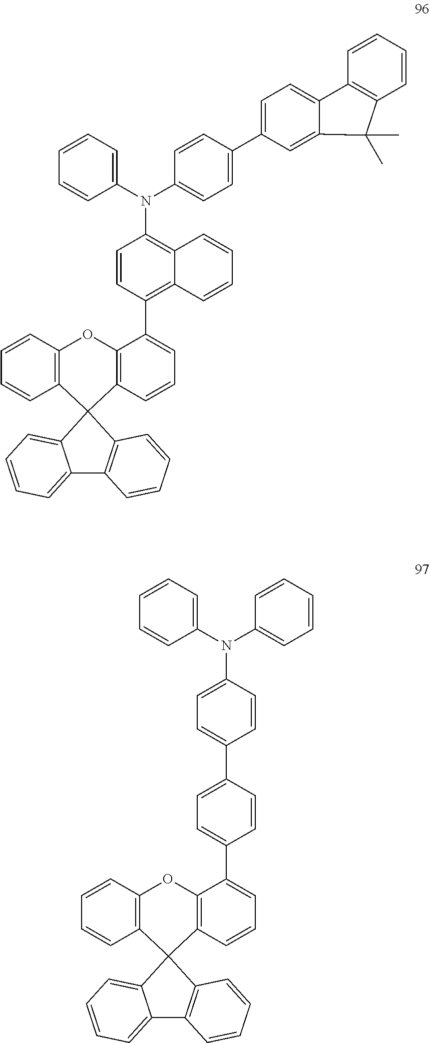 [SMILES: CC1(C)c2ccccc2-c2ccc(-c3ccc(N(c4ccccc4)c4ccc(-c5cccc6c5Oc5ccccc5C65c6ccccc6-c6ccccc65)c5ccccc45)cc3)cc21 ; c1ccc(N(c2ccccc2)c2ccc(-c3ccc(-c4cccc5c4Oc4ccccc4C54c5ccccc5-c5ccccc54)cc3)cc2)cc1]

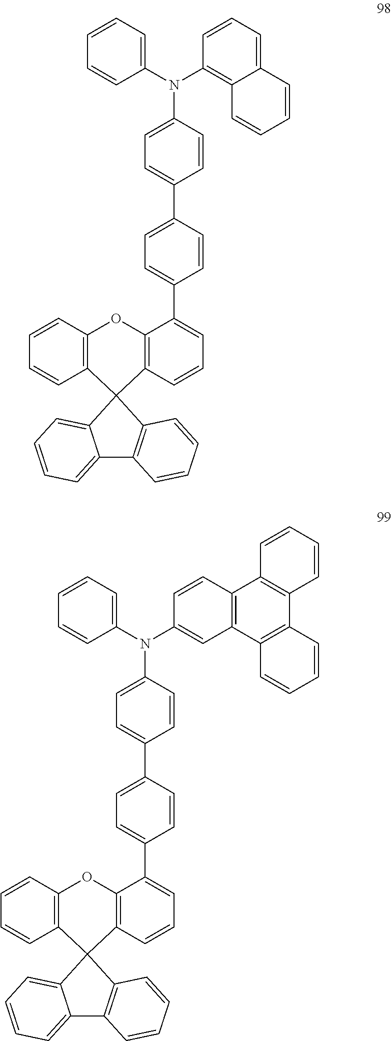 [SMILES: c1ccc(N(c2ccc(-c3ccc(-c4cccc5c4Oc4ccccc4C54c5ccccc5-c5ccccc54)cc3)cc2)c2cccc3ccccc23)cc1 ; c1ccc(N(c2ccc(-c3ccc(-c4cccc5c4Oc4ccccc4C54c5ccccc5-c5ccccc54)cc3)cc2)c2ccc3c4ccccc4c4ccccc4c3c2)cc1]

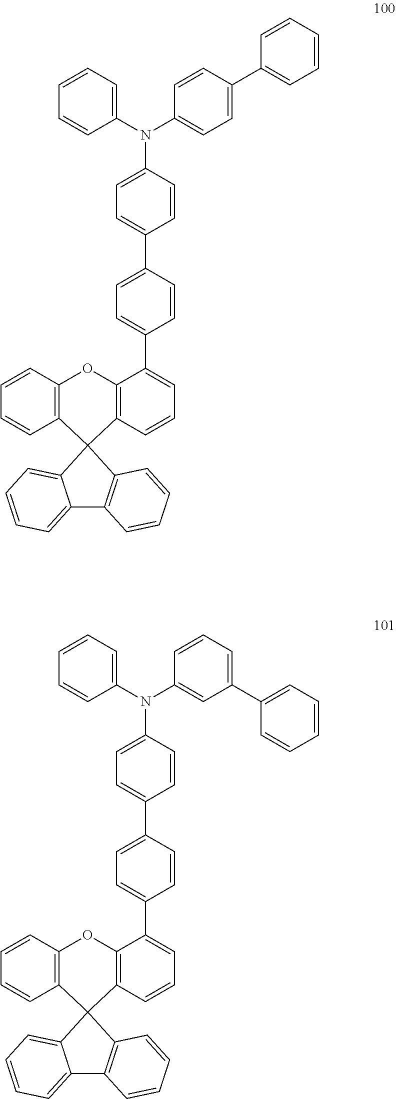 [SMILES: c1ccc(-c2ccc(N(c3ccccc3)c3ccc(-c4ccc(-c5cccc6c5Oc5ccccc5C65c6ccccc6-c6ccccc65)cc4)cc3)cc2)cc1 ; c1ccc(-c2cccc(N(c3ccccc3)c3ccc(-c4ccc(-c5cccc6c5Oc5ccccc5C65c6ccccc6-c6ccccc65)cc4)cc3)c2)cc1]

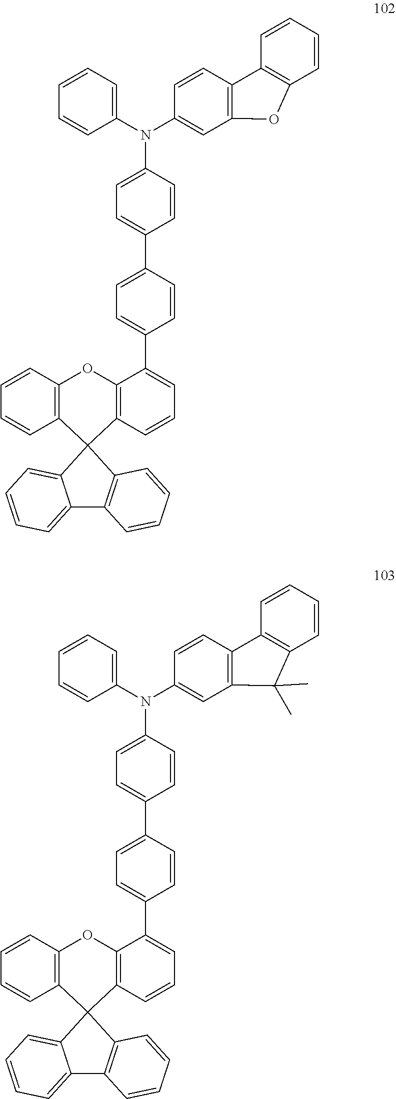 [SMILES: c1ccc(N(c2ccc(-c3ccc(-c4cccc5c4Oc4ccccc4C54c5ccccc5-c5ccccc54)cc3)cc2)c2ccc3c(c2)oc2ccccc23)cc1 ; CC1(C)c2ccccc2-c2ccc(N(c3ccccc3)c3ccc(-c4ccc(-c5cccc6c5Oc5ccccc5C65c6ccccc6-c6ccccc65)cc4)cc3)cc21]

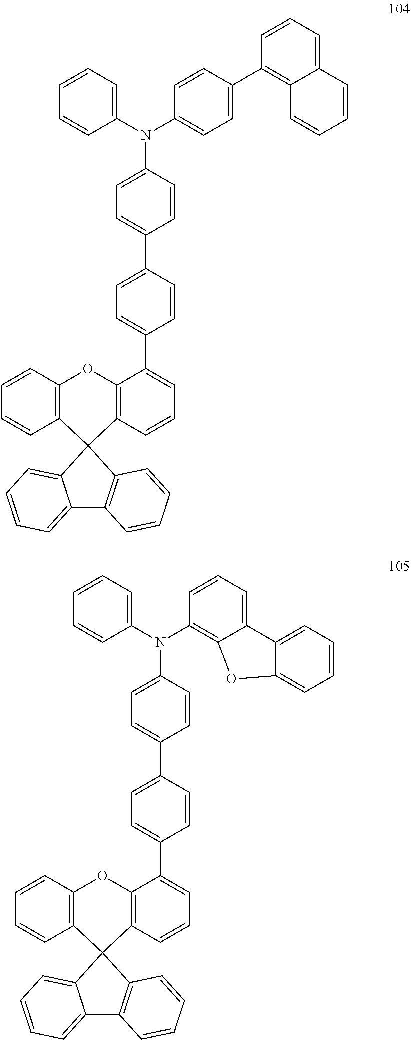 [SMILES: c1ccc(N(c2ccc(-c3ccc(-c4cccc5c4Oc4ccccc4C54c5ccccc5-c5ccccc54)cc3)cc2)c2ccc(-c3cccc4ccccc34)cc2)cc1 ; c1ccc(N(c2ccc(-c3ccc(-c4cccc5c4Oc4ccccc4C54c5ccccc5-c5ccccc54)cc3)cc2)c2cccc3c2oc2ccccc23)cc1]

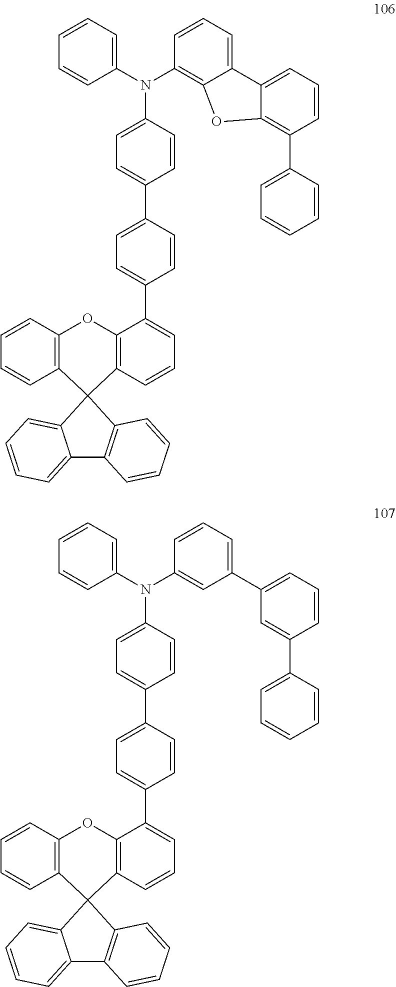 [SMILES: c1ccc(-c2cccc3c2oc2c(N(c4ccccc4)c4ccc(-c5ccc(-c6cccc7c6Oc6ccccc6C76c7ccccc7-c7ccccc76)cc5)cc4)cccc23)cc1 ; c1ccc(-c2cccc(-c3cccc(N(c4ccccc4)c4ccc(-c5ccc(-c6cccc7c6Oc6ccccc6C76c7ccccc7-c7ccccc76)cc5)cc4)c3)c2)cc1]

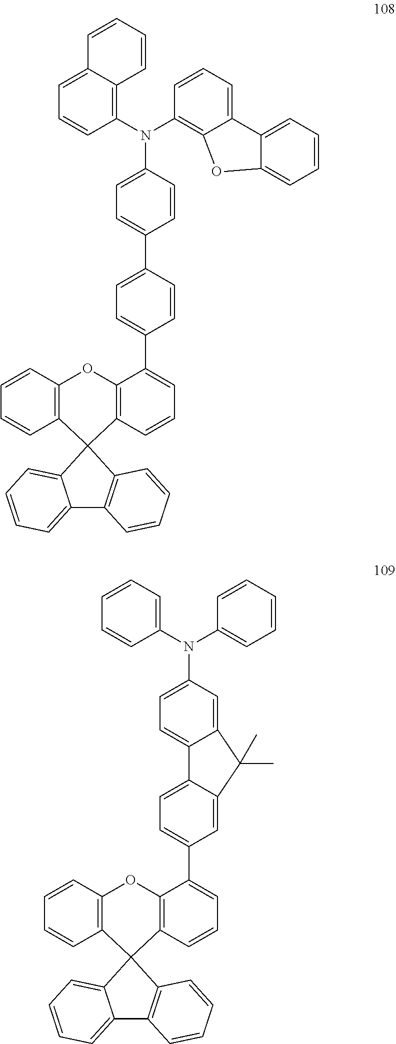 [SMILES: c1ccc2c(c1)Oc1c(-c3ccc(-c4ccc(N(c5cccc6ccccc56)c5cccc6c5oc5ccccc56)cc4)cc3)cccc1C21c2ccccc2-c2ccccc21 ; CC1(C)c2cc(-c3cccc4c3Oc3ccccc3C43c4ccccc4-c4ccccc43)ccc2-c2ccc(N(c3ccccc3)c3ccccc3)cc21]

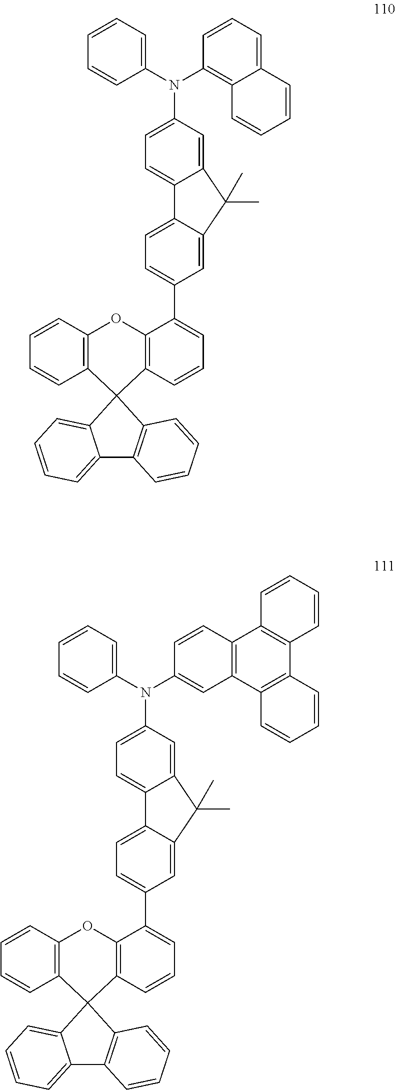 [SMILES: CC1(C)c2cc(-c3cccc4c3Oc3ccccc3C43c4ccccc4-c4ccccc43)ccc2-c2ccc(N(c3ccccc3)c3cccc4ccccc34)cc21 ; CC1(C)c2cc(-c3cccc4c3Oc3ccccc3C43c4ccccc4-c4ccccc43)ccc2-c2ccc(N(c3ccccc3)c3ccc4c5ccccc5c5ccccc5c4c3)cc21]

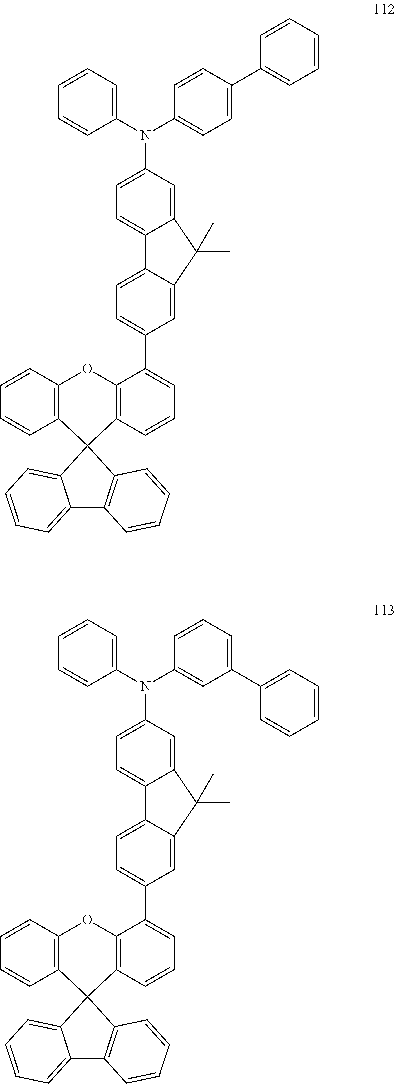 [SMILES: CC1(C)c2cc(-c3cccc4c3Oc3ccccc3C43c4ccccc4-c4ccccc43)ccc2-c2ccc(N(c3ccccc3)c3ccc(-c4ccccc4)cc3)cc21 ; CC1(C)c2cc(-c3cccc4c3Oc3ccccc3C43c4ccccc4-c4ccccc43)ccc2-c2ccc(N(c3ccccc3)c3cccc(-c4ccccc4)c3)cc21]

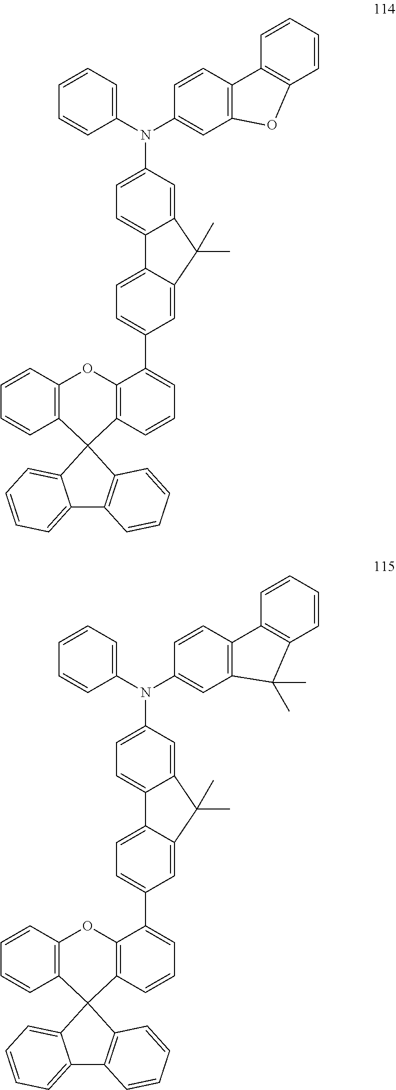 [SMILES: CC1(C)c2cc(-c3cccc4c3Oc3ccccc3C43c4ccccc4-c4ccccc43)ccc2-c2ccc(N(c3ccccc3)c3ccc4c(c3)oc3ccccc34)cc21 ; CC1(C)c2ccccc2-c2ccc(N(c3ccccc3)c3ccc4c(c3)C(C)(C)c3cc(-c5cccc6c5Oc5ccccc5C65c6ccccc6-c6ccccc65)ccc3-4)cc21]

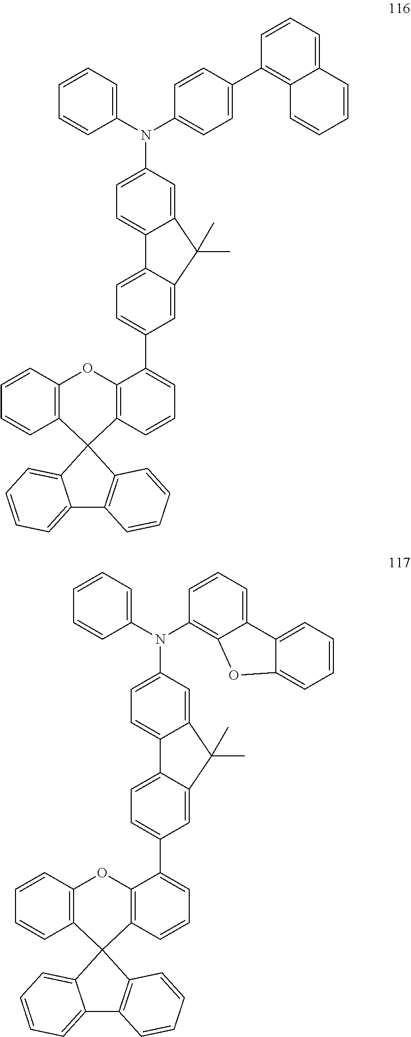 [SMILES: CC1(C)c2cc(-c3cccc4c3Oc3ccccc3C43c4ccccc4-c4ccccc43)ccc2-c2ccc(N(c3ccccc3)c3ccc(-c4cccc5ccccc45)cc3)cc21 ; CC1(C)c2cc(-c3cccc4c3Oc3ccccc3C43c4ccccc4-c4ccccc43)ccc2-c2ccc(N(c3ccccc3)c3cccc4c3oc3ccccc34)cc21]

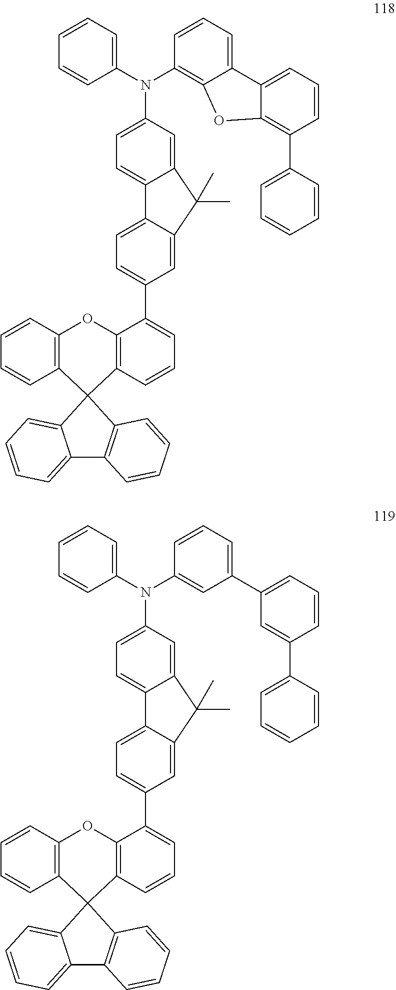 [SMILES: CC1(C)c2cc(-c3cccc4c3Oc3ccccc3C43c4ccccc4-c4ccccc43)ccc2-c2ccc(N(c3ccccc3)c3cccc4c3oc3c(-c5ccccc5)cccc34)cc21 ; CC1(C)c2cc(-c3cccc4c3Oc3ccccc3C43c4ccccc4-c4ccccc43)ccc2-c2ccc(N(c3ccccc3)c3cccc(-c4cccc(-c5ccccc5)c4)c3)cc21]

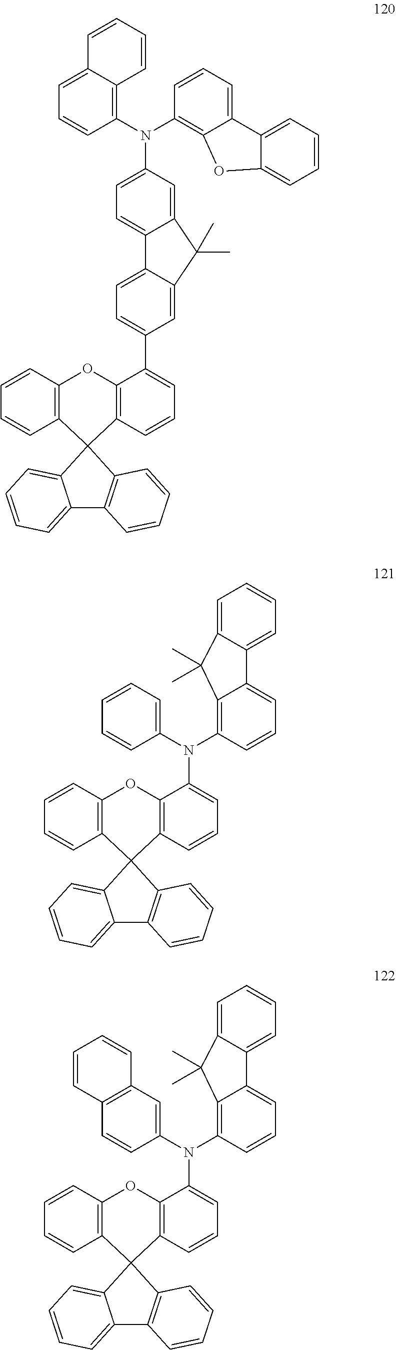 [SMILES: CC1(C)c2cc(-c3cccc4c3Oc3ccccc3C43c4ccccc4-c4ccccc43)ccc2-c2ccc(N(c3cccc4ccccc34)c3cccc4c3oc3ccccc34)cc21 ; CC1(C)c2ccccc2-c2cccc(N(c3ccccc3)c3cccc4c3Oc3ccccc3C43c4ccccc4-c4ccccc43)c21 ; CC1(C)c2ccccc2-c2cccc(N(c3ccc4ccccc4c3)c3cccc4c3Oc3ccccc3C43c4ccccc4-c4ccccc43)c21]

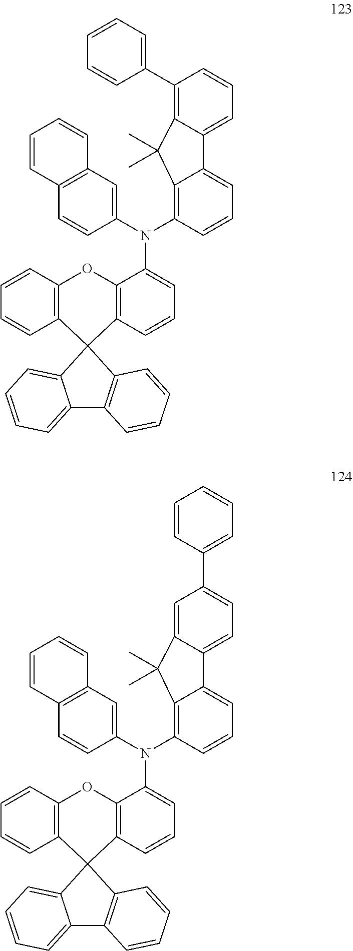 [SMILES: CC1(C)c2c(-c3ccccc3)cccc2-c2cccc(N(c3ccc4ccccc4c3)c3cccc4c3Oc3ccccc3C43c4ccccc4-c4ccccc43)c21 ; CC1(C)c2cc(-c3ccccc3)ccc2-c2cccc(N(c3ccc4ccccc4c3)c3cccc4c3Oc3ccccc3C43c4ccccc4-c4ccccc43)c21]

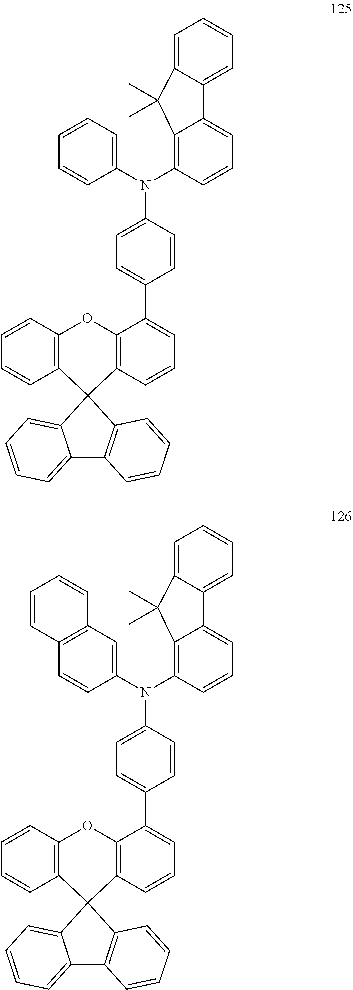 [SMILES: CC1(C)c2ccccc2-c2cccc(N(c3ccccc3)c3ccc(-c4cccc5c4Oc4ccccc4C54c5ccccc5-c5ccccc54)cc3)c21 ; CC1(C)c2ccccc2-c2cccc(N(c3ccc(-c4cccc5c4Oc4ccccc4C54c5ccccc5-c5ccccc54)cc3)c3ccc4ccccc4c3)c21]

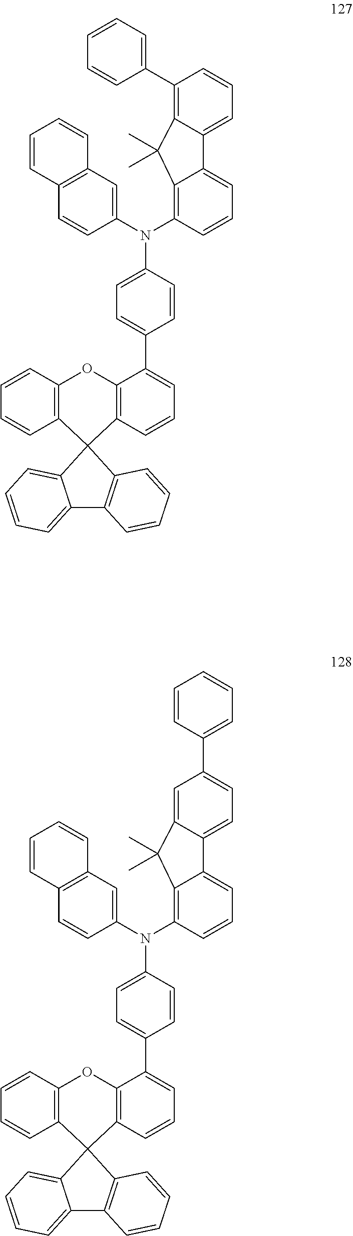 [SMILES: CC1(C)c2c(-c3ccccc3)cccc2-c2cccc(N(c3ccc(-c4cccc5c4Oc4ccccc4C54c5ccccc5-c5ccccc54)cc3)c3ccc4ccccc4c3)c21 ; CC1(C)c2cc(-c3ccccc3)ccc2-c2cccc(N(c3ccc(-c4cccc5c4Oc4ccccc4C54c5ccccc5-c5ccccc54)cc3)c3ccc4ccccc4c3)c21]

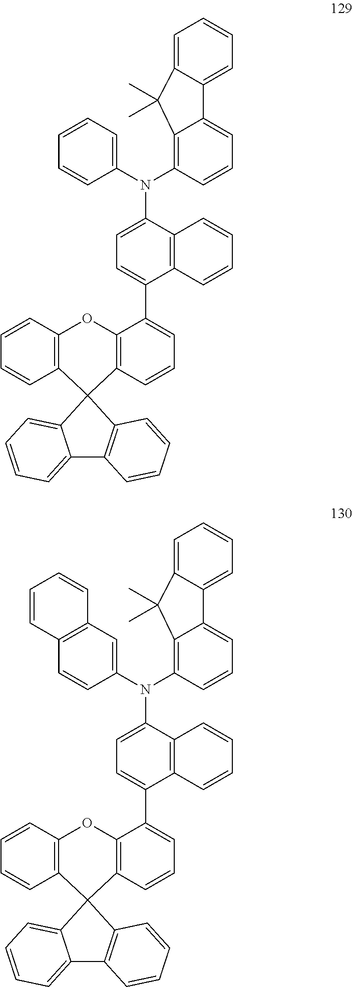 [SMILES: CC1(C)c2ccccc2-c2cccc(N(c3ccccc3)c3ccc(-c4cccc5c4Oc4ccccc4C54c5ccccc5-c5ccccc54)c4ccccc34)c21 ; CC1(C)c2ccccc2-c2cccc(N(c3ccc4ccccc4c3)c3ccc(-c4cccc5c4Oc4ccccc4C54c5ccccc5-c5ccccc54)c4ccccc34)c21]

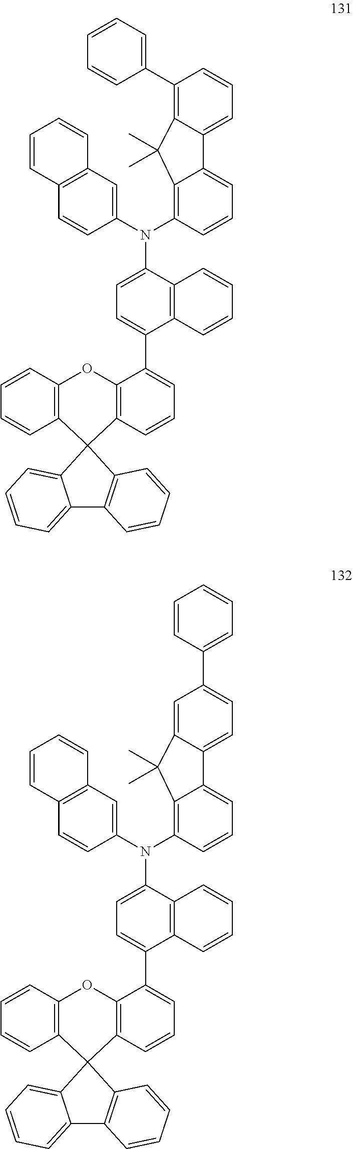 [SMILES: CC1(C)c2c(-c3ccccc3)cccc2-c2cccc(N(c3ccc4ccccc4c3)c3ccc(-c4cccc5c4Oc4ccccc4C54c5ccccc5-c5ccccc54)c4ccccc34)c21 ; CC1(C)c2cc(-c3ccccc3)ccc2-c2cccc(N(c3ccc4ccccc4c3)c3ccc(-c4cccc5c4Oc4ccccc4C54c5ccccc5-c5ccccc54)c4ccccc34)c21]

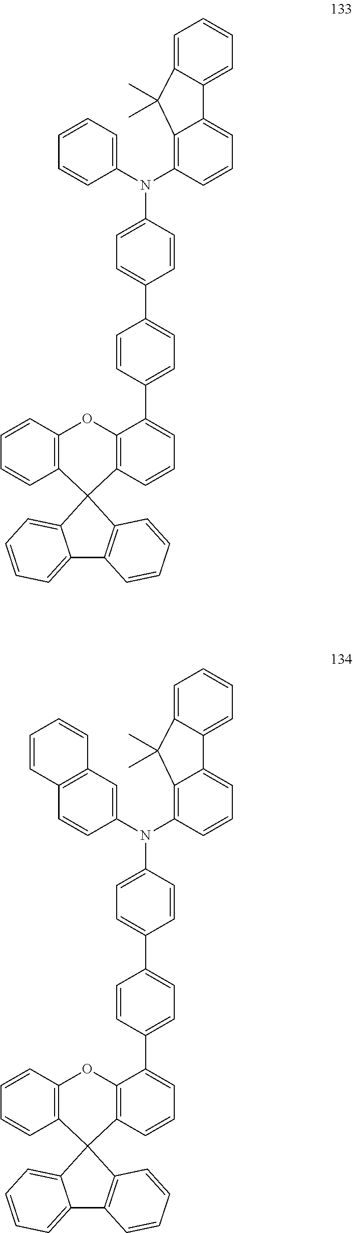 [SMILES: CC1(C)c2ccccc2-c2cccc(N(c3ccccc3)c3ccc(-c4ccc(-c5cccc6c5Oc5ccccc5C65c6ccccc6-c6ccccc65)cc4)cc3)c21 ; CC1(C)c2ccccc2-c2cccc(N(c3ccc(-c4ccc(-c5cccc6c5Oc5ccccc5C65c6ccccc6-c6ccccc65)cc4)cc3)c3ccc4ccccc4c3)c21]

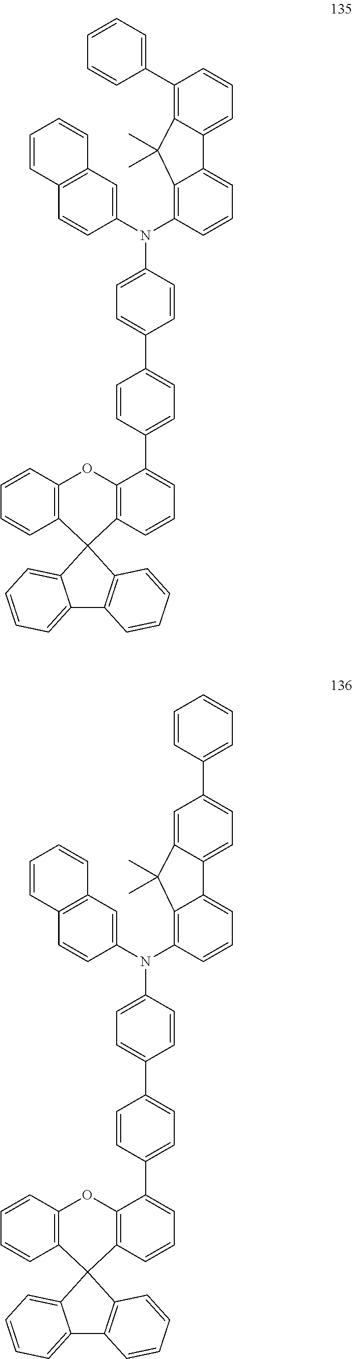 [SMILES: CC1(C)c2c(-c3ccccc3)cccc2-c2cccc(N(c3ccc(-c4ccc(-c5cccc6c5Oc5ccccc5C65c6ccccc6-c6ccccc65)cc4)cc3)c3ccc4ccccc4c3)c21 ; CC1(C)c2cc(-c3ccccc3)ccc2-c2cccc(N(c3ccc(-c4ccc(-c5cccc6c5Oc5ccccc5C65c6ccccc6-c6ccccc65)cc4)cc3)c3ccc4ccccc4c3)c21]

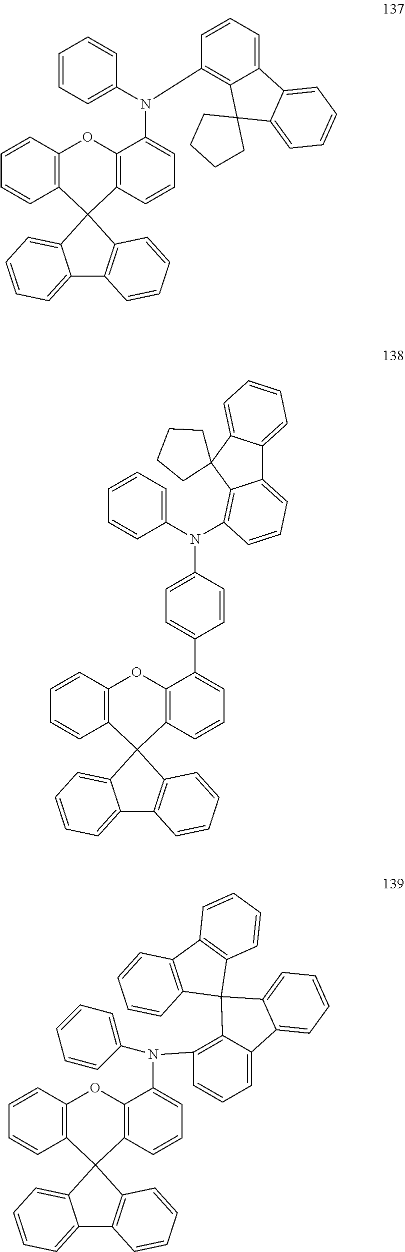 [SMILES: c1ccc(N(c2cccc3c2Oc2ccccc2C32c3ccccc3-c3ccccc32)c2cccc3c2C2(CCCC2)c2ccccc2-3)cc1 ; c1ccc(N(c2ccc(-c3cccc4c3Oc3ccccc3C43c4ccccc4-c4ccccc43)cc2)c2cccc3c2C2(CCCC2)c2ccccc2-3)cc1 ; c1ccc(N(c2cccc3c2Oc2ccccc2C32c3ccccc3-c3ccccc32)c2cccc3c2C2(c4ccccc4-c4ccccc42)c2ccccc2-3)cc1]

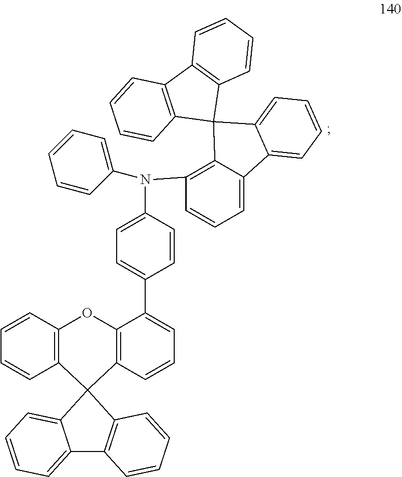 [SMILES: c1ccc(N(c2ccc(-c3cccc4c3Oc3ccccc3C43c4ccccc4-c4ccccc43)cc2)c2cccc3c2C2(c4ccccc4-c4ccccc42)c2ccccc2-3)cc1]

the compounds 1S-140S are obtained by replacing

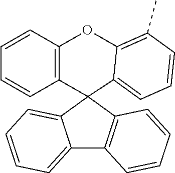

in Compounds 1-140 with

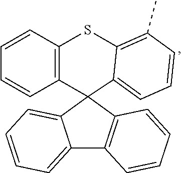

wherein the dashed line represents the linkage site.

For example, the structure of Compound 2 is

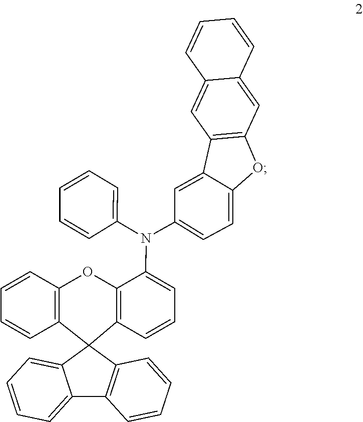 [SMILES: c1ccc(N(c2ccc3oc4cc5ccccc5cc4c3c2)c2cccc3c2Oc2ccccc2C32c3ccccc3-c3ccccc32)cc1]

and the structure of Compound 2S is

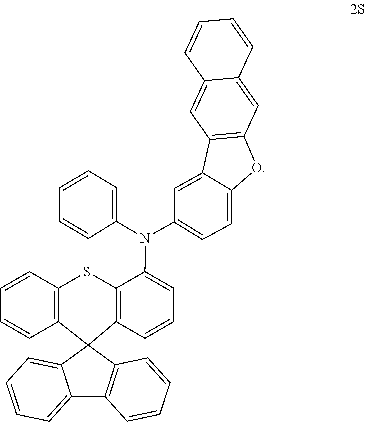 [SMILES: c1ccc(N(c2ccc3oc4cc5ccccc5cc4c3c2)c2cccc3c2Sc2ccccc2C32c3ccccc3-c3ccccc32)cc1]

In a fifth aspect, the present application provides a display apparatus, and the display apparatus includes the organic electroluminescent device as described in the fourth aspect.

Compared with the prior art, the present application has the following beneficial effects.

A composition with specific component is obtained by designing a specific component of the composition and further using the combination of at least two specific compounds, and an organic electroluminescent device with a lower driving voltage, a higher current efficiency and a longer lifetime can be fabricated by using this composition as the material of the emissive layer of the organic electroluminescent device.

Other aspects can be understood after reading and understanding the detailed description.

### DETAILED DESCRIPTION

In order to facilitate the understanding of the present application, embodiments of the present application are provided below. It should be understood by those skilled in the art that the embodiments are merely an aid to understanding the present application and should not be regarded as a specific limitation of the present application.

### Synthesis Example 1

This synthesis example provides Compound H-28-D1 and a synthesis method thereof, and the synthesis method is as follows.

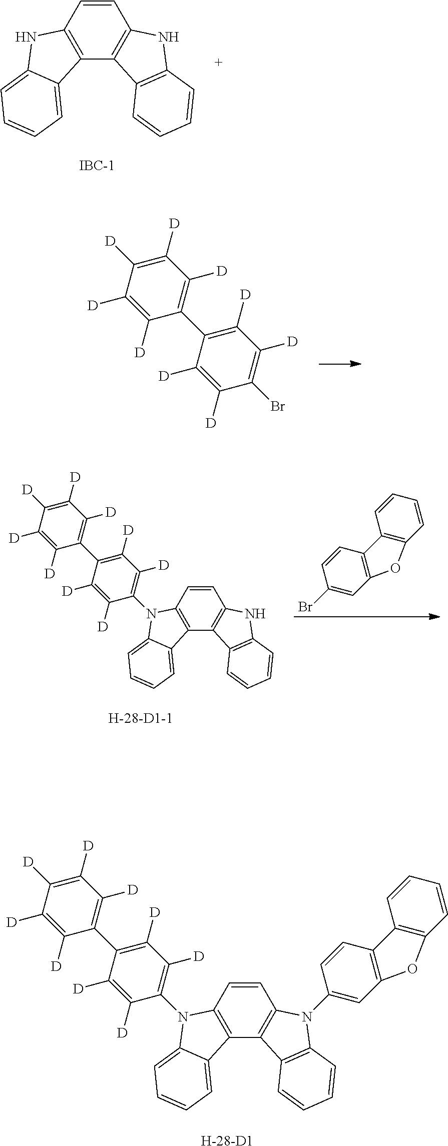

### (1) Synthesis of the Intermediate H-28-D1-1

Under the nitrogen protection, 80 mL of dry toluene, Compound IBC-1 (2.56 g), brominated deuterated biphenyl (2.5 g), Pd(dba)₂ (bis(dibenzylideneacetone)palladium, 0.0575 g), a toluene solution of tri-tert-butylphosphine with a mass percent of 10% (0.4 g), and sodium tert-butoxide (1.44 g) were added to a 250 mL three-necked flask. The mixture was heated slowly to reflux and reacted for 8 hours, cooled down to room temperature and added with water for phase separation. The organic layer was washed with water until neutral, dried with magnesium sulfate, filtered to remove magnesium sulfate and concentrated to dryness. Purification was performed by silica gel column chromatography using a petroleum ether:dichloromethane mixture (10:1, v/v) as a eluent, yielding 3.1 g of intermediate H-28-D1-1.

Mass spectrometry analysis of H-28-D1-1: A mass-to-charge ratio (m/z) of 417.22 was detected.

### (2) Synthesis of Compound H-28-D1

The procedure was carried out similarly to the synthesis of intermediate H-28-D1-1, except that the compound IBC-1 was replaced with an equimolar amount of intermediate H-28-D1-1, and brominated deuterated biphenyl was substituted with an equimolar amount of 3-bromodibenzo[b,d]furan. Compound H-28-D1 was obtained.

Mass spectrometry analysis of H-28-D1: A mass-to-charge ratio (m/z) of 583.26 was detected.

### Synthesis Examples 2-8

Synthesis Examples 2-8 each provide a compound and a synthesis method thereof. In each example, an intermediate was synthesized from Reactant 1 and Reactant 2, followed by a reaction of the intermediate with the corresponding brominated compound to yield the target compound (see Table 1 below in detail). The specific synthesis methods followed the method described in Synthesis Example 1.

Mass spectrometry analyses were performed on the intermediates and compounds from Synthesis Examples 2-8. The measured mass-to-charge ratios (m/z) are listed in Table 1 and Table 2.

TABLE 1

| Synthesis | | | | m/z |
|---|---|---|---|---|
| Example | | | | of |
| No. | Reactant 1 | Reactant 2 | Intermediate | Intermediate |
| Synthesis Example 2 | 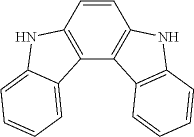 [SMILES: c1ccc2c(c1)[nH]c1ccc3[nH]c4ccccc4c3c12] | 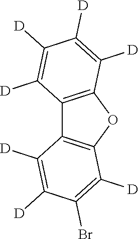 [SMILES: [2H]c1c([2H])c([2H])c2c(oc3c([2H])c(Br)c([2H])c([2H])c32)c1[2H]] | 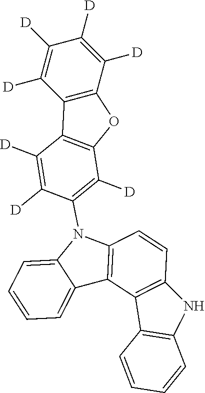 [SMILES: [2H]c1c([2H])c([2H])c2c(oc3c([2H])c(-n4c5ccccc5c5c6c(ccc54)[nH]c4ccccc46)c([2H])c([2H])c32)c1[2H]] | 429.19 |
| | IBC-1 | | H-28-D2-1 | |
| Synthesis Example 3 | 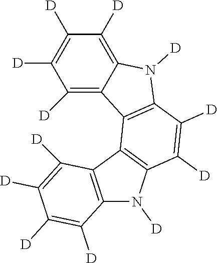 | 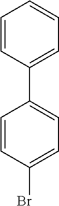 [SMILES: Brc1ccc(-c2ccccc2)cc1] | 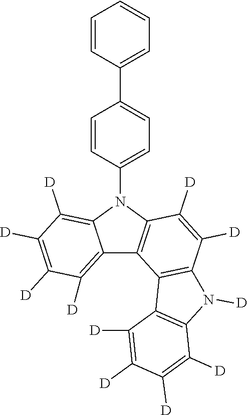 | 419.23 |
| | IBC-1-D | | H-28-DE-1 | |
| Synthesis Example 4 | 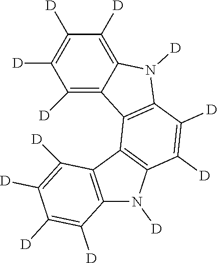 | 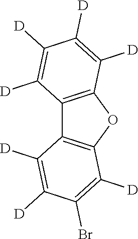 [SMILES: [2H]c1c([2H])c([2H])c2c(oc3c([2H])c(Br)c([2H])c([2H])c32)c1[2H]] | 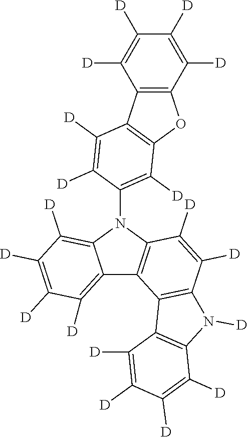 | 440.25 |
| | IBC-1-D | | H-28-DT-1 | |
| Synthesis Example 5 | 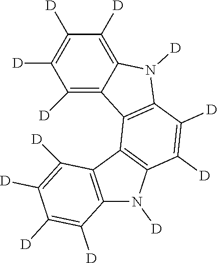 | 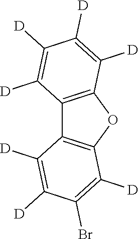 [SMILES: [2H]c1c([2H])c([2H])c2c(oc3c([2H])c(Br)c([2H])c([2H])c32)c1[2H]] | 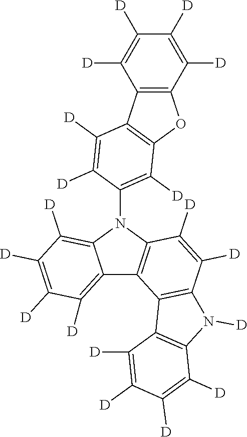 | 440.25 |
| | IBC-1-D | | H-28-DT-1 | |
| Synthesis Example 6 | 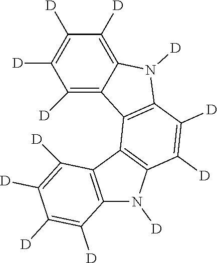 | 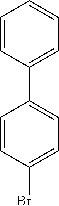 [SMILES: Brc1ccc(-c2ccccc2)cc1] | 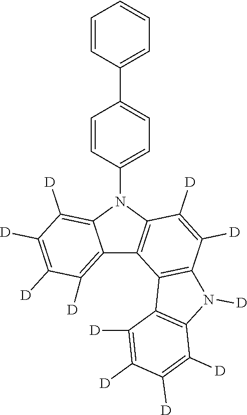 | 419.23 |
| | IBC-1-D | | H-28-DE-1 | |
| Synthesis Example 7 | 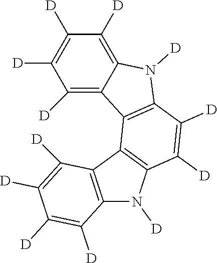 | 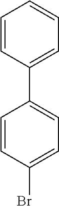 [SMILES: Brc1ccc(-c2ccccc2)cc1] | 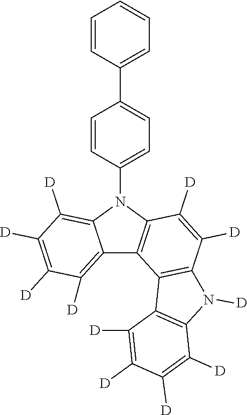 | 419.23 |
| | IBC-1-D | | H-28-DE-1 | |
| Synthesis Example 8 | 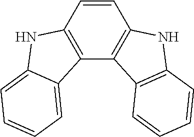 [SMILES: c1ccc2c(c1)[nH]c1ccc3[nH]c4ccccc4c3c12] | 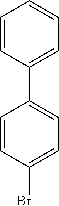 [SMILES: Brc1ccc(-c2ccccc2)cc1] | 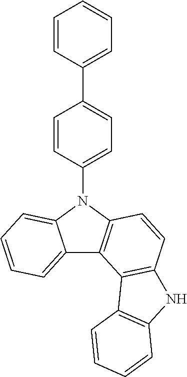 [SMILES: c1ccc(-c2ccc(-n3c4ccccc4c4c5c(ccc43)[nH]c3ccccc35)cc2)cc1] | 408.16 |
| | IBC-1 | | H-29-1 | |

TABLE 2

| Synthesis | | | |
|---|---|---|---|
| Example | | | m/z of |
| No. | Bromide | Compound | Compound |
| Synthesis Example 2 | 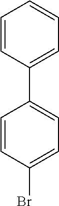 [SMILES: Brc1ccc(-c2ccccc2)cc1] | 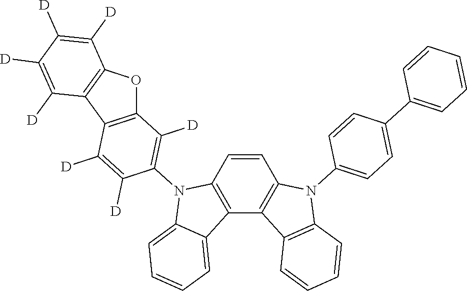 [SMILES: [2H]c1c([2H])c([2H])c2c(oc3c([2H])c(-n4c5ccccc5c5c6c7ccccc7n(-c7ccc(-c8ccccc8)cc7)c6ccc54)c([2H])c([2H])c32)c1[2H]] | 581.25 |
| | | H-28-D2 | |
| Synthesis Example 3 | 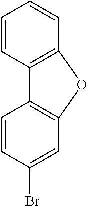 [SMILES: Brc1ccc2c(c1)oc1ccccc12] | 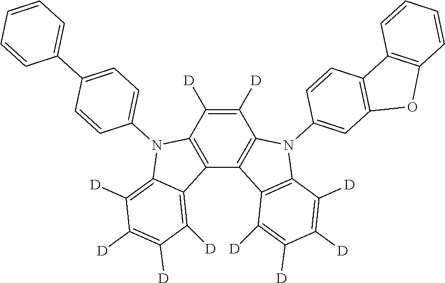 [SMILES: [2H]c1c([2H])c([2H])c2c(c1[2H])c1c3c4c([2H])c([2H])c([2H])c([2H])c4n(-c4ccc5c(c4)oc4ccccc45)c3c([2H])c([2H])c1n2-c1ccc(-c2ccccc2)cc1] | 584.27 |
| | | H-28-DE | |
| Synthesis Example 4 | 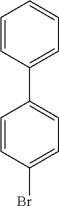 [SMILES: Brc1ccc(-c2ccccc2)cc1] | 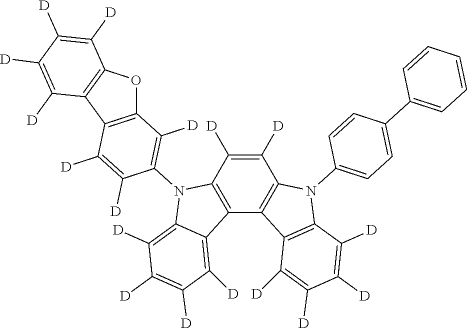 [SMILES: [2H]c1c([2H])c([2H])c2c(oc3c([2H])c(-n4c5c([2H])c([2H])c([2H])c([2H])c5c5c6c7c([2H])c([2H])c([2H])c([2H])c7n(-c7ccc(-c8ccccc8)cc7)c6c([2H])c([2H])c54)c([2H])c([2H])c32)c1[2H]] | 591.31 |
| | | H-28-DT | |
| Synthesis Example 5 | 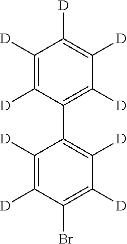 [SMILES: [2H]c1c([2H])c([2H])c(-c2c([2H])c([2H])c(Br)c([2H])c2[2H])c([2H])c1[2H]] | 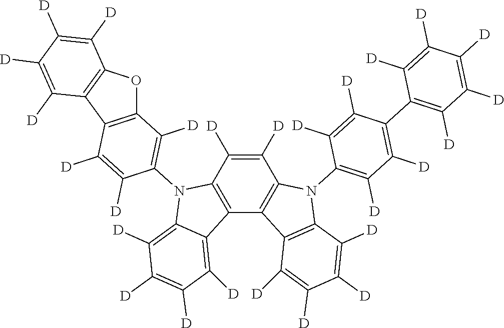 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c7c(oc8c([2H])c([2H])c([2H])c([2H])c87)c6[2H])c5c([2H])c([2H])c43)c([2H])c2[2H])c([2H])c1[2H]] | 600.37 |
| | | H-28-DF | |
| Synthesis Example 6 | 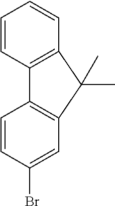 [SMILES: CC1(C)c2ccccc2-c2ccc(Br)cc21] | 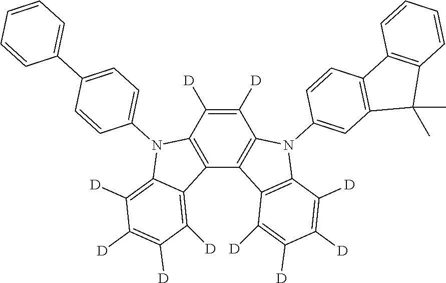 [SMILES: [2H]c1c([2H])c([2H])c2c(c1[2H])c1c3c4c([2H])c([2H])c([2H])c([2H])c4n(-c4ccc5c(c4)C(C)(C)c4ccccc4-5)c3c([2H])c([2H])c1n2-c1ccc(-c2ccccc2)cc1] | 610.32 |
| | | H-26-DE | |
| Synthesis Example 7 | 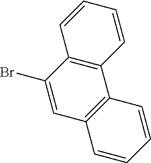 [SMILES: Brc1cc2ccccc2c2ccccc12] | 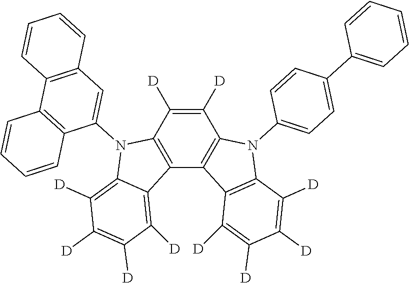 [SMILES: [2H]c1c([2H])c([2H])c2c(c1[2H])c1c3c4c([2H])c([2H])c([2H])c([2H])c4n(-c4cc5ccccc5c5ccccc45)c3c([2H])c([2H])c1n2-c1ccc(-c2ccccc2)cc1] | 594.29 |
| | | H-29-DE | |
| Synthesis Example 8 | 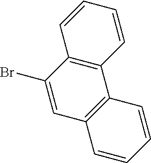 [SMILES: Brc1cc2ccccc2c2ccccc12] | 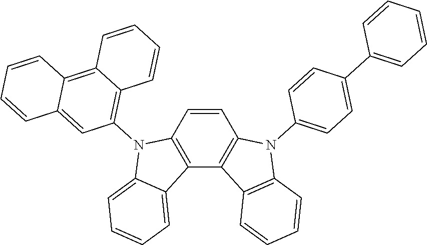 [SMILES: c1ccc(-c2ccc(-n3c4ccccc4c4c5c6ccccc6n(-c6cc7ccccc7c7ccccc67)c5ccc43)cc2)cc1] | 584.23 |
| | | H-29 | |

Other compounds with no specific synthesis steps listed can be prepared by common knowledge in the field in conjunction with the above examples.

The specific structures of the compounds used in the following Device Examples and Device Comparative Examples are as follows.

The specific structures of the compounds adopted in the following examples are shown below:

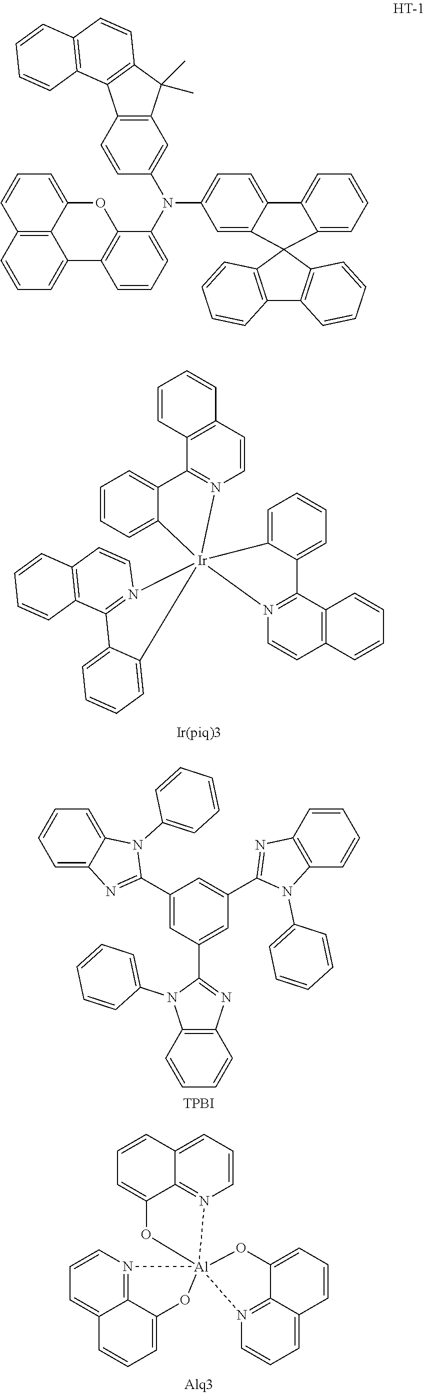

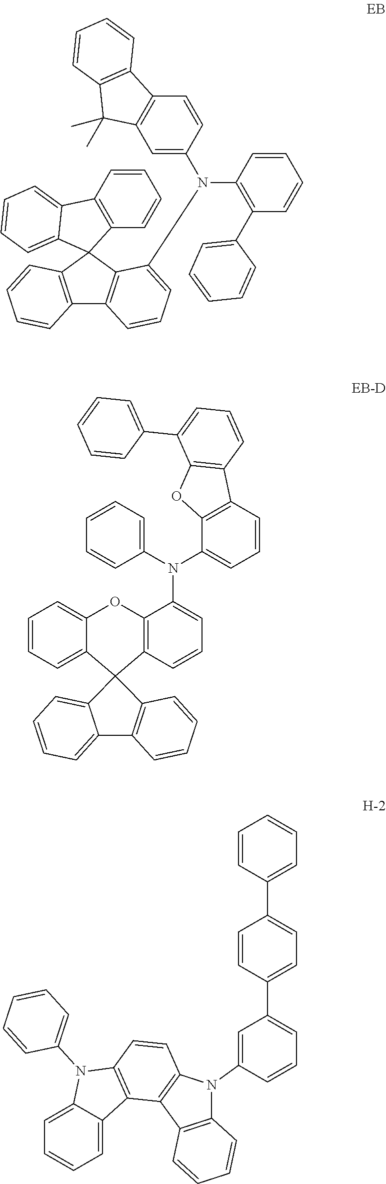 [SMILES: CC1(C)c2ccccc2-c2ccc(N(c3ccccc3-c3ccccc3)c3cccc4c3C3(c5ccccc5-c5ccccc53)c3ccccc3-4)cc21 ; c1ccc(-c2cccc3c2oc2c(N(c4ccccc4)c4cccc5c4Oc4ccccc4C54c5ccccc5-c5ccccc54)cccc23)cc1 ; c1ccc(-c2ccc(-c3cccc(-n4c5ccccc5c5c6c7ccccc7n(-c7ccccc7)c6ccc54)c3)cc2)cc1]

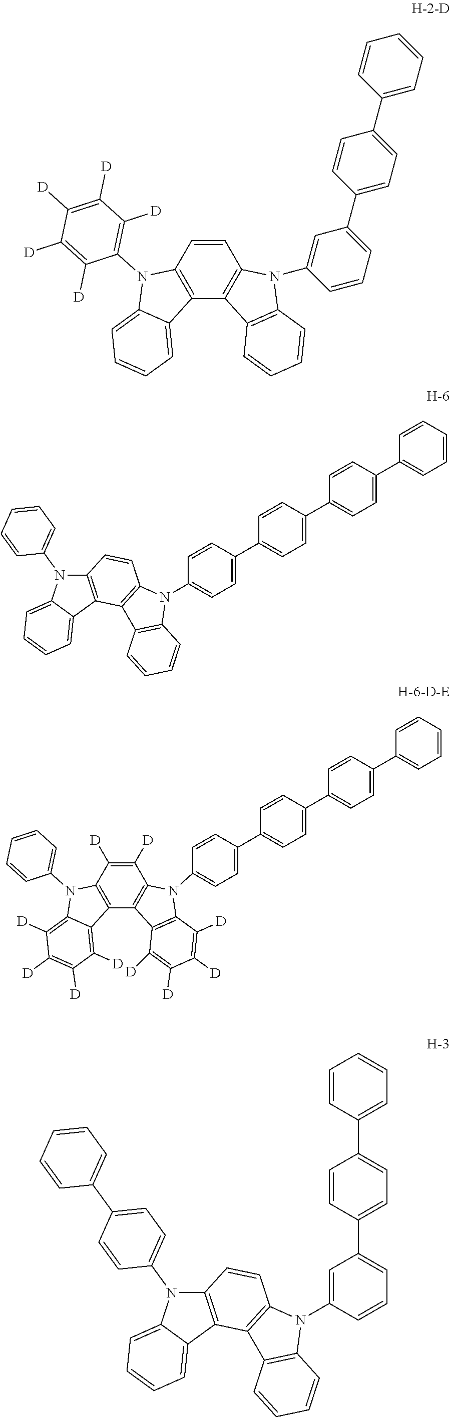 [SMILES: [2H]c1c([2H])c([2H])c(-n2c3ccccc3c3c4c5ccccc5n(-c5cccc(-c6ccc(-c7ccccc7)cc6)c5)c4ccc32)c([2H])c1[2H] ; c1ccc(-c2ccc(-c3ccc(-c4ccc(-n5c6ccccc6c6c7c8ccccc8n(-c8ccccc8)c7ccc65)cc4)cc3)cc2)cc1 ; [2H]c1c([2H])c([2H])c2c(c1[2H])c1c3c4c([2H])c([2H])c([2H])c([2H])c4n(-c4ccc(-c5ccc(-c6ccc(-c7ccccc7)cc6)cc5)cc4)c3c([2H])c([2H])c1n2-c1ccccc1 ; c1ccc(-c2ccc(-c3cccc(-n4c5ccccc5c5c6c7ccccc7n(-c7ccc(-c8ccccc8)cc7)c6ccc54)c3)cc2)cc1]

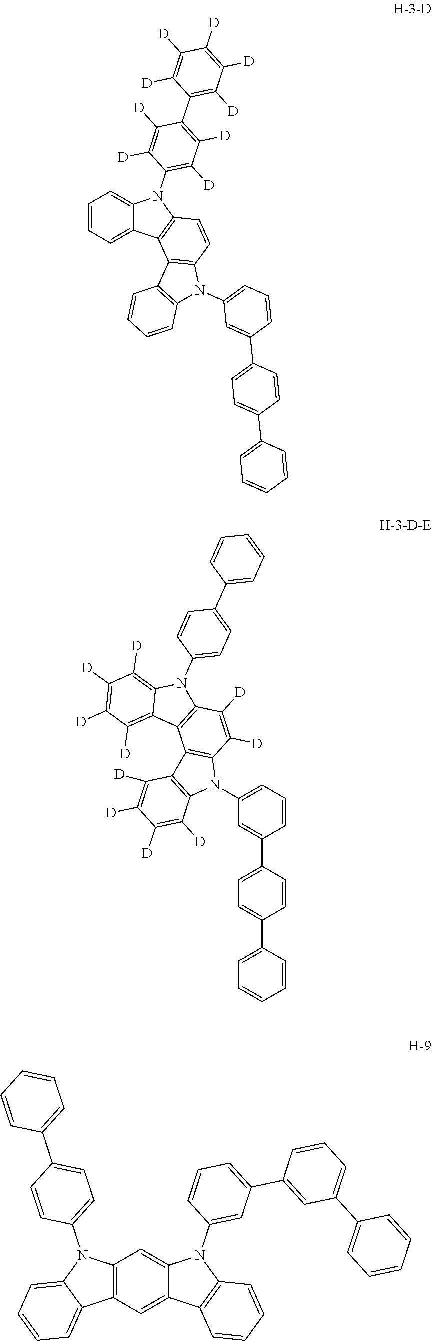 [SMILES: [2H]c1c([2H])c([2H])c(-c2c([2H])c([2H])c(-n3c4ccccc4c4c5c6ccccc6n(-c6cccc(-c7ccc(-c8ccccc8)cc7)c6)c5ccc43)c([2H])c2[2H])c([2H])c1[2H] ; [2H]c1c([2H])c([2H])c2c(c1[2H])c1c3c4c([2H])c([2H])c([2H])c([2H])c4n(-c4cccc(-c5ccc(-c6ccccc6)cc5)c4)c3c([2H])c([2H])c1n2-c1ccc(-c2ccccc2)cc1 ; c1ccc(-c2ccc(-n3c4ccccc4c4cc5c6ccccc6n(-c6cccc(-c7cccc(-c8ccccc8)c7)c6)c5cc43)cc2)cc1]

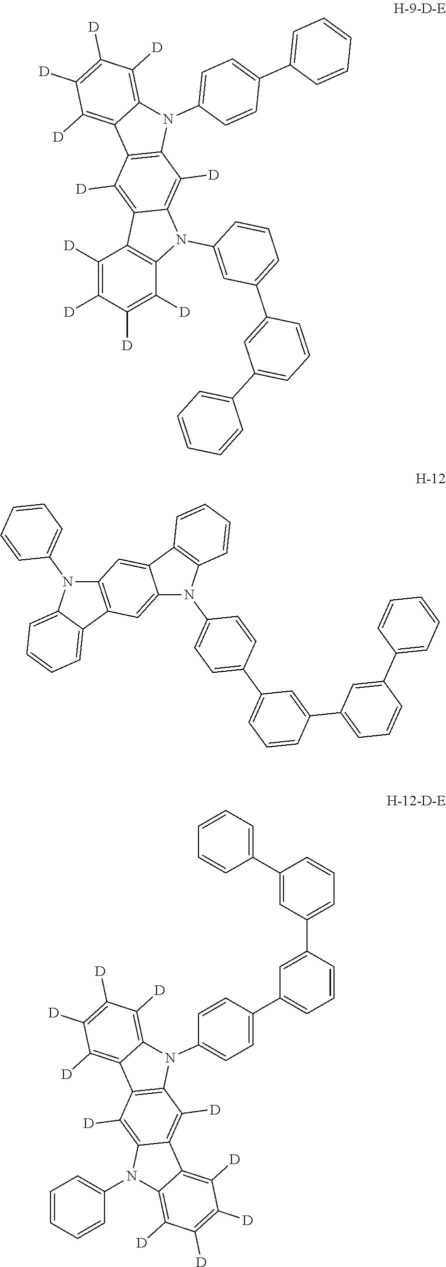 [SMILES: [2H]c1c([2H])c([2H])c2c(c1[2H])c1c([2H])c3c4c([2H])c([2H])c([2H])c([2H])c4n(-c4cccc(-c5cccc(-c6ccccc6)c5)c4)c3c([2H])c1n2-c1ccc(-c2ccccc2)cc1 ; c1ccc(-c2cccc(-c3cccc(-c4ccc(-n5c6ccccc6c6cc7c(cc65)c5ccccc5n7-c5ccccc5)cc4)c3)c2)cc1 ; [2H]c1c([2H])c([2H])c2c(c1[2H])c1c([2H])c3c(c([2H])c1n2-c1ccccc1)c1c([2H])c([2H])c([2H])c([2H])c1n3-c1ccc(-c2cccc(-c3cccc(-c4ccccc4)c3)c2)cc1]

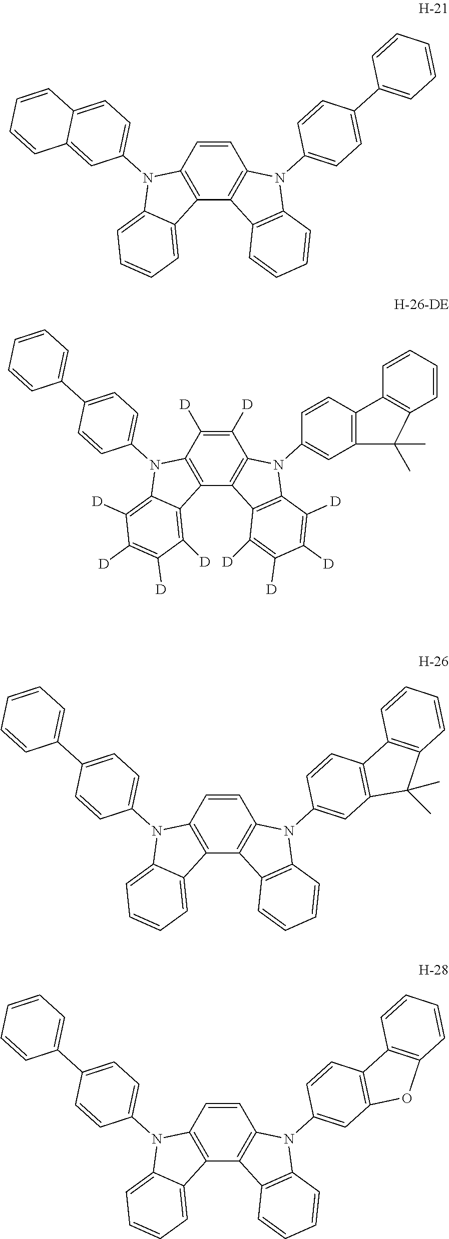 [SMILES: c1ccc(-c2ccc(-n3c4ccccc4c4c5c6ccccc6n(-c6ccc7ccccc7c6)c5ccc43)cc2)cc1 ; [2H]c1c([2H])c([2H])c2c(c1[2H])c1c3c4c([2H])c([2H])c([2H])c([2H])c4n(-c4ccc5c(c4)C(C)(C)c4ccccc4-5)c3c([2H])c([2H])c1n2-c1ccc(-c2ccccc2)cc1 ; CC1(C)c2ccccc2-c2ccc(-n3c4ccccc4c4c5c6ccccc6n(-c6ccc(-c7ccccc7)cc6)c5ccc43)cc21 ; c1ccc(-c2ccc(-n3c4ccccc4c4c5c6ccccc6n(-c6ccc7c(c6)oc6ccccc67)c5ccc43)cc2)cc1]

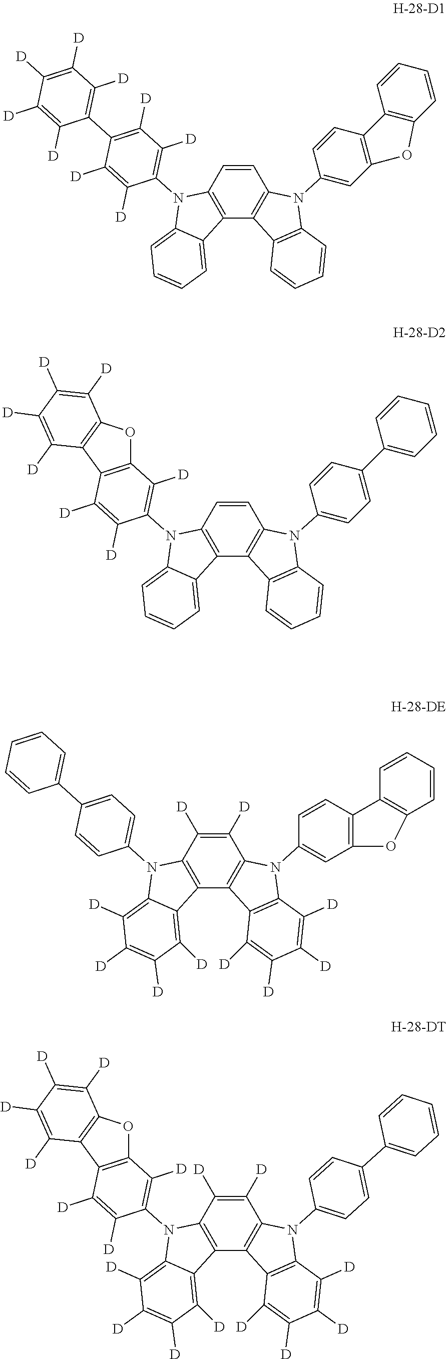

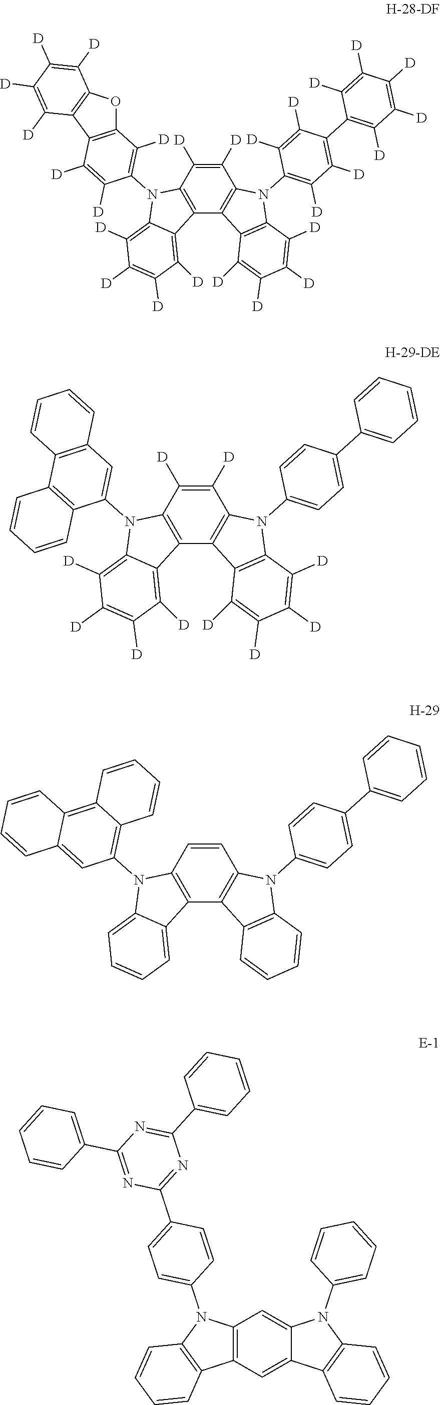

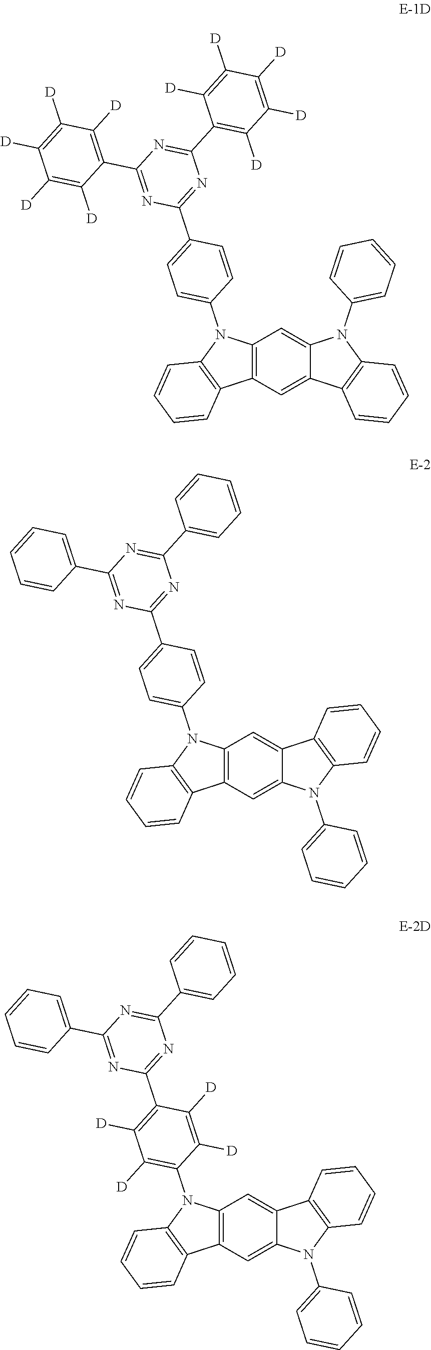 [SMILES: [2H]c1c([2H])c([2H])c(-c2nc(-c3ccc(-n4c5ccccc5c5cc6c7ccccc7n(-c7ccccc7)c6cc54)cc3)nc(-c3c([2H])c([2H])c([2H])c([2H])c3[2H])n2)c([2H])c1[2H] ; c1ccc(-c2nc(-c3ccccc3)nc(-c3ccc(-n4c5ccccc5c5cc6c(cc54)c4ccccc4n6-c4ccccc4)cc3)n2)cc1 ; [2H]c1c([2H])c(-n2c3ccccc3c3cc4c(cc32)c2ccccc2n4-c2ccccc2)c([2H])c([2H])c1-c1nc(-c2ccccc2)nc(-c2ccccc2)n1]

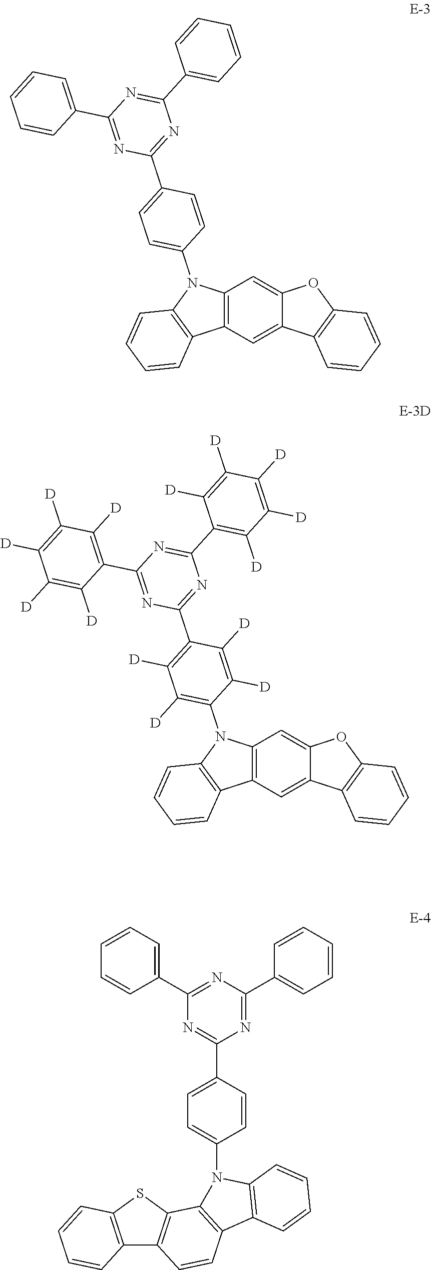 [SMILES: c1ccc(-c2nc(-c3ccccc3)nc(-c3ccc(-n4c5ccccc5c5cc6c(cc54)oc4ccccc46)cc3)n2)cc1 ; [2H]c1c([2H])c([2H])c(-c2nc(-c3c([2H])c([2H])c([2H])c([2H])c3[2H])nc(-c3c([2H])c([2H])c(-n4c5ccccc5c5cc6c(cc54)oc4ccccc46)c([2H])c3[2H])n2)c([2H])c1[2H] ; c1ccc(-c2nc(-c3ccccc3)nc(-c3ccc(-n4c5ccccc5c5ccc6c7ccccc7sc6c54)cc3)n2)cc1]

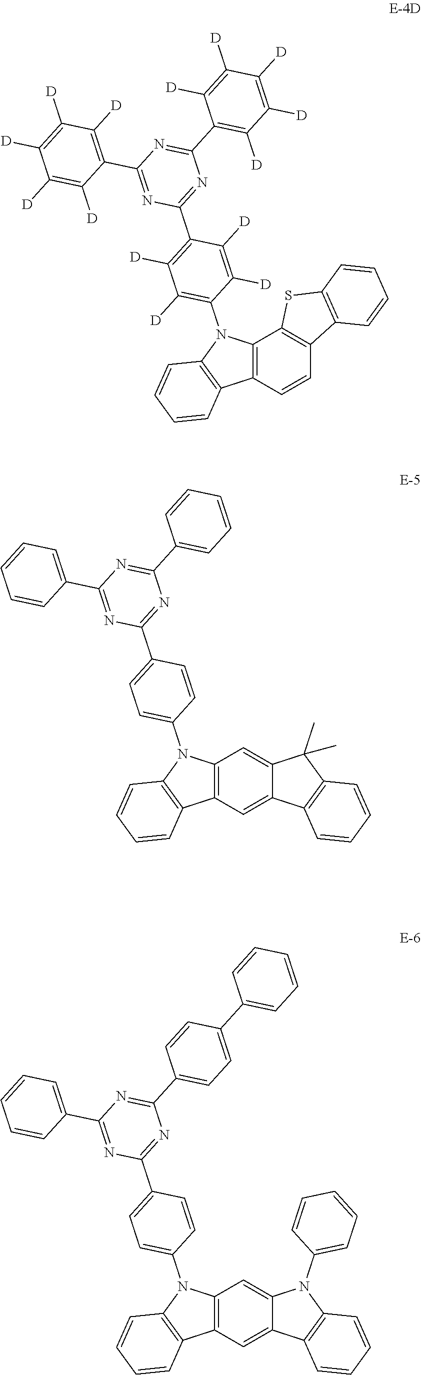 [SMILES: [2H]c1c([2H])c([2H])c(-c2nc(-c3c([2H])c([2H])c([2H])c([2H])c3[2H])nc(-c3c([2H])c([2H])c(-n4c5ccccc5c5ccc6c7ccccc7sc6c54)c([2H])c3[2H])n2)c([2H])c1[2H] ; CC1(C)c2ccccc2-c2cc3c4ccccc4n(-c4ccc(-c5nc(-c6ccccc6)nc(-c6ccccc6)n5)cc4)c3cc21 ; c1ccc(-c2ccc(-c3nc(-c4ccccc4)nc(-c4ccc(-n5c6ccccc6c6cc7c8ccccc8n(-c8ccccc8)c7cc65)cc4)n3)cc2)cc1]

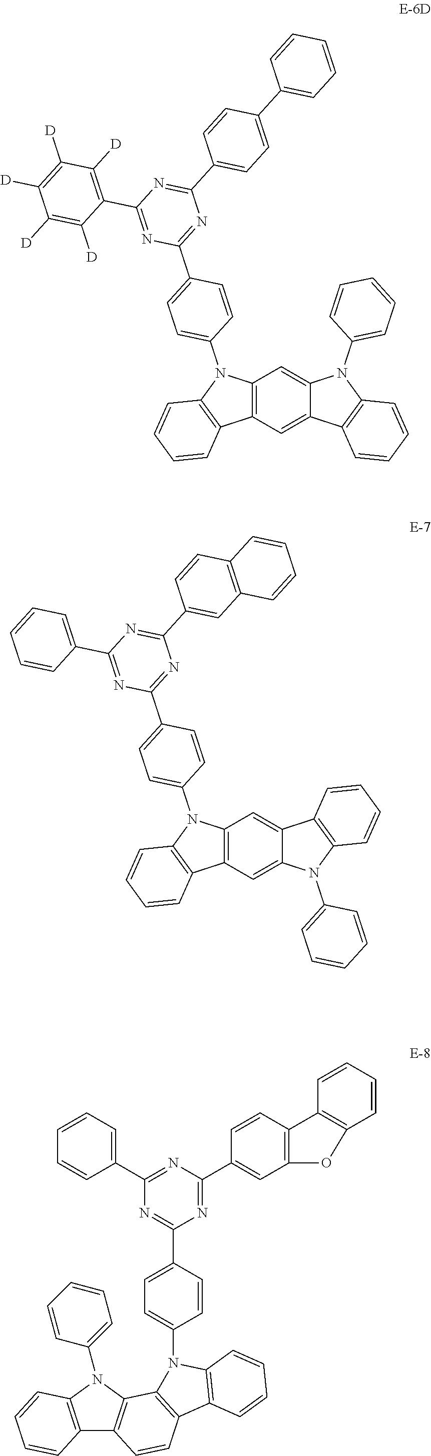 [SMILES: [2H]c1c([2H])c([2H])c(-c2nc(-c3ccc(-c4ccccc4)cc3)nc(-c3ccc(-n4c5ccccc5c5cc6c7ccccc7n(-c7ccccc7)c6cc54)cc3)n2)c([2H])c1[2H] ; c1ccc(-c2nc(-c3ccc(-n4c5ccccc5c5cc6c(cc54)c4ccccc4n6-c4ccccc4)cc3)nc(-c3ccc4ccccc4c3)n2)cc1 ; c1ccc(-c2nc(-c3ccc(-n4c5ccccc5c5ccc6c7ccccc7n(-c7ccccc7)c6c54)cc3)nc(-c3ccc4c(c3)oc3ccccc34)n2)cc1]

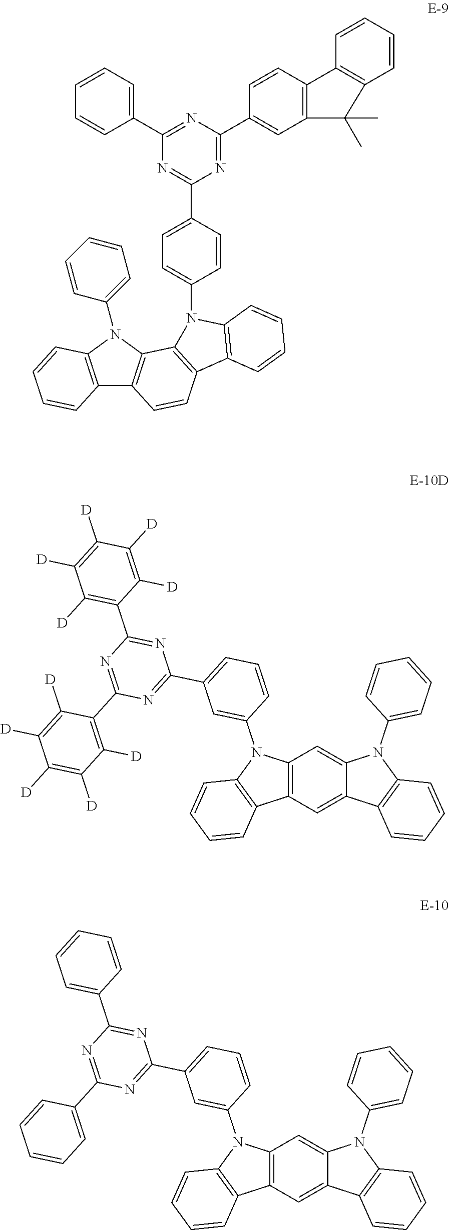 [SMILES: CC1(C)c2ccccc2-c2ccc(-c3nc(-c4ccccc4)nc(-c4ccc(-n5c6ccccc6c6ccc7c8ccccc8n(-c8ccccc8)c7c65)cc4)n3)cc21 ; [2H]c1c([2H])c([2H])c(-c2nc(-c3cccc(-n4c5ccccc5c5cc6c7ccccc7n(-c7ccccc7)c6cc54)c3)nc(-c3c([2H])c([2H])c([2H])c([2H])c3[2H])n2)c([2H])c1[2H] ; c1ccc(-c2nc(-c3ccccc3)nc(-c3cccc(-n4c5ccccc5c5cc6c7ccccc7n(-c7ccccc7)c6cc54)c3)n2)cc1]

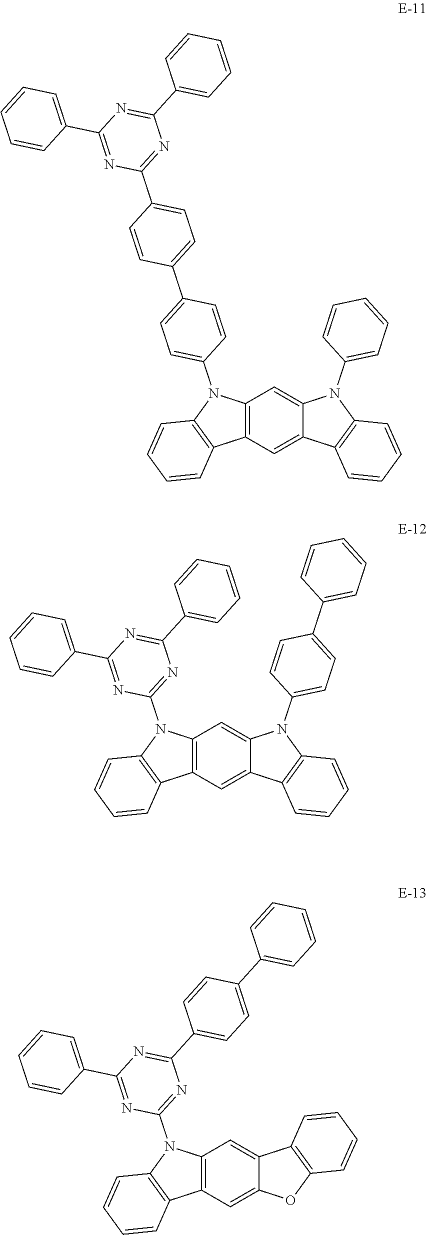 [SMILES: c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1 ; c1ccc(-c2ccc(-n3c4ccccc4c4cc5c6ccccc6n(-c6nc(-c7ccccc7)nc(-c7ccccc7)n6)c5cc43)cc2)cc1 ; c1ccc(-c2ccc(-c3nc(-c4ccccc4)nc(-n4c5ccccc5c5cc6oc7ccccc7c6cc54)n3)cc2)cc1]

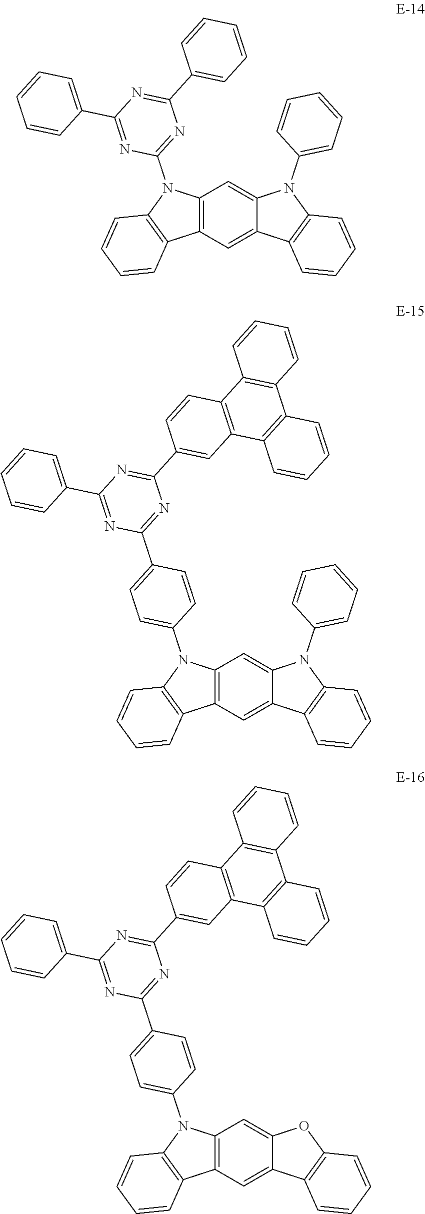 [SMILES: c1ccc(-c2nc(-c3ccccc3)nc(-n3c4ccccc4c4cc5c6ccccc6n(-c6ccccc6)c5cc43)n2)cc1 ; c1ccc(-c2nc(-c3ccc(-n4c5ccccc5c5cc6c7ccccc7n(-c7ccccc7)c6cc54)cc3)nc(-c3ccc4c5ccccc5c5ccccc5c4c3)n2)cc1 ; c1ccc(-c2nc(-c3ccc(-n4c5ccccc5c5cc6c(cc54)oc4ccccc46)cc3)nc(-c3ccc4c5ccccc5c5ccccc5c4c3)n2)cc1]

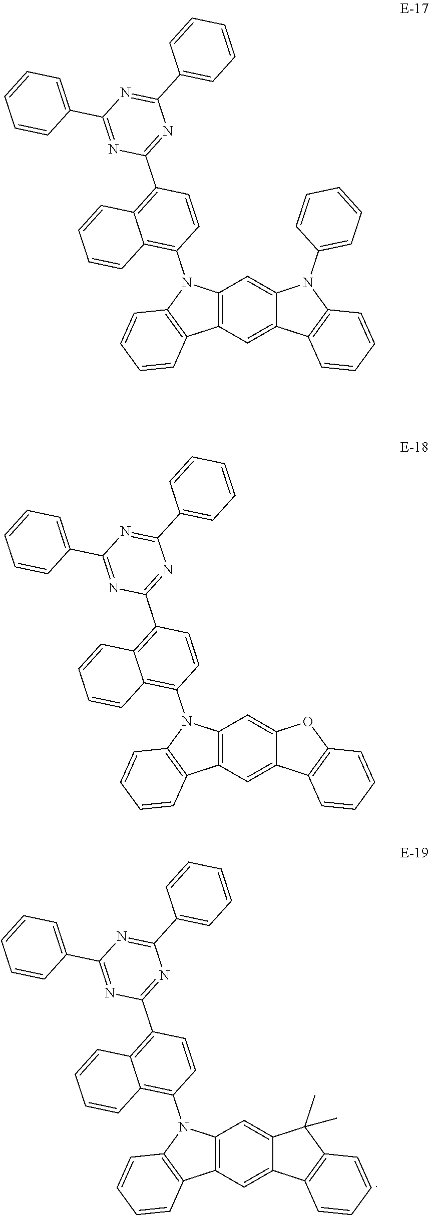 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc(-n4c5ccccc5c5cc6c(cc54)oc4ccccc46)c4ccccc34)n2)cc1 ; CC1(C)c2ccccc2-c2cc3c4ccccc4n(-c4ccc(-c5nc(-c6ccccc6)nc(-c6ccccc6)n5)c5ccccc45)c3cc21]

### Device Example 1

This device example provides an organic electroluminescent device, wherein the composition provided in the present application is selected as the red-emitting host material.

The structure of the organic electroluminescent device is as follows: ITO/HT-1 (20 nm)/red-emitting host material (35 nm): Ir(piq)₃ [10%]/TPBI (10 nm)/Alq3 (15 nm)/LiF (0.5 nm)/Al (150 nm); wherein ‘Ir(piq)₃ [10%]’ refers to the doping ratio of the red dopant, that is, a volume ratio of the red-emitting host material to Ir(piq)₃ is 90:10.

The fabrication process of the organic electroluminescent device is as follows:
a glass substrate coated with an ITO transparent conductive layer was ultrasonically cleaned in a commercial detergent, rinsed with deionized water, degreased in an acetone:ethanol mixed solvent via ultrasonication, baked in a clean environment to remove moisture completely, treated with UV-ozone cleaning, and the surface bombarded with a low-energy cation beam;
the above glass substrate with an anode was placed in a vacuum chamber, and the chamber was evacuated to 1×10⁻⁵-9×10⁻⁴ Pa, and a hole transport layer (HT-1) was vacuum-evaporated onto the anode at a evaporation rate of 0.1 nm/s, forming a 20 nm-thick film;
the red-emitting host material and dopant Ir(piq)₃ were co-evaporated on the hole transport layer as an emissive layer, and the evaporation rate was maintained at 0.1 nm/s, and the total thickness was controlled at 35 nm; in this device example, under the condition that the host of red light is compound H-2 and compound H-2D, the compound H-2 and compound H-2D were placed in separate evaporation sources, and the heating rates were adjusted to ensure a 1:1 volumetric ratio of H-2 to H-2D evaporated onto the substrate, which is used as the red-emitting host material;
an electron transport layer (TPBI) and Alq3 were sequentially evaporated on the emissive layer at a rate of 0.1 nm/s, with a thicknesses of 10 nm and 15 nm, respectively; and
a 0.5 nm-thick LiF layer and a 150 nm-thick Al layer were vacuum-evaporated as an electron injection layer and cathode on the electron transport layer, respectively.

### Device Comparative Examples 1-2

Device Comparative Examples 1-2 each provide an organic electroluminescent device, which differs from Device Example 1 only in that the red-emitting host material was different (see Table 3 below in detail), and other preparation steps and conditions were the same as those of Device Example 1.

### Performance Test

OLED-1000 Multi-channel Accelerated Lifetime & Optical Property Test System for OLED manufactured by Hangzhou EVERFINE was used to test and measure the luminance, driving voltage, current efficiency and the lifetime LT90 of the fabricated organic electroluminescent devices. The lifetime test LT90 is the time that the luminance is reduced to 90% of the initial luminance at room temperature (25-27° C.), while the current density at the initial luminance is kept constant (1000 cd/m²). In the following tables, driving voltage, current efficiency, and LT90 lifetime are relative values. The test results are detailed in Table 3 below.

TABLE 3

| | Red-emitting host material | | | | | |
|---|---|---|---|---|---|---|
| | (compound represented by | | Required | | | |
| | Formula I) | | luminance/ | Driving | Current | LT90 |
| | Component A | Component B | (cd/m²) | voltage | efficiency | lifetime |
| Device | H-2 | H-2D | 1000 | 1 | 1 | 1 |
| Example 1 | | | | | | |
| Device | H-2 | / | 1000 | 1.22 | 0.86 | 0.55 |
| Comparative | | | | | | |
| Example 1 | | | | | | |
| Device | H-2D | / | 1000 | 1.21 | 0.89 | 0.62 |
| Comparative | | | | | | |
| Example 2 | | | | | | |

Note:
In the table, ‘/’ represents that the red-emitting host material of the device example does not contain the compound, and similarly hereinafter.

As can be seen from Table 3, in the present application, by using the two compounds represented by Formula I as an emissive layer material and further controlling one of the two compounds containing deuterium atoms represented by Formula I, the driving voltage of the organic electroluminescent device can be further reduced, and the current efficiency and lifetime of the organic electroluminescent device can be improved.

### Device Examples 2-10

Device Examples 2-10 each provide an organic electroluminescent device, which differs from Device Example 1 only in that the red-emitting host material is different, and if the red-emitting host material is two or more compounds, each compounds are placed in different evaporation sources for heating, and the heating rate is controlled so that the ratio of each volume that is evaporated onto the substrate is in the same proportion, as a red-emitting host material (see Tables 4 and 5 below in detail), and other preparation steps and conditions are the same as in Device Example 1.

### Device Comparative Examples 3-4

Device Comparative Examples 3-4 each provide an organic electroluminescent device, which differs from Device Example 1 only in that the red-emitting host material is different, and if the red-emitting host material is two or more compounds, each compounds are placed in different evaporation sources for heating, and the heating rate is controlled so that the ratio of each volume that is evaporated onto the substrate is in the same proportion, as a red-emitting host material (see Table 4 below in detail), and other preparation steps and conditions are the same as in Device Example 1.

The performance of the organic electroluminescent devices provided by Device Examples 2-10 and Device Comparative Examples was tested, and the test method was the same as above. The test results are detailed in Tables 4 and 5 below.

TABLE 4

| | Red-emitting host material | | | | | | |
|---|---|---|---|---|---|---|---|
| | (compound represented by Formula I) | | | Required | | | |
| | Component | Component | Component | luminance/ | Driving | Current | LT90 |
| | A | B | C | (cd/m²) | voltage | efficiency | lifetime |
| Device | H-3 | H-3-D | H-3-D-E | 1000 | 1 | 1 | 1 |
| Example 2 | | | | | | | |
| Device | H-3 | H-3-D | / | 1000 | 1.02 | 0.87 | 0.91 |
| Example 3 | | | | | | | |
| Device | H-3 | / | H-3-D-E | 1000 | 0.89 | 1.11 | 1.26 |
| Example 4 | | | | | | | |
| Device | H-3 | / | / | 1000 | 1.22 | 0.89 | 0.42 |
| Comparative | | | | | | | |
| Example 3 | | | | | | | |
| Device | / | / | H-3-D-E | 1000 | 1.16 | 0.89 | 0.68 |
| Comparative | | | | | | | |
| Example 4 | | | | | | | |

As can be seen from Table 4, in the present application, a composition designed with at least one compound containing deuterium atoms represented by Formula I can further reduce the driving voltage of the organic electroluminescent device, and improve the current efficiency and lifetime of the organic electroluminescent device.

As can be seen from Device Comparative Example 3 and Device Comparative Example 4, the use of the compound containing deuterium atoms represented by Formula I can significantly improve the lifetime of the organic electroluminescent device.

Compared with Device Example 3, by using Compound H-3-D-E represented by Formula I that has a deuterium atom linked to the indolocarbazole ring, Compound H-3, and optional Compound H-3-D as the red-emitting host material (Device Example 2 and Device Example 4), the driving voltage of the organic electroluminescent device can be further reduced, and the current efficiency and the lifetime of the organic electroluminescent device can be improved, and especially, the organic electroluminescent device fabricated by using Compound H-3-D-E (with a deuterium atom linked to the indolocarbazole ring) and Compound H-3 as the red-emitting host material has more excellent performance. This is because when acting as the host material, the HOMO is primarily localized on the central indolocarbazole ring. When the HOMO loses electrons, the central indolocarbazole ring connected to D atoms becomes more stable, thereby reducing material degradation.

In addition, the multicomponent compound is used as the red-emitting host material in the present application to provide better film formation, which results in higher charge mobility and further improves the driving voltage and current efficiency of the organic electroluminescent device, and meanwhile, the lifetime of the organic electroluminescent device is improved because the film formed by the multicomponent compound is more stable.

TABLE 5

| | Red-emitting host material | | | | | |
|---|---|---|---|---|---|---|
| | (compound represented by | | Required | | | |
| | Formula I) | | luminance/ | Driving | Current | LT90 |
| | Component A | Component B | (cd/m²) | voltage | efficiency | lifetime |
| Device | H-28 | H-28D2 | 1000 | 1.12 | 0.76 | 0.95 |
| Example 5 | | | | | | |
| Device | H-28 | H-28D1 | 1000 | 1.16 | 0.79 | 0.93 |
| Example 6 | | | | | | |
| Device | H-28 | H-28DE | 1000 | 0.97 | 1.03 | 0.91 |
| Example 7 | | | | | | |
| Device | H-28D1 | H-28DE | 1000 | 0.99 | 1.05 | 0.89 |
| Example 8 | | | | | | |
| Device | H-28 | H-28DT | 1000 | 1 | 1 | 1.2 |
| Example 9 | | | | | | |
| Device | H-28 | H-28DF | 1000 | 0.98 | 0.88 | 0.80 |
| Example 10 | | | | | | |

As can be seen from Device Example 9 and Device Example 10, the compound with a high content of deuterium atoms in compounds represented by Formula I is selected (Compound H-28DF used in Device Example 10), the organic electroluminescent device fabricated has a poorer comprehensive performance.

As can be seen from Device Example 7 and Device Example 8, the comprehensive performance of the organic electroluminescent device obtained in Device Example 7 and Device Example 8 is relatively close, but the lower-cost Compound H-28 without deuterium atoms is used in Device Example 7, which is in line with the requirements for industrialized production.

By comparing Device Examples 5-7 with Device Example 8, if compounds represented by Formula I in the composition both contain deuterium atoms (Device Example 8), conversely, the performance of the organic electroluminescent device is poorer. Meanwhile, as can be seen from Tables 3-5, if the composition in the present application consists of two compounds represented by Formula I, one compound containing deuterium atoms represented by Formula I and one compound without deuterium atoms represented by Formula I are preferred, and the compound represented by Formula I with linking deuterium atoms to the indolocarbazole ring and the compound represented by Formula I without deuterium atoms are more preferred, and organic electroluminescent device with excellent comprehensive performance can be obtained.

### Device Example 11

This device example provides an organic electroluminescent device, wherein the composition provided in the present application is selected as the red-emitting host material.

The structure of the organic electroluminescent device is as follows: ITO/HT-1 (20 nm)/red-emitting host material (35 nm): Ir(piq)₃[10%]/TPBI (10 nm)/Alq3 (15 nm)/LiF (0.5 nm)/Al (150 nm); wherein ‘Ir(piq)₃[10%]’ refers to the doping ratio of the red dopant, that is, a volume ratio of the red-emitting host material to Ir(piq)₃ is 90:10.

The fabrication process of the organic electroluminescent device is as follows:
a glass substrate coated with an ITO transparent conductive layer was ultrasonically cleaned in a commercial detergent, rinsed with deionized water, degreased in an acetone:ethanol mixed solvent via ultrasonication, baked in a clean environment to remove moisture completely, treated with UV-ozone cleaning, and the surface bombarded with a low-energy cation beam;
the above glass substrate with an anode was placed in a vacuum chamber, and the chamber was evacuated to 1×10⁻⁵-9×10⁻⁴ Pa, and a hole transport layer (HT-1) was vacuum-evaporated onto the anode at a evaporation rate of 0.1 nm/s, forming a 20 nm-thick film;
the red-emitting host material and dopant Ir(piq)₃ were co-evaporated on the hole transport layer as an emissive layer, and the evaporation rate was maintained at 0.1 nm/s, and the total thickness was controlled at 35 nm; in this example, and if the red-emitting host material is two or more compounds, each compounds are placed in different evaporation sources for heating, and the heating rate is controlled so that the ratio of each volume that is evaporated onto the substrate is in the same proportion, which is used as the red-emitting host material (see Table 6 below in detail);
an electron transport layer (TPBI) and Alq3 were sequentially evaporated on the emissive layer at a rate of 0.1 nm/s, with a thicknesses of 10 nm and 15 nm, respectively; and;
a 0.5 nm-thick LiF layer and a 150 nm-thick Al layer were vacuum-evaporated as an electron injection layer and cathode on the electron transport layer, respectively.

### Device Example 12-25

Device Examples 12-25 each provide an organic electroluminescent device, which differs from Device Example 11 only in that the red-emitting host material is different, and if the red-emitting host material is two or more compounds, each compounds are placed in different evaporation sources for heating, so that the ratio of each volume that is evaporated onto the substrate is in the same proportion, as a red-emitting host material (see Table 6 below in detail), and other preparation steps and conditions are the same as in Device Example 11.

### Device Comparative Example 5-10

Device Comparative Example 5-10 each provide an organic electroluminescent device, which differs from Device Example 11 only in that the red-emitting host material is different, and if the red-emitting host material is two or more compounds, each compounds are placed in different evaporation sources for heating, and the heating rate is controlled so that the ratio of each volume that is evaporated onto the substrate is in the same proportion, as a red-emitting host material (see Table 6 below in detail), and other preparation steps and conditions are the same as in Device Example 11.

The performance of the organic electroluminescent devices provided by Device Examples 12-25 and Device Comparative Examples 5-10 was tested, and the test method was the same as above. The test results are detailed in Table 6 below.

TABLE 6

| | Red-emitting host material | | | | Required | | | |
|---|---|---|---|---|---|---|---|---|
| | Component | Component | Component | Component | luminance/ | Driving | Current | LT90 |
| | A | B | C | D | (cd/m²) | voltage | efficiency | lifetime |
| Device | H-3 | H-3-D-E | E-1 | E-1D | 1000 | 0.86 | 1.26 | 2.58 |
| Example 11 | | | | | | | | |
| Device | H-3 | H-3-D-E | E-1 | / | 1000 | 0.87 | 1.21 | 2.09 |
| Example 12 | | | | | | | | |
| Device | H-3 | H-3-D-E | / | E-1D | 1000 | 0.79 | 1.18 | 2.11 |
| Example 13 | | | | | | | | |
| Device | H-3 | H-3-D-E | E-2 | E-3 | 1000 | 0.76 | 1.31 | 1.92 |
| Example 14 | | | | | | | | |
| Device | H-3 | H-3-D-E | E-5 | E-3D | 1000 | 0.56 | 1.16 | 1.67 |
| Example 15 | | | | | | | | |
| Device | H-3 | H-3-D-E | E-8 | E-1D | 1000 | 0.39 | 1.31 | 1.81 |
| Example 16 | | | | | | | | |
| Device | H-3 | H-3-D-E | E-11 | E-10D | 1000 | 0.67 | 1.56 | 2.02 |
| Example 17 | | | | | | | | |
| Device | H-3 | H-3-D-E | E-12 | E-13 | 1000 | 0.88 | 2.03 | 1.03 |
| Example 18 | | | | | | | | |
| Device | H-3 | H-3-D-E | E-14 | E-13 | 1000 | 0.92 | 2.19 | 1.00 |
| Example 19 | | | | | | | | |
| Device | H-3 | H-3-D-E | E-15 | E-16 | 1000 | 1.02 | 1.29 | 3.19 |
| Example 20 | | | | | | | | |
| Device | H-3 | H-3-D-E | E-17 | E-18 | 1000 | 1.06 | 1.16 | 3.01 |
| Example 21 | | | | | | | | |
| Device | H-3 | / | E-1 | E-1D | 1000 | 1 | 1 | 1 |
| Comparative | | | | | | | | |
| Example 5 | | | | | | | | |
| Device | / | H-3-D-E | E-1 | E-1D | 1000 | 0.99 | 1.02 | 1.11 |
| Comparative | | | | | | | | |
| Example 6 | | | | | | | | |
| Device | H-9 | H-9-D-E | E-10 | E-10D | 1000 | 0.98 | 1.33 | 2.02 |
| Example 22 | | | | | | | | |
| Device | H-9 | H-9-D-E | E-10 | E-19 | 1000 | 0.92 | 1.19 | 2.91 |
| Example 23 | | | | | | | | |
| Device | H-26 | H-3 | E-3 | E-3D | 1000 | 0.61 | 1.68 | 2.37 |
| Example 24 | | | | | | | | |
| Device | H-26 | H-29 | E-3 | E-3D | 1000 | 0.72 | 1.88 | 2.20 |
| Example 25 | | | | | | | | |
| Device | H-26 | / | E-3 | E-3D | 1000 | 0.88 | 1.02 | 1.26 |
| Comparative | | | | | | | | |
| Example 7 | | | | | | | | |
| Device | / | H-3 | E-3 | E-3D | 1000 | 0.91 | 0.99 | 1.16 |
| Comparative | | | | | | | | |
| Example 8 | | | | | | | | |
| Device | / | H-29 | E-3 | E-3D | 1000 | 0.71 | 1.08 | 1.12 |
| Comparative | | | | | | | | |
| Example 9 | | | | | | | | |
| Device | H-3 | / | E-1 | / | 1000 | 1.11 | 0.92 | 0.88 |
| Comparative | | | | | | | | |
| Example 10 | | | | | | | | |

As can be seen from Device Examples 11-13, the performance of the organic electroluminescent device fabricated by using a composition including four compounds in the present application is superior to the performance of the organic electroluminescent device fabricated by including a combination of three compounds.

As can be seen from Device Examples 11-13 and Device Comparative Examples 5-6, if the composition includes only one compound represented by Formula I (Device Comparative Examples 5-6), the organic electroluminescent device fabricated has poor comprehensive performance.

As can be seen by Device Examples 20, 21, and 23, if R₁₀₁ in the compound represented by Formula II is a triphenylenyl or if Ar₁₁ in the compound represented by Formula II is naphthylene, the lifetime of the organic electroluminescent device can be further improved.

As can be seen by the comparison of Device Example 16 and Device Example 11, if the composition contains Compound E-8, a driving voltage of the organic electroluminescent device can be further reduced and the current efficiency can be improved.

As can be seen from Device Examples 18-19, if Ar₁₁ in the compound represented by Formula II is a single bond, the current efficiency of the organic electroluminescent device can be improved.

As can be seen from Device Comparative Example 5 and Device Comparative Example 10, the performance of the device containing one compound represented by Formula I and two compounds represented by Formula II is superior to that of the device containing one compound represented by Formula I and one compound represented by Formula II.

### Device Example 26

This device example provides an organic electroluminescent device, wherein the composition provided in the present application is selected as the red-emitting host material.

The structure of the organic electroluminescent device is as follows: ITO/HT-1 (20 nm)/electron blocking layer (5 nm)/red-emitting host material (35 nm): Ir(piq)₃[10%]/TPBI (10 nm)/Alq3 (15 nm)/LiF (0.5 nm)/Al (150 nm); where ‘Ir(piq)₃[10%]’ refers to the doping ratio of the red dopant, that is, a volume ratio of the red-emitting host material to Ir(piq)₃ is 90:10.

The fabrication process of the organic electroluminescent device is as follows:
a glass substrate coated with an ITO transparent conductive layer was ultrasonically cleaned in a commercial detergent, rinsed with deionized water, degreased in an acetone:ethanol mixed solvent via ultrasonication, baked in a clean environment to remove moisture completely, treated with UV-ozone cleaning, and the surface bombarded with a low-energy cation beam;
the above glass substrate with an anode was placed in a vacuum chamber, and the chamber was evacuated to 1×10⁻⁵-9×10⁻⁴ Pa, and a hole transport layer (HT-1) was vacuum-evaporated onto the anode at a evaporation rate of 0.1 nm/s, forming a 20 nm-thick film;
EB was vacuum-evaporated as an electron blocking layer on the hole transport layer with an evaporation rate of 0.1 nm/s, and a thickness of the evaporation film was 5 nm;
the red-emitting host material and dopant Ir(piq)₃ were co-evaporated on the electron blocking layer as an emissive layer of the organic electroluminescent device with an evaporation rate of 0.1 nm/s, and a thickness of the total evaporation film was 35 nm; in this device example, and if the red-emitting host material is H-3, H-3-D-E, E-1, and E-1D, each compounds are placed in different evaporation sources for heating, and the heating rate is controlled so that the ratio of each volume that is evaporated onto the substrate is in the same proportion, which is used as the red-emitting host material;
an electron transport layer (TPBI) and Alq3 were sequentially evaporated on the emissive layer at a rate of 0.1 nm/s, with a thicknesses of 10 nm and 15 nm, respectively; and
a 0.5 nm-thick LiF layer and a 150 nm-thick Al layer were vacuum-evaporated as an electron injection layer and cathode on the electron transport layer, respectively.

### Device Example 27

Device Example 27 provide an organic electroluminescent device, which differs from Device Example 26 only in that the material of the electron blocking layer was different (see Table 7 below in detail), and other preparation steps and conditions were the same as those of Device Example 1.

The performance of the organic electroluminescent devices provided by Device Examples 26-27 was tested in the same manner as above. The test results are detailed in Table 7 below.

TABLE 7

| | Electron | | | | |
|---|---|---|---|---|---|
| | blocking | Required | | | |
| | layer | luminance/ | Driving | Current | LT90 |
| | material | (cd/m²) | voltage | efficiency | lifetime |
| Device | EB | 1000 | 1 | 1 | 1 |
| Example 26 | | | | | |
| Device | EB-D | 1000 | 0.92 | 1.11 | 1.09 |
| Example 27 | | | | | |

As can be seen from Table 7, by selecting spirofluorene compounds having a specific structure as the electron blocking layer material in the present application, and matching the compositions provided in the present application as the emissive layer material, the organic electroluminescent device obtained by the present application has a higher current efficiency and a longer service lifetime.

The applicant declares that the detailed process flow of the present application are illustrated by the above examples in the present application, but the present application is not limited to the above detailed process flow, that is, the present application does not necessarily rely on the above detailed process flow to be implemented. Those skilled in the art should understand that any improvement of the present application, the equivalent substitution of each raw material of the product, the addition of auxiliary ingredients, and the selection of specific methods in the present application shall fall within the protection scope and disclosure scope of the present application.

1. A composition, comprising two compounds represented by Formula I;
or the composition comprises one compound represented by Formula I and two compounds represented by Formula II;
the compound represented by Formula I is obtained by fusing a group represented by Formula I-A with any two adjacent carbon atoms on ring A in the group represented by Formula I-B; 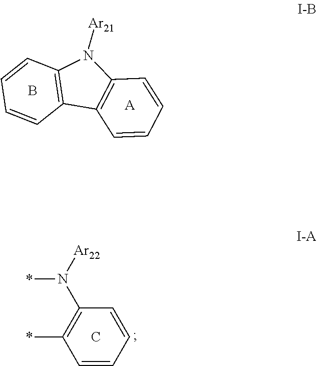
wherein “*” represents a fusion site;
Ar₂₁ and Ar₂₂ are each independently selected from C6 to C30 aryl or C6 to C20 heteroaryl;
the hydrogen atom in the compound represented by Formula I may be substituted with at least one of —F, —CN, C6 to C20 aryl, C1 to C6 alkyl, or C1 to C6 alkoxy;
the compound represented by Formula I meets at least one of the following conditions:
(1) the compound represented by Formula I contains no deuterium atom;
(2) hydrogen atoms on at least one of the ring A, ring B, and ring C in the compound represented by Formula I are all substituted with deuterium atoms;
(3) at least one hydrogen atom in Ar₂₁ of the compound represented by Formula I is substituted with a deuterium atom;
(4) at least one hydrogen atom in Ar₂₂ of the compound represented by Formula I is substituted with a deuterium atom;
(5) a hydrogen atom in the compound represented by Formula I is substituted with C6 to C20 aryl, and at least one hydrogen atom in the C6 to C20 aryl is substituted with a deuterium atom; and
(6) a hydrogen atom in the compound represented by Formula I is substituted with C1 to C6 alkyl and/or C1 to C6 alkoxy, and all hydrogen atoms in C1 to C6 alkyl and/or C1 to C6 alkoxy are substituted with deuterium atoms;
the compound represented by Formula II is obtained by fusing a group represented by Formula I-C with any two adjacent carbon atoms on ring E in the group represented by Formula I-D; 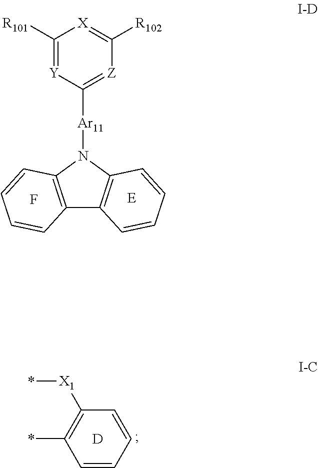
wherein “*” represents a fusion site;
Ar₁₁ is selected from any one of a single bond, phenylene, naphthylene, or biphenylene;
R₁₀₁ and R₁₀₂ are each independently selected from H, C6 to C30 aryl, or C6 to C20 heteroaryl;
X, Y, and Z are each independently selected from N or CR₃₀₄, wherein R₃₀₄ is selected from any one of H, phenyl, biphenyl, naphthyl, 9,9-dimethylfluorenyl, dibenzofuranyl, dibenzothienyl, and at least one of X, Y, and Z is N;
X₁ is selected from O, S, 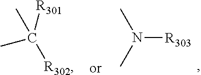
wherein R₃₀₁ and R₃₀₂ are each independently selected from C1 to C5 alkyl or phenyl, and R₃₀₃ is selected from phenyl or biphenyl, and the dashed line represents a linkage site;
the hydrogen atom in the compound represented by Formula II may be substituted with at least one of —F, —CN, C6 to C20 aryl, C1 to C6 alkyl, or C1 to C6 alkoxy;
the compound represented by Formula II meets at least one of the following conditions:
(a) the compound represented by Formula II contains no deuterium atom;
(b) hydrogen atoms on at least one of the ring F, ring D, and ring E in the compound represented by Formula II are all substituted with deuterium atoms;
(c) at least one hydrogen atom in R₁₀₁ of the compound represented by Formula II is substituted with a deuterium atom;
(d) at least one hydrogen atom in R₁₀₂ of the compound represented by Formula II is substituted with a deuterium atom;
(e) at least one hydrogen atom in Ar₁₁ of the compound represented by Formula II is substituted with a deuterium atom;
(f) a hydrogen atoms in R₃₀₁ and R₃₀₂ in the compound represented by Formula II are all substituted with deuterium atoms;
(g) at least one hydrogen atom in R₃₀₃ group of the compound represented by Formula II is substituted with a deuterium atom;
(h) R₃₀₄ in the compound represented by Formula II is a deuterium atom;
(i) in the compound represented by Formula II, when R₃₀₄ is selected from any one of phenyl, biphenyl, naphthyl, 9,9-dimethylfluorenyl, dibenzofuranyl, or dibenzothienyl, at least one hydrogen atom in the phenyl, biphenyl, naphthyl, 9,9-dimethylfluorenyl, dibenzofuranyl, or dibenzothienyl is substituted with a deuterium atom;
(j) a hydrogen atom in the compound represented by Formula II is substituted with C6 to C20 aryl, and at least one hydrogen atom in the C6 to C20 aryl is substituted with a deuterium atom; and
(k) a hydrogen atom in the compound represented by Formula II is substituted with C1 to C6 alkyl and/or C1 to C6 alkoxy, and all hydrogen atoms in C1 to C6 alkyl and/or C1 to C6 alkoxy are substituted with deuterium atoms.

2. The composition according to claim 1, wherein the composition includes two compounds represented by Formula I, and the composition further includes at least one compound represented by Formula II.

3. The composition according to claim 1, wherein the composition includes two compounds represented by Formula I and one compound represented by Formula II.

4. The composition according to claim 1, wherein the composition includes two compounds represented by Formula I and two compounds represented by Formula II.

5. The composition according to claim 1, wherein in the composition, at least one compound represented by Formula I meets at least one of conditions (2) to (6);
optionally, in the composition, at least one compound represented by Formula I meets at least one of conditions (2) to (6), and the compound represented by Formula II meets condition (a).

6. The composition according to claim 1, wherein the composition includes a compound containing deuterium atoms represented by Formula I and a compound without deuterium atoms represented by Formula II;
the compound containing a deuterium atom represented by Formula I is selected from any one of Compound I-1-D, Compound I-2-D, or Compound I-3-D:

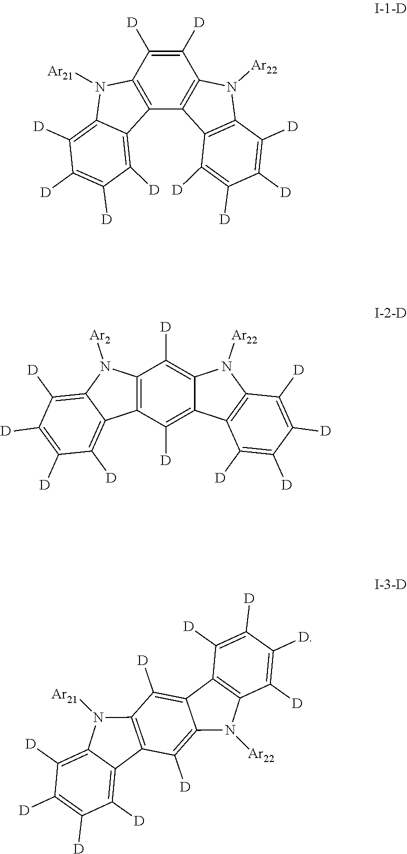

7. The composition according to claim 1, wherein the Ar₂₁ and Ar₂₂ are each independently selected from any one or a combination of at least two of phenyl, naphthyl, fluorenyl, anthryl, phenanthryl, biphenyl, dibenzofuranyl, dibenzothienyl, terphenyl, or quaterphenyl;
optionally, the Ar₂₁ is selected from phenyl, biphenyl, or terphenyl;
optionally, the Ar₂₂ is selected from biphenyl, terphenyl, or quaterphenyl;
optionally, the Ar₂₁ is phenyl and the Ar₂₂ is selected from biphenyl, terphenyl, or quaterphenyl;
optionally, the Ar₂₁ is biphenyl, and the Ar₂₂ is selected from biphenyl or terphenyl;
optionally, the R₁₀₁ and the R₁₀₂ are each independently selected from H, phenyl, naphthyl, triphenylenyl, fluoranthenyl, fluorenyl, anthryl, phenanthryl, biphenyl, naphthyl, dibenzofuranyl, or dibenzothienyl;
optionally, the R₁₀₁ and the R₁₀₂ are each independently selected from phenyl, biphenyl, or triphenylenyl;
optionally, the R₃₀₃ is selected from phenyl;
optionally, the Ar₁₁ is selected from any one of a single bond, phenylene, or naphthylene.

8. The composition according to claim 1, wherein the compound represented by Formula I has a structure as represented by Formula I-1, Formula I-2, or Formula I-3: 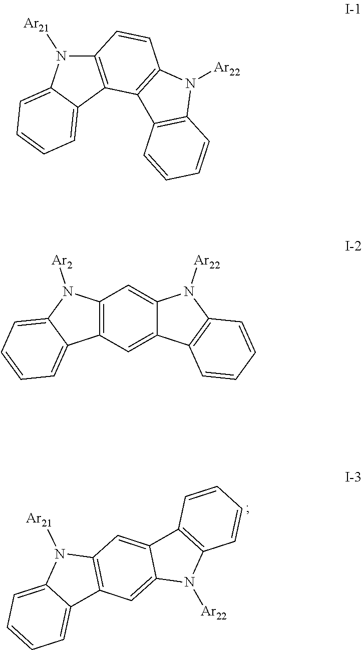
wherein Ar₂₁ and Ar₂₂ are defined the same as in claim 1;
optionally, the compound represented by Formula I meets condition (3) and/or condition (4);
optionally, the compound represented by Formula I is selected from any one of Compound I-1-D, Compound I-2-D, or Compound I-3-D: 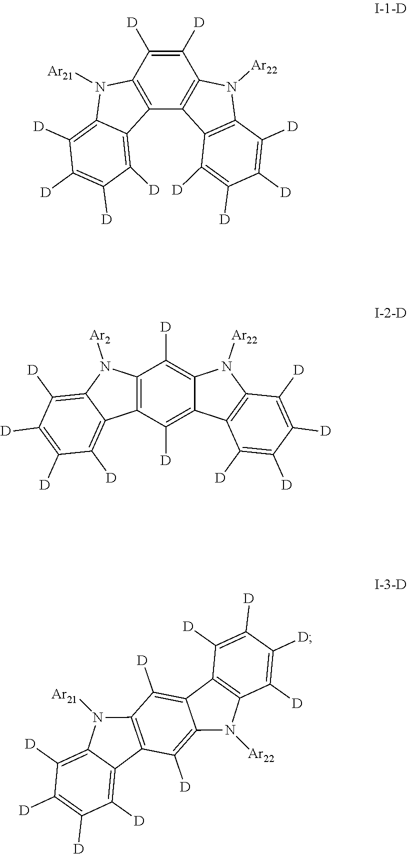
wherein Ar₂₁ and Ar₂₂ are defined the same as in claim 1, and the hydrogen atoms in Ar₂₁ and Ar₂₂ are not substituted with deuterium atoms.

9. The composition according to claim 1, wherein the compound represented by Formula I is selected from any one of the following compounds: 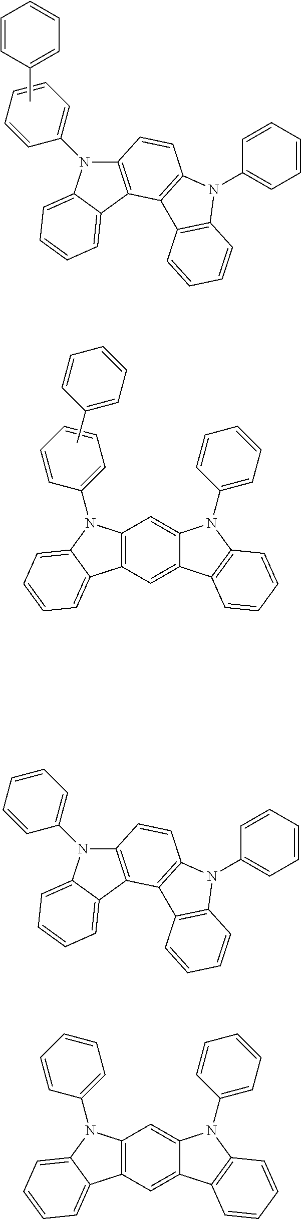 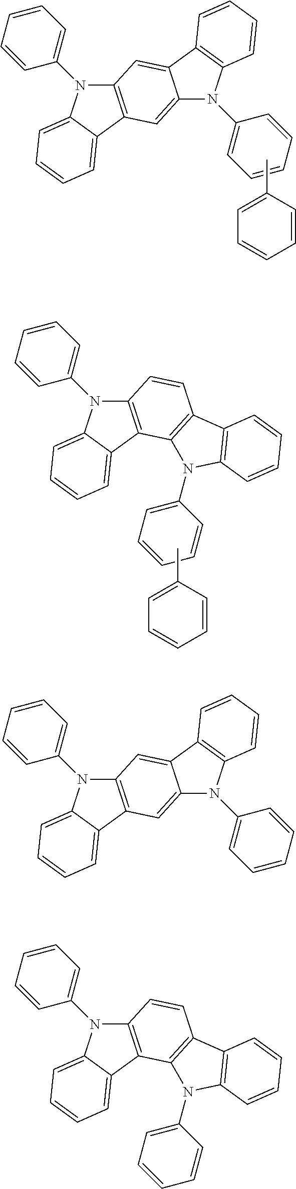 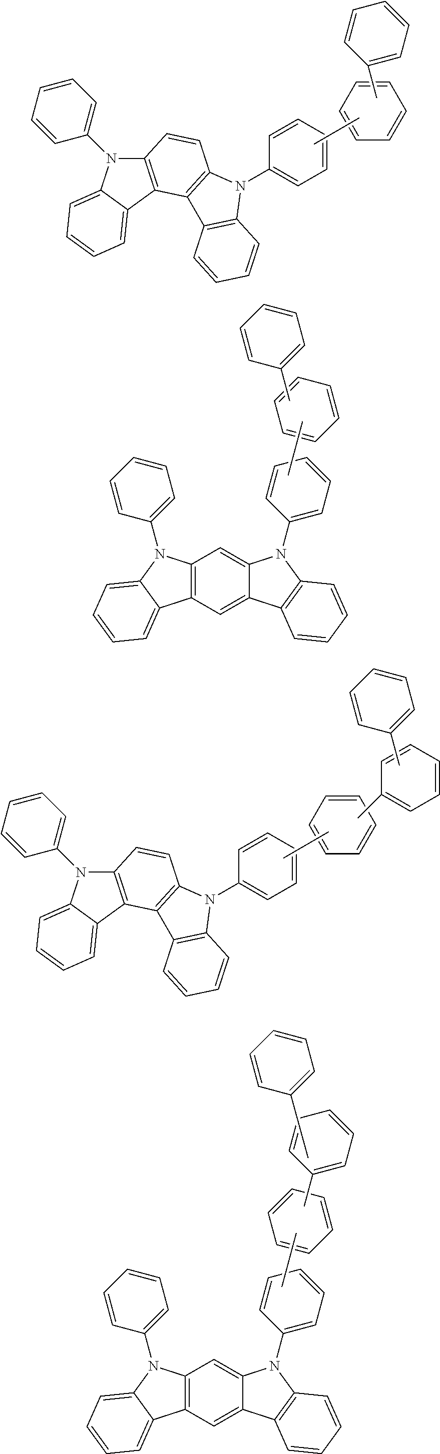 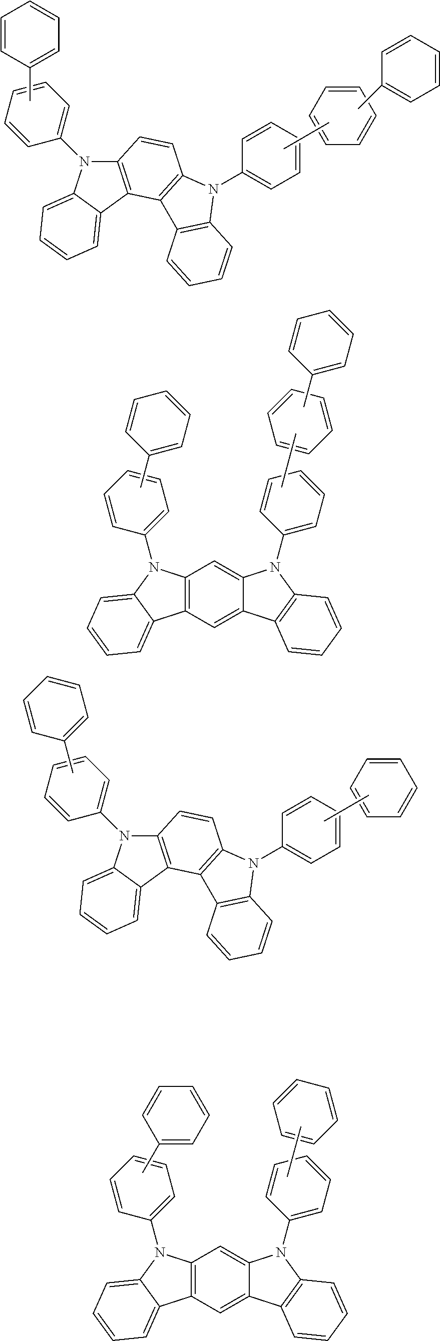 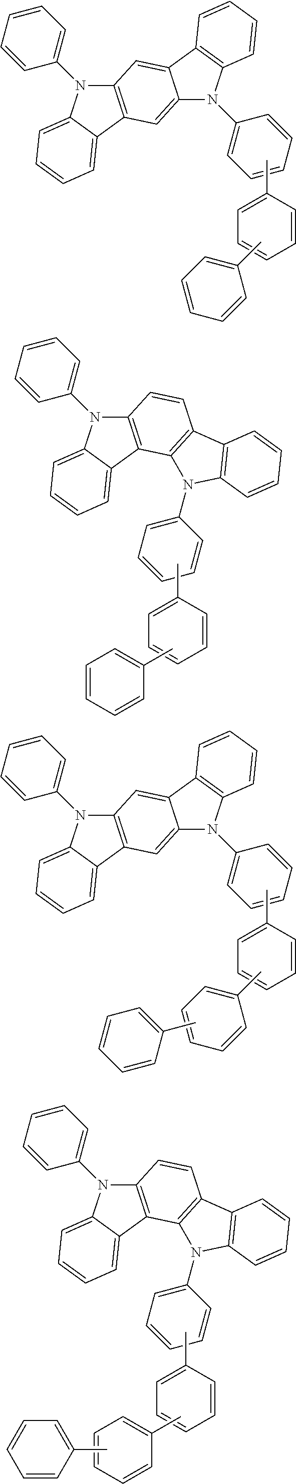 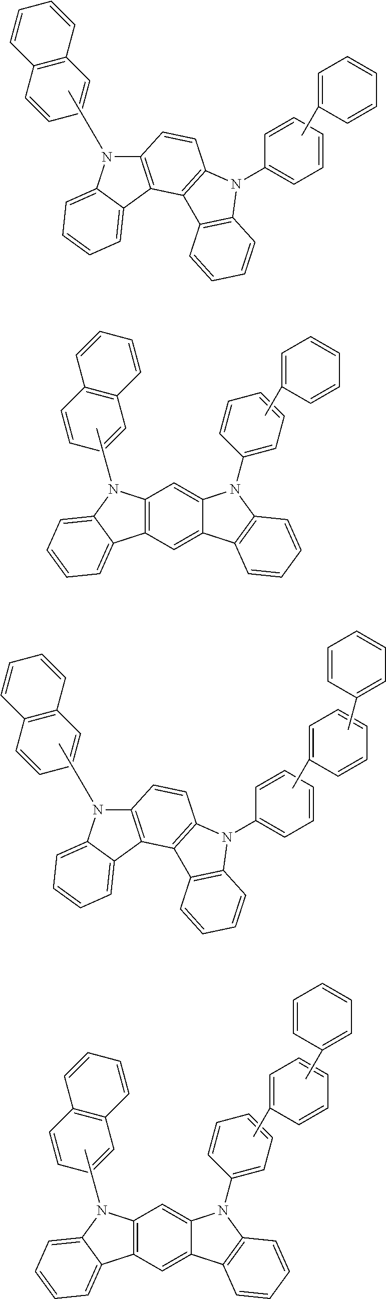 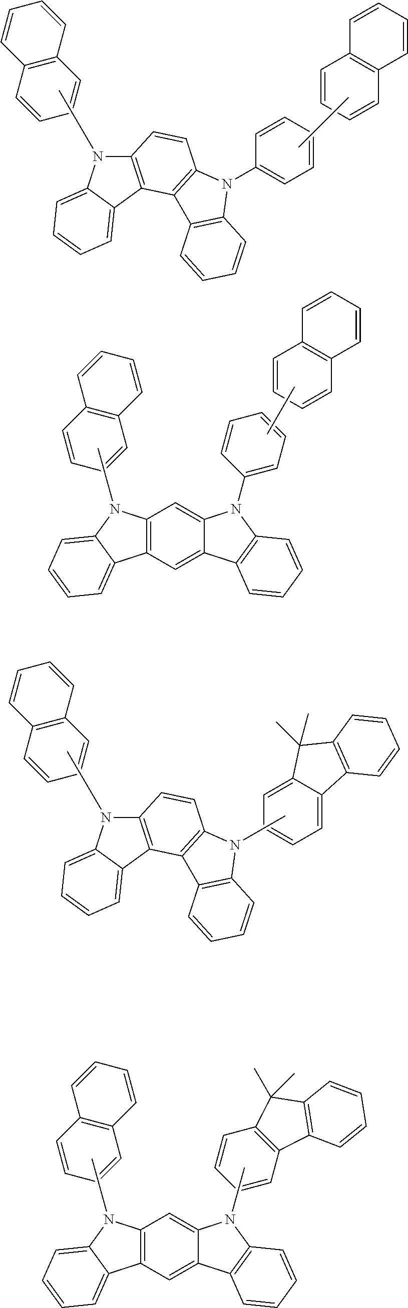 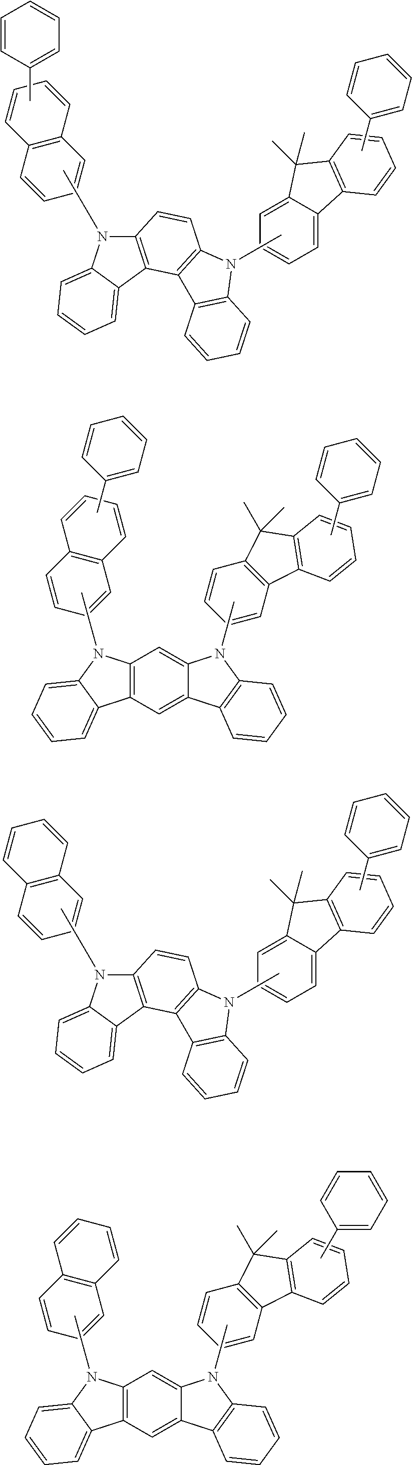 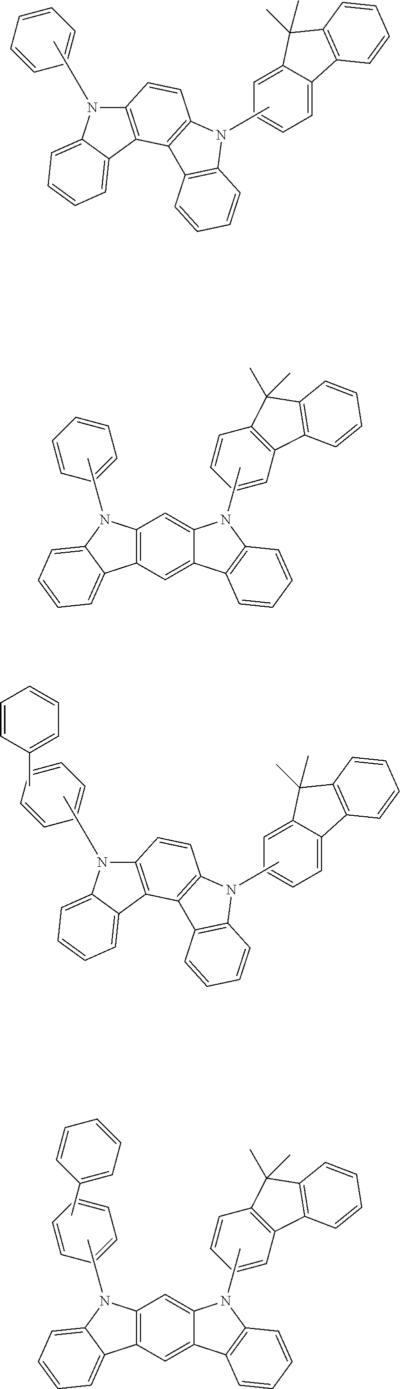 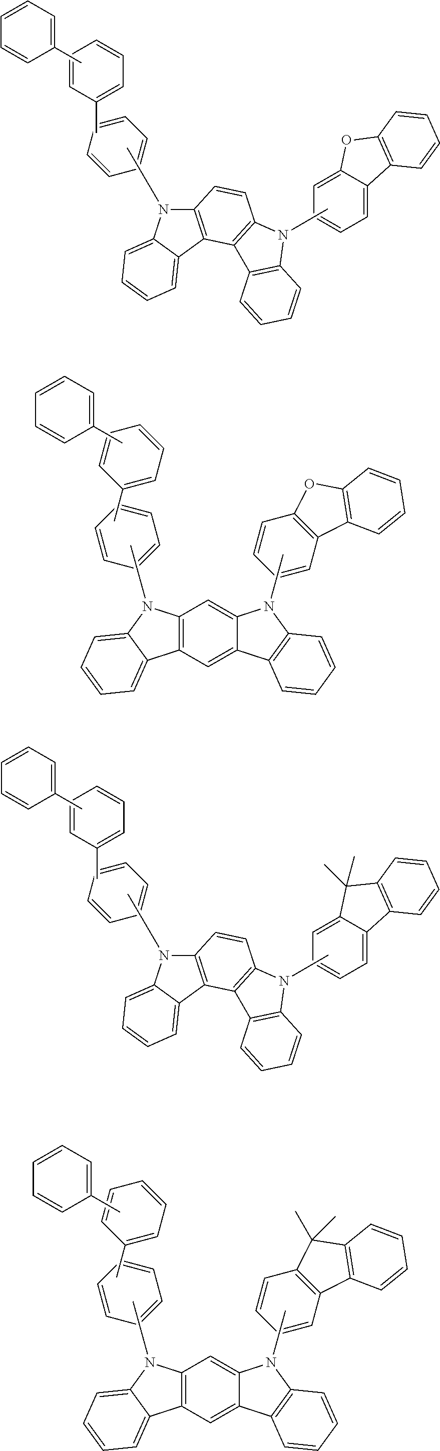 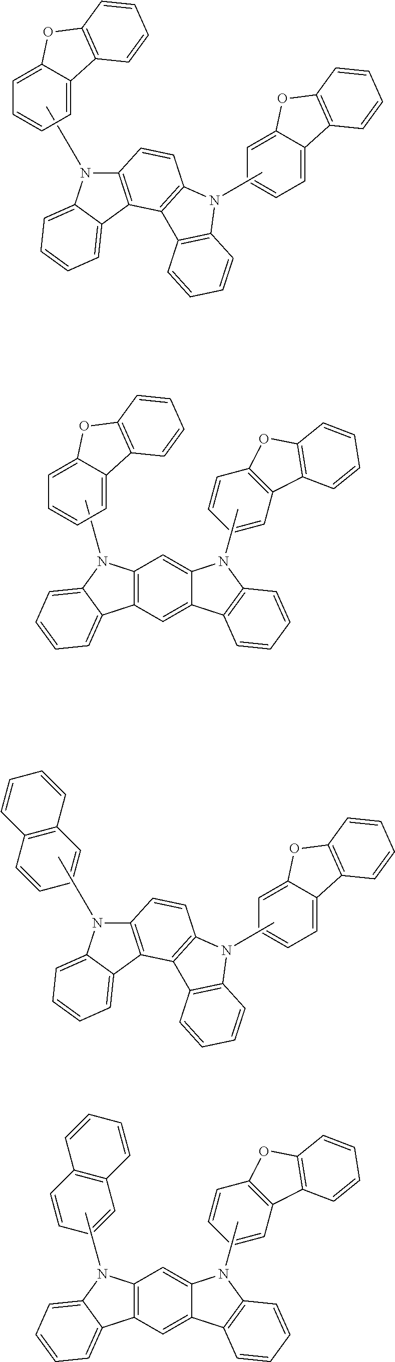 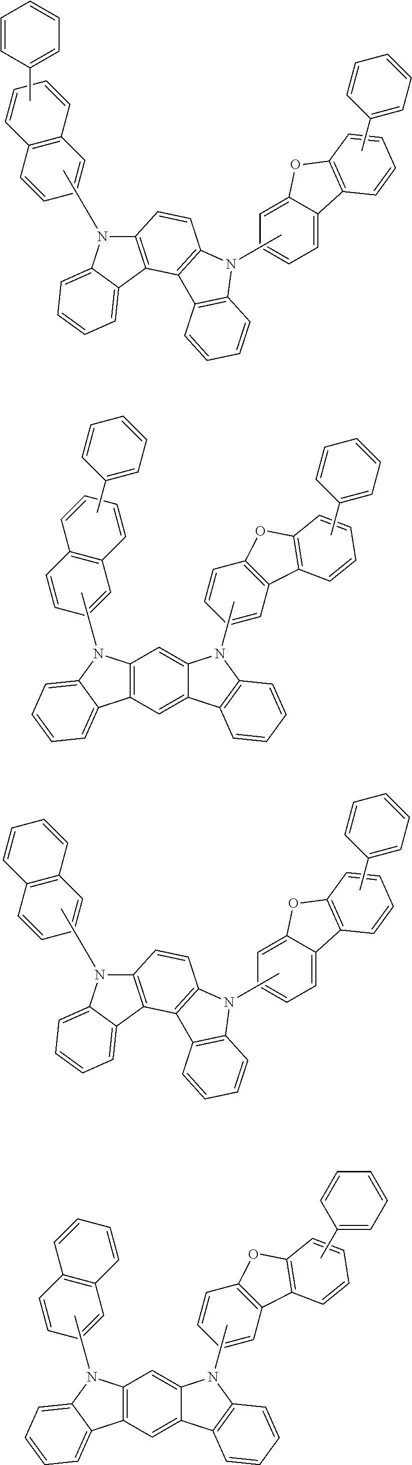 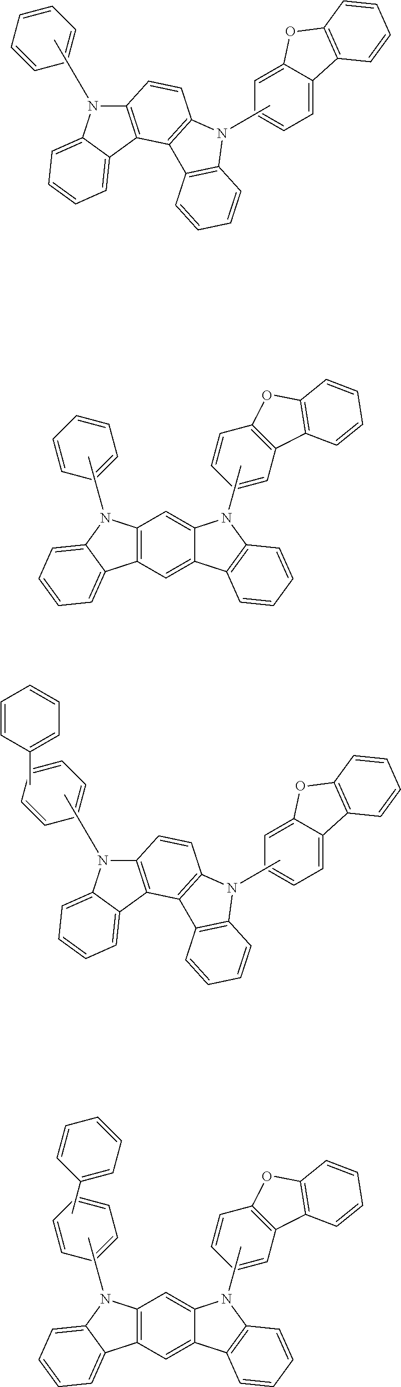 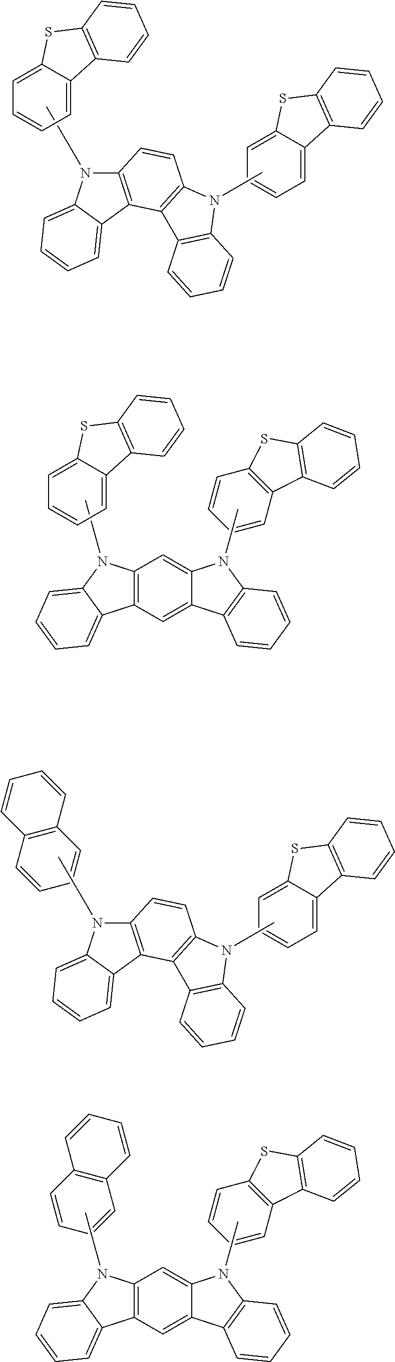 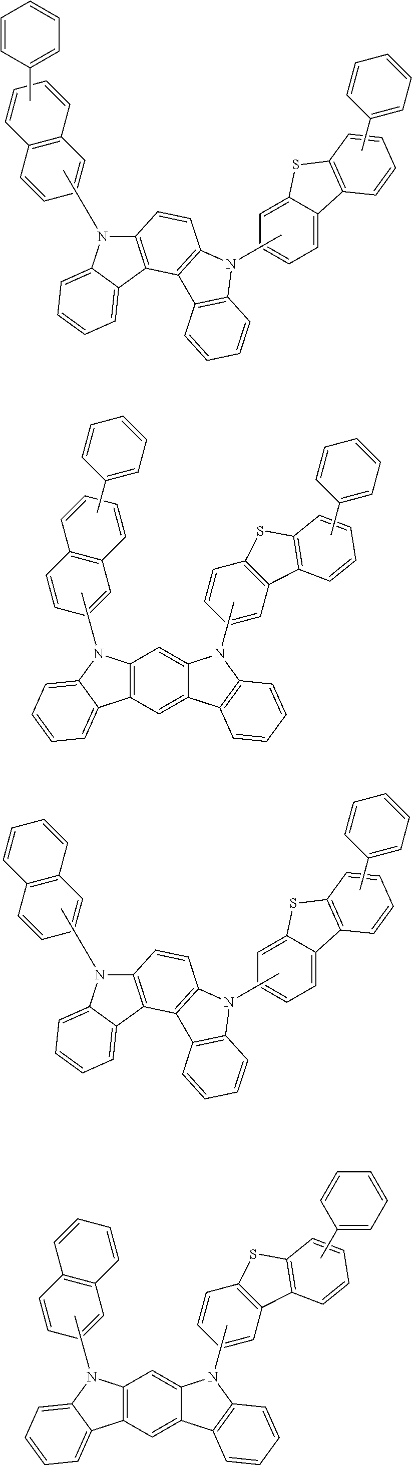 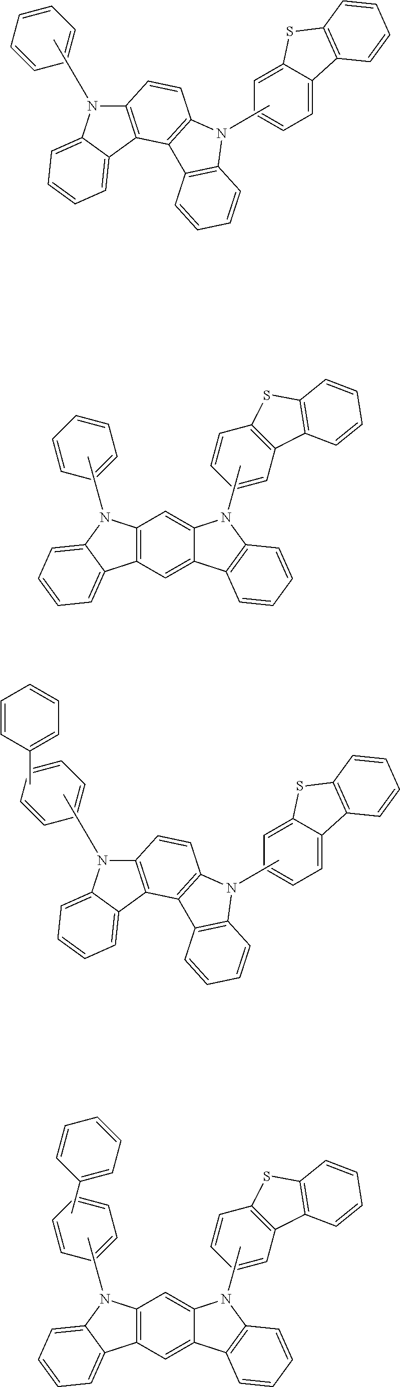 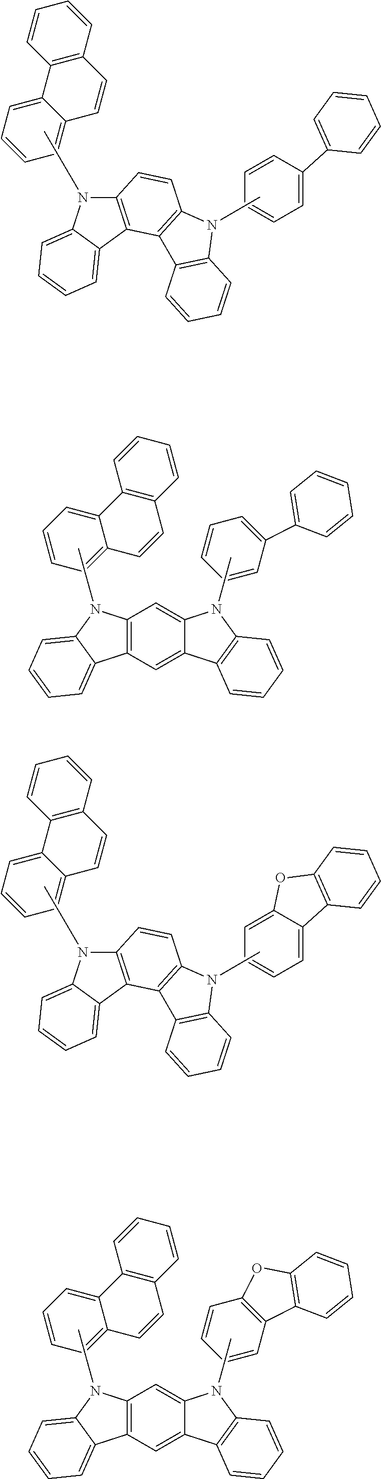 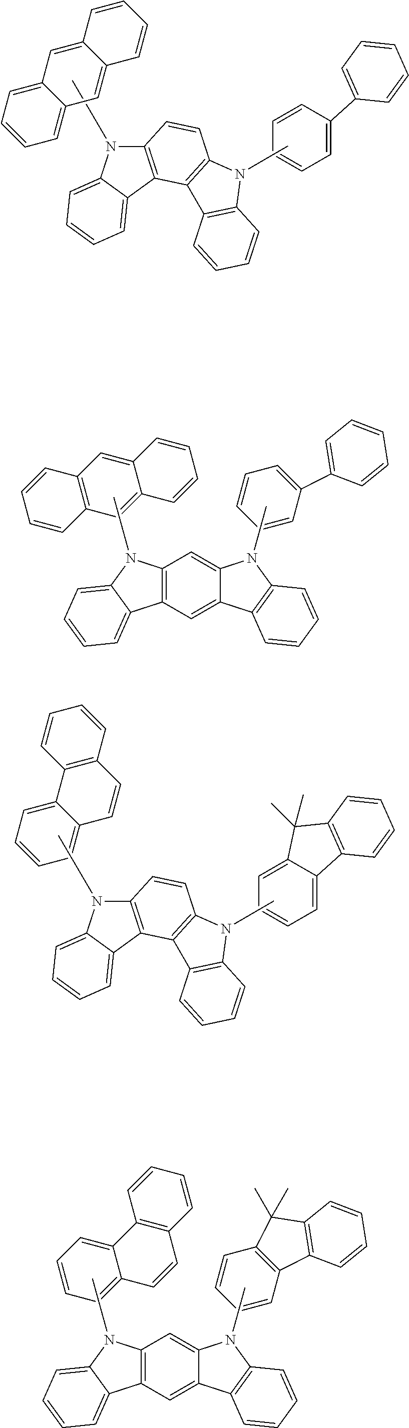 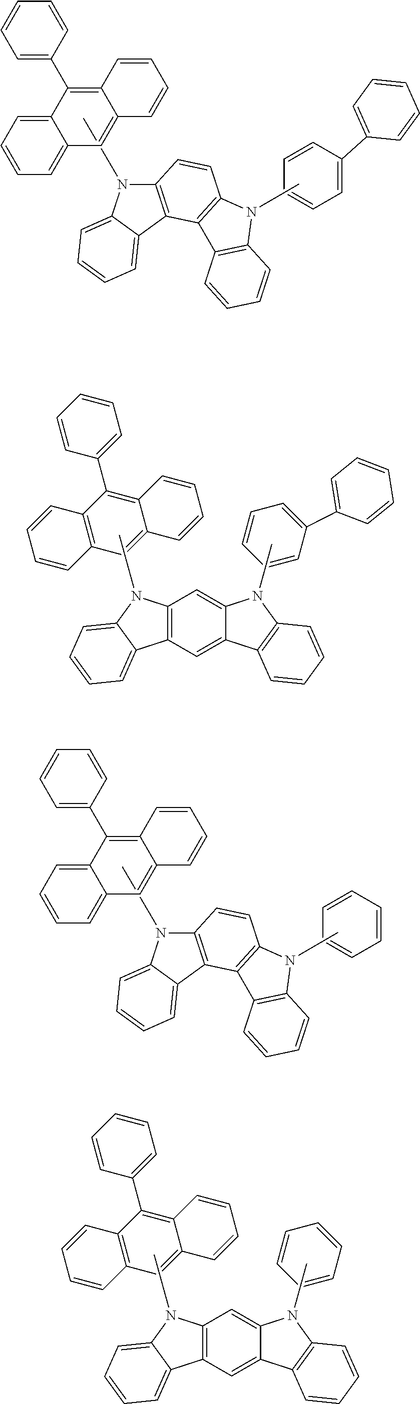 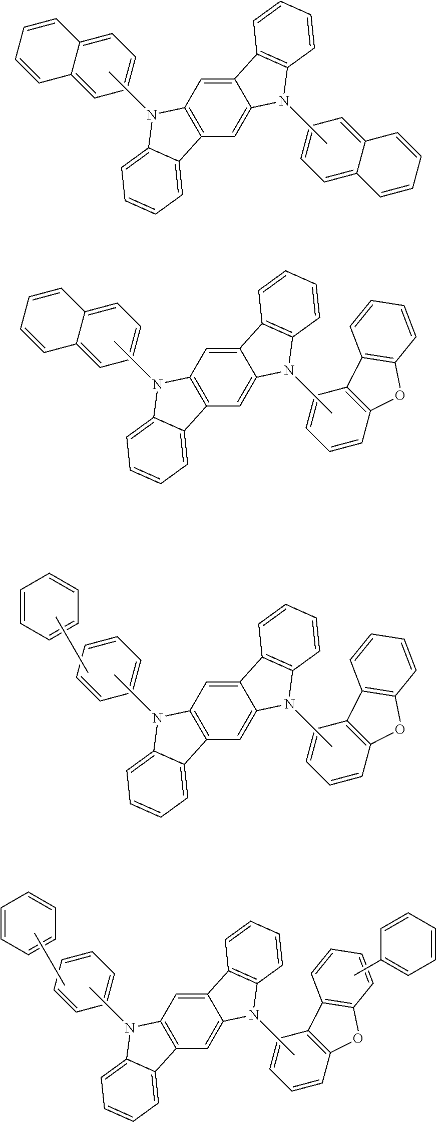 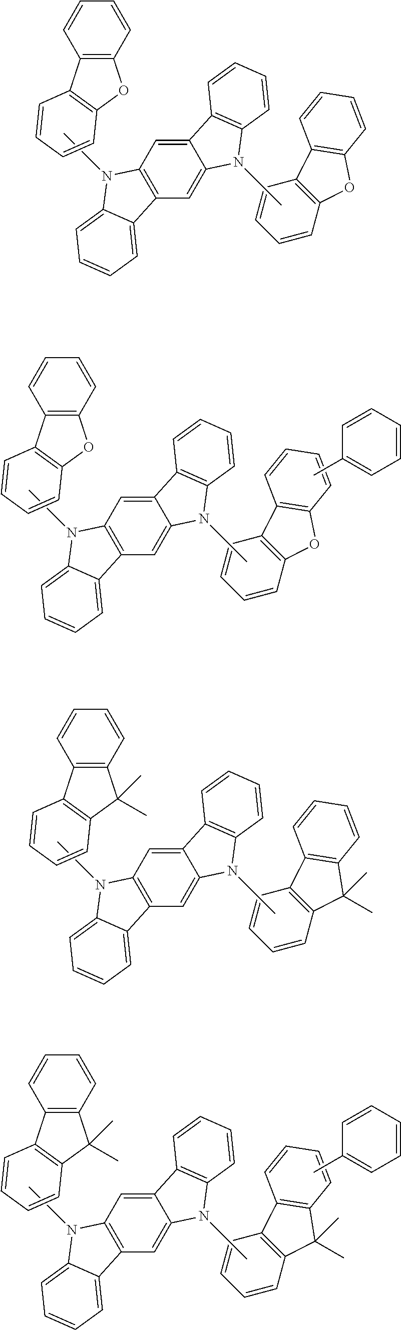
wherein a hydrogen atom in the above compound can be substituted with a deuterium atom;
optionally, the compound represented by Formula I is selected from any one of Compound H-1 to Compound H-44: 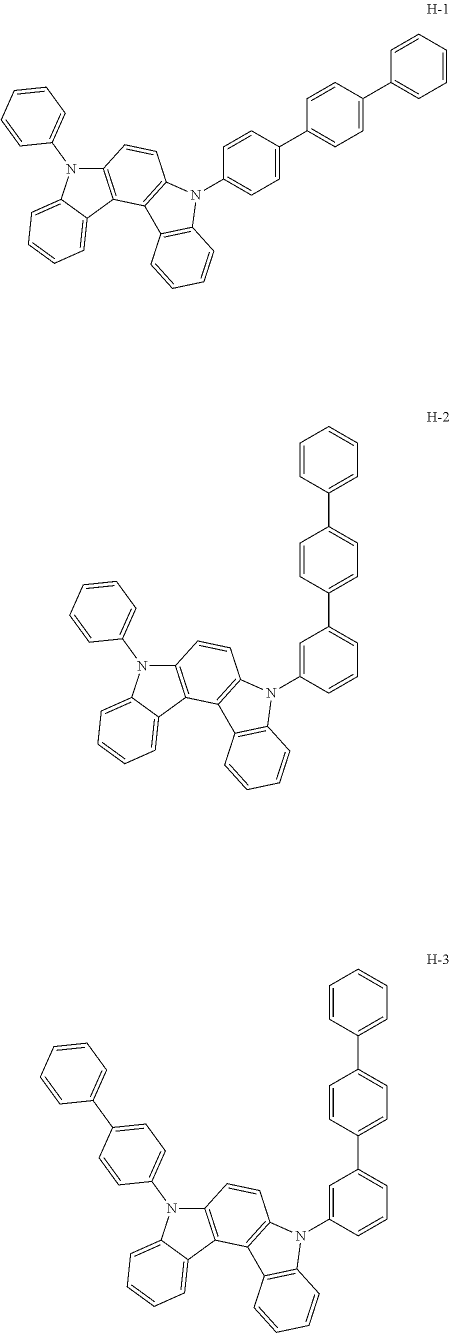 [SMILES: c1ccc(-c2ccc(-c3ccc(-n4c5ccccc5c5c6c7ccccc7n(-c7ccccc7)c6ccc54)cc3)cc2)cc1 ; c1ccc(-c2ccc(-c3cccc(-n4c5ccccc5c5c6c7ccccc7n(-c7ccccc7)c6ccc54)c3)cc2)cc1 ; c1ccc(-c2ccc(-c3cccc(-n4c5ccccc5c5c6c7ccccc7n(-c7ccc(-c8ccccc8)cc7)c6ccc54)c3)cc2)cc1] 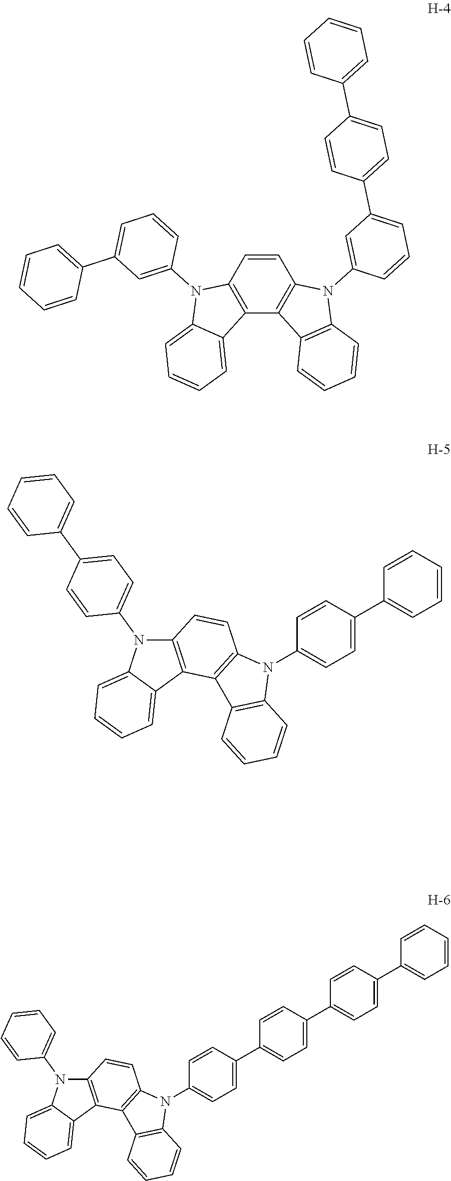 [SMILES: c1ccc(-c2ccc(-c3cccc(-n4c5ccccc5c5c6c7ccccc7n(-c7cccc(-c8ccccc8)c7)c6ccc54)c3)cc2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-c3ccc(-c4ccc(-n5c6ccccc6c6c7c8ccccc8n(-c8ccccc8)c7ccc65)cc4)cc3)cc2)cc1] 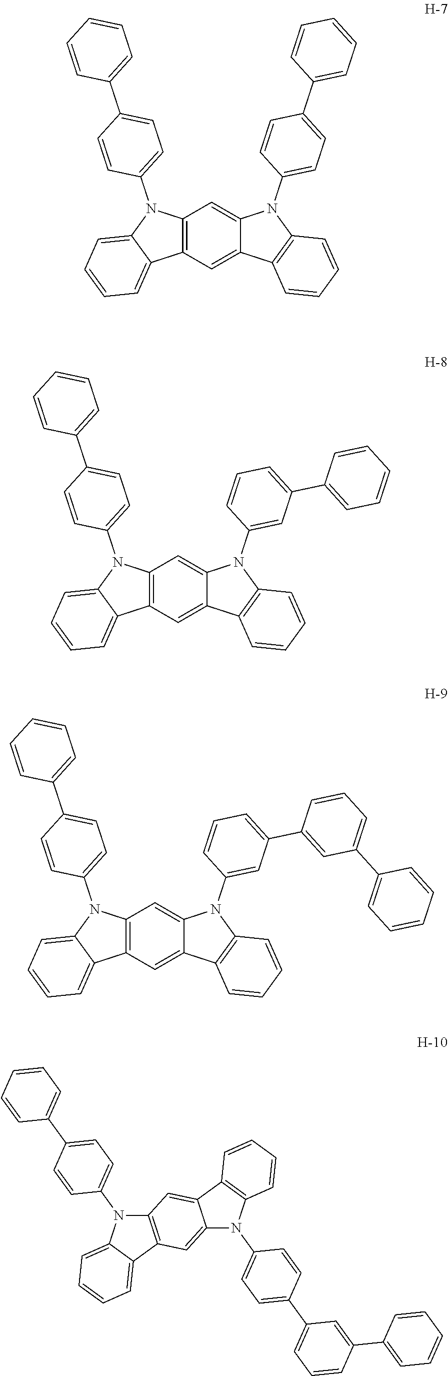 [SMILES: c1ccc(-c2ccc(-n3c4ccccc4c4cc5c6ccccc6n(-c6ccc(-c7ccccc7)cc6)c5cc43)cc2)cc1 ; c1ccc(-c2ccc(-n3c4ccccc4c4cc5c6ccccc6n(-c6cccc(-c7ccccc7)c6)c5cc43)cc2)cc1 ; c1ccc(-c2ccc(-n3c4ccccc4c4cc5c6ccccc6n(-c6cccc(-c7cccc(-c8ccccc8)c7)c6)c5cc43)cc2)cc1 ; c1ccc(-c2ccc(-n3c4ccccc4c4cc5c(cc43)c3ccccc3n5-c3ccc(-c4cccc(-c5ccccc5)c4)cc3)cc2)cc1] 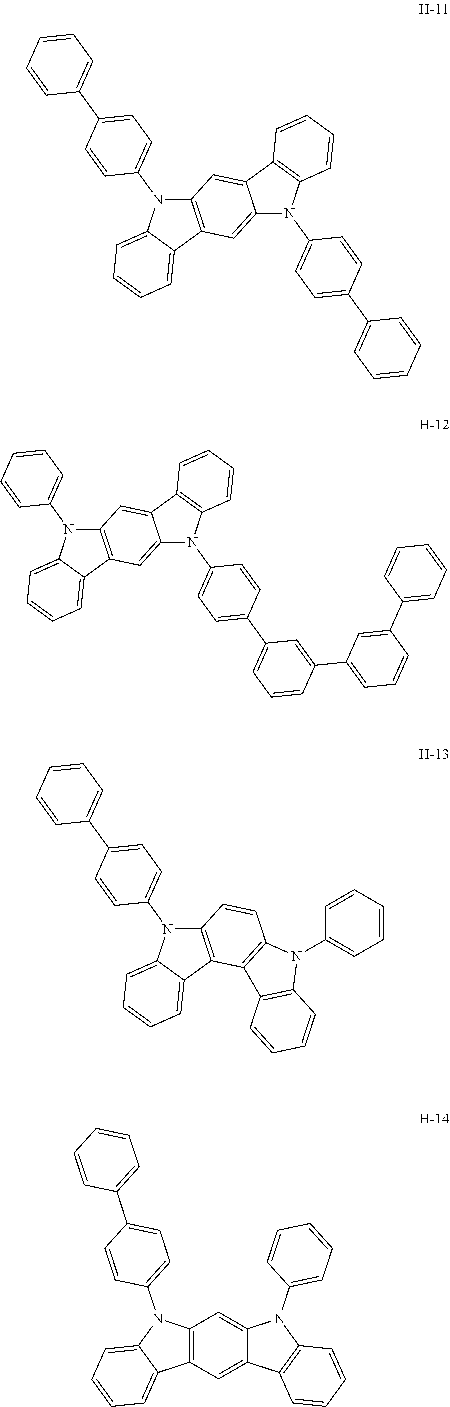 [SMILES: c1ccc(-c2ccc(-n3c4ccccc4c4cc5c(cc43)c3ccccc3n5-c3ccc(-c4ccccc4)cc3)cc2)cc1 ; c1ccc(-c2cccc(-c3cccc(-c4ccc(-n5c6ccccc6c6cc7c(cc65)c5ccccc5n7-c5ccccc5)cc4)c3)c2)cc1 ; c1ccc(-c2ccc(-n3c4ccccc4c4c5c6ccccc6n(-c6ccccc6)c5ccc43)cc2)cc1 ; c1ccc(-c2ccc(-n3c4ccccc4c4cc5c6ccccc6n(-c6ccccc6)c5cc43)cc2)cc1] 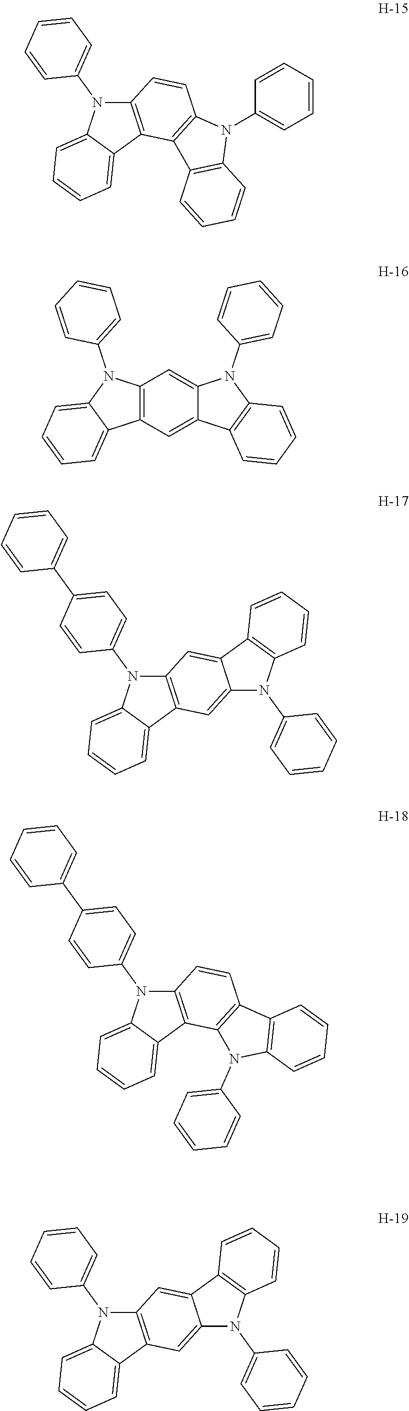 [SMILES: c1ccc(-n2c3ccccc3c3c4c5ccccc5n(-c5ccccc5)c4ccc32)cc1 ; c1ccc(-n2c3ccccc3c3cc4c5ccccc5n(-c5ccccc5)c4cc32)cc1 ; c1ccc(-c2ccc(-n3c4ccccc4c4cc5c(cc43)c3ccccc3n5-c3ccccc3)cc2)cc1 ; c1ccc(-c2ccc(-n3c4ccccc4c4c3ccc3c5ccccc5n(-c5ccccc5)c34)cc2)cc1 ; c1ccc(-n2c3ccccc3c3cc4c(cc32)c2ccccc2n4-c2ccccc2)cc1] 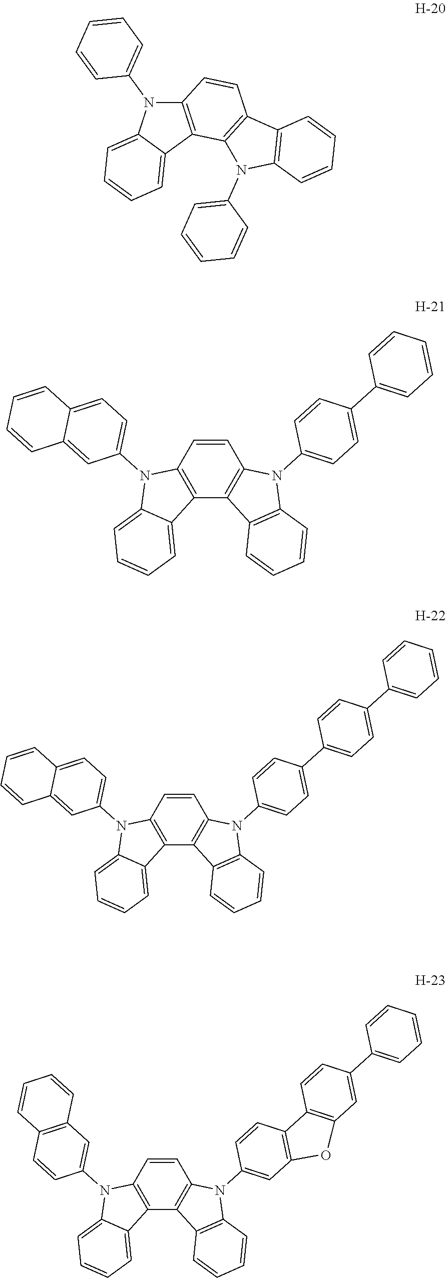 [SMILES: c1ccc(-n2c3ccccc3c3c2ccc2c4ccccc4n(-c4ccccc4)c23)cc1 ; c1ccc(-c2ccc(-n3c4ccccc4c4c5c6ccccc6n(-c6ccc7ccccc7c6)c5ccc43)cc2)cc1 ; c1ccc(-c2ccc(-c3ccc(-n4c5ccccc5c5c6c7ccccc7n(-c7ccc8ccccc8c7)c6ccc54)cc3)cc2)cc1 ; c1ccc(-c2ccc3c(c2)oc2cc(-n4c5ccccc5c5c6c7ccccc7n(-c7ccc8ccccc8c7)c6ccc54)ccc23)cc1] 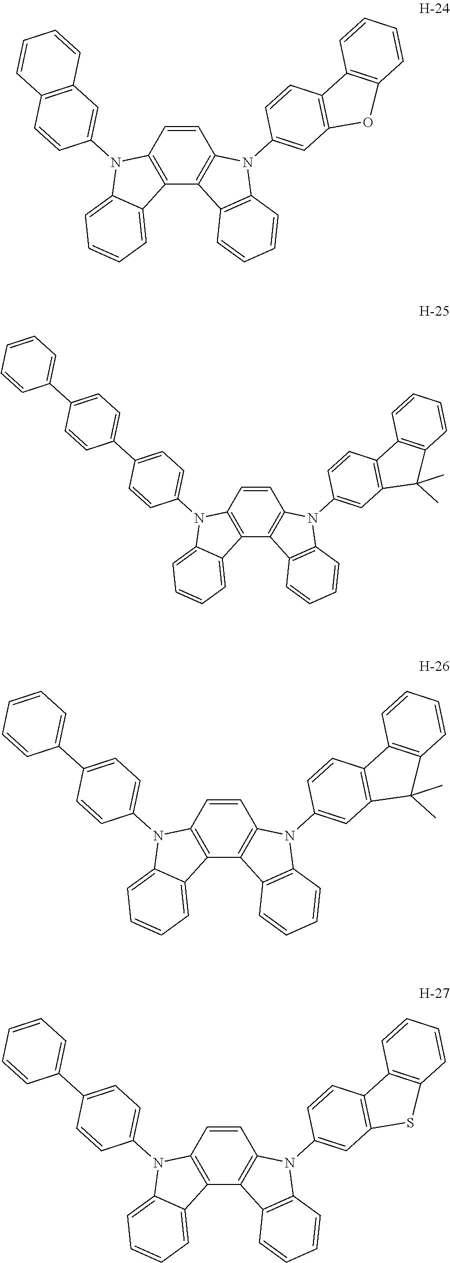 [SMILES: c1ccc2cc(-n3c4ccccc4c4c5c6ccccc6n(-c6ccc7c(c6)oc6ccccc67)c5ccc43)ccc2c1 ; CC1(C)c2ccccc2-c2ccc(-n3c4ccccc4c4c5c6ccccc6n(-c6ccc(-c7ccc(-c8ccccc8)cc7)cc6)c5ccc43)cc21 ; CC1(C)c2ccccc2-c2ccc(-n3c4ccccc4c4c5c6ccccc6n(-c6ccc(-c7ccccc7)cc6)c5ccc43)cc21 ; c1ccc(-c2ccc(-n3c4ccccc4c4c5c6ccccc6n(-c6ccc7c(c6)sc6ccccc67)c5ccc43)cc2)cc1] 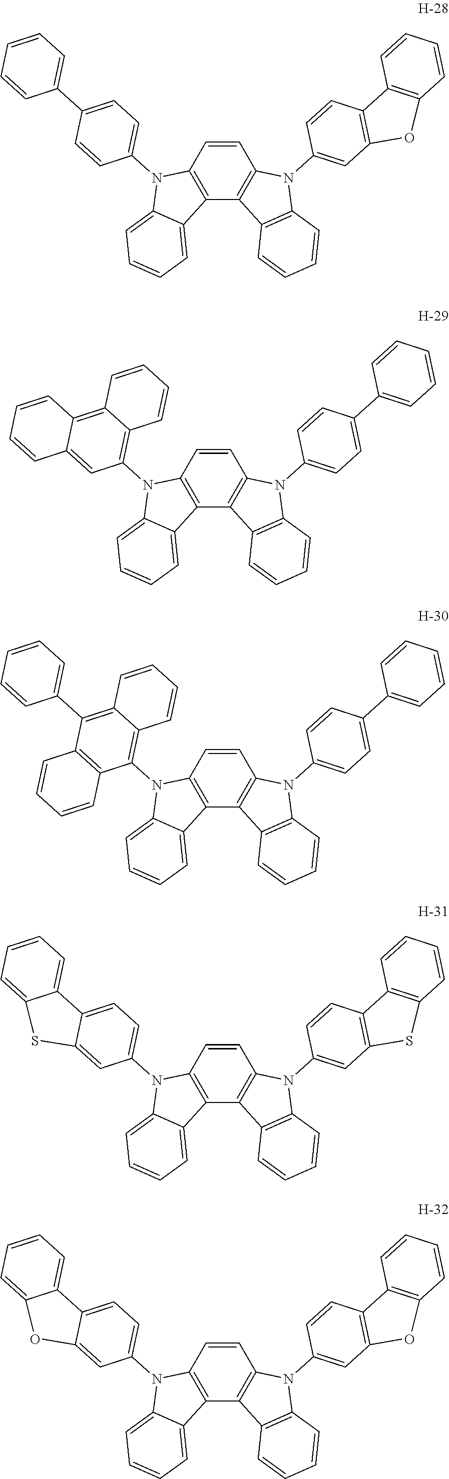 [SMILES: c1ccc(-c2ccc(-n3c4ccccc4c4c5c6ccccc6n(-c6ccc7c(c6)oc6ccccc67)c5ccc43)cc2)cc1 ; c1ccc(-c2ccc(-n3c4ccccc4c4c5c6ccccc6n(-c6cc7ccccc7c7ccccc67)c5ccc43)cc2)cc1 ; c1ccc(-c2ccc(-n3c4ccccc4c4c5c6ccccc6n(-c6c7ccccc7c(-c7ccccc7)c7ccccc67)c5ccc43)cc2)cc1 ; c1ccc2c(c1)sc1cc(-n3c4ccccc4c4c5c6ccccc6n(-c6ccc7c(c6)sc6ccccc67)c5ccc43)ccc12 ; c1ccc2c(c1)oc1cc(-n3c4ccccc4c4c5c6ccccc6n(-c6ccc7c(c6)oc6ccccc67)c5ccc43)ccc12] 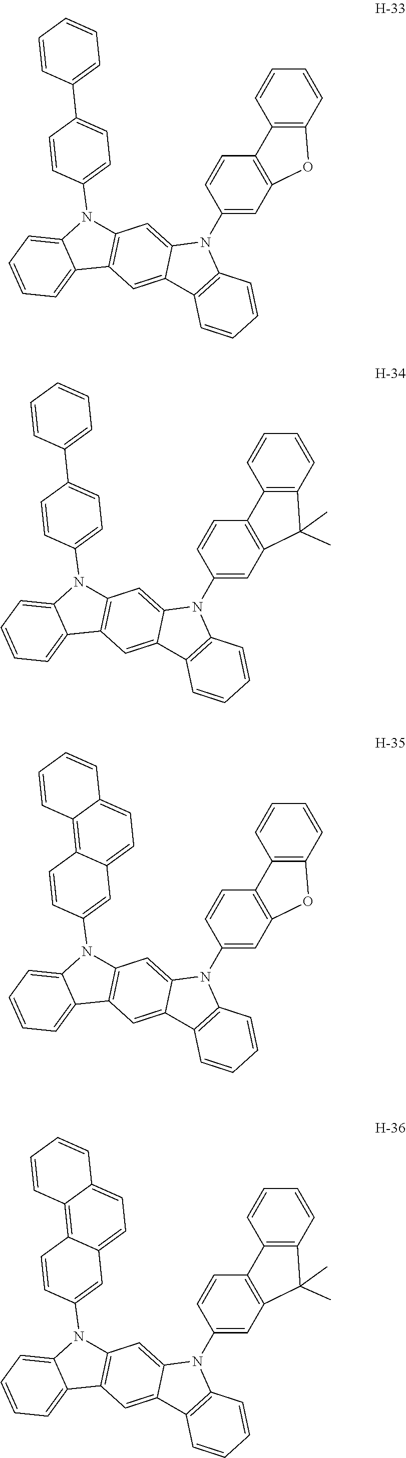 [SMILES: c1ccc(-c2ccc(-n3c4ccccc4c4cc5c6ccccc6n(-c6ccc7c(c6)oc6ccccc67)c5cc43)cc2)cc1 ; CC1(C)c2ccccc2-c2ccc(-n3c4ccccc4c4cc5c6ccccc6n(-c6ccc(-c7ccccc7)cc6)c5cc43)cc21 ; c1ccc2c(c1)ccc1cc(-n3c4ccccc4c4cc5c6ccccc6n(-c6ccc7c(c6)oc6ccccc67)c5cc43)ccc12 ; CC1(C)c2ccccc2-c2ccc(-n3c4ccccc4c4cc5c6ccccc6n(-c6ccc7c(ccc8ccccc87)c6)c5cc43)cc21] 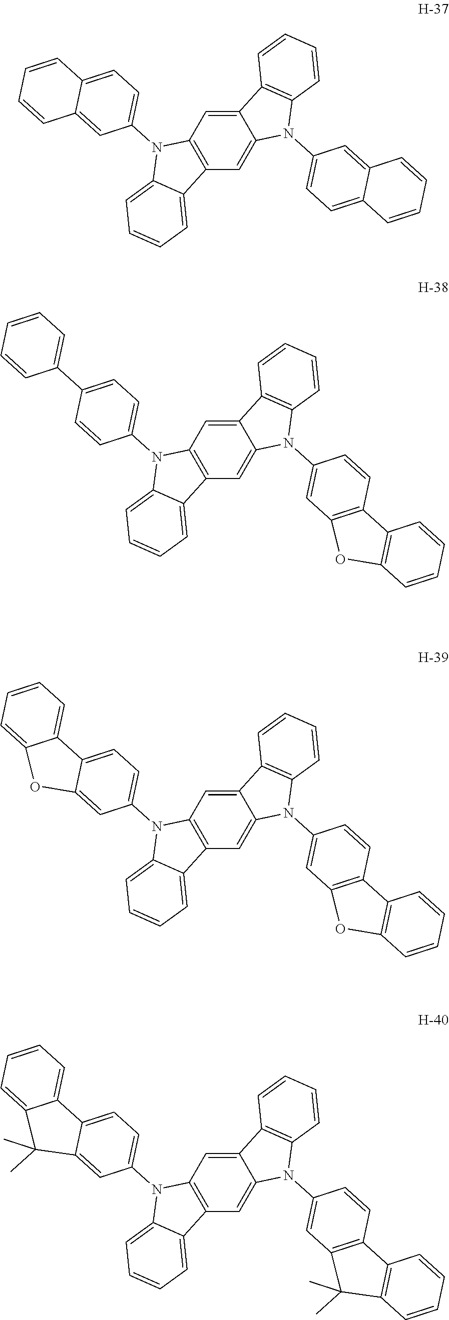 [SMILES: c1ccc2cc(-n3c4ccccc4c4cc5c(cc43)c3ccccc3n5-c3ccc4ccccc4c3)ccc2c1 ; c1ccc(-c2ccc(-n3c4ccccc4c4cc5c(cc43)c3ccccc3n5-c3ccc4c(c3)oc3ccccc34)cc2)cc1 ; c1ccc2c(c1)oc1cc(-n3c4ccccc4c4cc5c(cc43)c3ccccc3n5-c3ccc4c(c3)oc3ccccc34)ccc12 ; CC1(C)c2ccccc2-c2ccc(-n3c4ccccc4c4cc5c(cc43)c3ccccc3n5-c3ccc4c(c3)C(C)(C)c3ccccc3-4)cc21] 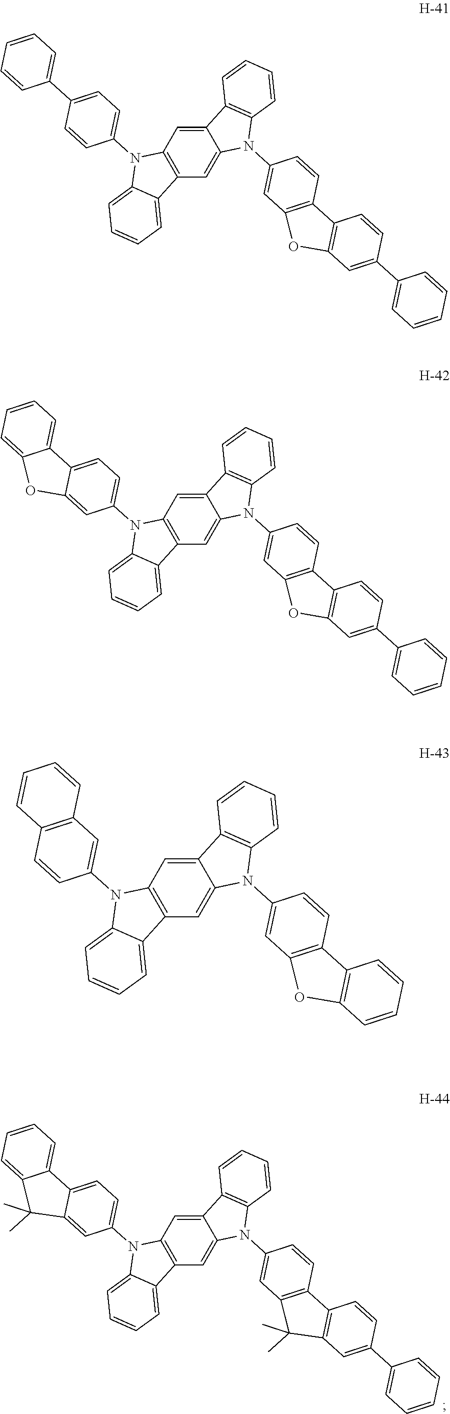 [SMILES: c1ccc(-c2ccc(-n3c4ccccc4c4cc5c(cc43)c3ccccc3n5-c3ccc4c(c3)oc3cc(-c5ccccc5)ccc34)cc2)cc1 ; c1ccc(-c2ccc3c(c2)oc2cc(-n4c5ccccc5c5cc6c(cc54)c4ccccc4n6-c4ccc5c(c4)oc4ccccc45)ccc23)cc1 ; c1ccc2cc(-n3c4ccccc4c4cc5c(cc43)c3ccccc3n5-c3ccc4c(c3)oc3ccccc34)ccc2c1 ; CC1(C)c2ccccc2-c2ccc(-n3c4ccccc4c4cc5c(cc43)c3ccccc3n5-c3ccc4c(c3)C(C)(C)c3cc(-c5ccccc5)ccc3-4)cc21]
wherein hydrogen atoms in the Compound H-1 to Compound H-44 can be substituted with deuterium atoms.

10. The composition according to claim 1, wherein the compound represented by Formula II is selected from any one of the following compounds: 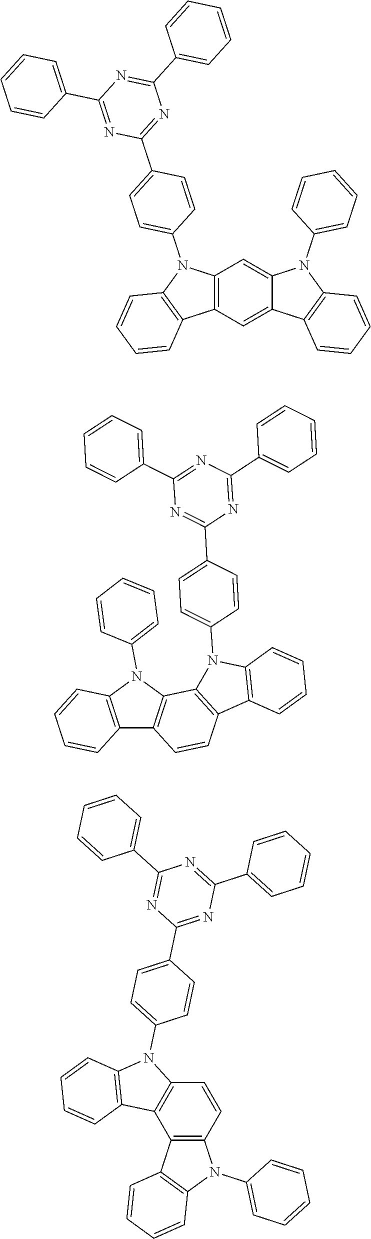 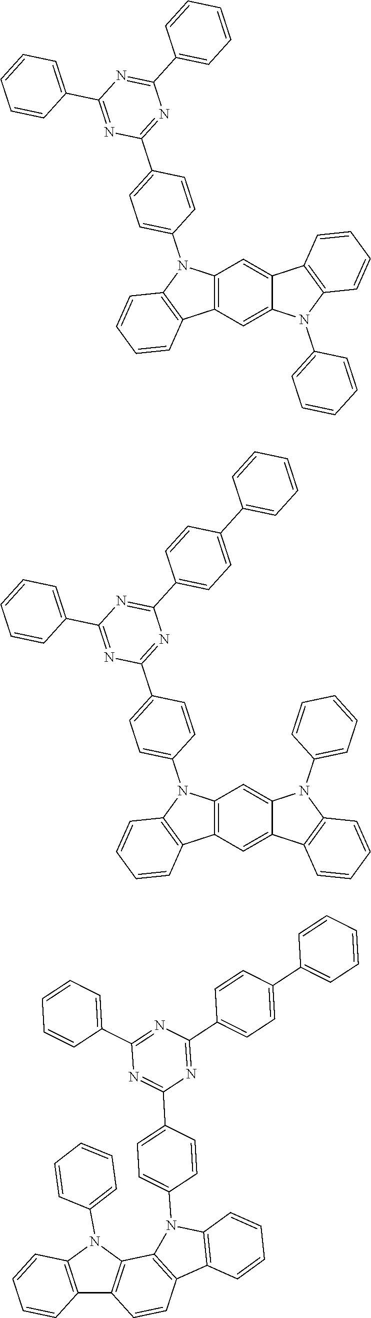 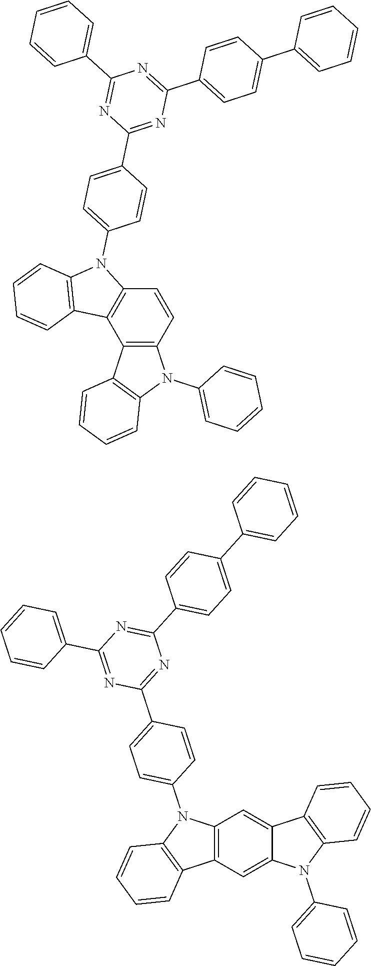 [SMILES: c1ccc(-c2ccc(-c3nc(-c4ccccc4)nc(-c4ccc(-n5c6ccccc6c6c7c8ccccc8n(-c8ccccc8)c7ccc65)cc4)n3)cc2)cc1 ; c1ccc(-c2ccc(-c3nc(-c4ccccc4)nc(-c4ccc(-n5c6ccccc6c6cc7c(cc65)c5ccccc5n7-c5ccccc5)cc4)n3)cc2)cc1] 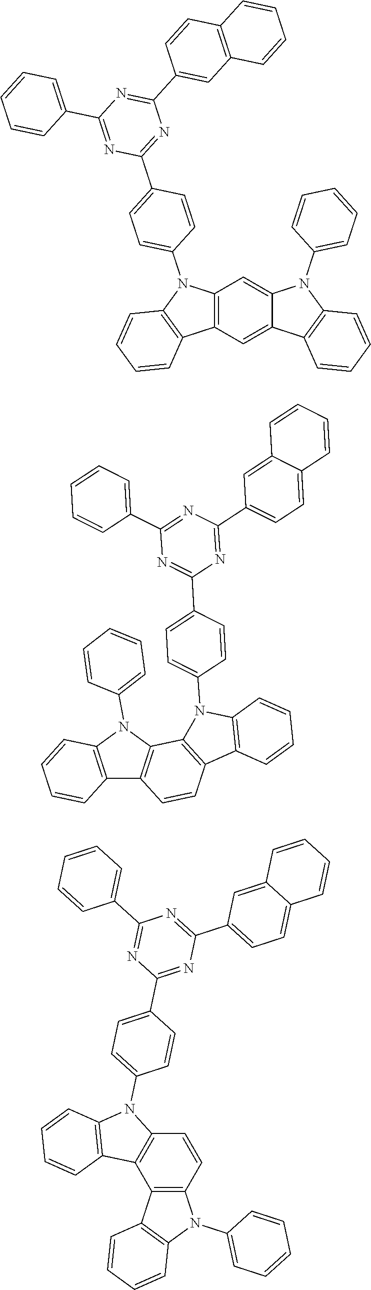 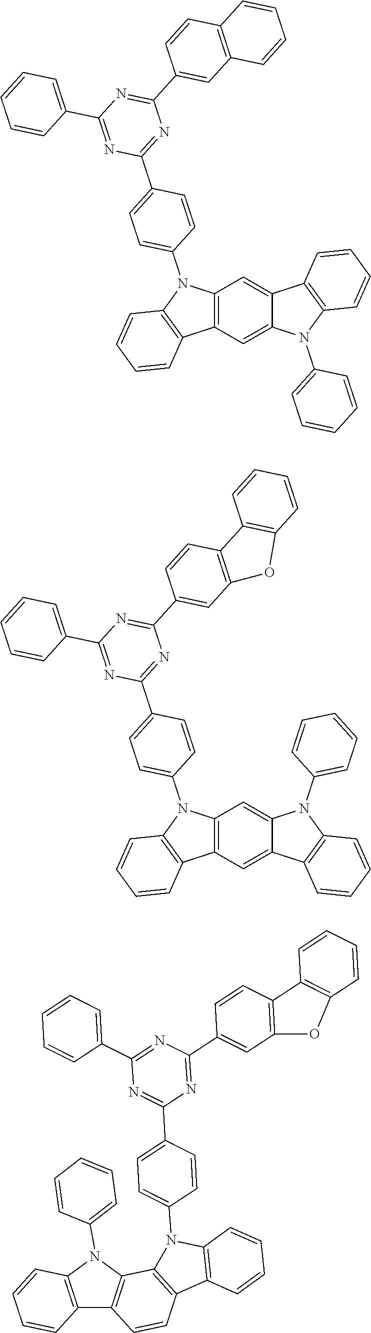 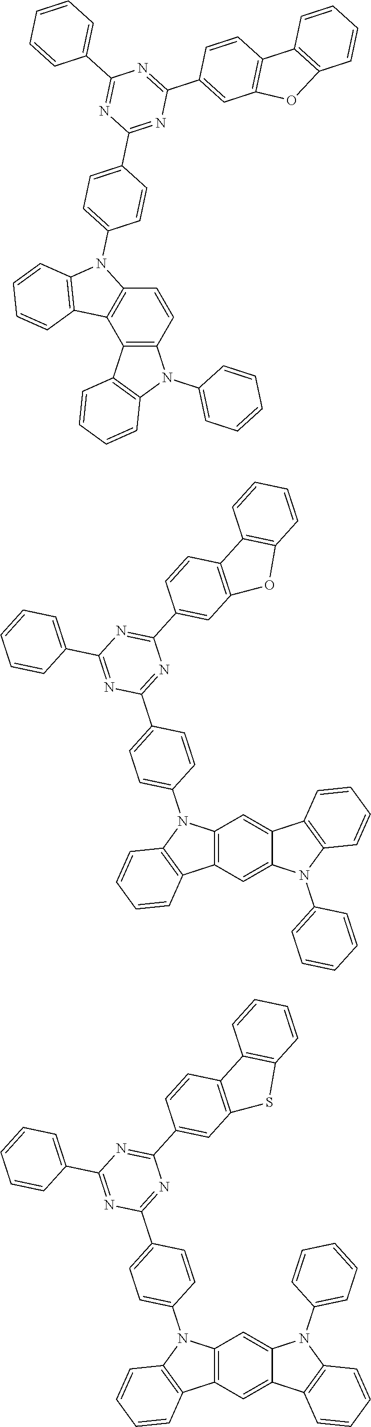 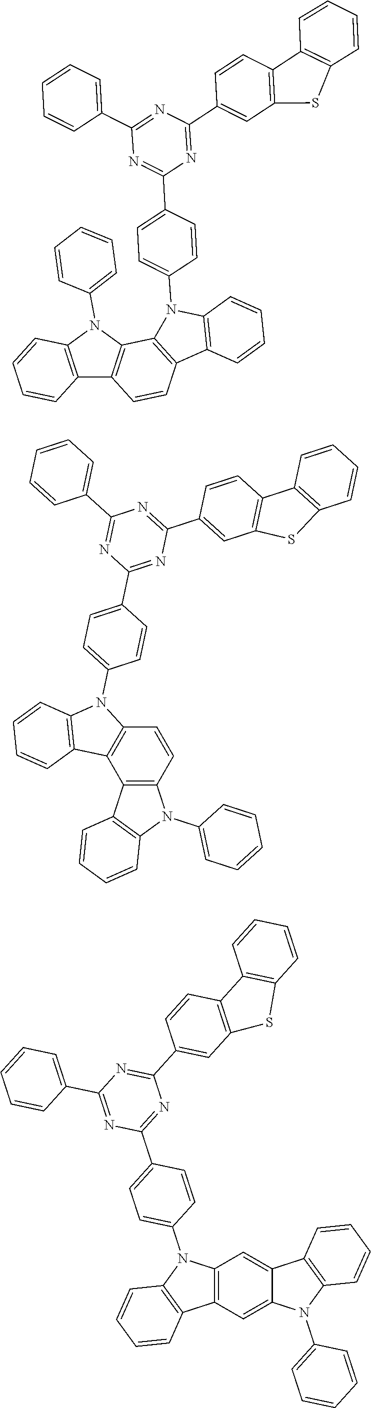 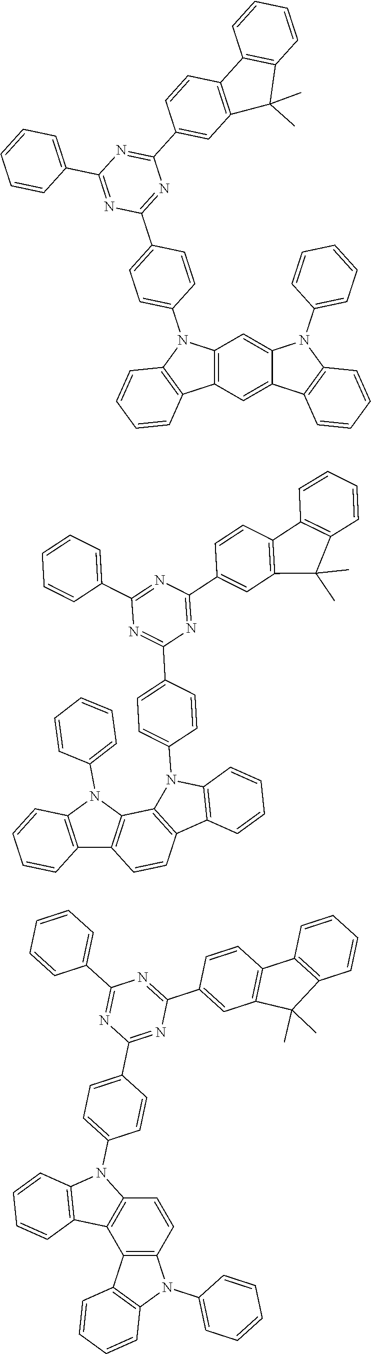 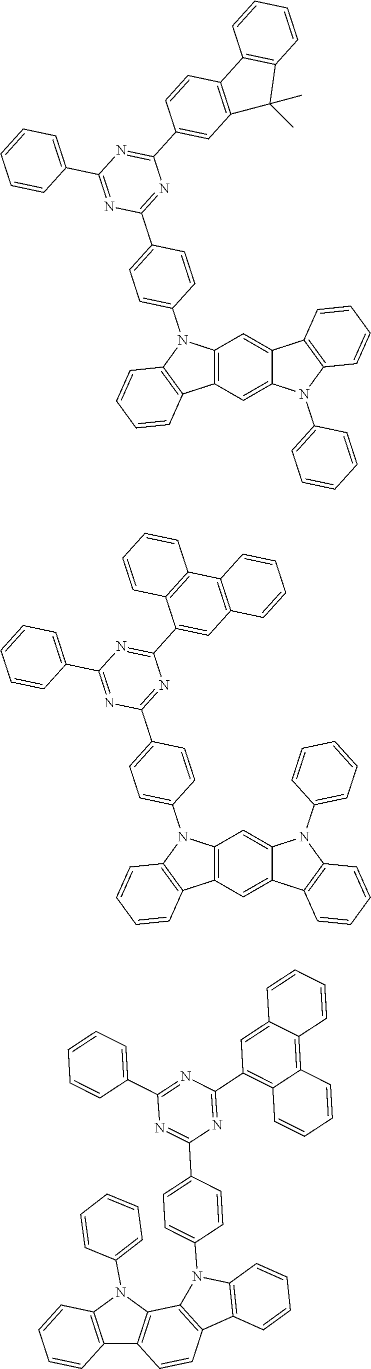 [SMILES: CC1(C)c2ccccc2-c2ccc(-c3nc(-c4ccccc4)nc(-c4ccc(-n5c6ccccc6c6cc7c(cc65)c5ccccc5n7-c5ccccc5)cc4)n3)cc21 ; c1ccc(-c2nc(-c3ccc(-n4c5ccccc5c5cc6c7ccccc7n(-c7ccccc7)c6cc54)cc3)nc(-c3cc4ccccc4c4ccccc34)n2)cc1 ; c1ccc(-c2nc(-c3ccc(-n4c5ccccc5c5ccc6c7ccccc7n(-c7ccccc7)c6c54)cc3)nc(-c3cc4ccccc4c4ccccc34)n2)cc1] 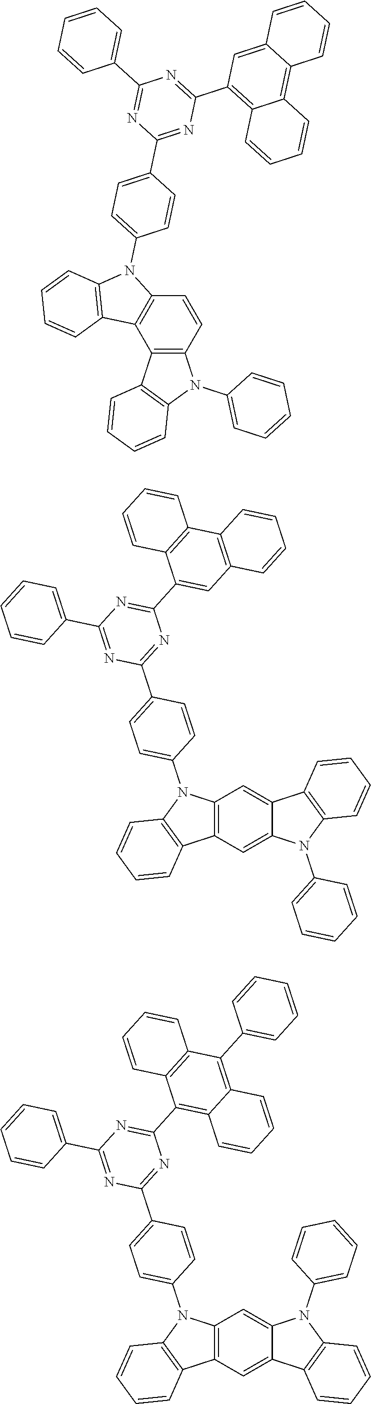 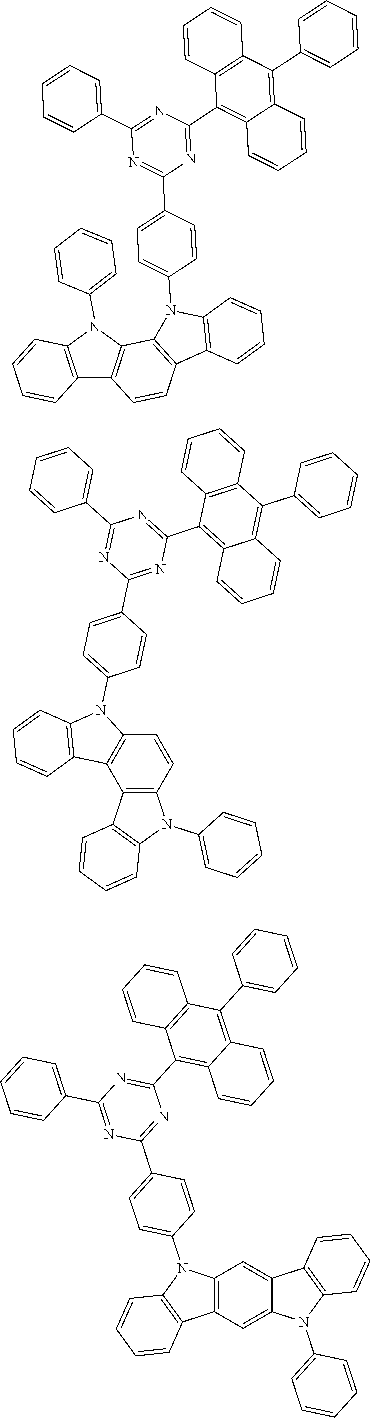 [SMILES: c1ccc(-c2nc(-c3ccc(-n4c5ccccc5c5ccc6c7ccccc7n(-c7ccccc7)c6c54)cc3)nc(-c3c4ccccc4c(-c4ccccc4)c4ccccc34)n2)cc1 ; c1ccc(-c2nc(-c3ccc(-n4c5ccccc5c5c6c7ccccc7n(-c7ccccc7)c6ccc54)cc3)nc(-c3c4ccccc4c(-c4ccccc4)c4ccccc34)n2)cc1 ; c1ccc(-c2nc(-c3ccc(-n4c5ccccc5c5cc6c(cc54)c4ccccc4n6-c4ccccc4)cc3)nc(-c3c4ccccc4c(-c4ccccc4)c4ccccc34)n2)cc1] 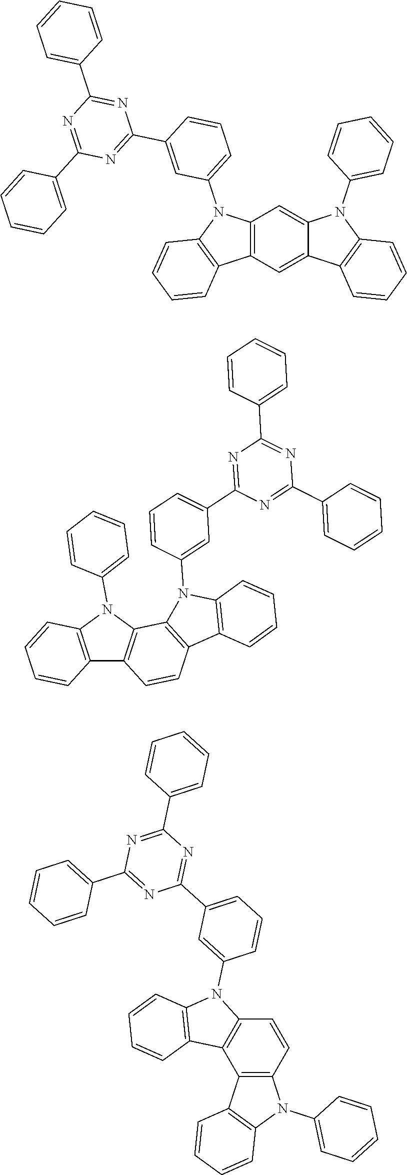 [SMILES: c1ccc(-c2nc(-c3ccccc3)nc(-c3cccc(-n4c5ccccc5c5cc6c7ccccc7n(-c7ccccc7)c6cc54)c3)n2)cc1 ; c1ccc(-c2nc(-c3ccccc3)nc(-c3cccc(-n4c5ccccc5c5ccc6c7ccccc7n(-c7ccccc7)c6c54)c3)n2)cc1 ; c1ccc(-c2nc(-c3ccccc3)nc(-c3cccc(-n4c5ccccc5c5c6c7ccccc7n(-c7ccccc7)c6ccc54)c3)n2)cc1] 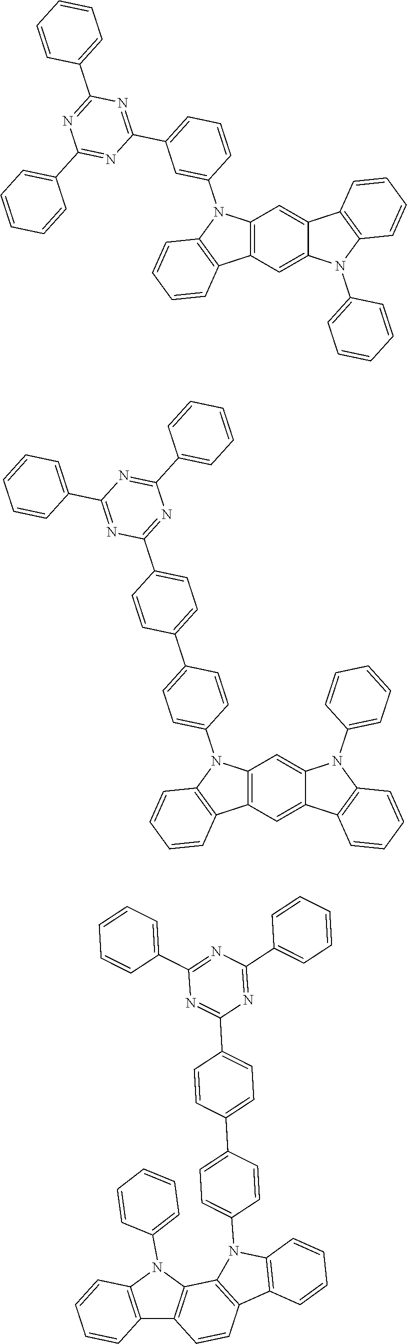 [SMILES: c1ccc(-c2nc(-c3ccccc3)nc(-c3cccc(-n4c5ccccc5c5cc6c(cc54)c4ccccc4n6-c4ccccc4)c3)n2)cc1 ; c1ccc(-c2nc(-c3ccccc3)nc(-c3ccc(-c4ccc(-n5c6ccccc6c6cc7c8ccccc8n(-c8ccccc8)c7cc65)cc4)cc3)n2)cc1 ; c1ccc(-c2nc(-c3ccccc3)nc(-c3ccc(-c4ccc(-n5c6ccccc6c6ccc7c8ccccc8n(-c8ccccc8)c7c65)cc4)cc3)n2)cc1] 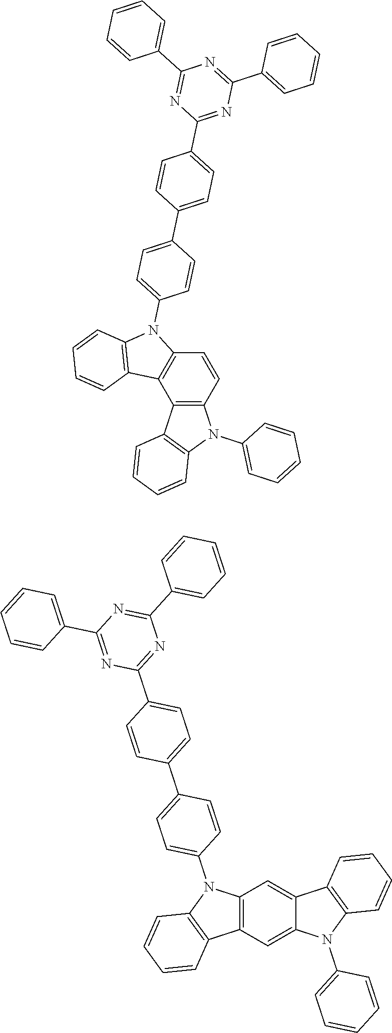 [SMILES: c1ccc(-c2nc(-c3ccccc3)nc(-c3ccc(-c4ccc(-n5c6ccccc6c6c7c8ccccc8n(-c8ccccc8)c7ccc65)cc4)cc3)n2)cc1 ; c1ccc(-c2nc(-c3ccccc3)nc(-c3ccc(-c4ccc(-n5c6ccccc6c6cc7c(cc65)c5ccccc5n7-c5ccccc5)cc4)cc3)n2)cc1] 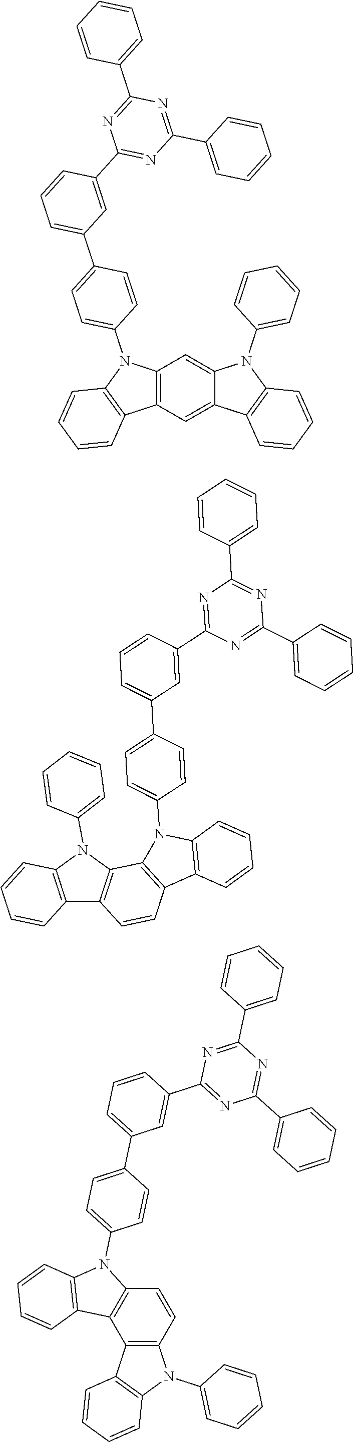 [SMILES: c1ccc(-c2nc(-c3ccccc3)nc(-c3cccc(-c4ccc(-n5c6ccccc6c6cc7c8ccccc8n(-c8ccccc8)c7cc65)cc4)c3)n2)cc1 ; c1ccc(-c2nc(-c3ccccc3)nc(-c3cccc(-c4ccc(-n5c6ccccc6c6ccc7c8ccccc8n(-c8ccccc8)c7c65)cc4)c3)n2)cc1 ; c1ccc(-c2nc(-c3ccccc3)nc(-c3cccc(-c4ccc(-n5c6ccccc6c6c7c8ccccc8n(-c8ccccc8)c7ccc65)cc4)c3)n2)cc1] 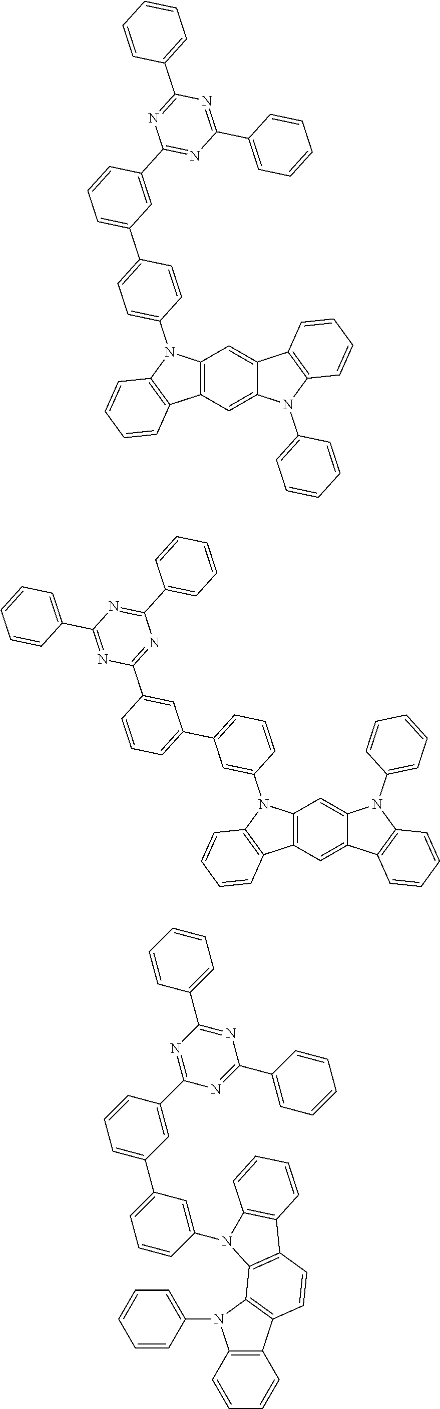 [SMILES: c1ccc(-c2nc(-c3ccccc3)nc(-c3cccc(-c4ccc(-n5c6ccccc6c6cc7c(cc65)c5ccccc5n7-c5ccccc5)cc4)c3)n2)cc1 ; c1ccc(-c2nc(-c3ccccc3)nc(-c3cccc(-c4cccc(-n5c6ccccc6c6cc7c8ccccc8n(-c8ccccc8)c7cc65)c4)c3)n2)cc1 ; c1ccc(-c2nc(-c3ccccc3)nc(-c3cccc(-c4cccc(-n5c6ccccc6c6ccc7c8ccccc8n(-c8ccccc8)c7c65)c4)c3)n2)cc1] 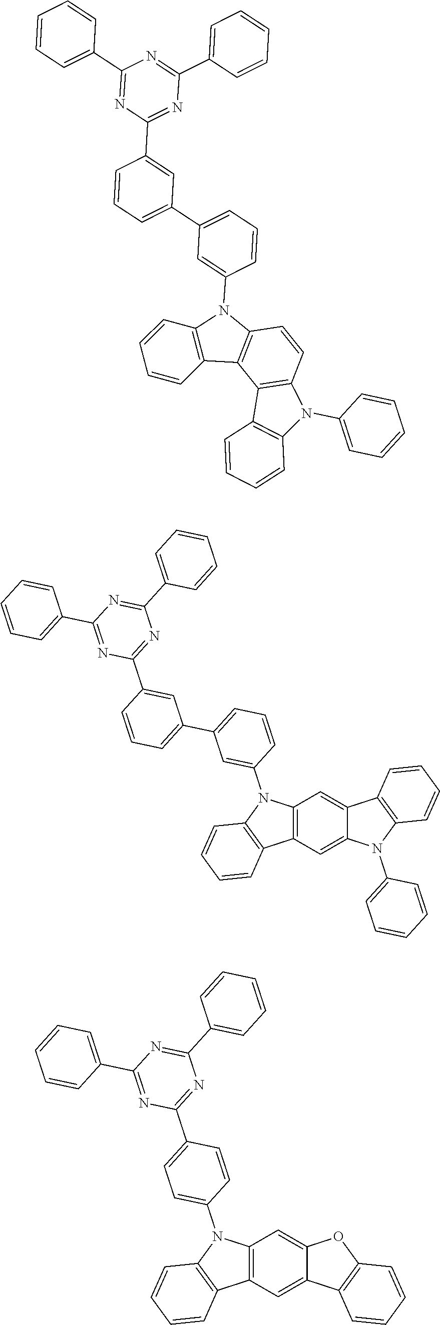 [SMILES: c1ccc(-c2nc(-c3ccccc3)nc(-c3cccc(-c4cccc(-n5c6ccccc6c6c7c8ccccc8n(-c8ccccc8)c7ccc65)c4)c3)n2)cc1 ; c1ccc(-c2nc(-c3ccccc3)nc(-c3cccc(-c4cccc(-n5c6ccccc6c6cc7c(cc65)c5ccccc5n7-c5ccccc5)c4)c3)n2)cc1 ; c1ccc(-c2nc(-c3ccccc3)nc(-c3ccc(-n4c5ccccc5c5cc6c(cc54)oc4ccccc46)cc3)n2)cc1] 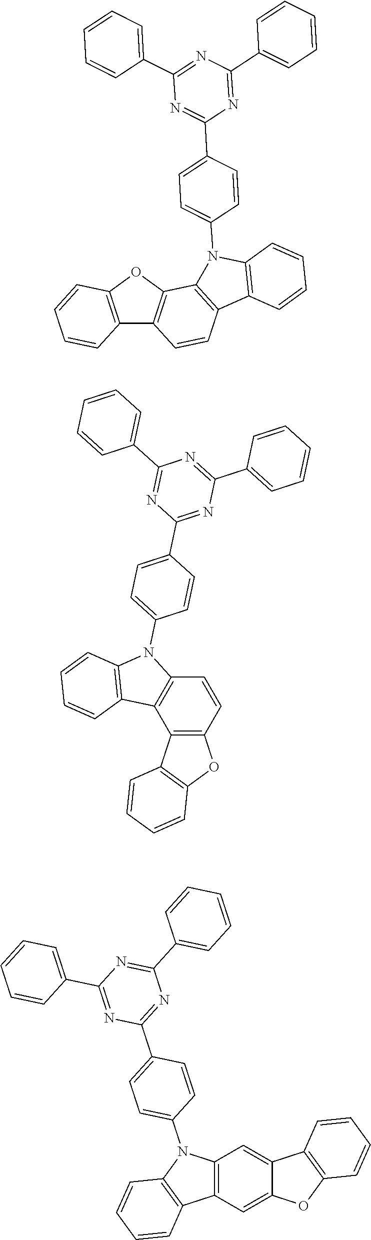 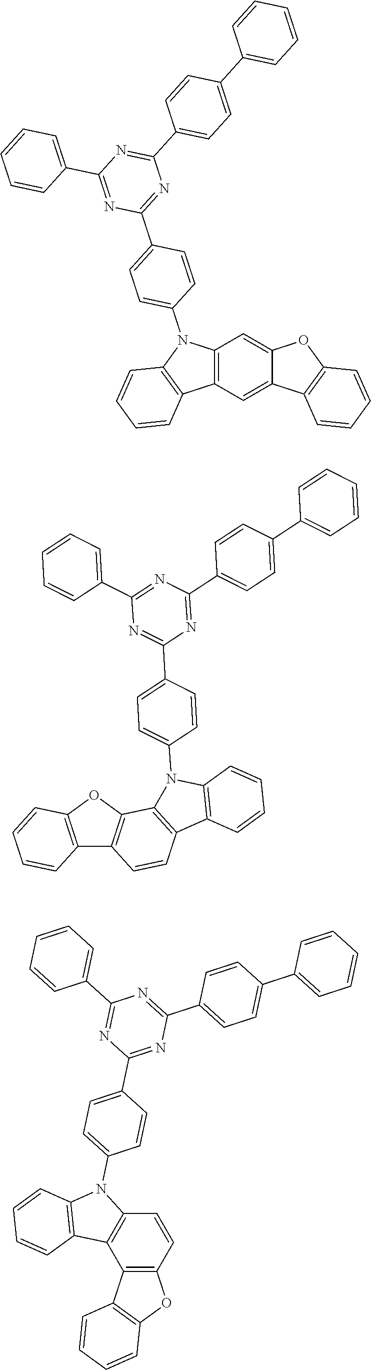 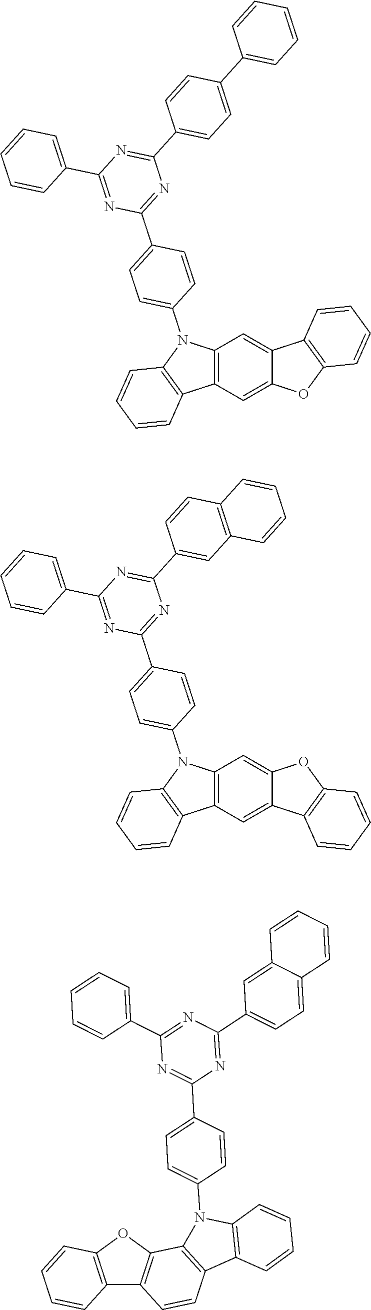 [SMILES: c1ccc(-c2ccc(-c3nc(-c4ccccc4)nc(-c4ccc(-n5c6ccccc6c6cc7oc8ccccc8c7cc65)cc4)n3)cc2)cc1 ; c1ccc(-c2nc(-c3ccc(-n4c5ccccc5c5cc6c(cc54)oc4ccccc46)cc3)nc(-c3ccc4ccccc4c3)n2)cc1 ; c1ccc(-c2nc(-c3ccc(-n4c5ccccc5c5ccc6c7ccccc7oc6c54)cc3)nc(-c3ccc4ccccc4c3)n2)cc1] 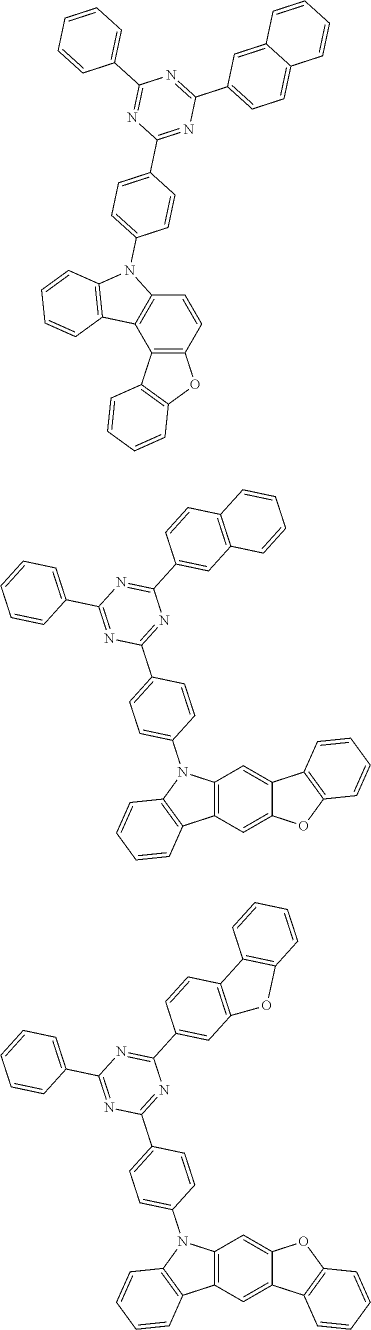 [SMILES: c1ccc(-c2nc(-c3ccc(-n4c5ccccc5c5c6c(ccc54)oc4ccccc46)cc3)nc(-c3ccc4ccccc4c3)n2)cc1 ; c1ccc(-c2nc(-c3ccc(-n4c5ccccc5c5cc6oc7ccccc7c6cc54)cc3)nc(-c3ccc4ccccc4c3)n2)cc1 ; c1ccc(-c2nc(-c3ccc(-n4c5ccccc5c5cc6c(cc54)oc4ccccc46)cc3)nc(-c3ccc4c(c3)oc3ccccc34)n2)cc1] 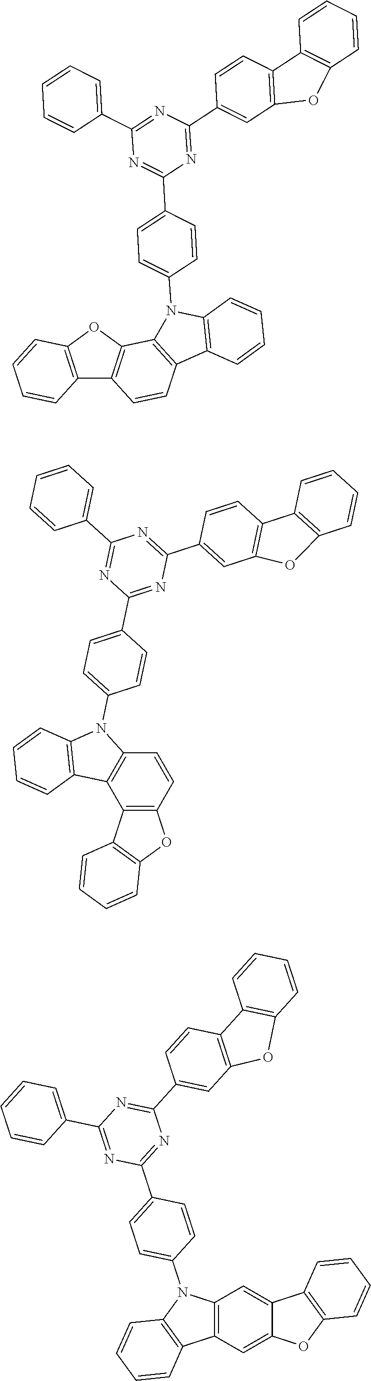 [SMILES: c1ccc(-c2nc(-c3ccc(-n4c5ccccc5c5ccc6c7ccccc7oc6c54)cc3)nc(-c3ccc4c(c3)oc3ccccc34)n2)cc1 ; c1ccc(-c2nc(-c3ccc(-n4c5ccccc5c5c6c(ccc54)oc4ccccc46)cc3)nc(-c3ccc4c(c3)oc3ccccc34)n2)cc1 ; c1ccc(-c2nc(-c3ccc(-n4c5ccccc5c5cc6oc7ccccc7c6cc54)cc3)nc(-c3ccc4c(c3)oc3ccccc34)n2)cc1] 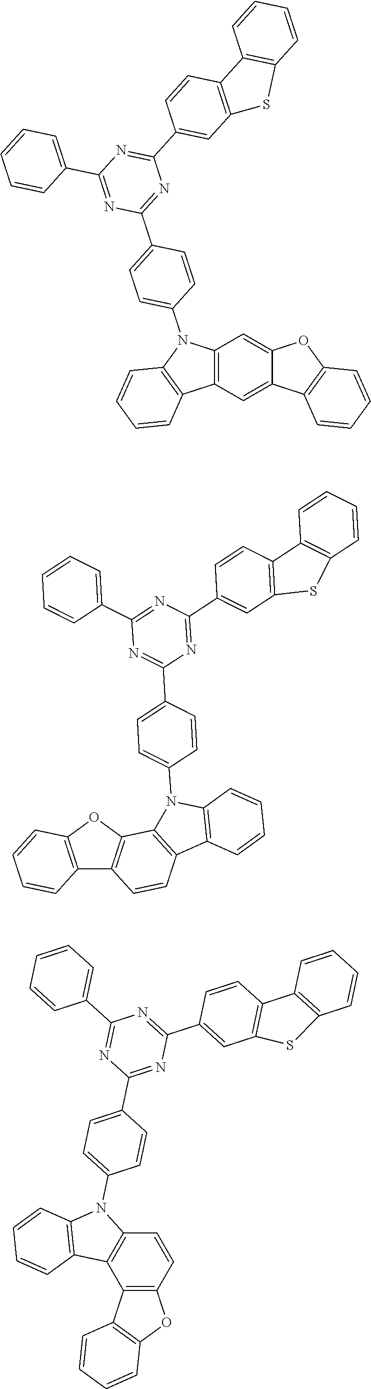 [SMILES: c1ccc(-c2nc(-c3ccc(-n4c5ccccc5c5cc6c(cc54)oc4ccccc46)cc3)nc(-c3ccc4c(c3)sc3ccccc34)n2)cc1 ; c1ccc(-c2nc(-c3ccc(-n4c5ccccc5c5ccc6c7ccccc7oc6c54)cc3)nc(-c3ccc4c(c3)sc3ccccc34)n2)cc1 ; c1ccc(-c2nc(-c3ccc(-n4c5ccccc5c5c6c(ccc54)oc4ccccc46)cc3)nc(-c3ccc4c(c3)sc3ccccc34)n2)cc1] 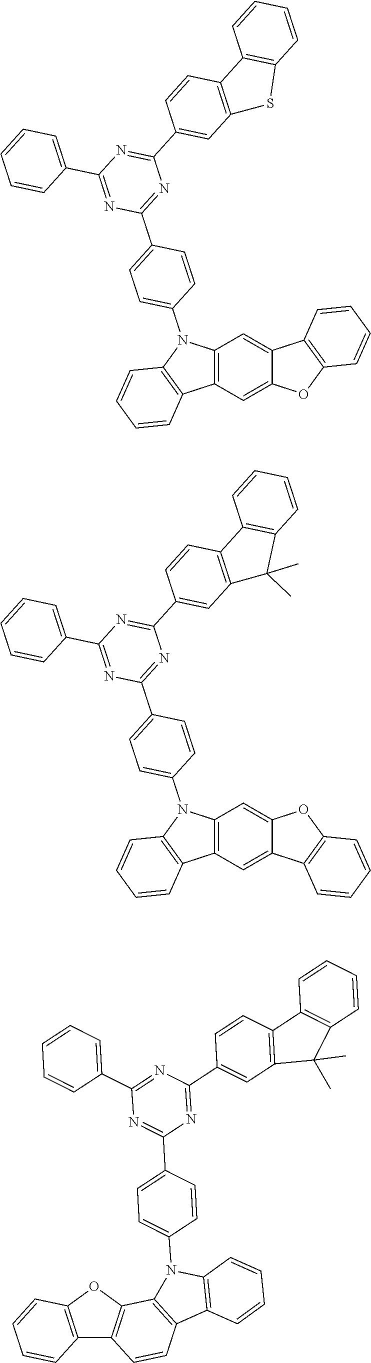 [SMILES: c1ccc(-c2nc(-c3ccc(-n4c5ccccc5c5cc6oc7ccccc7c6cc54)cc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)oc5ccccc57)cc4)n3)cc21 ; CC1(C)c2ccccc2-c2ccc(-c3nc(-c4ccccc4)nc(-c4ccc(-n5c6ccccc6c6ccc7c8ccccc8oc7c65)cc4)n3)cc21] 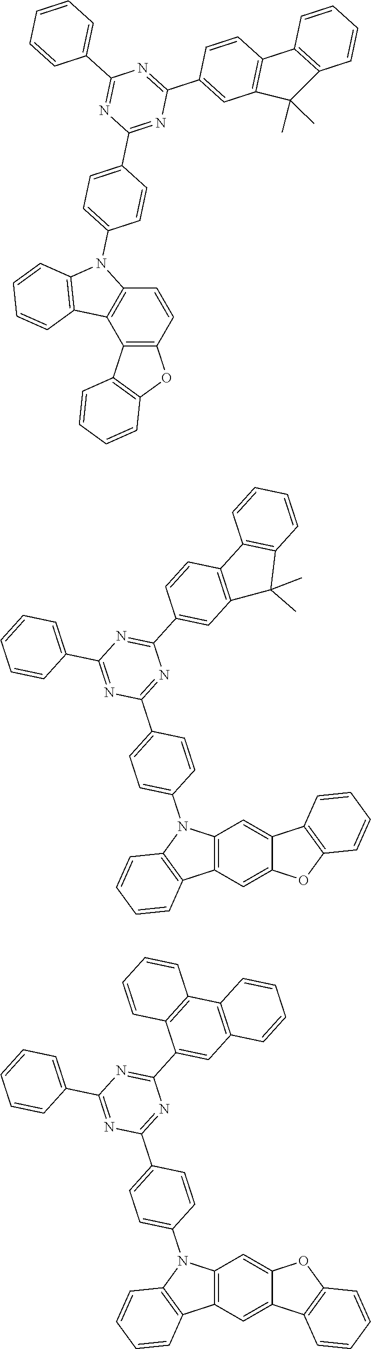 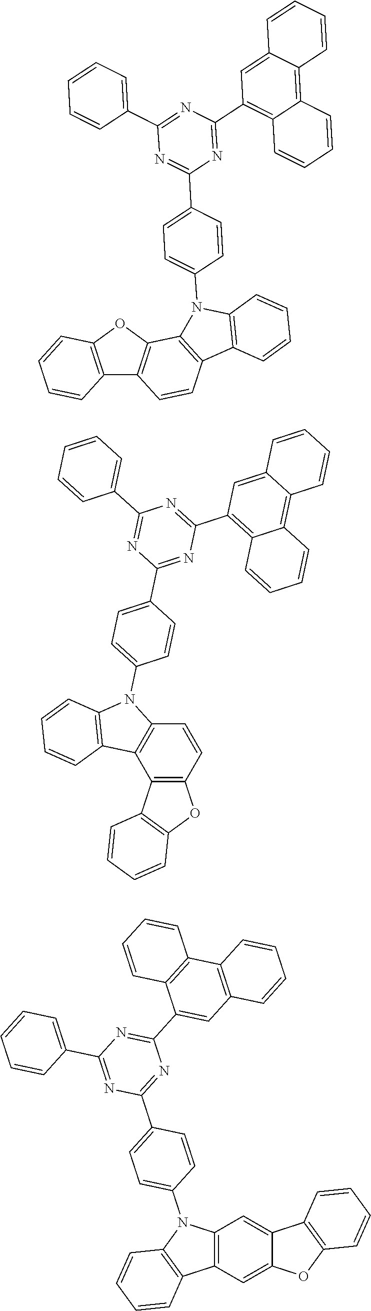 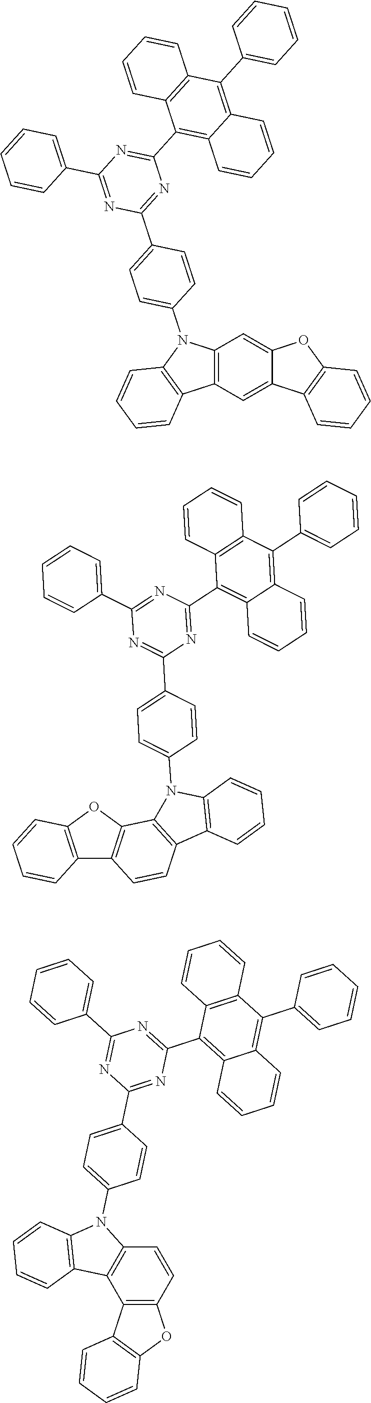 [SMILES: c1ccc(-c2nc(-c3ccc(-n4c5ccccc5c5cc6c(cc54)oc4ccccc46)cc3)nc(-c3c4ccccc4c(-c4ccccc4)c4ccccc34)n2)cc1 ; c1ccc(-c2nc(-c3ccc(-n4c5ccccc5c5ccc6c7ccccc7oc6c54)cc3)nc(-c3c4ccccc4c(-c4ccccc4)c4ccccc34)n2)cc1 ; c1ccc(-c2nc(-c3ccc(-n4c5ccccc5c5c6c(ccc54)oc4ccccc46)cc3)nc(-c3c4ccccc4c(-c4ccccc4)c4ccccc34)n2)cc1] 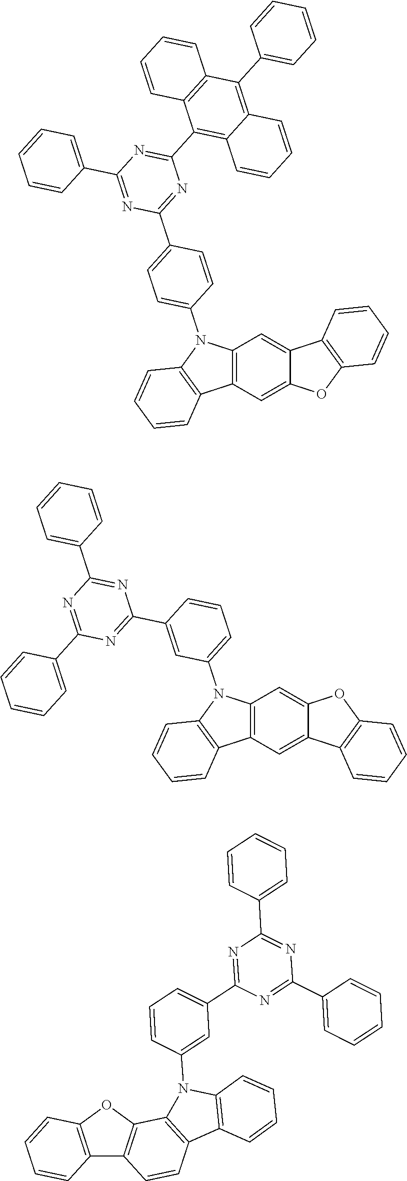 [SMILES: c1ccc(-c2nc(-c3ccc(-n4c5ccccc5c5cc6oc7ccccc7c6cc54)cc3)nc(-c3c4ccccc4c(-c4ccccc4)c4ccccc34)n2)cc1 ; c1ccc(-c2nc(-c3ccccc3)nc(-c3cccc(-n4c5ccccc5c5cc6c(cc54)oc4ccccc46)c3)n2)cc1 ; c1ccc(-c2nc(-c3ccccc3)nc(-c3cccc(-n4c5ccccc5c5ccc6c7ccccc7oc6c54)c3)n2)cc1] 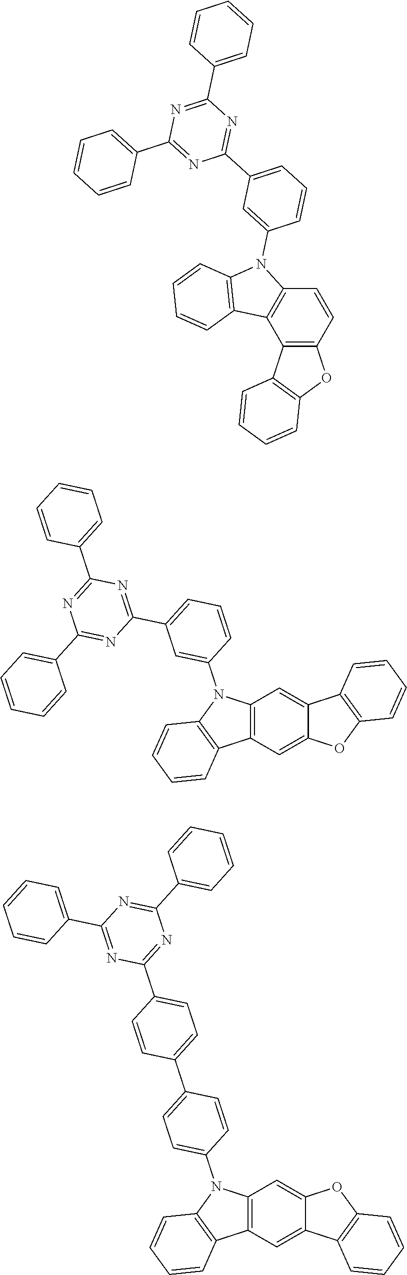 [SMILES: c1ccc(-c2nc(-c3ccccc3)nc(-c3cccc(-n4c5ccccc5c5c6c(ccc54)oc4ccccc46)c3)n2)cc1 ; c1ccc(-c2nc(-c3ccccc3)nc(-c3cccc(-n4c5ccccc5c5cc6oc7ccccc7c6cc54)c3)n2)cc1 ; c1ccc(-c2nc(-c3ccccc3)nc(-c3ccc(-c4ccc(-n5c6ccccc6c6cc7c(cc65)oc5ccccc57)cc4)cc3)n2)cc1] 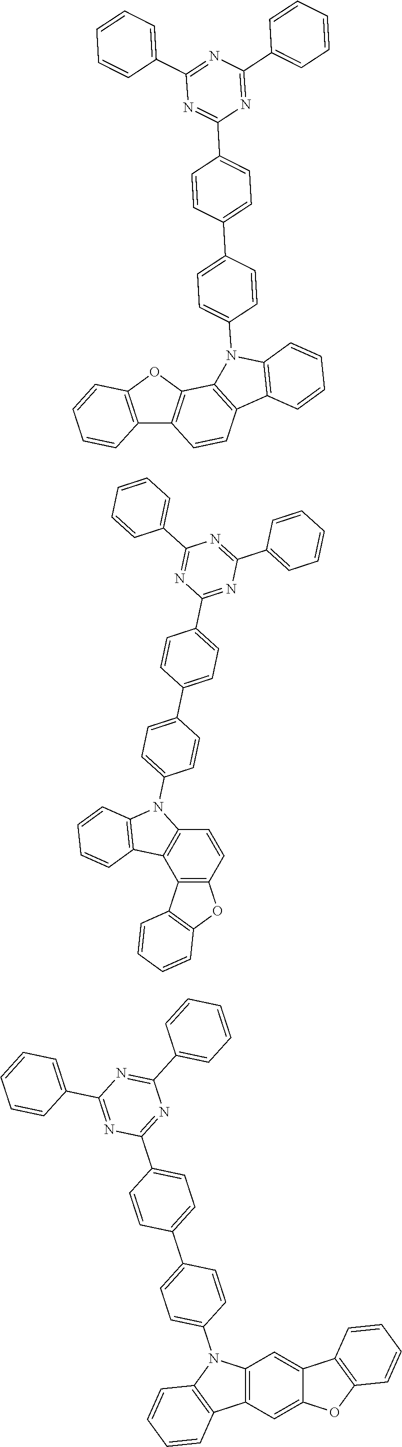 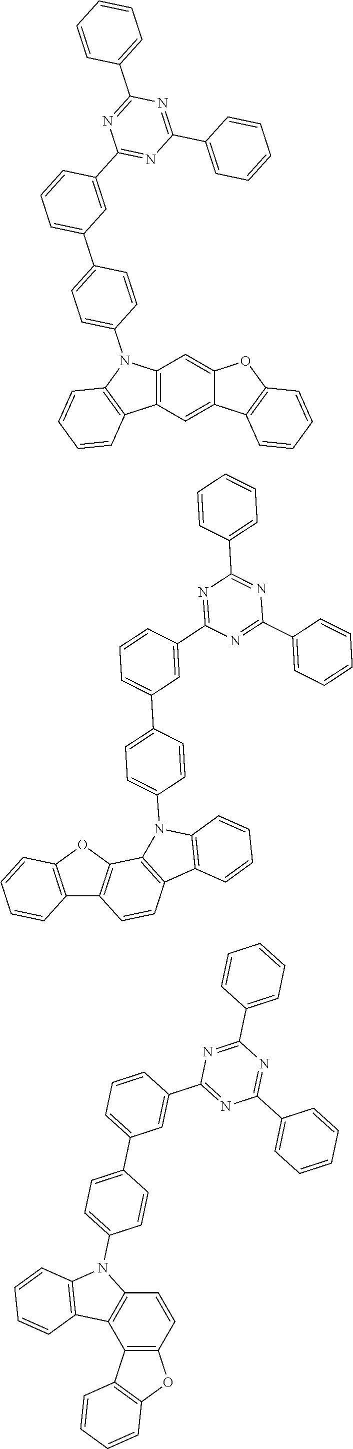 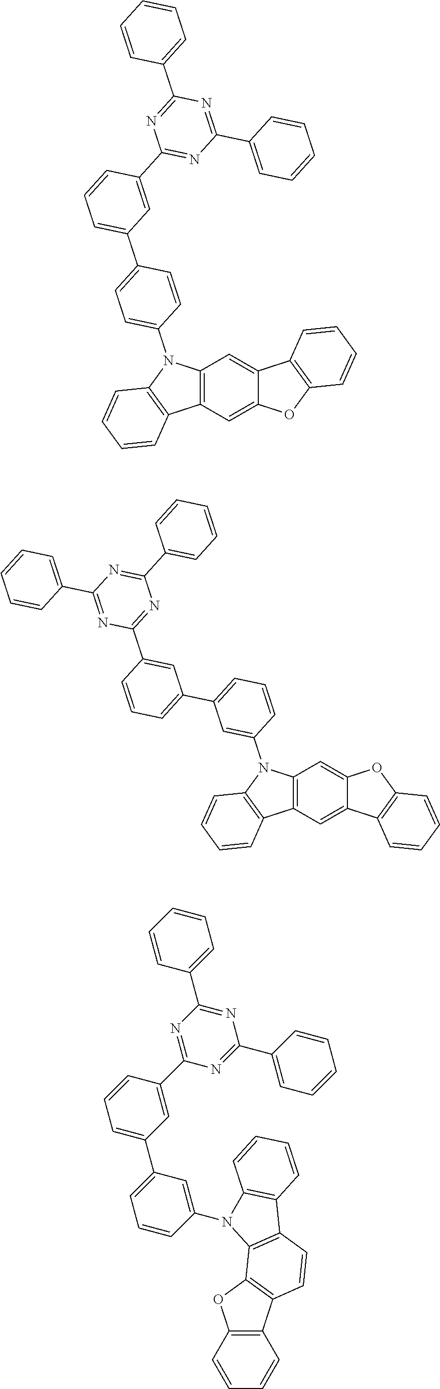 [SMILES: c1ccc(-c2nc(-c3ccccc3)nc(-c3cccc(-c4ccc(-n5c6ccccc6c6cc7oc8ccccc8c7cc65)cc4)c3)n2)cc1 ; c1ccc(-c2nc(-c3ccccc3)nc(-c3cccc(-c4cccc(-n5c6ccccc6c6cc7c(cc65)oc5ccccc57)c4)c3)n2)cc1 ; c1ccc(-c2nc(-c3ccccc3)nc(-c3cccc(-c4cccc(-n5c6ccccc6c6ccc7c8ccccc8oc7c65)c4)c3)n2)cc1] 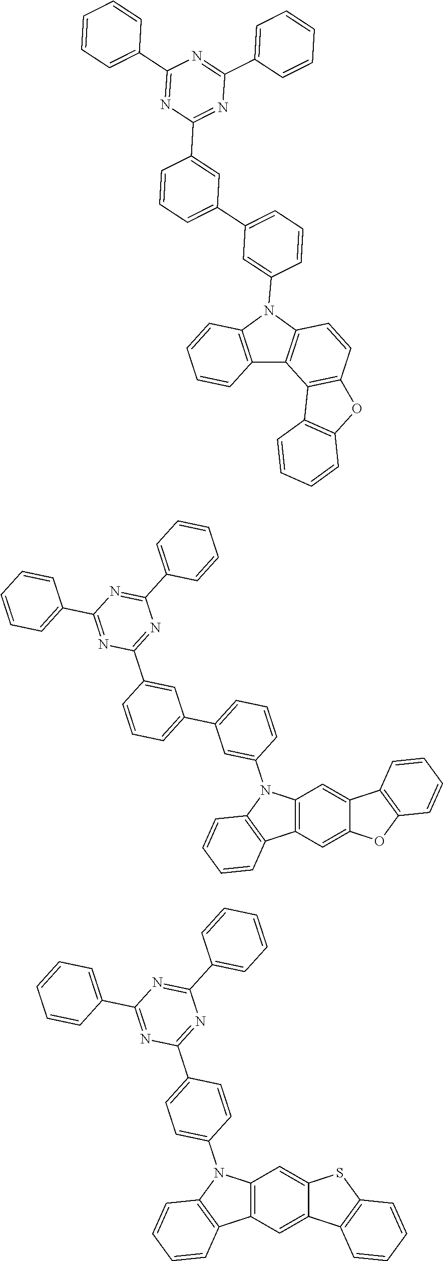 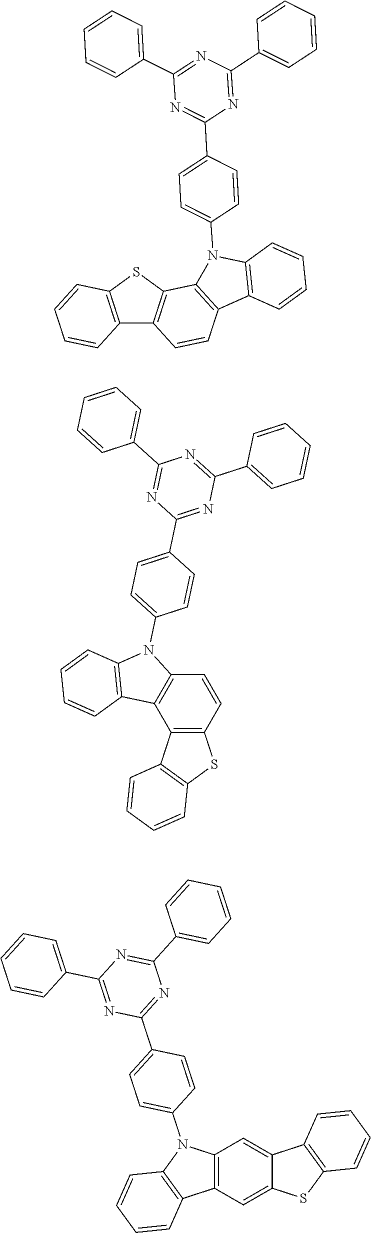 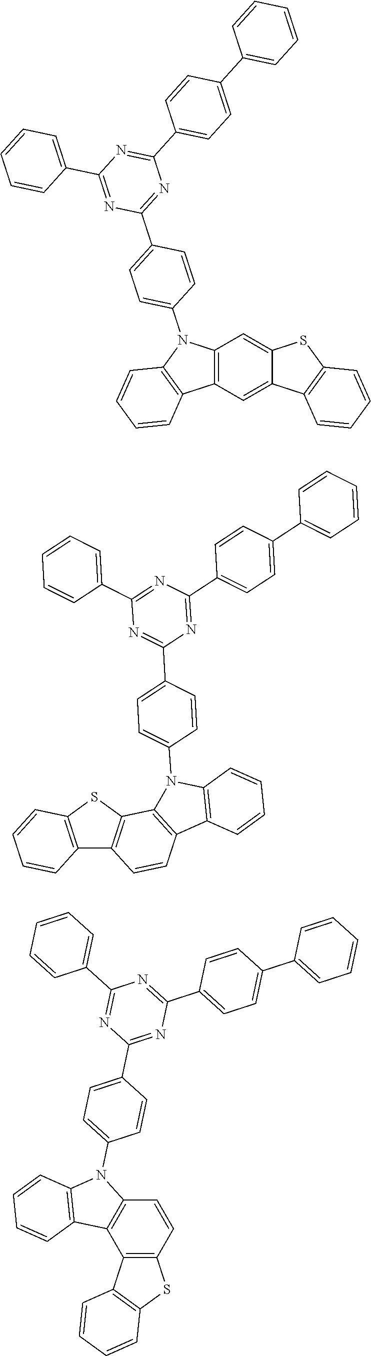 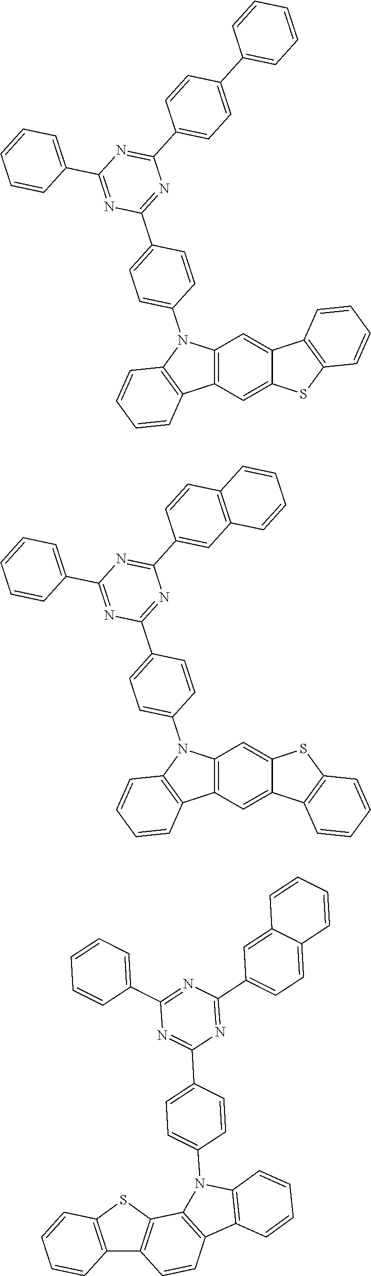 [SMILES: c1ccc(-c2ccc(-c3nc(-c4ccccc4)nc(-c4ccc(-n5c6ccccc6c6cc7sc8ccccc8c7cc65)cc4)n3)cc2)cc1 ; c1ccc(-c2nc(-c3ccc(-n4c5ccccc5c5cc6c(cc54)sc4ccccc46)cc3)nc(-c3ccc4ccccc4c3)n2)cc1 ; c1ccc(-c2nc(-c3ccc(-n4c5ccccc5c5ccc6c7ccccc7sc6c54)cc3)nc(-c3ccc4ccccc4c3)n2)cc1] 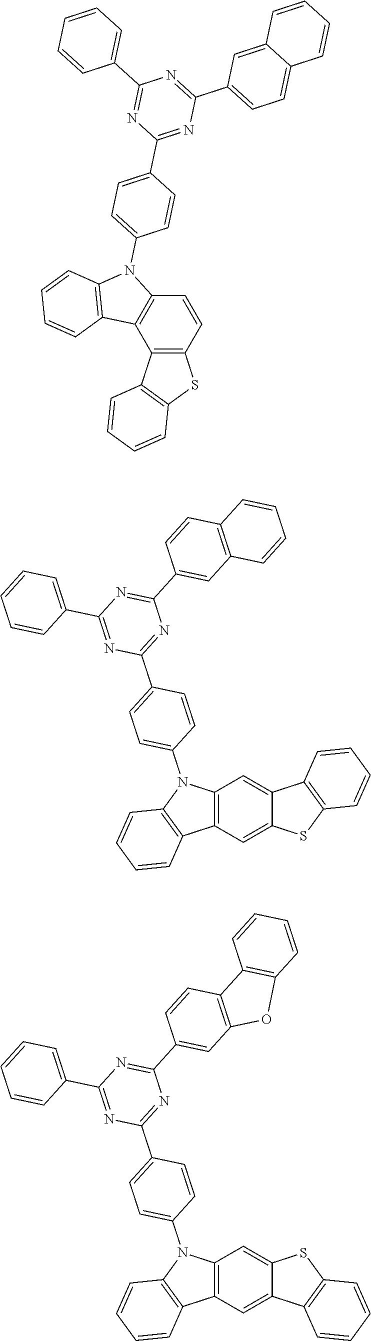 [SMILES: c1ccc(-c2nc(-c3ccc(-n4c5ccccc5c5c6c(ccc54)sc4ccccc46)cc3)nc(-c3ccc4ccccc4c3)n2)cc1 ; c1ccc(-c2nc(-c3ccc(-n4c5ccccc5c5cc6sc7ccccc7c6cc54)cc3)nc(-c3ccc4ccccc4c3)n2)cc1 ; c1ccc(-c2nc(-c3ccc(-n4c5ccccc5c5cc6c(cc54)sc4ccccc46)cc3)nc(-c3ccc4c(c3)oc3ccccc34)n2)cc1] 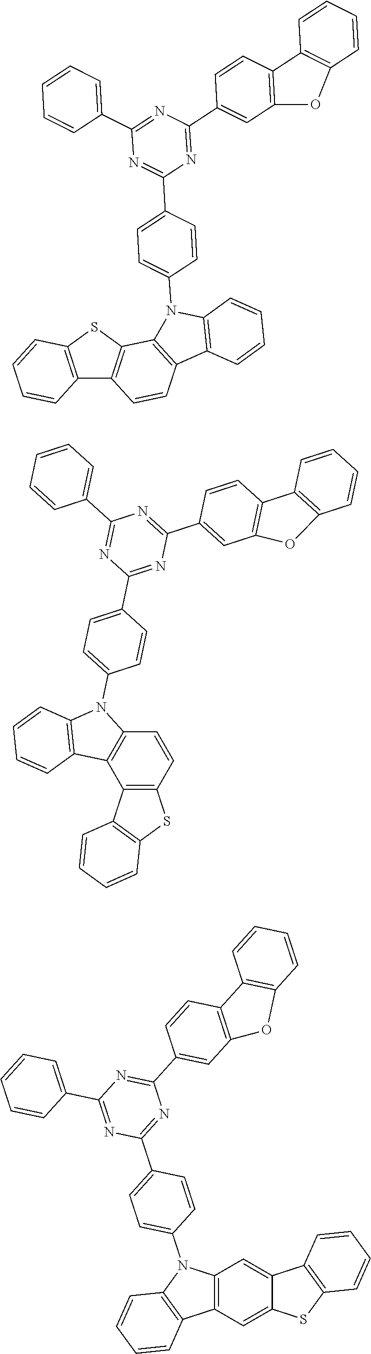 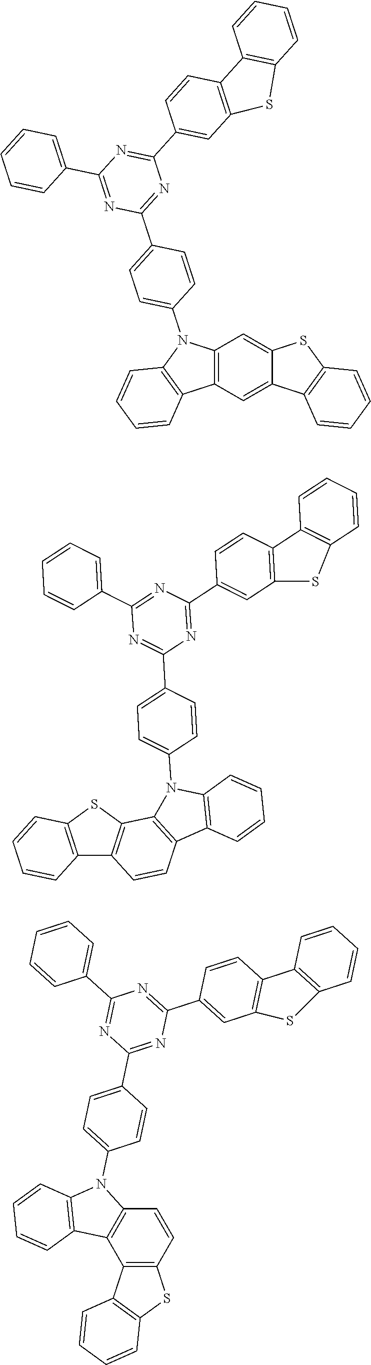 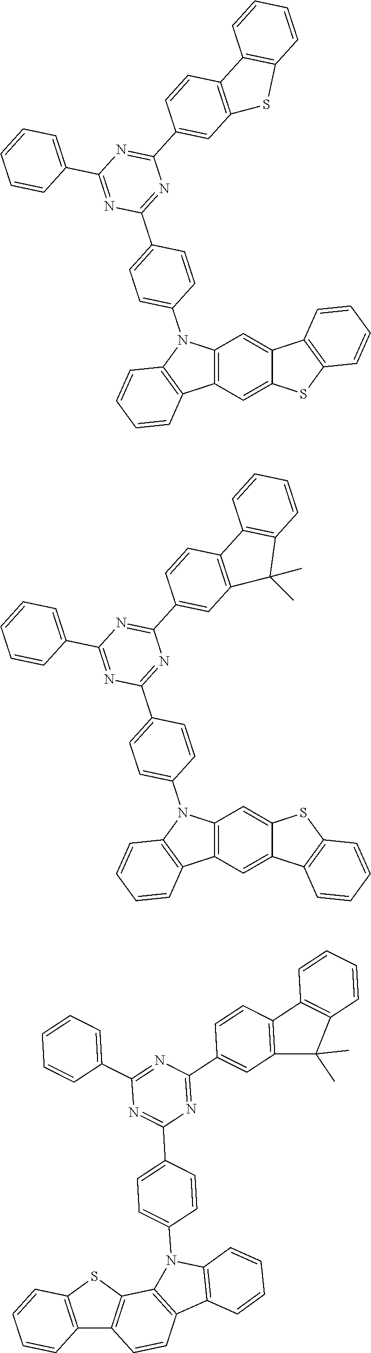 [SMILES: c1ccc(-c2nc(-c3ccc(-n4c5ccccc5c5cc6sc7ccccc7c6cc54)cc3)nc(-c3ccc4c(c3)sc3ccccc34)n2)cc1 ; CC1(C)c2ccccc2-c2ccc(-c3nc(-c4ccccc4)nc(-c4ccc(-n5c6ccccc6c6cc7c(cc65)sc5ccccc57)cc4)n3)cc21 ; CC1(C)c2ccccc2-c2ccc(-c3nc(-c4ccccc4)nc(-c4ccc(-n5c6ccccc6c6ccc7c8ccccc8sc7c65)cc4)n3)cc21] 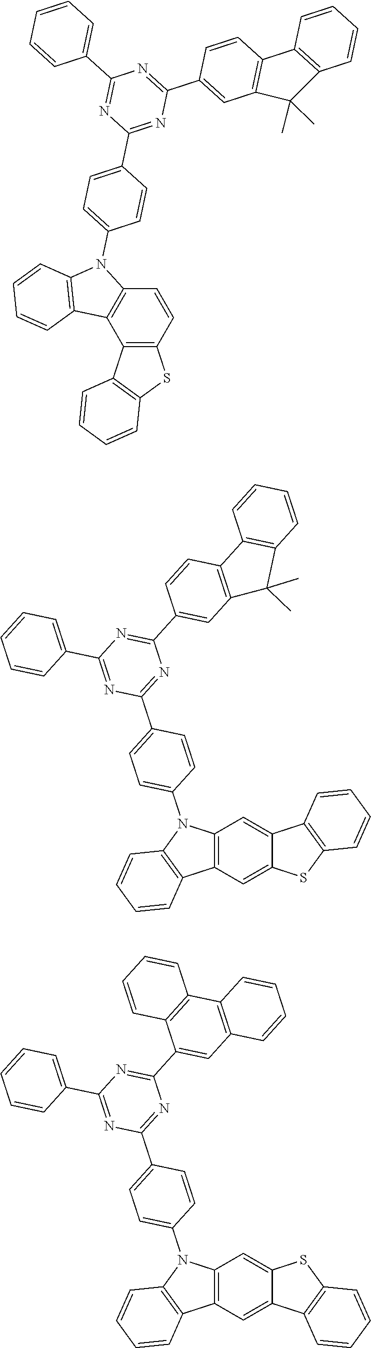 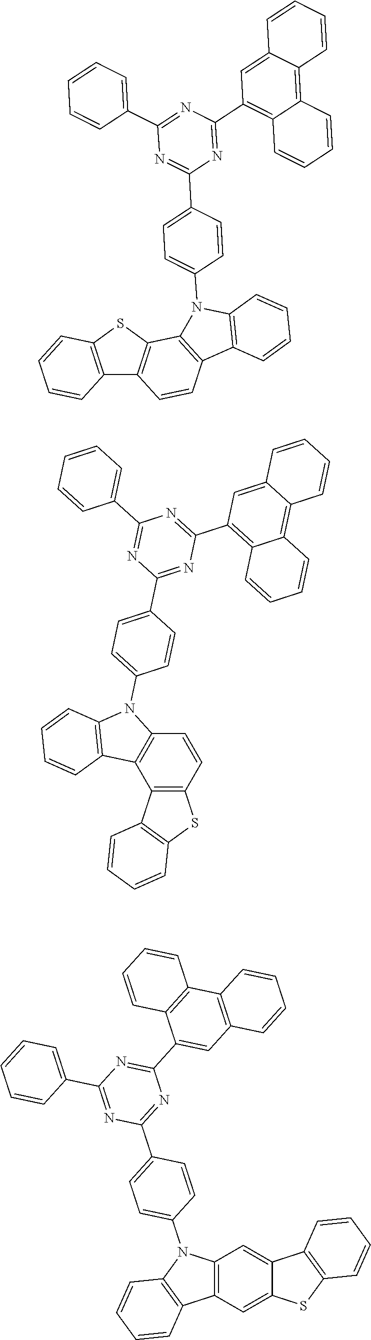 [SMILES: c1ccc(-c2nc(-c3ccc(-n4c5ccccc5c5ccc6c7ccccc7sc6c54)cc3)nc(-c3cc4ccccc4c4ccccc34)n2)cc1 ; c1ccc(-c2nc(-c3ccc(-n4c5ccccc5c5c6c(ccc54)sc4ccccc46)cc3)nc(-c3cc4ccccc4c4ccccc34)n2)cc1 ; c1ccc(-c2nc(-c3ccc(-n4c5ccccc5c5cc6sc7ccccc7c6cc54)cc3)nc(-c3cc4ccccc4c4ccccc34)n2)cc1] 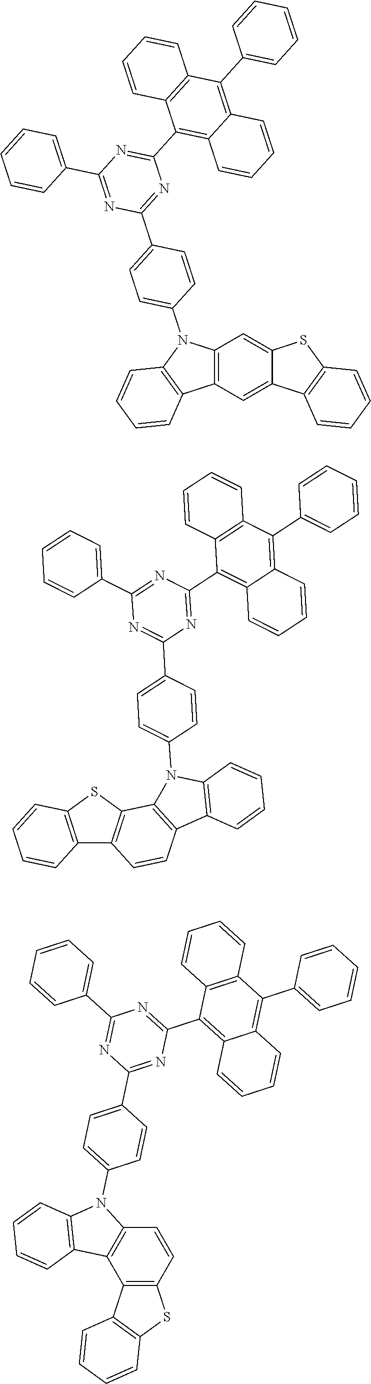 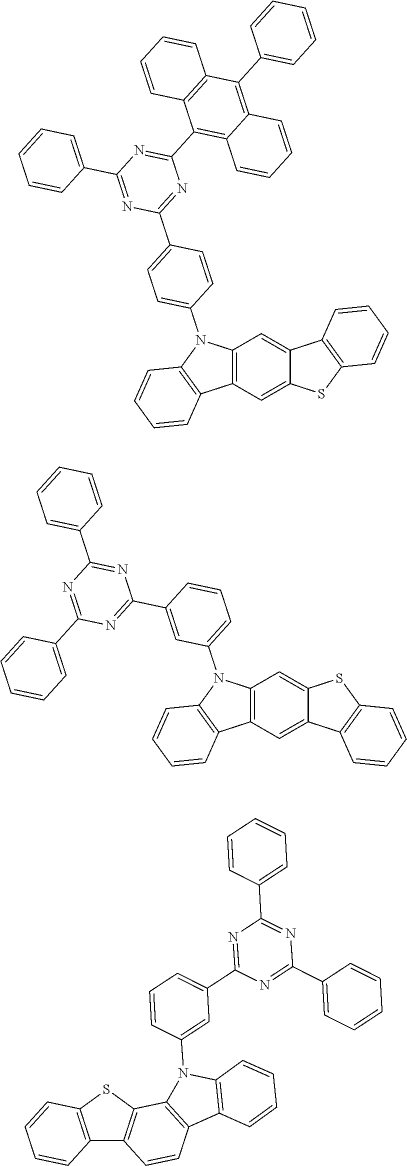 [SMILES: c1ccc(-c2nc(-c3ccc(-n4c5ccccc5c5cc6sc7ccccc7c6cc54)cc3)nc(-c3c4ccccc4c(-c4ccccc4)c4ccccc34)n2)cc1 ; c1ccc(-c2nc(-c3ccccc3)nc(-c3cccc(-n4c5ccccc5c5cc6c(cc54)sc4ccccc46)c3)n2)cc1 ; c1ccc(-c2nc(-c3ccccc3)nc(-c3cccc(-n4c5ccccc5c5ccc6c7ccccc7sc6c54)c3)n2)cc1] 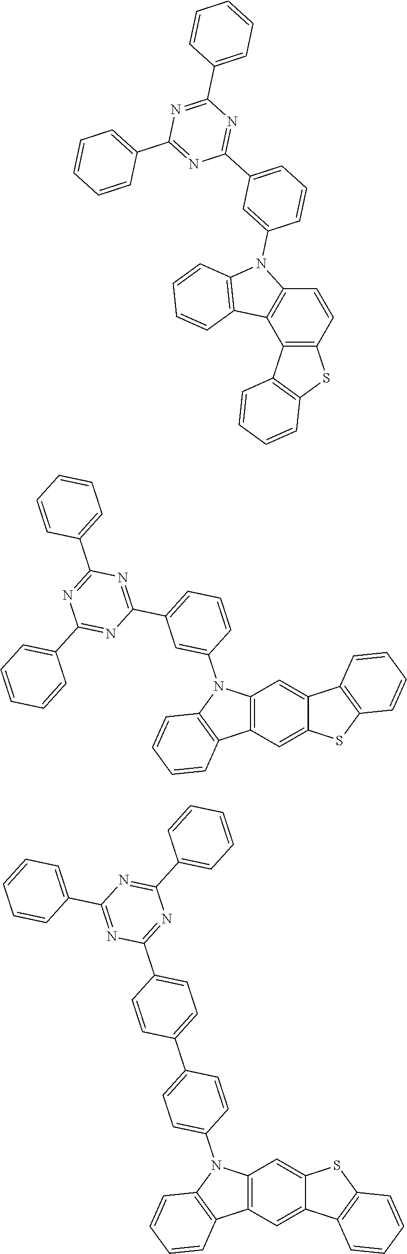 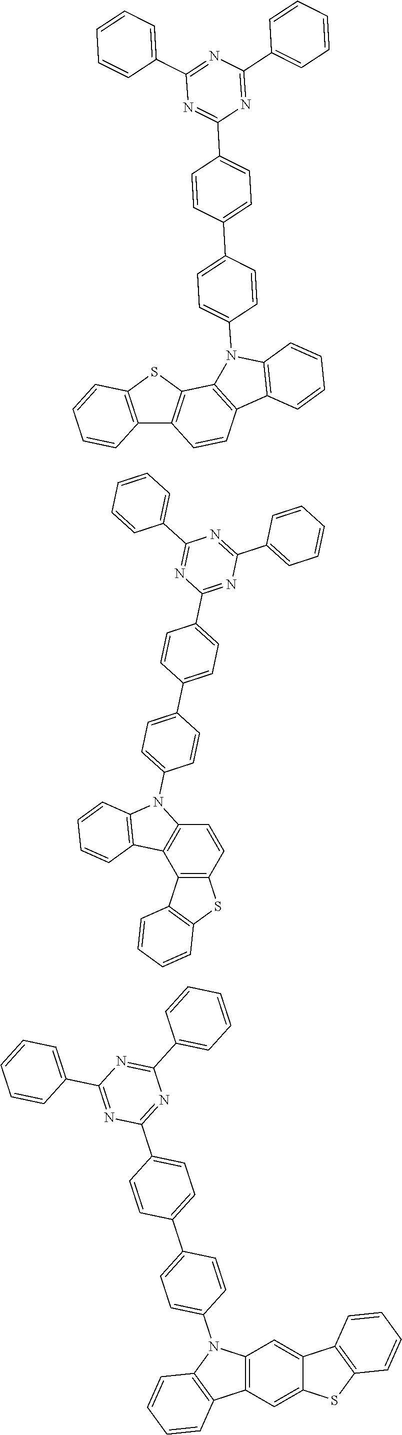 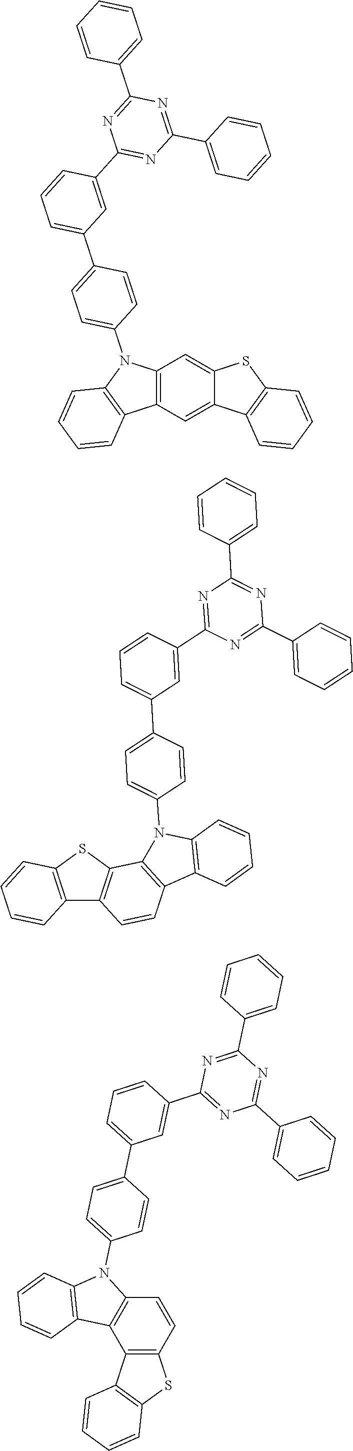 [SMILES: c1ccc(-c2nc(-c3ccccc3)nc(-c3cccc(-c4ccc(-n5c6ccccc6c6cc7c(cc65)sc5ccccc57)cc4)c3)n2)cc1 ; c1ccc(-c2nc(-c3ccccc3)nc(-c3cccc(-c4ccc(-n5c6ccccc6c6ccc7c8ccccc8sc7c65)cc4)c3)n2)cc1 ; c1ccc(-c2nc(-c3ccccc3)nc(-c3cccc(-c4ccc(-n5c6ccccc6c6c7c(ccc65)sc5ccccc57)cc4)c3)n2)cc1] 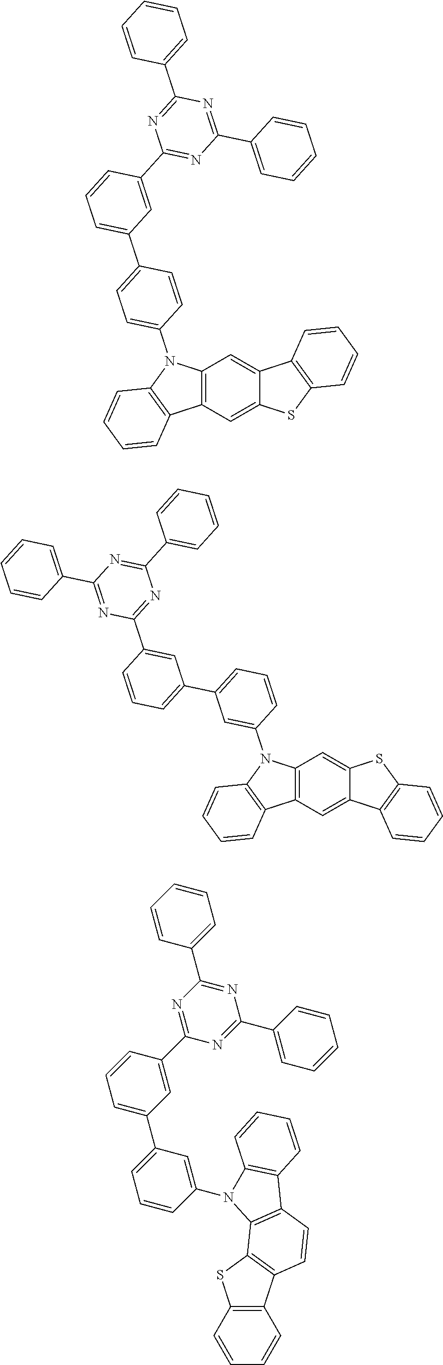 [SMILES: c1ccc(-c2nc(-c3ccccc3)nc(-c3cccc(-c4ccc(-n5c6ccccc6c6cc7sc8ccccc8c7cc65)cc4)c3)n2)cc1 ; c1ccc(-c2nc(-c3ccccc3)nc(-c3cccc(-c4cccc(-n5c6ccccc6c6cc7c(cc65)sc5ccccc57)c4)c3)n2)cc1 ; c1ccc(-c2nc(-c3ccccc3)nc(-c3cccc(-c4cccc(-n5c6ccccc6c6ccc7c8ccccc8sc7c65)c4)c3)n2)cc1] 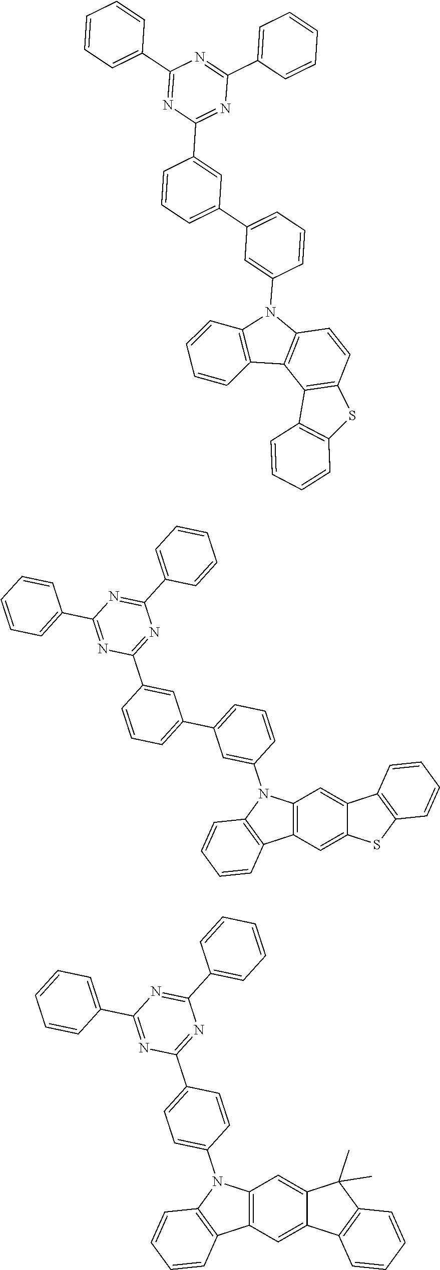 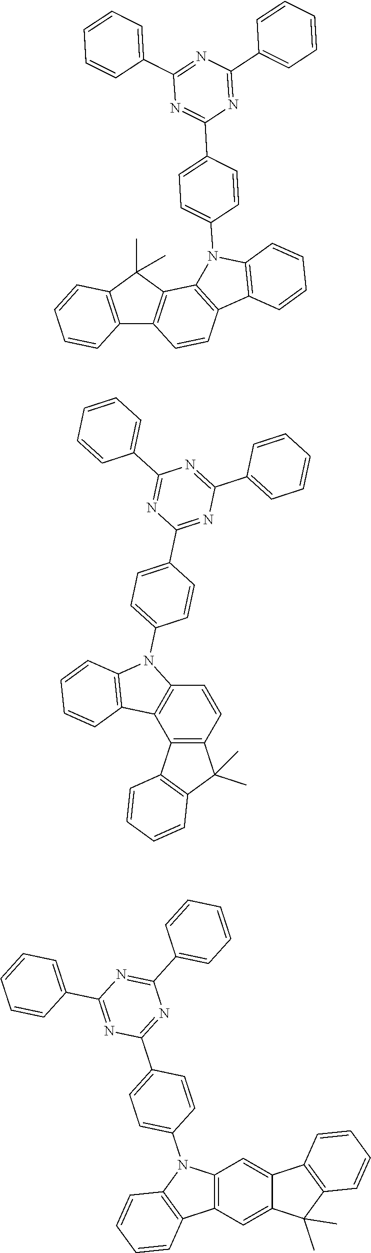 [SMILES: CC1(C)c2ccccc2-c2ccc3c4ccccc4n(-c4ccc(-c5nc(-c6ccccc6)nc(-c6ccccc6)n5)cc4)c3c21 ; CC1(C)c2ccccc2-c2c1ccc1c2c2ccccc2n1-c1ccc(-c2nc(-c3ccccc3)nc(-c3ccccc3)n2)cc1 ; CC1(C)c2ccccc2-c2cc3c(cc21)c1ccccc1n3-c1ccc(-c2nc(-c3ccccc3)nc(-c3ccccc3)n2)cc1] 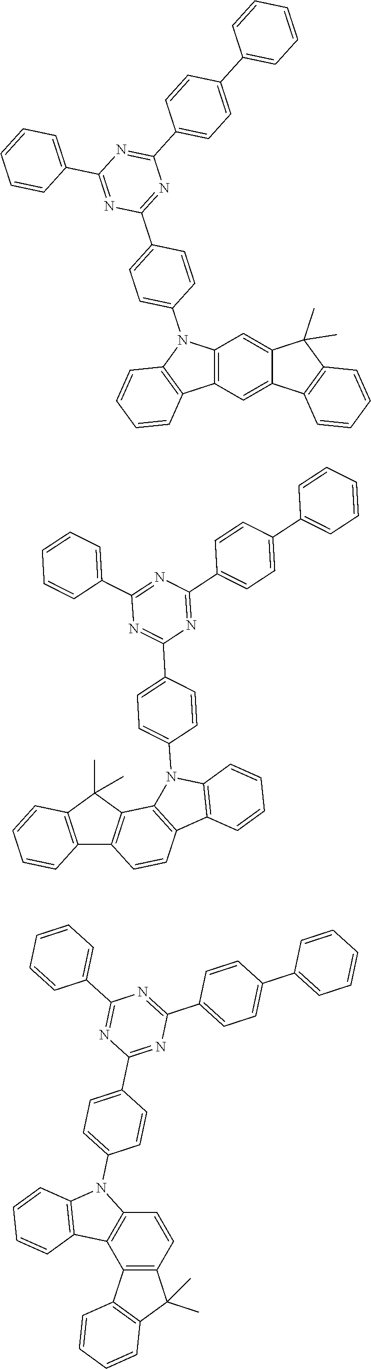 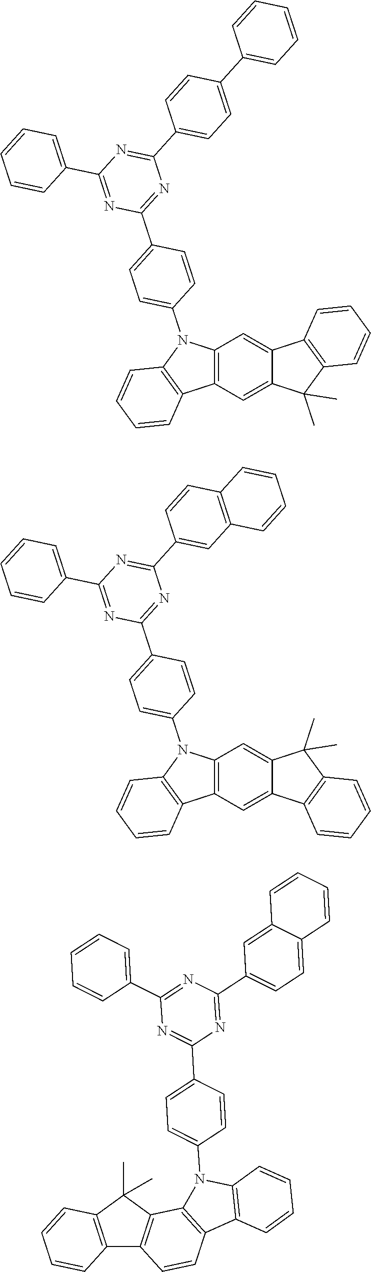 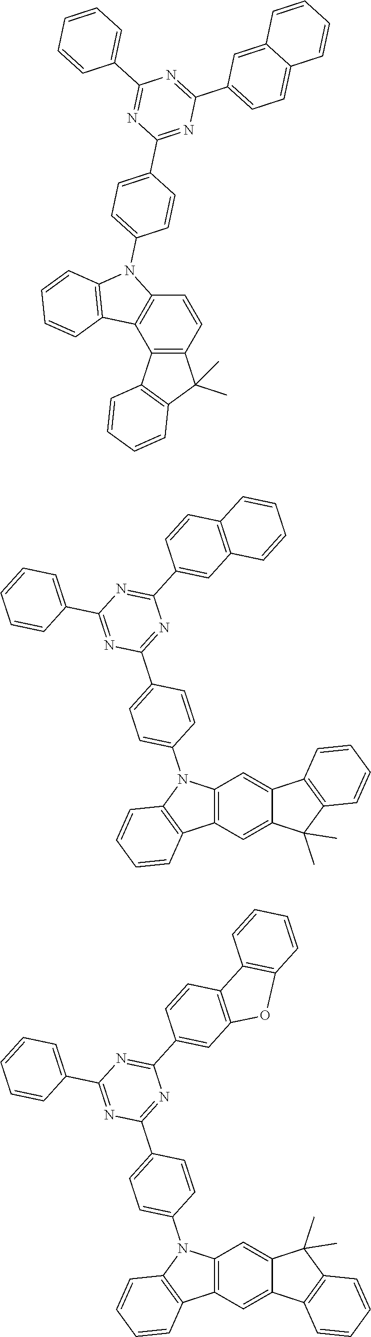 [SMILES: CC1(C)c2ccccc2-c2c1ccc1c2c2ccccc2n1-c1ccc(-c2nc(-c3ccccc3)nc(-c3ccc4ccccc4c3)n2)cc1 ; CC1(C)c2ccccc2-c2cc3c(cc21)c1ccccc1n3-c1ccc(-c2nc(-c3ccccc3)nc(-c3ccc4ccccc4c3)n2)cc1 ; CC1(C)c2ccccc2-c2cc3c4ccccc4n(-c4ccc(-c5nc(-c6ccccc6)nc(-c6ccc7c(c6)oc6ccccc67)n5)cc4)c3cc21] 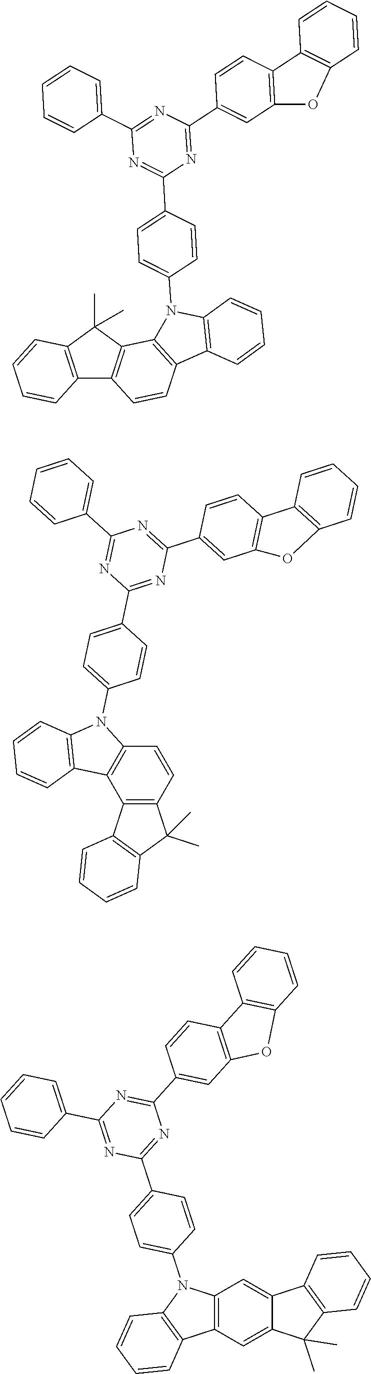 [SMILES: CC1(C)c2ccccc2-c2ccc3c4ccccc4n(-c4ccc(-c5nc(-c6ccccc6)nc(-c6ccc7c(c6)oc6ccccc67)n5)cc4)c3c21 ; CC1(C)c2ccccc2-c2c1ccc1c2c2ccccc2n1-c1ccc(-c2nc(-c3ccccc3)nc(-c3ccc4c(c3)oc3ccccc34)n2)cc1 ; CC1(C)c2ccccc2-c2cc3c(cc21)c1ccccc1n3-c1ccc(-c2nc(-c3ccccc3)nc(-c3ccc4c(c3)oc3ccccc34)n2)cc1] 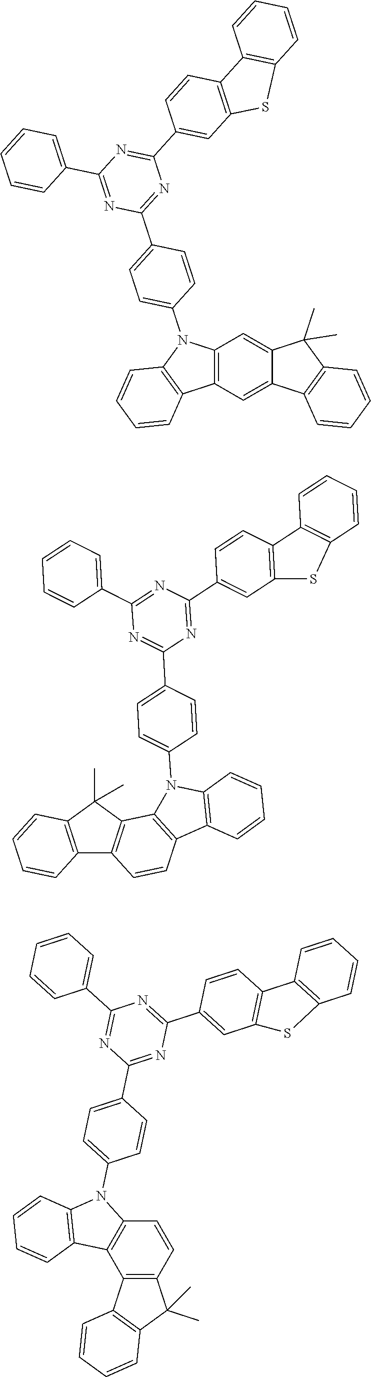 [SMILES: CC1(C)c2ccccc2-c2cc3c4ccccc4n(-c4ccc(-c5nc(-c6ccccc6)nc(-c6ccc7c(c6)sc6ccccc67)n5)cc4)c3cc21 ; CC1(C)c2ccccc2-c2ccc3c4ccccc4n(-c4ccc(-c5nc(-c6ccccc6)nc(-c6ccc7c(c6)sc6ccccc67)n5)cc4)c3c21 ; CC1(C)c2ccccc2-c2c1ccc1c2c2ccccc2n1-c1ccc(-c2nc(-c3ccccc3)nc(-c3ccc4c(c3)sc3ccccc34)n2)cc1] 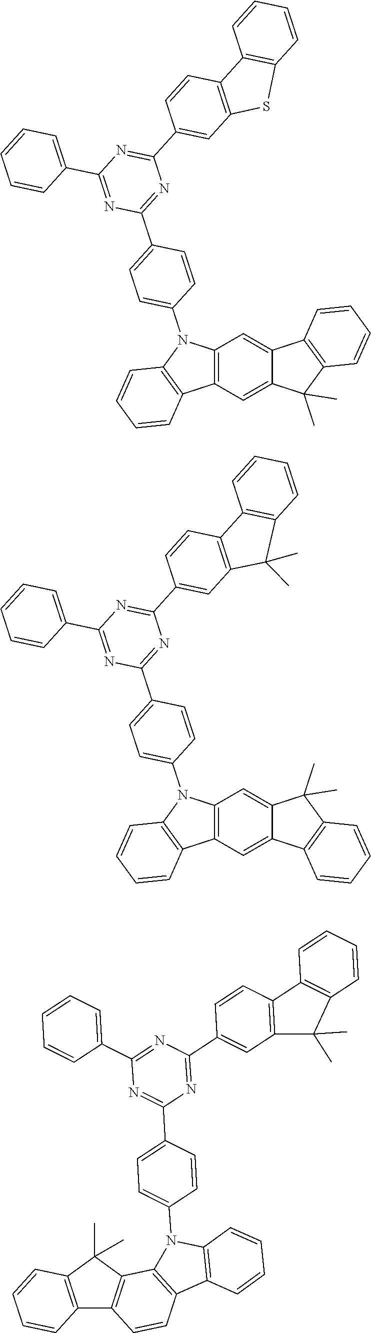 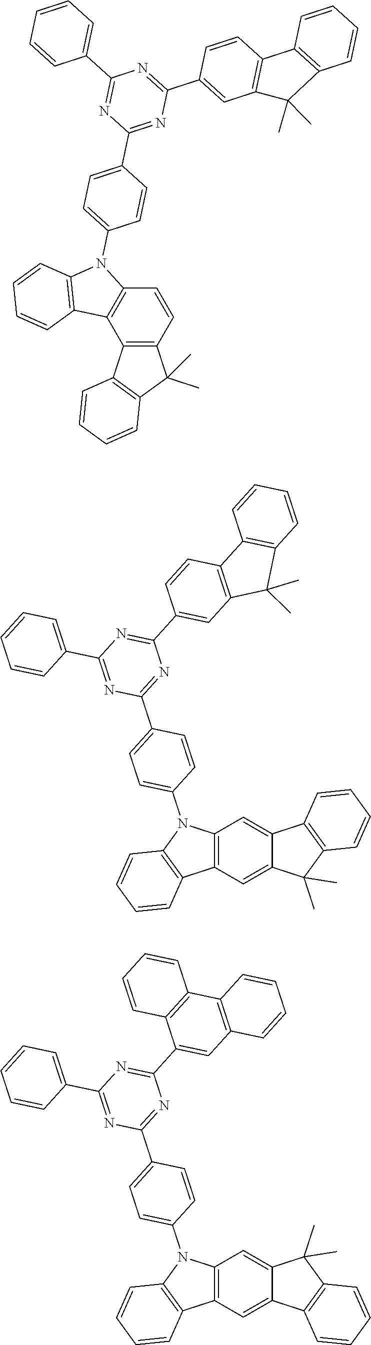 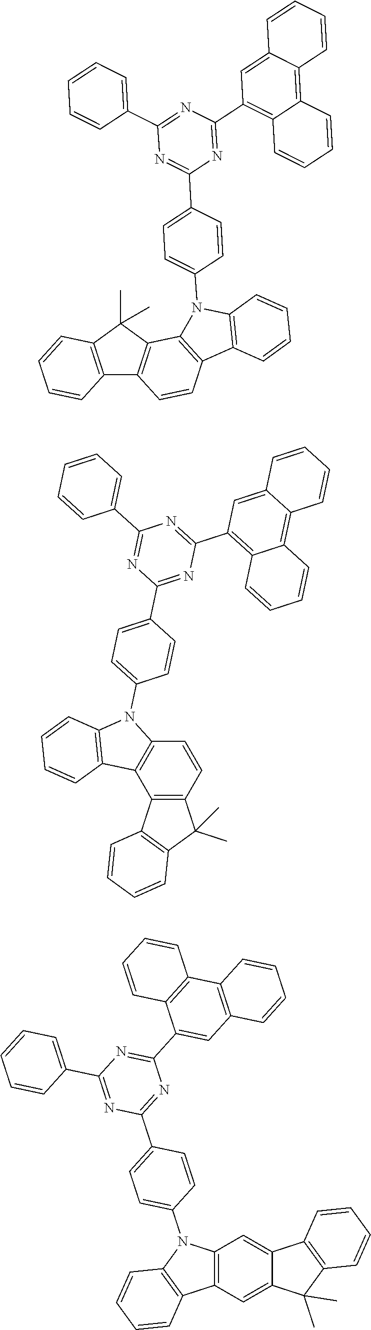 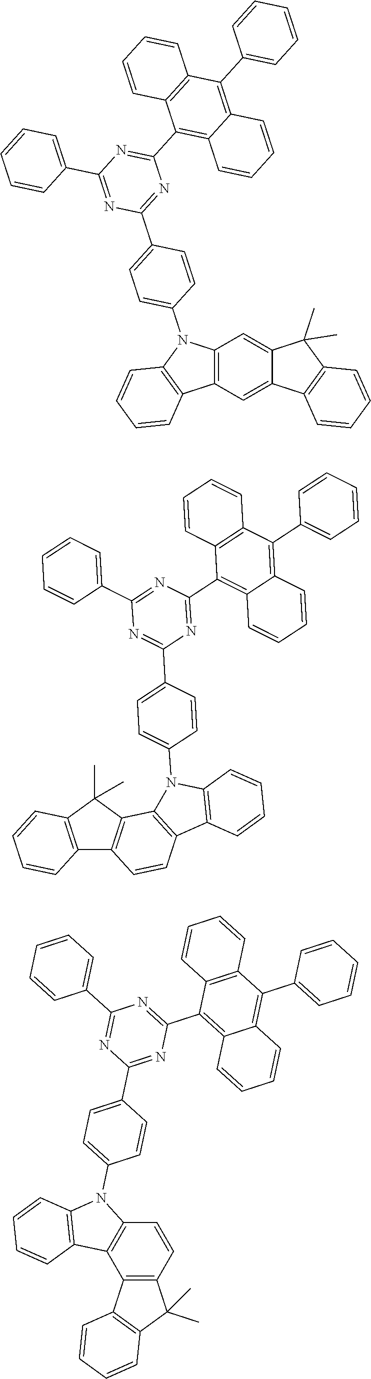 [SMILES: CC1(C)c2ccccc2-c2cc3c4ccccc4n(-c4ccc(-c5nc(-c6ccccc6)nc(-c6c7ccccc7c(-c7ccccc7)c7ccccc67)n5)cc4)c3cc21 ; CC1(C)c2ccccc2-c2ccc3c4ccccc4n(-c4ccc(-c5nc(-c6ccccc6)nc(-c6c7ccccc7c(-c7ccccc7)c7ccccc67)n5)cc4)c3c21 ; CC1(C)c2ccccc2-c2c1ccc1c2c2ccccc2n1-c1ccc(-c2nc(-c3ccccc3)nc(-c3c4ccccc4c(-c4ccccc4)c4ccccc34)n2)cc1] 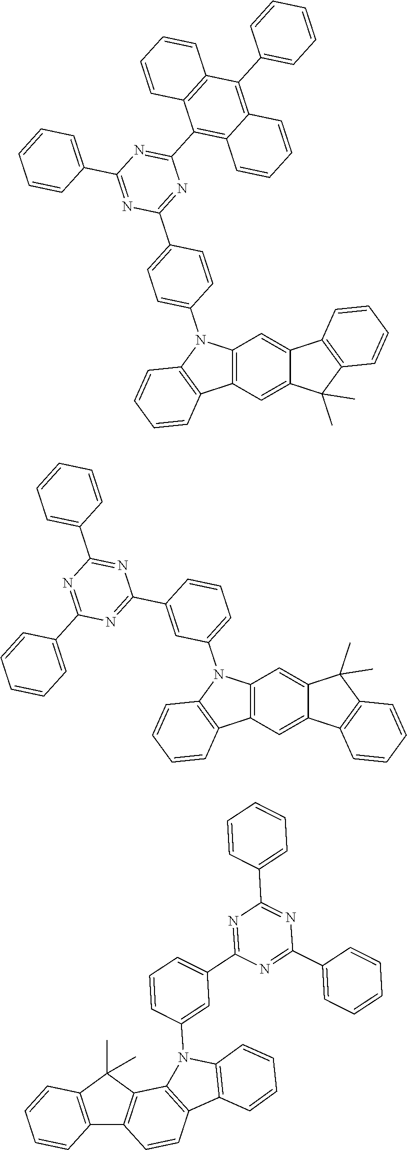 [SMILES: CC1(C)c2ccccc2-c2cc3c(cc21)c1ccccc1n3-c1ccc(-c2nc(-c3ccccc3)nc(-c3c4ccccc4c(-c4ccccc4)c4ccccc34)n2)cc1 ; CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2ccc3c4ccccc4n(-c4cccc(-c5nc(-c6ccccc6)nc(-c6ccccc6)n5)c4)c3c21] 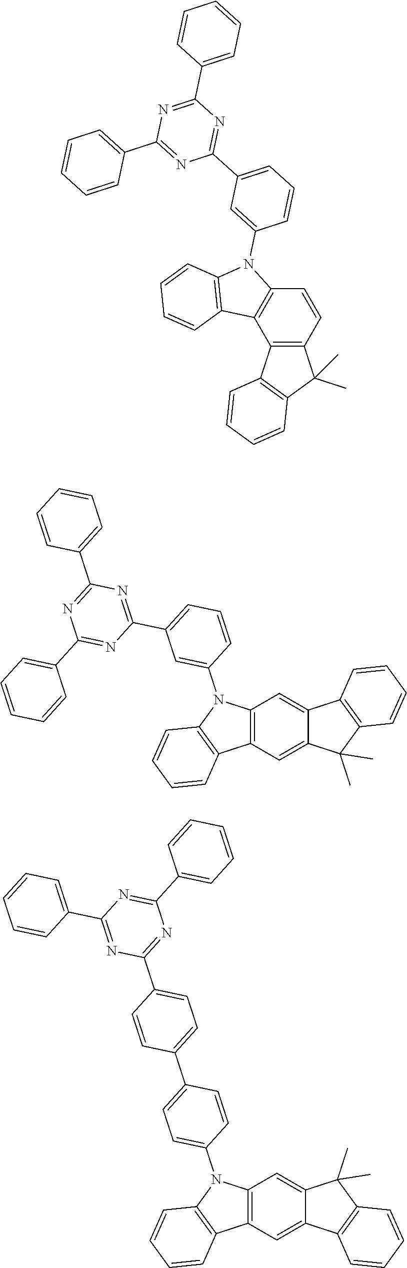 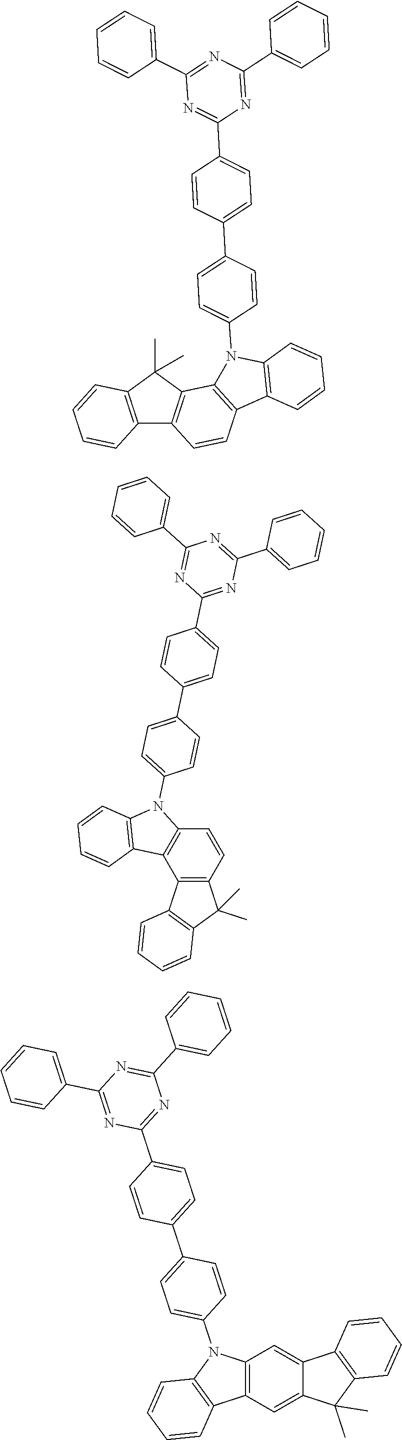 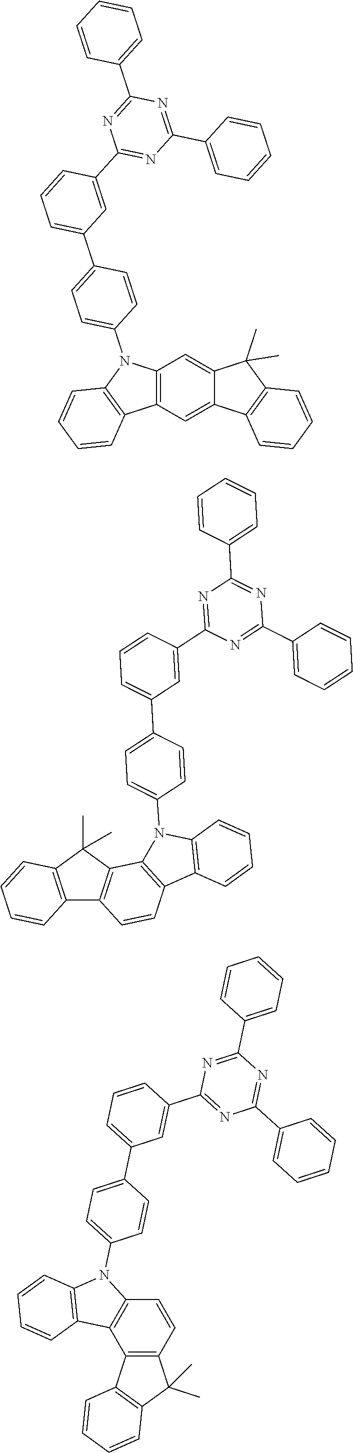 [SMILES: CC1(C)c2ccccc2-c2cc3c4ccccc4n(-c4ccc(-c5cccc(-c6nc(-c7ccccc7)nc(-c7ccccc7)n6)c5)cc4)c3cc21 ; CC1(C)c2ccccc2-c2ccc3c4ccccc4n(-c4ccc(-c5cccc(-c6nc(-c7ccccc7)nc(-c7ccccc7)n6)c5)cc4)c3c21 ; CC1(C)c2ccccc2-c2c1ccc1c2c2ccccc2n1-c1ccc(-c2cccc(-c3nc(-c4ccccc4)nc(-c4ccccc4)n3)c2)cc1] 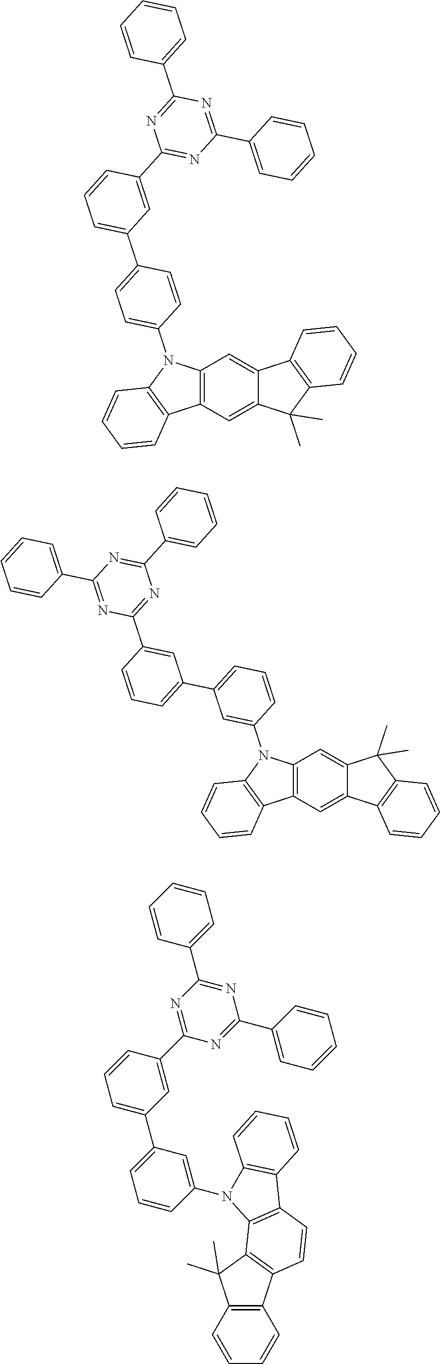 [SMILES: CC1(C)c2ccccc2-c2cc3c(cc21)c1ccccc1n3-c1ccc(-c2cccc(-c3nc(-c4ccccc4)nc(-c4ccccc4)n3)c2)cc1 ; CC1(C)c2ccccc2-c2cc3c4ccccc4n(-c4cccc(-c5cccc(-c6nc(-c7ccccc7)nc(-c7ccccc7)n6)c5)c4)c3cc21 ; CC1(C)c2ccccc2-c2ccc3c4ccccc4n(-c4cccc(-c5cccc(-c6nc(-c7ccccc7)nc(-c7ccccc7)n6)c5)c4)c3c21] 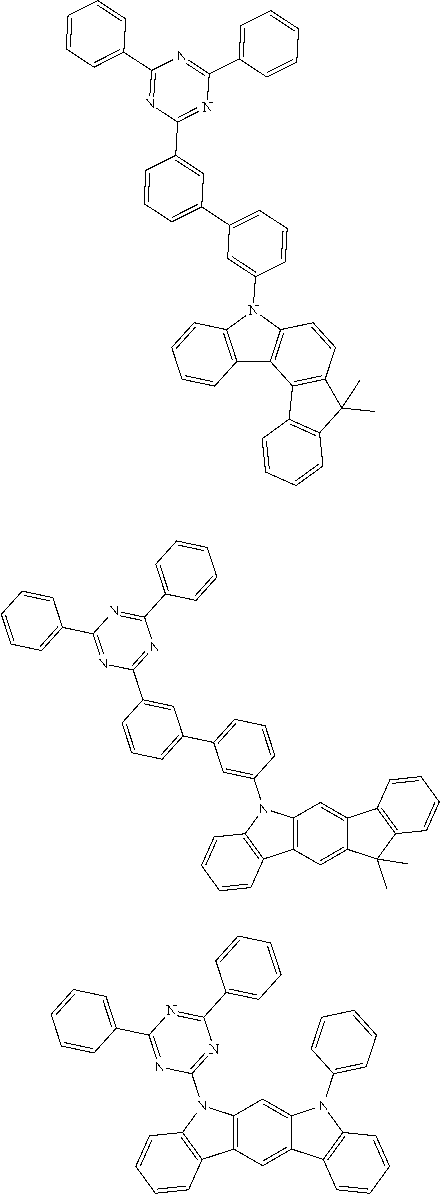 [SMILES: CC1(C)c2ccccc2-c2c1ccc1c2c2ccccc2n1-c1cccc(-c2cccc(-c3nc(-c4ccccc4)nc(-c4ccccc4)n3)c2)c1 ; CC1(C)c2ccccc2-c2cc3c(cc21)c1ccccc1n3-c1cccc(-c2cccc(-c3nc(-c4ccccc4)nc(-c4ccccc4)n3)c2)c1 ; c1ccc(-c2nc(-c3ccccc3)nc(-n3c4ccccc4c4cc5c6ccccc6n(-c6ccccc6)c5cc43)n2)cc1] 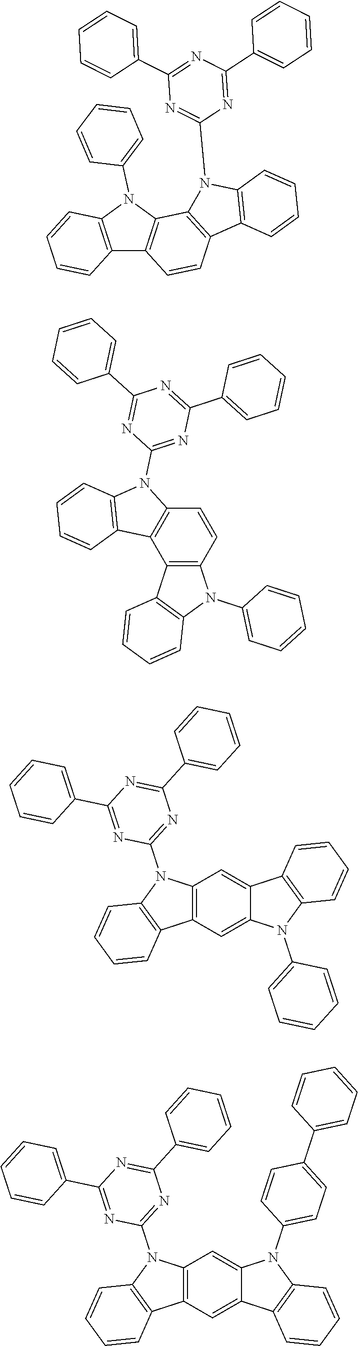 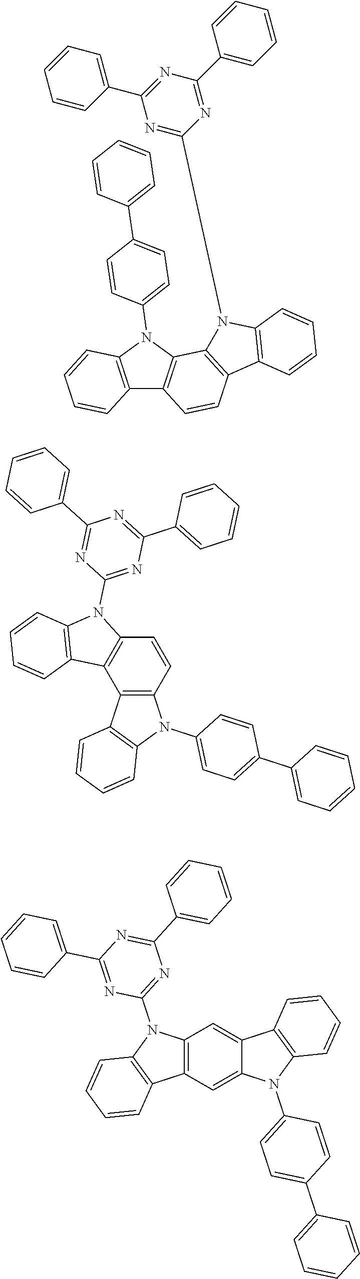 [SMILES: c1ccc(-c2ccc(-n3c4ccccc4c4ccc5c6ccccc6n(-c6nc(-c7ccccc7)nc(-c7ccccc7)n6)c5c43)cc2)cc1 ; c1ccc(-c2ccc(-n3c4ccccc4c4c5c6ccccc6n(-c6nc(-c7ccccc7)nc(-c7ccccc7)n6)c5ccc43)cc2)cc1 ; c1ccc(-c2ccc(-n3c4ccccc4c4cc5c(cc43)c3ccccc3n5-c3nc(-c4ccccc4)nc(-c4ccccc4)n3)cc2)cc1] 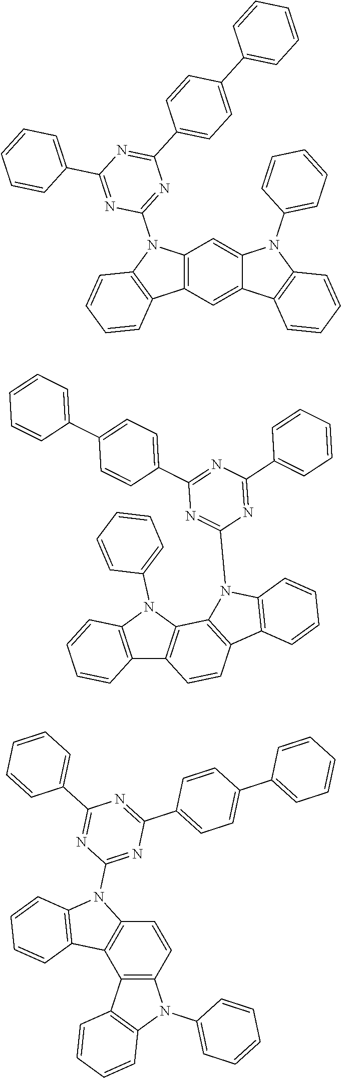 [SMILES: c1ccc(-c2ccc(-c3nc(-c4ccccc4)nc(-n4c5ccccc5c5cc6c7ccccc7n(-c7ccccc7)c6cc54)n3)cc2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2ccc(-c3nc(-c4ccccc4)nc(-n4c5ccccc5c5c6c7ccccc7n(-c7ccccc7)c6ccc54)n3)cc2)cc1] 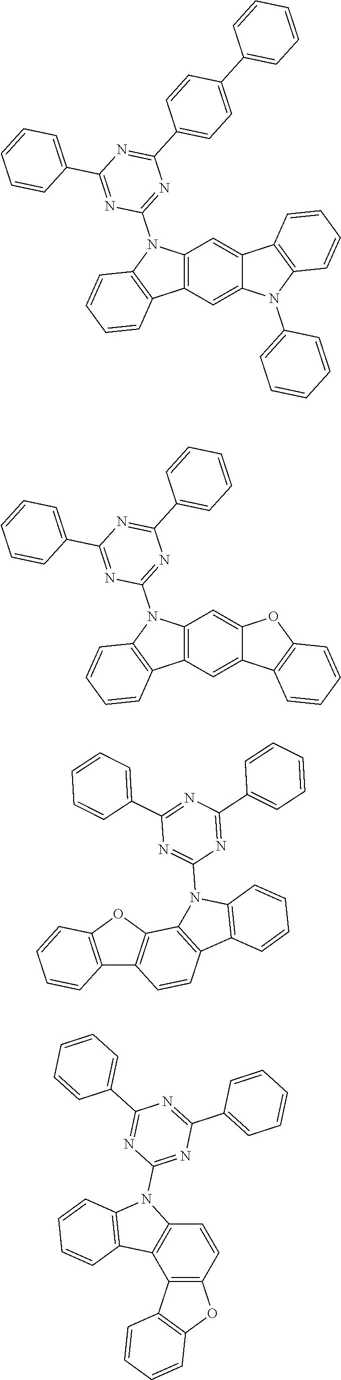 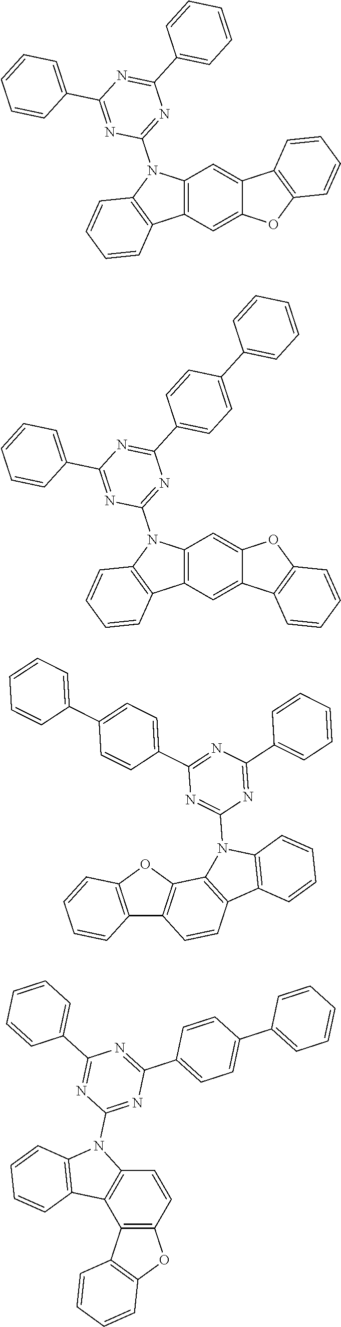 [SMILES: c1ccc(-c2nc(-c3ccccc3)nc(-n3c4ccccc4c4cc5oc6ccccc6c5cc43)n2)cc1 ; c1ccc(-c2ccc(-c3nc(-c4ccccc4)nc(-n4c5ccccc5c5cc6c(cc54)oc4ccccc46)n3)cc2)cc1 ; c1ccc(-c2ccc(-c3nc(-c4ccccc4)nc(-n4c5ccccc5c5ccc6c7ccccc7oc6c54)n3)cc2)cc1 ; c1ccc(-c2ccc(-c3nc(-c4ccccc4)nc(-n4c5ccccc5c5c6c(ccc54)oc4ccccc46)n3)cc2)cc1] 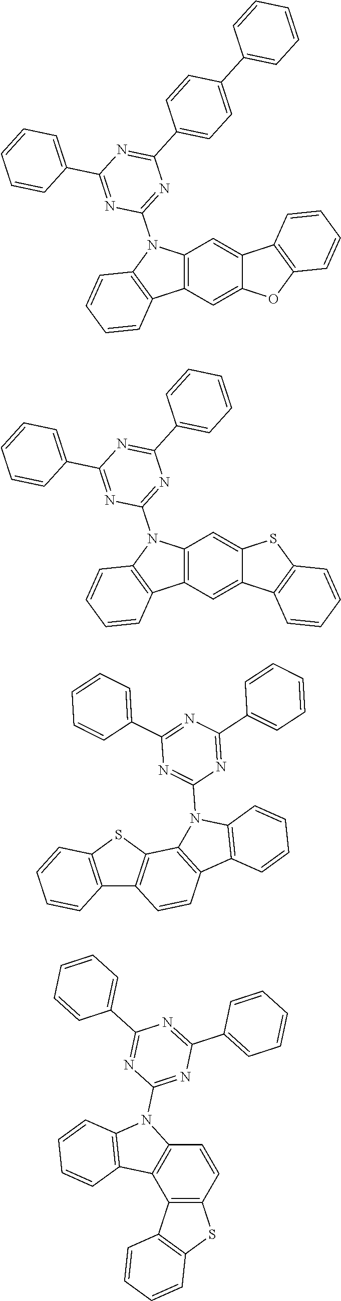 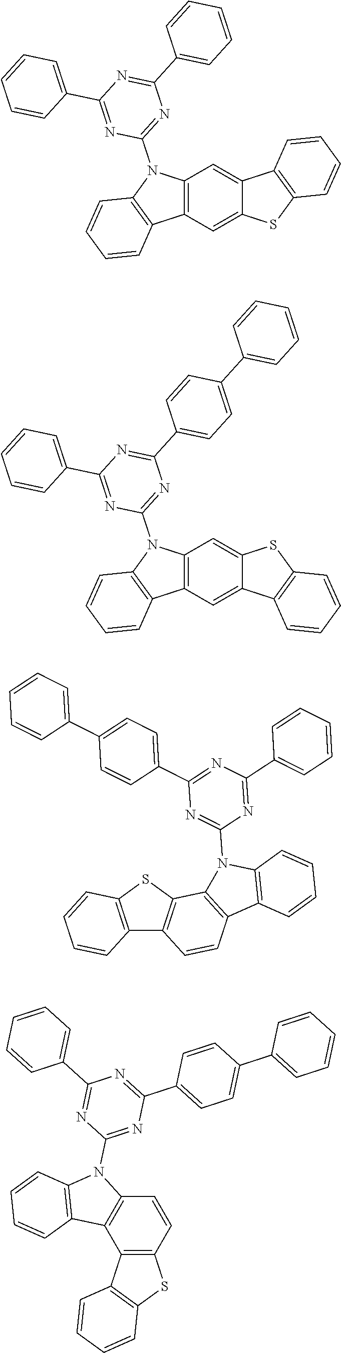 [SMILES: c1ccc(-c2nc(-c3ccccc3)nc(-n3c4ccccc4c4cc5sc6ccccc6c5cc43)n2)cc1 ; c1ccc(-c2ccc(-c3nc(-c4ccccc4)nc(-n4c5ccccc5c5cc6c(cc54)sc4ccccc46)n3)cc2)cc1 ; c1ccc(-c2ccc(-c3nc(-c4ccccc4)nc(-n4c5ccccc5c5ccc6c7ccccc7sc6c54)n3)cc2)cc1 ; c1ccc(-c2ccc(-c3nc(-c4ccccc4)nc(-n4c5ccccc5c5c6c(ccc54)sc4ccccc46)n3)cc2)cc1] 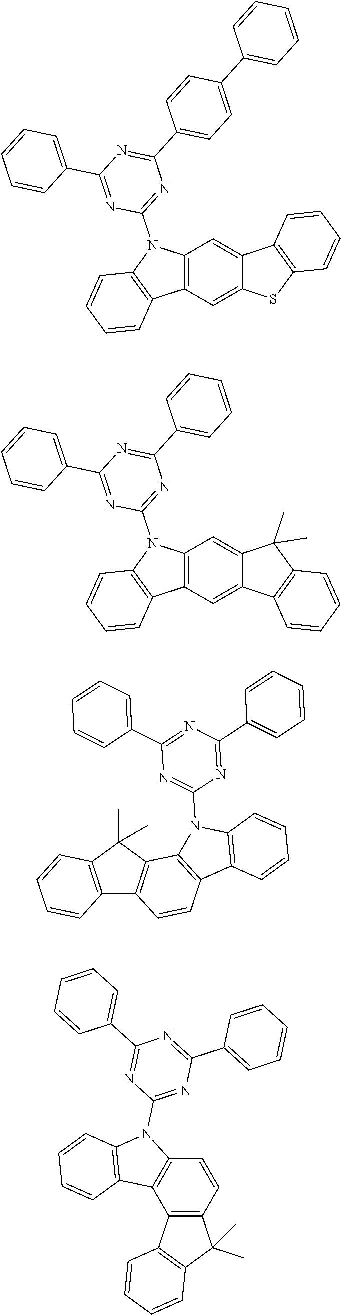 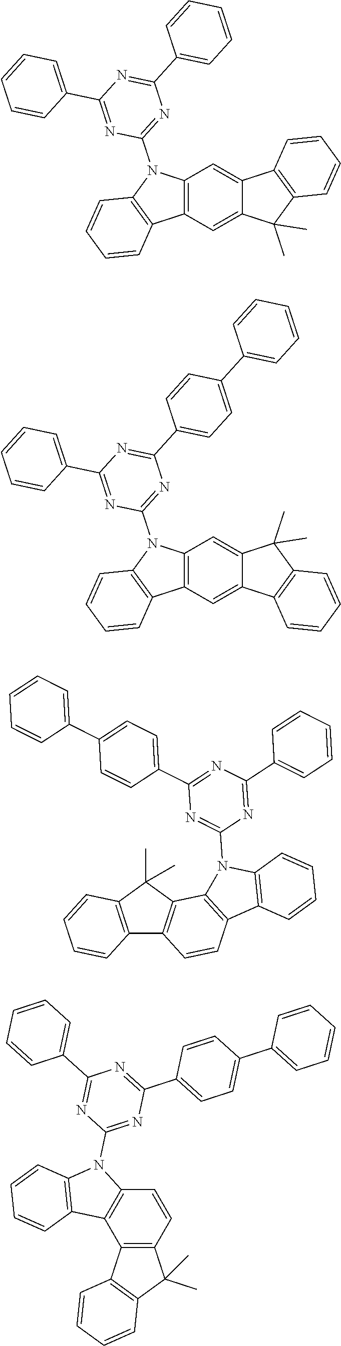 [SMILES: CC1(C)c2ccccc2-c2cc3c(cc21)c1ccccc1n3-c1nc(-c2ccccc2)nc(-c2ccccc2)n1 ; CC1(C)c2ccccc2-c2cc3c4ccccc4n(-c4nc(-c5ccccc5)nc(-c5ccc(-c6ccccc6)cc5)n4)c3cc21 ; CC1(C)c2ccccc2-c2ccc3c4ccccc4n(-c4nc(-c5ccccc5)nc(-c5ccc(-c6ccccc6)cc5)n4)c3c21 ; CC1(C)c2ccccc2-c2c1ccc1c2c2ccccc2n1-c1nc(-c2ccccc2)nc(-c2ccc(-c3ccccc3)cc2)n1] 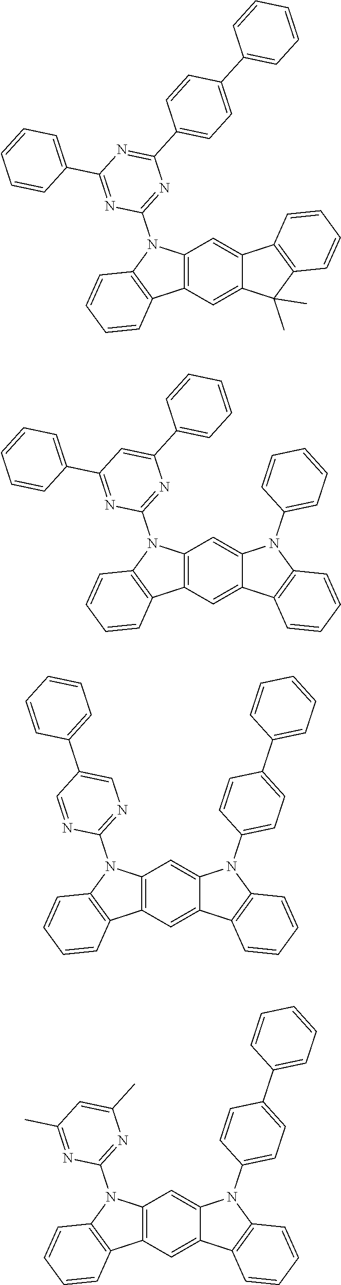 [SMILES: CC1(C)c2ccccc2-c2cc3c(cc21)c1ccccc1n3-c1nc(-c2ccccc2)nc(-c2ccc(-c3ccccc3)cc2)n1 ; c1ccc(-c2cc(-c3ccccc3)nc(-n3c4ccccc4c4cc5c6ccccc6n(-c6ccccc6)c5cc43)n2)cc1 ; c1ccc(-c2ccc(-n3c4ccccc4c4cc5c6ccccc6n(-c6ncc(-c7ccccc7)cn6)c5cc43)cc2)cc1 ; Cc1cc(C)nc(-n2c3ccccc3c3cc4c5ccccc5n(-c5ccc(-c6ccccc6)cc5)c4cc32)n1] 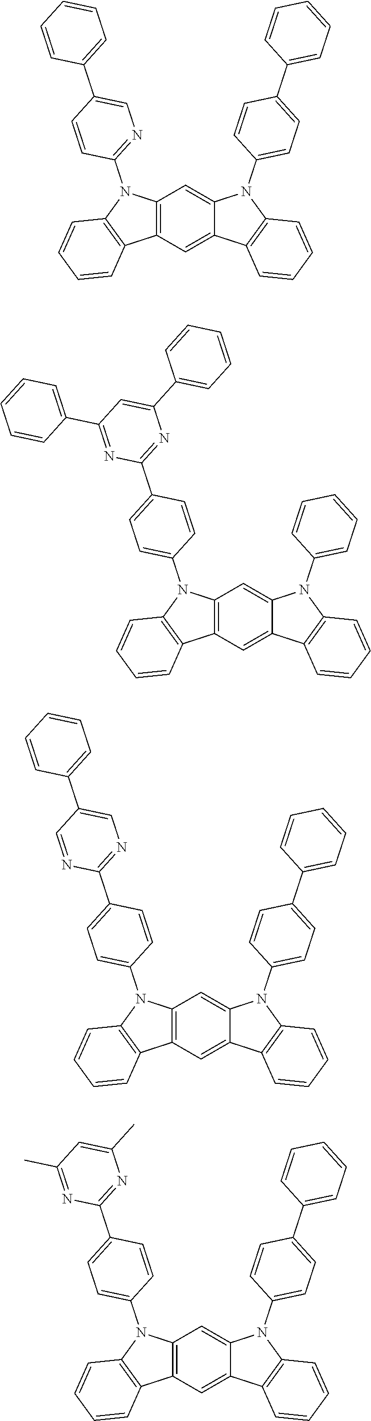 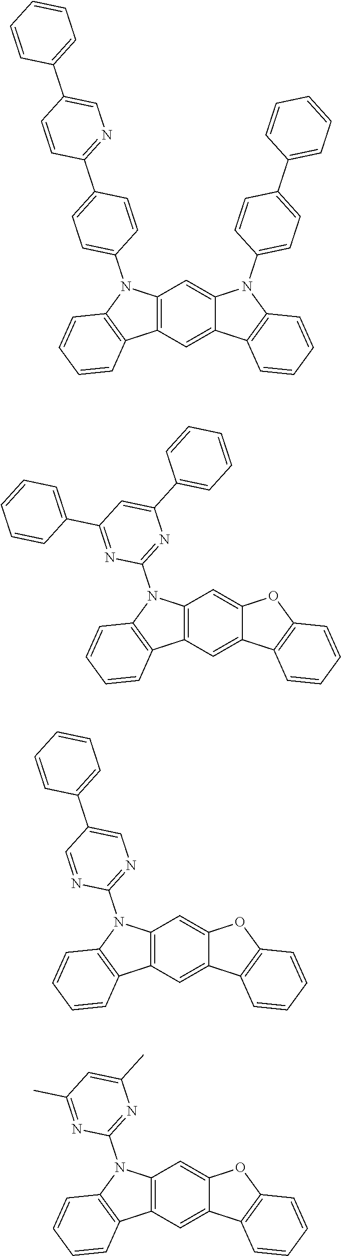 [SMILES: c1ccc(-c2ccc(-n3c4ccccc4c4cc5c6ccccc6n(-c6ccc(-c7ccc(-c8ccccc8)cn7)cc6)c5cc43)cc2)cc1 ; c1ccc(-c2cc(-c3ccccc3)nc(-n3c4ccccc4c4cc5c(cc43)oc3ccccc35)n2)cc1 ; c1ccc(-c2cnc(-n3c4ccccc4c4cc5c(cc43)oc3ccccc35)nc2)cc1 ; Cc1cc(C)nc(-n2c3ccccc3c3cc4c(cc32)oc2ccccc24)n1] 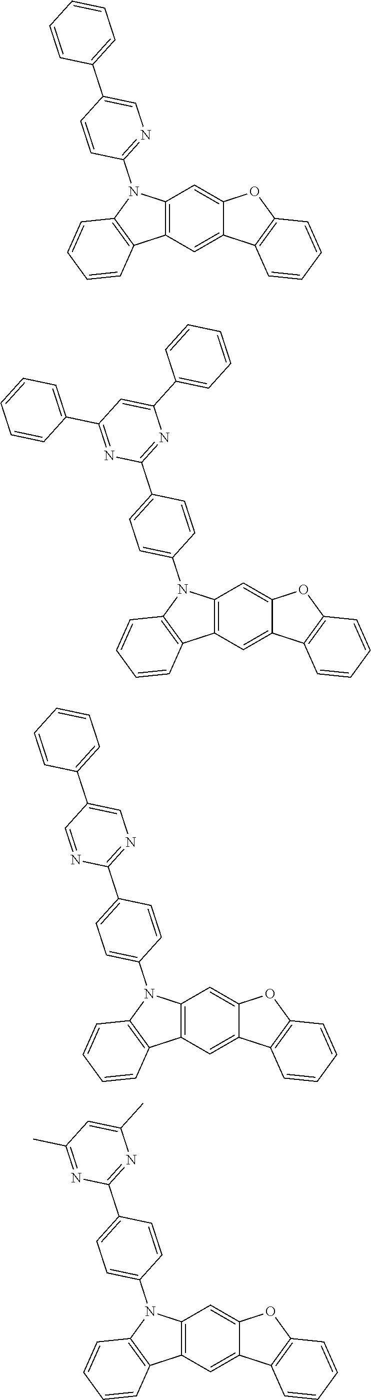 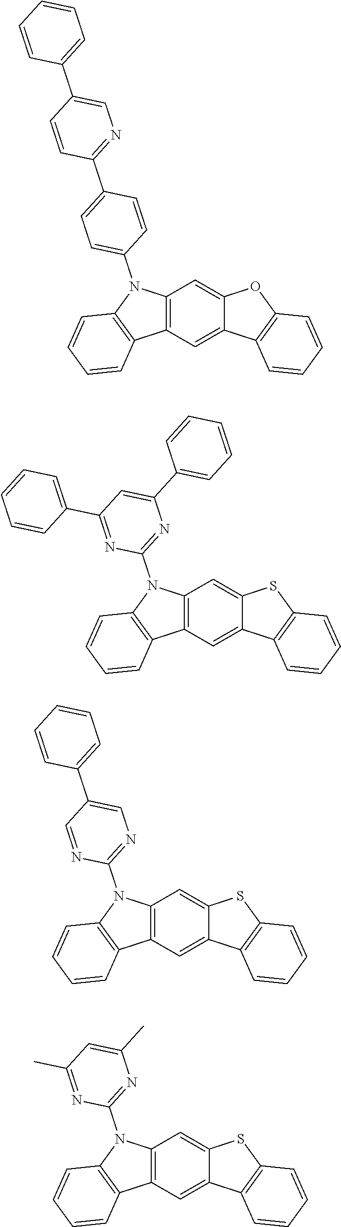 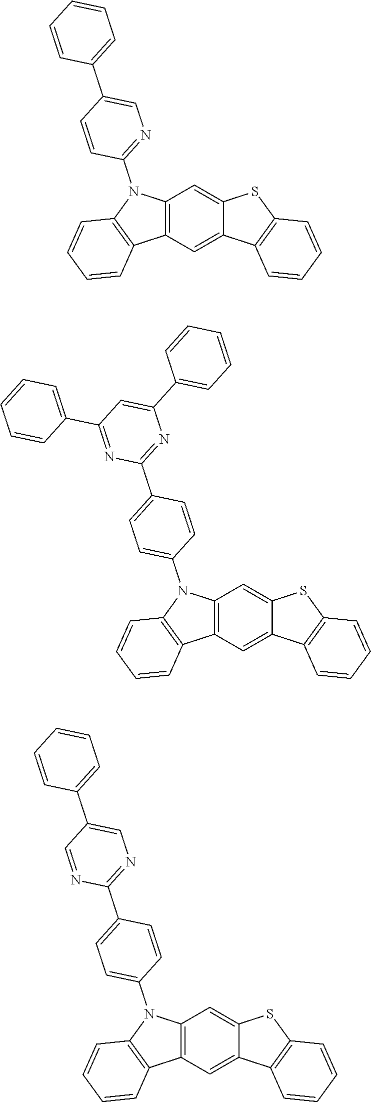 [SMILES: c1ccc(-c2ccc(-n3c4ccccc4c4cc5c(cc43)sc3ccccc35)nc2)cc1 ; c1ccc(-c2cc(-c3ccccc3)nc(-c3ccc(-n4c5ccccc5c5cc6c(cc54)sc4ccccc46)cc3)n2)cc1 ; c1ccc(-c2cnc(-c3ccc(-n4c5ccccc5c5cc6c(cc54)sc4ccccc46)cc3)nc2)cc1] 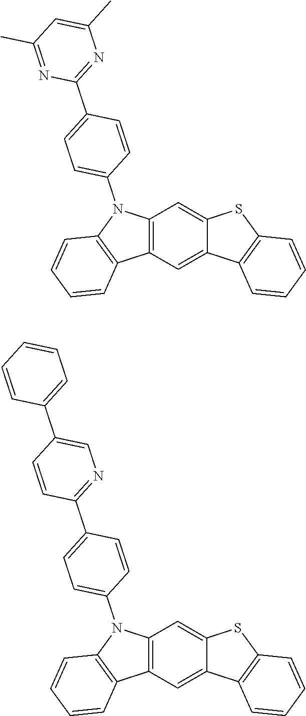 [SMILES: Cc1cc(C)nc(-c2ccc(-n3c4ccccc4c4cc5c(cc43)sc3ccccc35)cc2)n1 ; c1ccc(-c2ccc(-c3ccc(-n4c5ccccc5c5cc6c(cc54)sc4ccccc46)cc3)nc2)cc1] 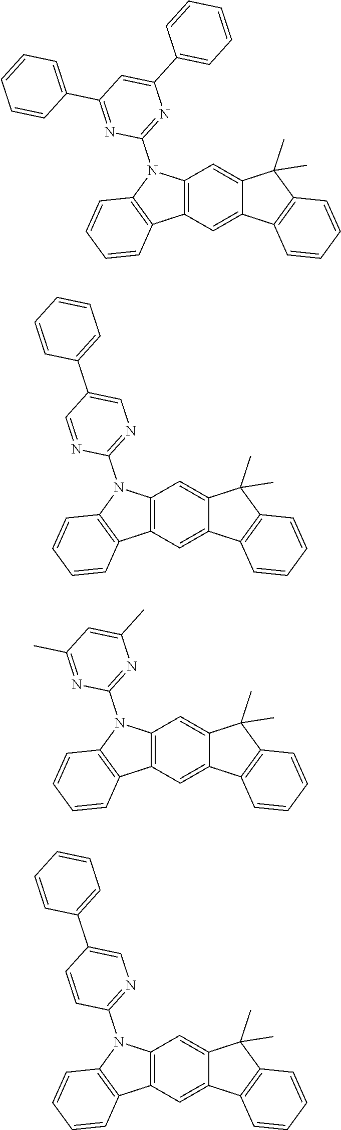 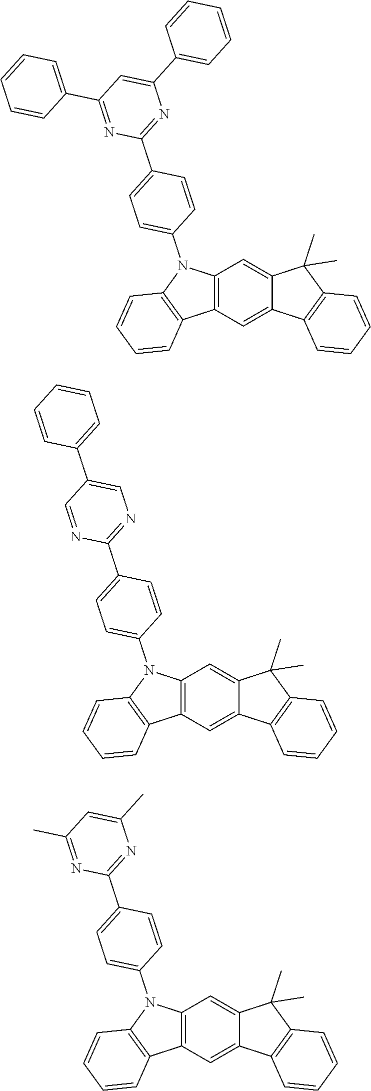 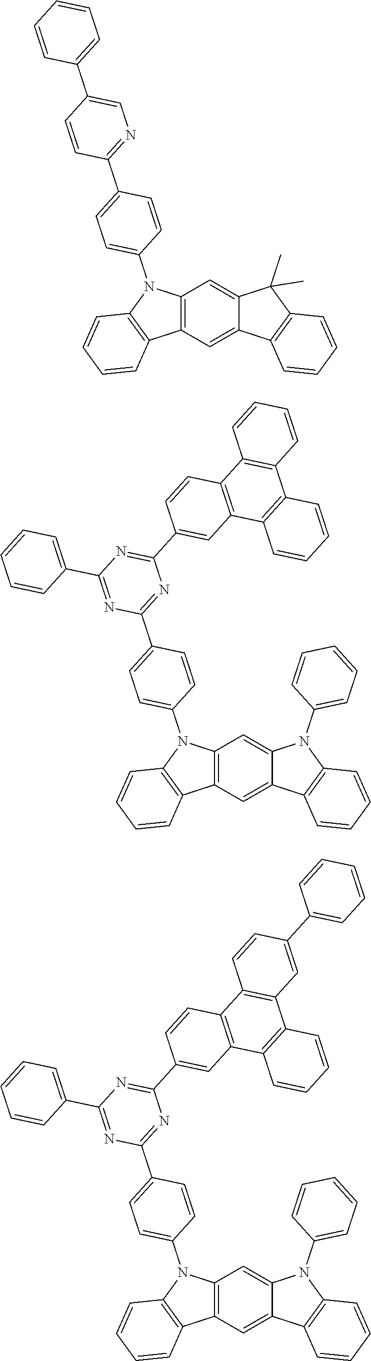 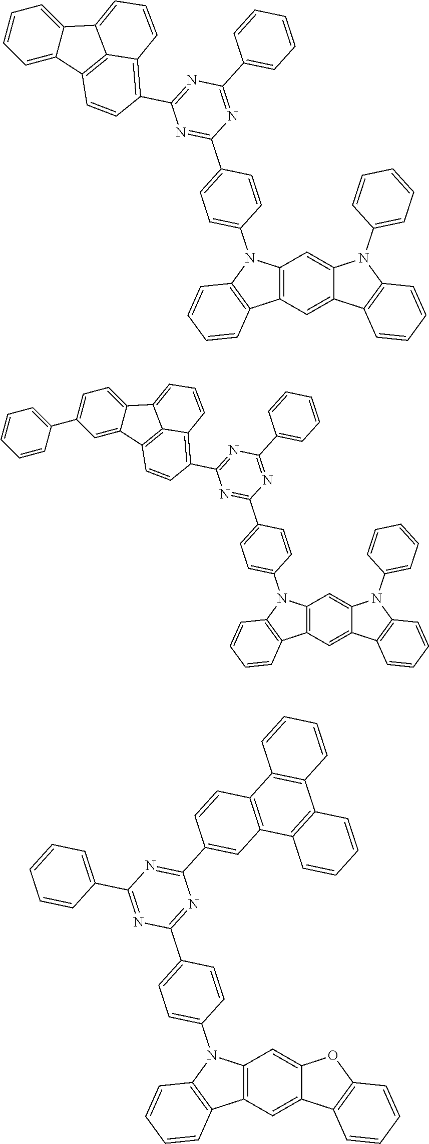 [SMILES: c1ccc(-c2nc(-c3ccc(-n4c5ccccc5c5cc6c7ccccc7n(-c7ccccc7)c6cc54)cc3)nc(-c3ccc4c5c(cccc35)-c3ccccc3-4)n2)cc1 ; c1ccc(-c2ccc3c(c2)-c2ccc(-c4nc(-c5ccccc5)nc(-c5ccc(-n6c7ccccc7c7cc8c9ccccc9n(-c9ccccc9)c8cc76)cc5)n4)c4cccc-3c24)cc1 ; c1ccc(-c2nc(-c3ccc(-n4c5ccccc5c5cc6c(cc54)oc4ccccc46)cc3)nc(-c3ccc4c5ccccc5c5ccccc5c4c3)n2)cc1] 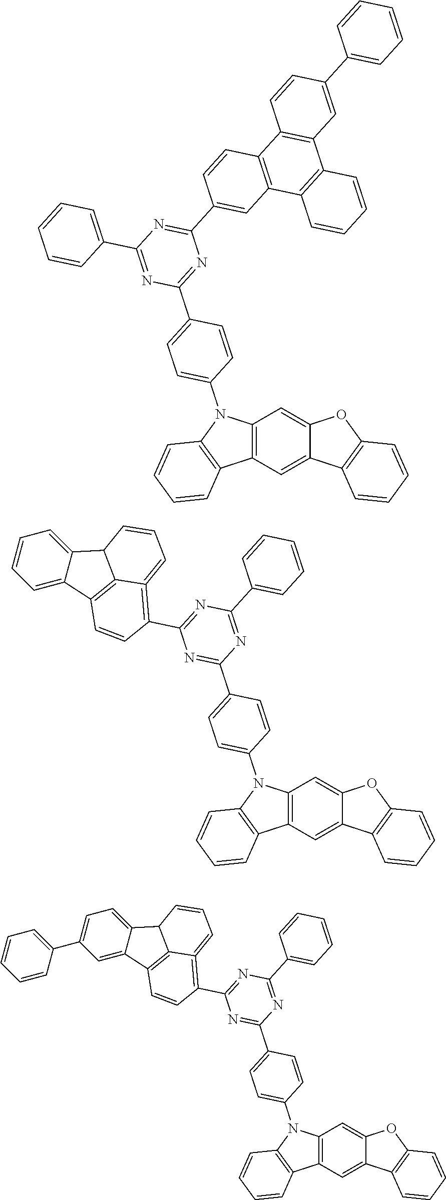 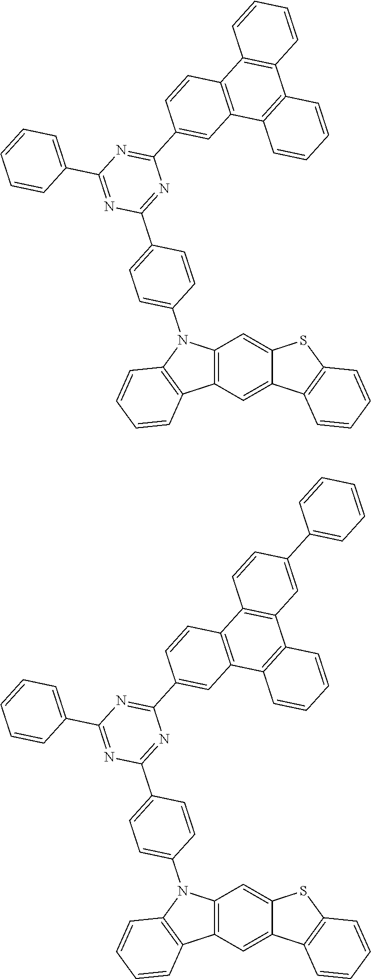 [SMILES: c1ccc(-c2nc(-c3ccc(-n4c5ccccc5c5cc6c(cc54)sc4ccccc46)cc3)nc(-c3ccc4c5ccccc5c5ccccc5c4c3)n2)cc1 ; c1ccc(-c2ccc3c4ccc(-c5nc(-c6ccccc6)nc(-c6ccc(-n7c8ccccc8c8cc9c(cc87)sc7ccccc79)cc6)n5)cc4c4ccccc4c3c2)cc1] 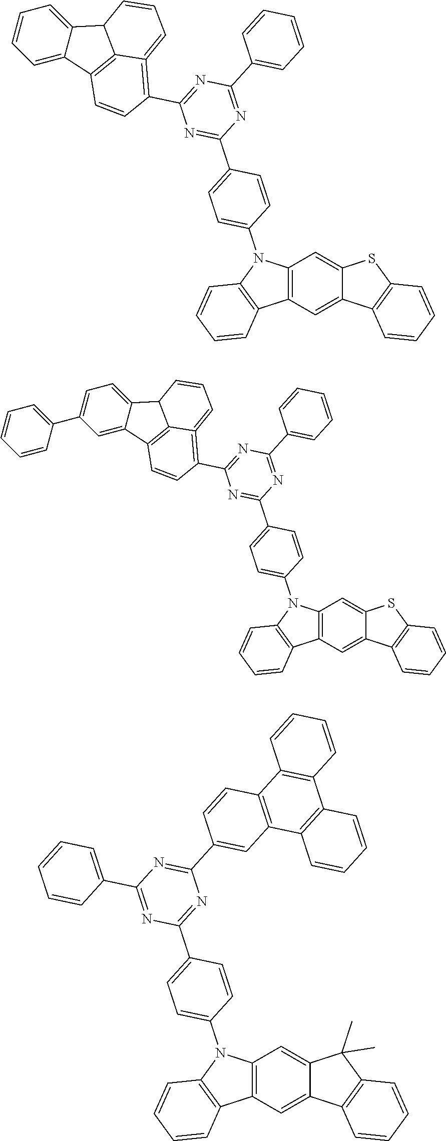 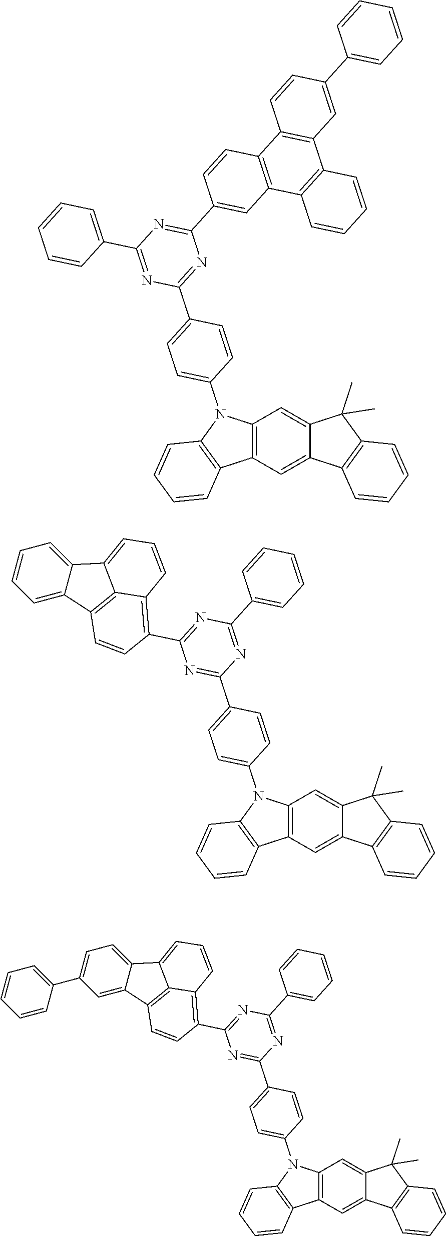 [SMILES: CC1(C)c2ccccc2-c2cc3c4ccccc4n(-c4ccc(-c5nc(-c6ccccc6)nc(-c6ccc7c8ccc(-c9ccccc9)cc8c8ccccc8c7c6)n5)cc4)c3cc21 ; CC1(C)c2ccccc2-c2cc3c4ccccc4n(-c4ccc(-c5nc(-c6ccccc6)nc(-c6ccc7c8c(cccc68)-c6ccccc6-7)n5)cc4)c3cc21 ; CC1(C)c2ccccc2-c2cc3c4ccccc4n(-c4ccc(-c5nc(-c6ccccc6)nc(-c6ccc7c8c(cccc68)-c6ccc(-c8ccccc8)cc6-7)n5)cc4)c3cc21] 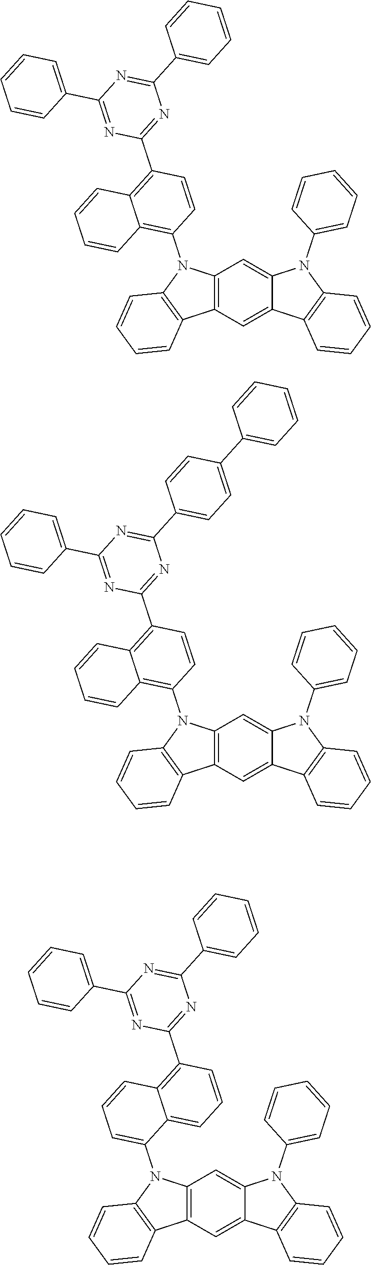 [SMILES: c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2ccc(-c3nc(-c4ccccc4)nc(-c4ccc(-n5c6ccccc6c6cc7c8ccccc8n(-c8ccccc8)c7cc65)c5ccccc45)n3)cc2)cc1 ; c1ccc(-c2nc(-c3ccccc3)nc(-c3cccc4c(-n5c6ccccc6c6cc7c8ccccc8n(-c8ccccc8)c7cc65)cccc34)n2)cc1] 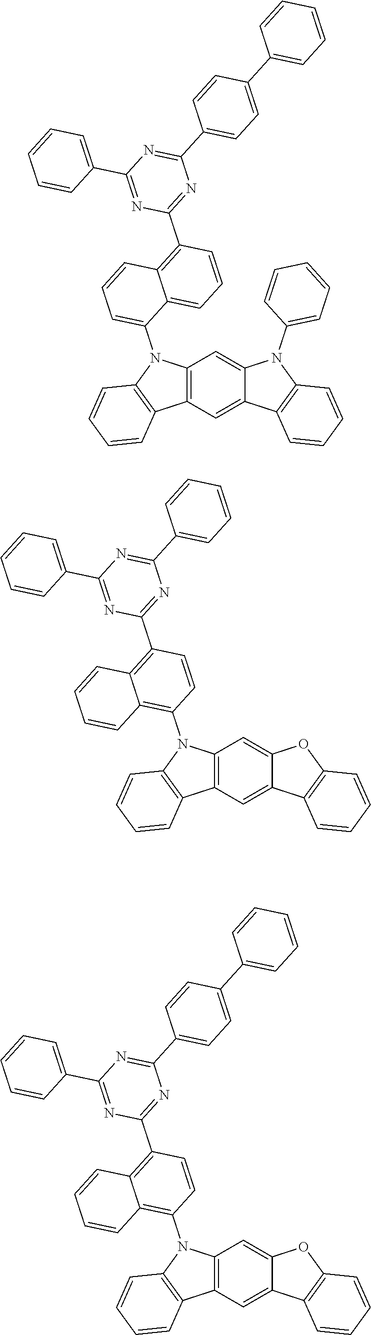 [SMILES: c1ccc(-c2ccc(-c3nc(-c4ccccc4)nc(-c4cccc5c(-n6c7ccccc7c7cc8c9ccccc9n(-c9ccccc9)c8cc76)cccc45)n3)cc2)cc1 ; c1ccc(-c2nc(-c3ccccc3)nc(-c3ccc(-n4c5ccccc5c5cc6c(cc54)oc4ccccc46)c4ccccc34)n2)cc1 ; c1ccc(-c2ccc(-c3nc(-c4ccccc4)nc(-c4ccc(-n5c6ccccc6c6cc7c(cc65)oc5ccccc57)c5ccccc45)n3)cc2)cc1] 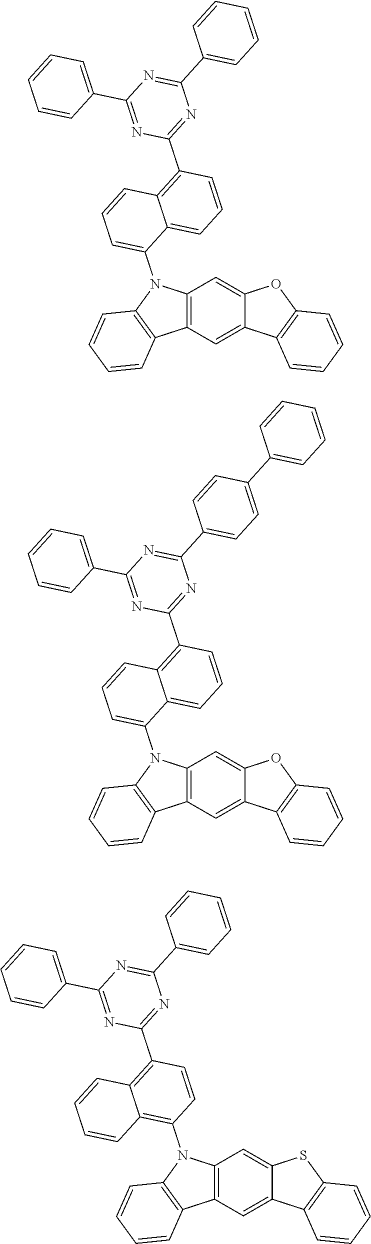 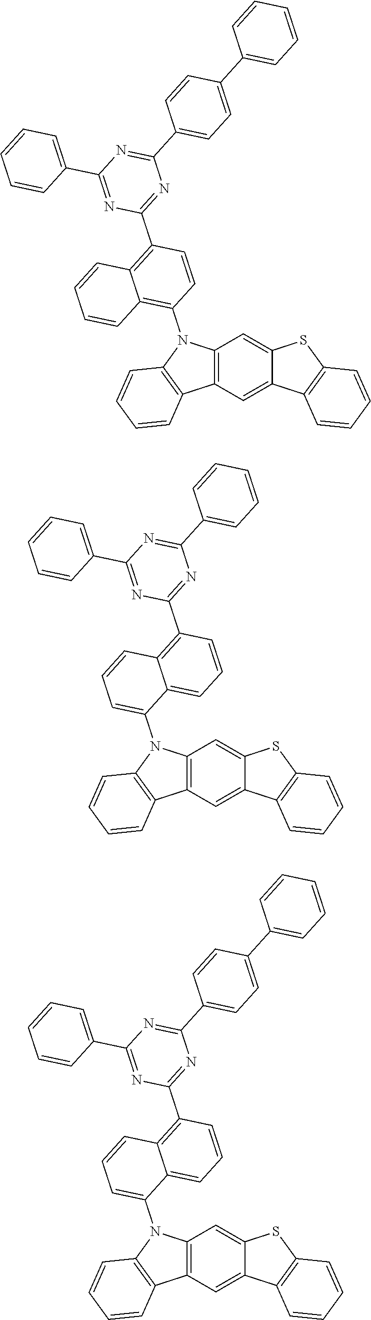 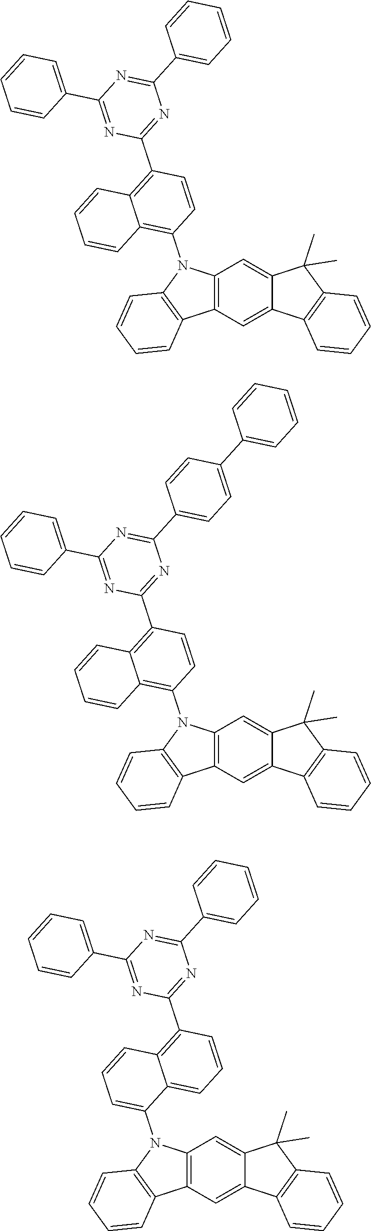 [SMILES: CC1(C)c2ccccc2-c2cc3c4ccccc4n(-c4ccc(-c5nc(-c6ccccc6)nc(-c6ccccc6)n5)c5ccccc45)c3cc21 ; CC1(C)c2ccccc2-c2cc3c4ccccc4n(-c4ccc(-c5nc(-c6ccccc6)nc(-c6ccc(-c7ccccc7)cc6)n5)c5ccccc45)c3cc21 ; CC1(C)c2ccccc2-c2cc3c4ccccc4n(-c4cccc5c(-c6nc(-c7ccccc7)nc(-c7ccccc7)n6)cccc45)c3cc21] 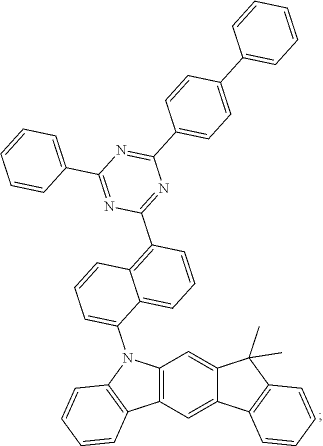 [SMILES: CC1(C)c2ccccc2-c2cc3c4ccccc4n(-c4cccc5c(-c6nc(-c7ccccc7)nc(-c7ccc(-c8ccccc8)cc7)n6)cccc45)c3cc21]
wherein hydrogen atoms in the above compounds can be substituted with deuterium atoms.

11. A compound, comprising the following compounds: 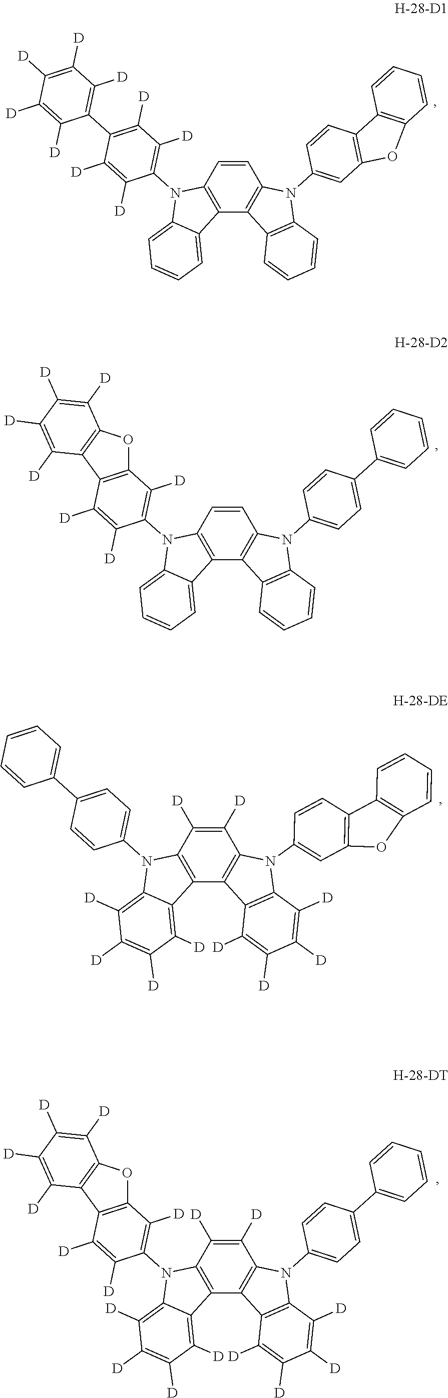 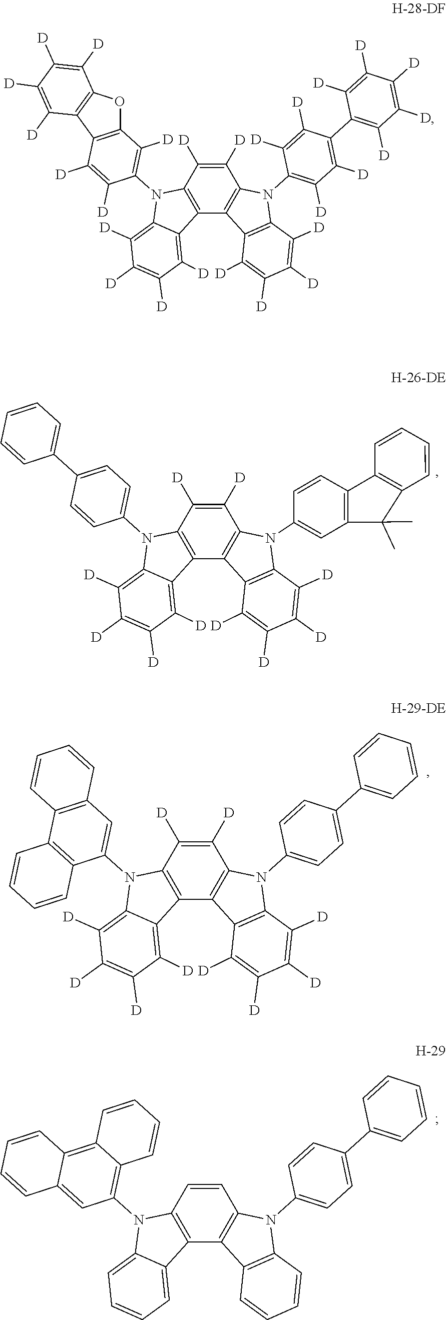
wherein the compound is used for preparing the composition according to claim 1.

12. An intermediate, comprising the following compounds: 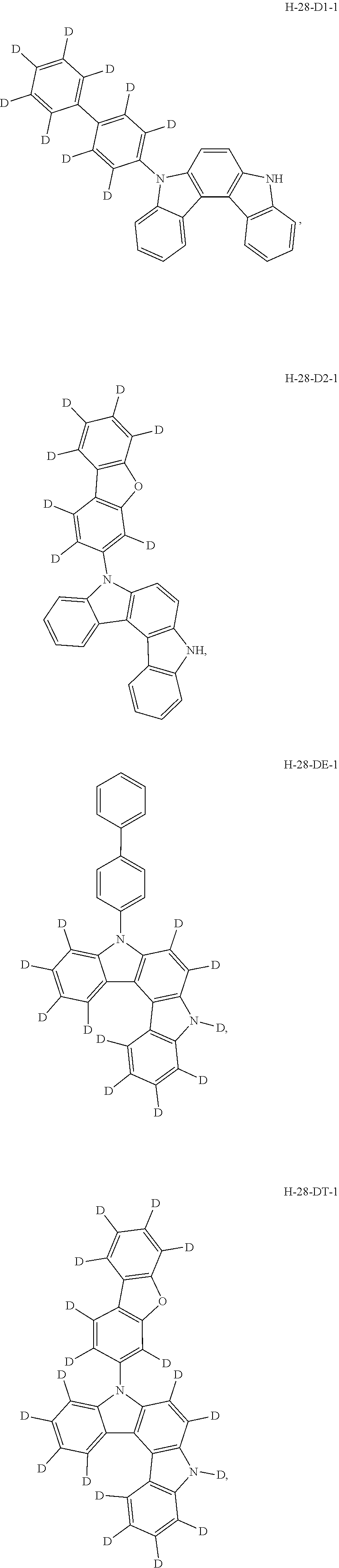 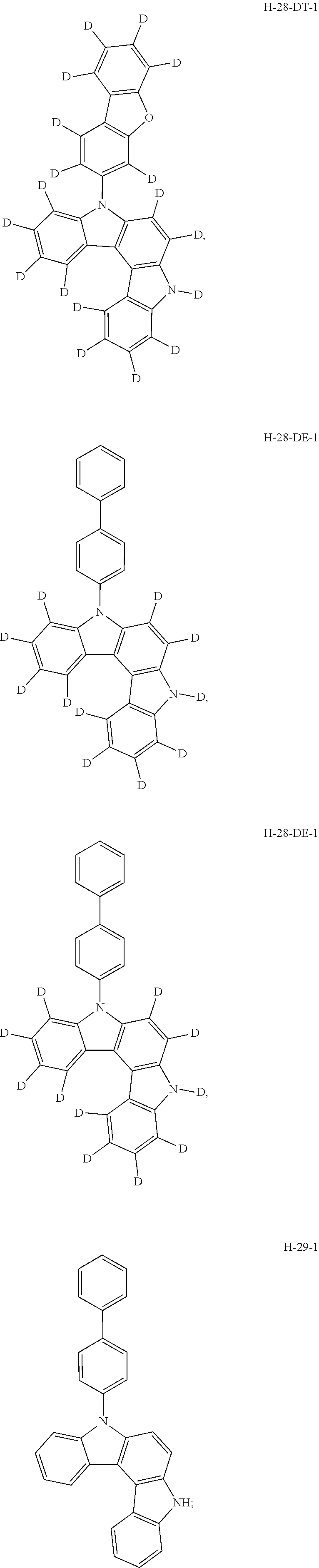
wherein the intermediate is used for preparing the compound represented by Formula I in the composition according to claim 1.

13. An organic electroluminescent device, comprising an anode, a cathode, and an organic thin film layer arranged between the anode and the cathode;
wherein a material of the organic thin film layer comprises the composition according to claim 1;
optionally, the organic thin film layer comprises an emissive layer, and a material of the emissive layer comprises the composition according to claim 1;
optionally, the organic thin film layer comprises a hole layer;
the hole layer comprises an electron blocking layer; and a material of the electron blocking layer includes a spirofluorene compound;
the spirofluorene compound has a specific structure as represented by Formula III below: 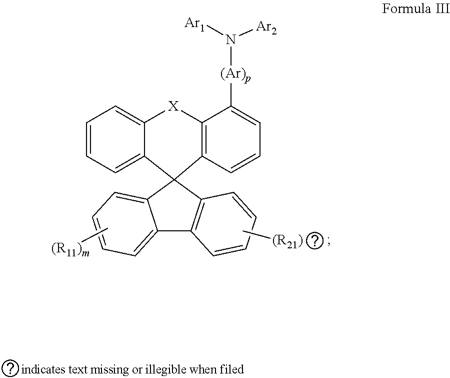
wherein X is selected from O or S;
R₁₁ and R₂₁ are each independently selected from hydrogen, deuterium, fluorine, CN, substituted or unsubstituted C1 to C20 linear or branched alkyl, substituted or unsubstituted C1 to C20 alkoxy, or substituted or unsubstituted C6 to C40 aryl;
Ar is selected from substituted or unsubstituted C6 to C40 arylidene;
Ar₁ and Ar₂ are each independently selected from substituted or unsubstituted C6 to C40 aryl, substituted or unsubstituted C12 to C40 oxa-aryl, substituted or unsubstituted C12 to C40 thioaryl, and at least one of Ar₁ or Ar₂ is selected from any one of phenyl, naphthyl, triphenylenyl, or fluoranthenyl;
p is selected from 0 or 1; and
m and n are each independently selected from an integer from 0 to 4.

14. A display apparatus, comprising the organic electroluminescent device according to claim 13.
